(19) United States
(12) Patent Application Publication
(10) Pub. No.: US 2026/0168486 A1
(43) Pub. Date: Jun. 18, 2026

(54) EXTERNALLY POSITIONED FLUID LEVEL DETECTION AND SUMP PUMP CONTROL SYSTEM

(71) Applicant: BARREL PROOF TECHNOLOGIES LLC, Murfreesboro, TN (US)

(72) Inventor: Brian Richard Anderson, Murfreesboro, TN (US)

(21) Appl. No.: 19/532,912

(22) Filed: Feb. 6, 2026

Related U.S. Application Data
(63) Continuation-in-part of application No. 19/241,167, filed on Jun. 17, 2025.
Application No. 19/241,167 is a continuation-in-part of application No. 19/235,377, filed on Jun. 11, 2025.
Application No. 19/235,377 is a continuation-in-part of application No. 19/182,441, filed on Apr. 17, 2025.
Application No. 19/182,441 is a continuation-in-part of application No. 19/084,671, filed on Mar. 19, 2025, now Pat. No. 12,609,185.
Application No. 19/182,441 is a continuation-in-part of application No. 19/080,723, filed on Mar. 14, 2025, now Pat. No. 12,422,295.
Application No. 19/084,671 is a continuation-in-part of application No. 19/013,859, filed on Jan. 8, 2025.
Application No. 19/080,723 is a continuation-in-part of application No. 19/013,859, filed on Jan. 8, 2025.
Application No. 19/013,859 is a continuation-in-part of application No. 18/818,539, filed on Aug. 28, 2024, now Pat. No. 12,228,525.
Application No. 18/818,539 is a continuation-in-part of application No. 18/800,279, filed on Aug. 12, 2024, now Pat. No. 12,228,445.
Application No. 18/818,539 is a continuation-in-part of application No. 18/424,758, filed on Jan. 27, 2024, now Pat. No. 12,117,329.
Application No. 18/800,279 is a continuation-in-part of application No. 18/424,758, filed on Jan. 27, 2024, now Pat. No. 12,117,329.

(51) Int. Cl. F04B 49/025; F04B 49/06

(52) U.S. Cl. CPC F04B 49/025; F04B 49/065

(57) ABSTRACT

Disclosed herein is a method for controlling a pump of a sump system. The method includes transmitting, into a container of the sump system, at least one signal from one or more sensor nodes positioned external to the container. The method further includes receiving a response associated with the transmitted at least one signal. The method further includes detecting a change of at least one characteristic between the at least one transmitted signal and a corresponding received response. The method further includes determining a fluid level factor based on the change of the at least one characteristic. The fluid level factor includes one or more of the fluid level of the container and a rate of change of the fluid level of the container. The method further includes causing the pump of the sump system to activate or deactivate based on the fluid level factor.

### CROSS-REFERENCE TO RELATED APPLICATIONS

This application is a Continuation-in-part of, and claims the benefit of U.S. Application Number 19/241,167, titled “NON-INVASIVE CONTAINER MONITORING USING A PASSIVE CERTIFICATION ELEMENT COUPLED TO A REMOVABLE SENSOR DEVICE” filed on Jun. 17, 2025, which is a Continuation-in-part of, and claims the benefit of U.S. application Ser. No. 19/235,377, titled “CONTAINER MONITORING SYSTEM WITH DIELECTRIC-BASED CONTAMINATION DETECTION” filed on Jun. 11, 2025, which is a Continuation-in-part of, and claims the benefit of U.S. application Ser. No. 19/182,441, titled “SYSTEM AND METHOD FOR DETERMINING CONTENT UTILIZING EXTERNALLY MOUNTED CONTAINER MONITORING SYSTEM” filed on Apr. 17, 2025, which is a Continuation-in-part of, and claims the benefit of U.S. application Ser. No. 19/080,723, titled “SYSTEM AND METHOD FOR DETERMINING CONTENT UTILIZING EXTERNALLY MOUNTED CONTAINER MONITORING SYSTEM” filed on Mar. 14, 2025, now issued U.S. Pat. No. 12,422,295, which is a Continuation-in-part of, and claims the benefit of U.S. application Ser. No. 19/013,859, titled “SYSTEM AND METHOD FOR DETERMINING FLUID LEVEL AND/OR ALCOHOL CONTENT UTILIZING EXTERNALLY MOUNTED CONTAINER MONITORING SYSTEM” filed on Jan. 8, 2025, which is a Continuation-in part of, and claims of the benefit of U.S. application Ser. No. 18/818,539, titled “SYSTEM AND METHOD FOR DETERMINING ALCOHOL CONTENT UTILIZING CONTAINER MONITORING SYSTEM,” filed on Aug. 28, 2024, now issued U.S. Pat. No. 12,228,445, which is a Continuation-in-part of, and claims the benefit and earlier filing date of U.S. application Ser. No. 18/424,758, titled “CONTAINER MONITORING SYSTEM AND METHOD THEREOF,” filed on Jan. 27, 2024, now issued U.S. Pat. No. 12,117,329. U.S. application Ser. No. 19/013,859 is also a continuation in part of, and claims the benefit of U.S. application Ser. No. 18/800,279, titled “SYSTEM AND METHOD FOR DETERMINING ALCOHOL CONTENT WITHIN CONTAINER UTILIZING CONTAINER MONITORING SYSTEM,” filed on Aug. 12, 2024, now issued U.S. Pat. No. 12,228,445, which is a Continuation-in-part of, and claims the benefit and earlier filing date of U.S. application Ser. No. 18/424,758, titled “CONTAINER MONITORING SYSTEM AND METHOD THEREOF,” filed on Jan. 27, 2024, now issued U.S. Pat. No. 12,117,329. This application incorporates by reference, herein, the entire contents of the above referred-to patent applications.

U.S. application Ser. No. 19/235,377 is also a Continuation-in-part of, and claims the benefit of U.S. application Ser. No. 19/084,671, titled “ARTIFICIAL INTELLIGENCE DRIVEN MONITORING SYSTEM FOR AGING WHISKEY” filed on Mar. 19, 2025, which is a Continuation-in-part of, and claims the benefit of U.S. application Ser. No. 19/013,859, titled “SYSTEM AND METHOD FOR DETERMINING FLUID LEVEL AND/OR ALCOHOL CONTENT UTILIZING EXTERNALLY MOUNTED CONTAINER MONITORING SYSTEM” filed on Jan. 8, 2025, which is a Continuation-in part of, and claims of the benefit of U.S. application Ser. No. 18/818,539, titled “SYSTEM AND METHOD FOR DETERMINING ALCOHOL CONTENT UTILIZING CONTAINER MONITORING SYSTEM,” filed on Aug. 28, 2024, now issued U.S. Pat. No. 12,228,445, which is a Continuation-in-part of, and claims the benefit and earlier filing date of U.S. application Ser. No. 18/424,758, titled “CONTAINER MONITORING SYSTEM AND METHOD THEREOF,” filed on Jan. 27, 2024, now issued U.S. Pat. No. 12,117,329. U.S. application Ser. No. 19/013,859 is also a continuation in part of, and claims the benefit of U.S. application Ser. No. 18/800,279, titled “SYSTEM AND METHOD FOR DETERMINING ALCOHOL CONTENT WITHIN CONTAINER UTILIZING CONTAINER MONITORING SYSTEM,” filed on Aug. 12, 2024, now issued U.S. Pat. No. 12,228,445, which is a Continuation-in-part of, and claims the benefit and earlier filing date of U.S. application Ser. No. 18/424,758, titled “CONTAINER MONITORING SYSTEM AND METHOD THEREOF,” filed on Jan. 27, 2024, now issued U.S. Pat. No. 12,117,329. This application incorporates by reference, herein, the entire contents of the above referred-to patent applications.

### TECHNICAL FIELD

This disclosure relates generally to the field of fluid management and measurement of liquid content within containers and alcohol determination based on the fluid content.

### BACKGROUND

Containers, such as barrels, have been used for centuries for the containment and processing of fermenting liquids. Whether the enclosed liquid is wine, beer or spirits, the wooden containers (or barrels) represent an industrial standard for the aging and fermentation of the contained liquid. In many cases, the fermenting liquid may be retained within the same wooden barrel for many years, wherein the increase in length of time (i.e., in storage) impairs different favor, quality and cost to the contained liquid. For example, spirits are measured by the duration of their aging process, wherein the longer the contained product is aged, the more expensive the value of the product becomes. For example, a 200-year-old Napoleon brandy is significantly more expensive than a 2-year-old brandy by the same manufacturer, as the brandy has been fermenting in the barrel for a significantly longer period of time.

However, issues regarding the use of wooden barrels are well-known in the art. For example, fermenting liquid within a barrel is prone to two types of losses. The first being evaporation of the liquid within the barrel and the second being absorption by the wooden elements comprising the barrels.

In many cases, the barrels, once filled, are retained within a known position, whether vertical or horizontal, for the duration of their intended aging process. During this time, inspection of the contained liquid (quality, level and alcohol content (or measurement)) may occur by the insertion of one or more types of measurement tools into the barrel.

However, insertion of the measurement tool may introduce air or other contaminants that may alter the quality of the contained liquid. In addition, the repeated insertion of the measurement tools increases the amount of labor required to monitor the critical aspects of the fermentation process (i.e., alcohol production).

Furthermore, the measurement of fluid loss within a barrel or container is an important factor in the whiskey industry as distillers are required to report to Tax and Trade Bureaus container fill volume.

In addition, alcohol content (or Proof) is extremely important to know as distillers are required to follow stringent rules for the classification of different spirits.

For example, to be classified as a Bourbon whiskey the liquid at bottling must have a minimum alcohol content of 40 percent by volume (ABV), Generally, typically bottled Bourbon is between 40 and 60 percent ABV, whereas the liquid entered into the barrel for aging should have an ABV of no greater 62.5 percent ABV. For a general Whiskey, the liquid at bottling must have a minimum alcohol content of 40 percent ABV.

As mentioned above, the conventional methods for determining fluid level and alcohol content is labor intensive as it requires the sampling of the aging fluid by drawing a sample from the container (i.e., opening the barrel, which may introduce air into the container), measuring the sample's temperature, using a hydrometer or alcoholmeter to measure alcohol content, and adjusting the reading based on temperature. Alternatively, more modern analytical methods, such as gas chromatography or near-infrared spectroscopy, may be utilized to determine alcohol content. However, while these methods may provide highly accurate reading, they are more expensive and require specialized equipment.

In still another aspect, wherein container fill level is required to prevent exceeding capacity and also prevent overflow and damage, the conventional methods of such determination is both time-consuming and invasive.

Hence, there is a need in the industry for a non-intrusive method and system for obtaining measurements of the level of the contents of a container in order to determine at least one of a level of fluid and a content, wherein invasive probes or manual inspection or, even guesswork is removed.

Beyond the specific challenges in barrel-based aging and fermentation, the broader industrial landscape faces similar monitoring challenges across diverse liquid storage and distribution systems. In military applications, fuel integrity at Forward Arming and Refueling Points (FARPs) is critical for mission success, yet traditional sampling methods create operational vulnerabilities and potential contamination risks. The pharmaceutical industry requires continuous verification of liquid formulations to prevent counterfeiting and ensure patient safety, but invasive testing can compromise sterile environments. Municipal water systems need real-time contamination detection capabilities to protect public health, yet the distributed nature of water infrastructure makes manual sampling impractical. Similarly, the oil and gas industry must monitor pipeline integrity and detect water ingress or paraffin buildup to prevent costly failures. Across these diverse applications, existing monitoring approaches typically rely on periodic manual sampling, which creates temporal gaps in oversight where contamination, tampering, or degradation can occur undetected. Furthermore, the lack of continuous data collection prevents the development of predictive models that could enable proactive maintenance and intervention. The increasing regulatory requirements for audit trails and compliance documentation further compound these challenges, as manual methods struggle to provide the comprehensive, tamper-evident records demanded by modern quality assurance standards.

### SUMMARY

Herein disclosed is a system for determining a fill level of at least one of a content within a container wherein the fill level of the at least one content is determined based on the reception of a time of return of a transmitted signals wherein the transmitted signals are frequency varying with the transmission window. The frequency variation may be continuous or patterned. The frequency variation may be continuous or patterned. The device herein disclosed is applicable to a wide spectrum of configurations to determine a content, whether liquid, mash or solid within the container wherein the container may be associated with a barrel or container associated with alcohol production, or containers involved in the retention of other types of materials or content, such as but not limited to, septic tanks, waste management systems, water towers, etc.

In accordance with further aspects of the invention, the disclosed monitoring system extends beyond traditional fluid level measurement to encompass comprehensive integrity assessment across diverse industrial applications. The system employs dielectric fingerprinting technology, wherein each liquid substrate exhibits a unique electromagnetic signature based on its molecular composition. By continuously monitoring these dielectric properties through non-invasive electromagnetic interrogation, the system detects deviations indicative of contamination, tampering, degradation, dilution, substitution, or hazardous events. This approach transforms passive storage containers into intelligent monitoring assets that can provide real-time alerts when liquid integrity is compromised.

The system architecture comprises modular sensor nodes that can be externally mounted to various container types without penetration or modification. These sensor nodes may incorporate dielectric sensors and radar antennas that measure the relative permittivity of contained liquids and may be capable of creating a continuous audit trail of liquid integrity. The measured dielectric signatures may be processed through industry-specific machine-learning models that distinguish between normal variations and genuine integrity threats. For military fuel monitoring applications, the models may identify water contamination signatures or other adulterant signatures. For pharmaceutical applications, they detect counterfeit formulations. For spirits monitoring, they recognize patterns of dilution or unauthorized access. A software-defined approach enables the same hardware platform to serve diverse industries through model adaptation rather than equipment modification.

The distributed monitoring platform integrates edge computing capabilities with cloud-based analytics to accommodate various deployment scenarios. In bandwidth-constrained or security-sensitive environments, sensor nodes perform local inference using compressed machine learning models, providing autonomous operation without continuous connectivity. When network access is available, the system synchronizes detected anomalies and refined baselines with centralized infrastructure, enabling fleet-wide visibility and continuous model improvement. In some aspects, the platform generates multi-tiered alerts ranging from informational notifications to critical interventions, with each alert including forensic metadata suitable for regulatory compliance and audit requirements.

### BRIEF DESCRIPTION OF THE DRAWINGS

The advantages, nature, and various additional features of the invention will appear more fully upon consideration of the illustrative embodiments described in detail in connection with the accompanying drawings, where like or similar reference numerals are used to identify like or similar elements throughout the drawings.

FIG. 1 illustrates a first conventional configuration for storing a plurality of barrels and the liquid contained therein.

FIG. 2 illustrates a first exemplary embodiment of a system for determining liquid content within a barrel in accordance with the principles of the invention.

FIG. 3 illustrates a block diagram of an exemplary embodiment of a processing system for determining liquid content within a barrel in accordance with the principles of the invention.

FIG. 4 illustrates a block diagram of an exemplary system for determining liquid content within a barrel in accordance with the principles of the invention.

FIG. 5A illustrates a flowchart of an exemplary processing in accordance with the principles of the invention.

FIG. 5B illustrates an exemplary timing chart in accordance with the principles of the invention.

FIG. 6 illustrates a graph of an exemplary signal return chart for determined liquid content within a barrel in accordance with the principles of the invention.

FIGS. 7A and 7B illustrate a first and second aspect of a second exemplary embodiment of a system for determining liquid content within a barrel in accordance with the principles of the invention.

FIG. 8 illustrates a graph of an exemplary signal return chart associated with the configurations shown in FIGS. 7A and 7B for determined liquid content within a barrel in accordance with the principles of the invention.

FIG. 9 illustrates a second conventional configuration for storing a plurality of barrels and the liquid contained therein.

FIG. 10 illustrates a flowchart of an exemplary processing for determining liquid content within a barrel in accordance with the principles of the invention.

FIGS. 11A-11C illustrate exemplary signal transmission and signal return graphs as a function of time in accordance with one aspect of the invention.

FIG. 12 illustrates an exemplary processing associated with the graphs shown in FIGS. 11A and 11B.

FIG. 13 illustrates a flowchart of an exemplary process associated with a determination of an alcohol content within a container in accordance with the principles of the invention.

FIG. 14 illustrates a flowchart of an exemplary process for determining and extrapolating alcohol content of a liquid within a container in accordance with the principles of the invention.

FIG. 15 illustrates a flowchart of an exemplary process for determining alcohol content of a liquid within a container in accordance with the principles of the invention.

FIG. 16 illustrates a flowchart of an exemplary process for adjusting a determined alcohol content based on environmental considerations.

FIG. 17 illustrates an exemplary plotting and extrapolating alcohol content of a liquid within a container in accordance with the principles of the invention.

FIG. 18 illustrates an exemplary graph of alcohol content determination in accordance with the principles of the invention.

FIG. 19 illustrates a flowchart of a second exemplary process associated with a determination of an alcohol content within a container in accordance with the principles of the invention.

FIGS. 20A-D illustrate exemplary charts of alcohol content as a function of change in frequency.

FIG. 21 illustrates an exemplary chart of measurement of alcohol content as a function of time.

FIG. 22 illustrates an exemplary example of a transmission sequence in accordance with the principles of the invention.

FIG. 23 illustrates an exemplary chart of the collection of one or more materials within a container monitored by the external monitoring system disclosed herein.

FIG. 24 illustrates an example configuration of a liquid-containing vessel having a plurality of externally mounted RF-responsive elements, and a schematic representation of a system for analyzing reflected signal characteristics as influenced by the surrounding environment in accordance with aspects of the present disclosure.

FIG. 25 illustrates a block diagram of an exemplary architecture of a RF sensing device that may be implemented in various embodiments of a non-invasive sensing system for monitoring internal conditions of containers in accordance with aspects of the present disclosure.

FIG. 26 illustrates signal response profile diagrams showing various radio frequency signal characteristics as functions of liquid level relative to an RF-responsive element in accordance with aspects of the present disclosure.

FIG. 27 illustrates a composite time-domain chart showing signal behavior and environmental change over time for a container monitoring system, including RSSI values for multiple RF-responsive elements and corresponding liquid level measurements in accordance with aspects of the present disclosure.

FIG. 28 illustrates a composite phase-domain chart showing signal behavior and environmental change over time for a container monitoring system, including phase response values for multiple RF-responsive elements and corresponding liquid level measurements in accordance with aspects of the present disclosure.

FIG. 29 illustrates frequency-domain plots showing how reflected radio frequency signal characteristics vary as a function of alcohol content within a liquid adjacent to an externally affixed RF-responsive element in accordance with aspects of the present disclosure.

FIG. 30 illustrates frequency-domain plots showing how reflected radio frequency signal characteristics vary as a function of moisture or seepage conditions detected by an externally affixed RF-responsive element in accordance with aspects of the present disclosure.

FIG. 31 illustrates a set of frequency-domain signal plots showing radio frequency signal characteristics as affected by both char level of a container's interior wall and the color/maturity of a contained liquid in accordance with aspects of the present disclosure.

FIG. 32A illustrates an AI-driven RF sensing system configured for analyzing environmental signal characteristics associated with a liquid-filled container in accordance with aspects of the present disclosure.

FIG. 32B illustrates another example of an RF-based sensing architecture for analyzing contents within a container, wherein a transmitted signal is directed toward the container and reflected back in accordance with aspects of the present disclosure.

FIG. 33 illustrates a training system for generating a machine-learning model capable of inferring internal conditions of a container using radio-frequency signal features in accordance with aspects of the present disclosure.

FIG. 34 illustrates an artificial neural network for processing radio-frequency-derived input data and generating predictions relating to characteristics of contents within a sealed container in accordance with aspects of the present disclosure.

FIG. 35 illustrates a distributed monitoring and analysis system for determining one or more characteristics of a liquid contained within a container in accordance with aspects of the present disclosure.

FIG. 36 illustrates a process for determining one or more characteristics of a liquid or other contents stored within a container in accordance with aspects of the present disclosure.

FIG. 37 illustrates an exemplary process for determining at least one characteristic of contents within a container using a machine-learning model in accordance with aspects of the present disclosure.

FIG. 38 illustrates an exemplary aspect of a barrel monitoring system deployed in a warehouse environment in accordance with aspects of the present disclosure.

FIG. 39 illustrates an exemplary rickhouse or barrel-aging facility configured for non-invasive wireless monitoring of a plurality of containers in accordance with aspects of the present disclosure.

FIG. 40 illustrates two examples of RF-responsive elements that may be affixed to the exterior surface of a container for non-invasively monitoring internal contents in accordance with aspects of the present disclosure.

FIG. 41 illustrates an example of an RF interrogator in accordance with aspects of the present disclosure.

FIG. 42 illustrates an exemplary embodiment of a wireless communication node, such as a 5G base station (gNodeB), configured to perform both conventional wireless communication and RF-based environmental sensing through a sensor interrogation module in accordance with aspects of the present disclosure.

FIG. 43 illustrates an example implementation of an integrity monitoring platform and other substrates contained within a sealed container in accordance with aspects of the present disclosure.

FIG. 44A illustrates an exploded view of a universal sensor node assembly that forms part of the integrity monitoring platform in accordance with aspects of the present disclosure.

FIG. 44B illustrates a modular sensor node assembly for the integrity monitoring platform depicting the plug-and-play architecture that enables field-configurable deployment across diverse liquid monitoring applications in accordance with aspects of the present disclosure.

FIG. 45 illustrates a communication architecture for secure data transmission utilized by the integrity monitoring platform in accordance with at least one aspect of the present disclosure.

FIG. 46 illustrates an exemplary embodiment of a cloud platform configured to process sensor data and generate integrity metrics for monitored liquids in accordance with at least some aspects of the present disclosure.

FIG. 47 illustrates a model-training pipeline for developing and maintaining machine learning models utilized by the integrity monitoring platform.

FIG. 48 illustrates a results processing and user interface architecture for transforming analytical outputs into actionable insights within an integrity monitoring platform in accordance with at least one aspect of the present disclosure.

FIG. 49 illustrates an exemplary time-series anomaly detection output generated by the integrity monitoring platform in accordance with at least one aspect of the present disclosure.

FIG. 50 illustrates an edge-only inference implementation of the integrity monitoring platform wherein analytical processing occurs locally within the sensor node rather than relying on the cloud layer in accordance with at least one aspect of the present disclosure.

FIG. 51 illustrates a functional block diagram of an exemplary workflow for detecting adulteration and analyzing compositional anomalies in monitored substrates based on deviations in dielectric fingerprints in accordance with at least one aspect of the present disclosure.

FIG. 52 depicts an exemplary deployment schematic for the integrity monitoring platform configured for real-time substrate integrity assessment across the distributed infrastructure in accordance with at least one aspect of the present disclosure.

FIG. 53 illustrates an exemplary machine learning model training pipeline that enables the integrity monitoring platform to develop industry-specific analytical models within the analytics layer in accordance with at least some aspects of the present disclosure.

FIG. 54 depicts an example method for monitoring liquid integrity through non-invasive dielectric property measurement in accordance with at least one aspect of the present disclosure.

FIG. 55A illustrates an exemplary aspect of a sump monitoring system equipped with a submersible pump in accordance with at least one aspect of the present disclosure.

FIG. 55B illustrates an exemplary aspect of a sump monitoring system equipped with a pedestal pump in accordance with at least one aspect of the present disclosure.

FIG. 55C illustrates an exemplary networked sump monitoring system architecture in accordance with at least one aspect of the present disclosure.

FIG. 56 depicts an example method for controlling a pump of a sump system in accordance with at least one aspect of the present disclosure.

It is to be understood that the figures, which are not drawn to scale, and descriptions of the present invention described herein have been simplified to illustrate the elements that are relevant for a clear understanding of the present invention, while eliminating, for purposes of clarity, many other elements. However, because these omitted elements are well-known in the art, and because they do not facilitate a better understanding of the present invention, a discussion of such elements are not provided herein. The disclosure, herein, is directed also to variations and modifications known to those skilled in the art.

### DETAILED DESCRIPTION

Note that the specific embodiments given in the drawings and following description do not limit the disclosure. On the contrary, they provide the foundation for one of ordinary skill to discern the alternative forms, equivalents, and modifications that are contemplated by the inventors and encompassed in the claim scope.

Numerous alternative forms, equivalents, and modifications will become apparent to those skilled in the art once the above disclosure is fully appreciated. It is intended that the claims be interpreted to embrace all such alternative forms, equivalents, and modifications where applicable.

Disclosed herein are an apparatus and associated method implementations related to determining a liquid level within a barrel based on a system, located external to the barrel, configured to transmit a signal into the barrel and processing signals, reflected by the contained liquid, wherein the characteristics of the reflected signal (e.g. distance and time traveled) may be used to determine the presence of the liquid; determining a level of fluid within the barrel as a function of at least one of the distance and time traveled by the transmitted/reflected signal, determining a fluid level within the barrel and determining, as a function of at least the determined level of the fluid within the barrel and the physical dimensions of the barrel, the volume of fluid within the barrel.

Disclosed herein are an apparatus and associated method implementations located external to the barrel for determining an alcohol content within a barrel based on a system configured to transmit a signal (i.e., a measurement signal) into the barrel and processing signals, reflected by the contained liquid, wherein the characteristics of the reflected signal (e.g. signal strength, frequency, phase, distance and/or time traveled) may be used to determine the presence of the liquid and the alcohol content of the liquid, wherein determination of a level of fluid within the barrel may be used to determine which of a plurality of signals are transmitted into the barrel.

In one aspect of the invention, the system disclosed may comprise a modular device consisting of a motherboard, a specialized breakout board (chips), a data transmission module, a power source and at least one transmit and/or receiving antenna. The system may be attached to the face of an enclosed container (e.g., a whiskey barrel, wine barrel, beer barrel) with an antenna array that is suitable for transmitting signals in at least one of a Millimeter Wave (MM Wave) range, or a radio frequency range (i.e., Institute of Electronic and Electrical Engineers (IEEE) designated bands HF through W, and other wavelength ranges). In one aspect of the invention, the system and method disclosed any utilize a millimeter wave transmission system in a wavelength band of 57-64 GHz. In another aspect of the invention, a transmission system may operate in one or more of an ISM (Industrial, scientific, and medical) wavelength band that would avoid interference with other types of electronic equipment.

In one aspect of the invention, each of the at least one antenna may be configured to emit or transmit a signal at a same known wavelength within one or more of the referred to wavelength bands. In one aspect of the invention, each of the at least one antenna may be configured to transmit a signal at a different known frequency (or wavelength) within one or more of the referred to wavelength bands. In one aspect of the invention, each of the at least one antenna may be configured to transmit at least one signal at one or more frequencies within one or more different known frequency or wavelength bands.

In one aspect of the invention, one or more characteristics (e.g., signal strength, frequency, phase, distance and/or time traveled) of the signals reflected by the contained fluid or liquid, may be used to determine a level of the contained fluid based at least on a position of one or more of the antennas receiving the reflected signals and subsequently the alcohol content of the liquid within the barrel.

In one aspect of the invention, the signal strength of the signals reflected by the contained fluid or liquid, may be used to determine the level of the contained fluid based at least on a position of one or more of the antennas receiving the reflected signals.

In one aspect of the invention, measurements regarding the signal strength and determined fluid level (and volume) may be relayed to a communications hub via one or more transmissions protocols and exported wirelessly (cellular, Wi-Fi) or over a wired Internet connection to a common database wherein reports may be derived. In another aspect of the invention, measurements regarding signal strength and determined fluid level (and/or volume) may be relayed by a near-field communication transmission (e.g., RFID, BLUETOOTH, etc.) that enable periodic monitoring of the determined fluid level and/or volume.

In one aspect of the invention, consultative data analysis reports may be created to assist a manufacturer/consumer with making actionable business decisions based upon results.

In accordance with the principles of the invention, the system and method disclosed may utilize a Millimeter wave transmission system (30 GHz-300 GHz) and an appropriately scaled (frequency selective) antennas to determine a level of the liquid inside of an enclosed container (e.g., a whiskey barrel).

In one aspect of the invention, by measuring the liquid level over time, a manufacturer/consumer may determine fluid internal volume at any given period. In accordance with the principles of the invention, while barrel technology is referred to, it would be understood by those skilled in the art that the system and method disclosed may be utilized to determine the fluid level in any enclosed system used containing liquid.

In one aspect of the invention, a method is disclosed for determination of an alcohol content of a liquid within an enclosed barrel wherein the alcohol content is based on an initial alcohol content and one or more environmental factors, such as location, temperature, environment conditions, etc.

In one aspect of the invention, a method is disclosed for the determination of an alcohol content of a contained fluid based on a determination of evaporation and/or absorption of the fluid and an extrapolation from a known initial level.

In one aspect of the invention, a method is disclosed wherein a determination of a loss of fluid within an enclosed container is utilized to determine an alcohol content of the fluid considering one or more environmental factors.

In accordance with the principles of the invention, while barrel technology is referred to, it would be understood by those skilled in the art that the system and method disclosed may be utilized to determine the fluid level in any enclosed system containing liquid.

In one aspect of the invention, a method is disclosed for the determination of an alcohol content of a contained fluid based on an evaluation of at least one variation in at least one characteristic (e.g., signal strength change, frequency shift, phase shift, change in distance and/or time traveled, etc.) of at least one reflection of a signal transmitted in at least one frequency or wavelength band.

Disclosed herein are an apparatus and associated method implementations related to determining a liquid level within a barrel based on a system, located external to the barrel, configured to transmit a signal into the barrel and processing signals reflected by the contained liquid. The characteristics of the reflected signal (e.g., distance and time traveled) may be used to determine the presence of the liquid, determining a level of fluid within the barrel as a function of at least one of the distance and time traveled by the transmitted/reflected signal, determining a fluid level within the barrel and determining, as a function of at least the determined level of the fluid within the barrel and the physical dimensions of the barrel, the volume of fluid within the barrel.

Disclosed herein are an apparatus and associated method implementations related to integrating RF-based environmental sensing capabilities within wireless communication infrastructure. The system is configured to transmit signals toward RF-responsive elements affixed to containers and analyze reflected or backscattered signals to determine characteristics of contained liquids or materials. The characteristics of the reflected signal (e.g., signal strength, frequency, phase, distance and/or time traveled) may be used to determine both the presence of the liquid and additional parameters such as alcohol content, with measurements potentially performed using existing cellular infrastructure to reduce deployment costs.

In one aspect of the invention, the system disclosed may comprise a wireless communication node that integrates conventional wireless communication functionality with specialized sensor interrogation capabilities. The system may include an antenna array and RF front end coupled to at least one antenna, a sensor interrogation module for environmental sensing, an RF transceiver for wireless communication, and a backhaul interface for transmitting collected data to external systems. The system may utilize beamforming capabilities to direct RF signals toward containers or RF-responsive elements positioned on their exterior surfaces, enabling non-invasive monitoring of internal contents while maintaining standard communication services.

In one aspect of the invention, the wireless communication node may be configured to transmit signals across various frequency bands, potentially including sub-6 GHz bands common in cellular deployments as well as millimeter wave ranges (30 GHz-300 GHz). The node may implement multiple frequency band operation to optimize both communication performance and sensing accuracy, with specific frequencies selected based on the materials being monitored and the desired sensing metrics. In some implementations, the node may coordinate resource allocation between communication and sensing functions through time-division or frequency-division multiplexing approaches.

In one aspect of the invention, one or more signal characteristics (e.g., signal strength, frequency, phase, distance and/or time traveled) of the signals reflected by RF-responsive elements on containers may be analyzed by the sensor interrogation module within the wireless communication node. This analysis may determine fluid levels, alcohol content, or other parameters of interest based on correlations between signal features and internal container conditions, potentially leveraging machine learning models trained on reference measurement data.

In one aspect of the invention, the wireless communication node may be a 5G base station (gNodeB) or similar wireless access point that directs beamformed signals toward containers or storage areas, utilizing the same RF infrastructure for both conventional communication services and specialized sensing applications. This dual-use approach enables efficient resource utilization and may simplify deployment in environments where both connectivity and monitoring are required.

In one aspect of the invention, measurements and inferences generated by the sensor interrogation module may be transmitted through the node's backhaul interface to centralized processing systems, management platforms, or application servers. These measurements may be relayed using standard cellular protocols, transmitted via wired Internet connections, or distributed through private networks, enabling integration with broader inventory management, quality control, or regulatory compliance systems.

In one aspect of the invention, the wireless communication node may implement various sensing modalities within the sensor interrogation module, including passive backscatter detection from RFID tags, active interrogation of semi-passive elements, or direct radar-based monitoring of liquid interfaces. The specific technique may be selected based on deployment requirements, container materials, and desired measurement accuracy, with the node potentially supporting multiple simultaneous sensing approaches.

In accordance with the principles of the invention, the wireless communication node may support both local processing of sensing data within the node and transmission of raw or preprocessed measurements to external systems for more sophisticated analysis. This flexible architecture enables deployments tailored to specific operational requirements, from edge-focused implementations with minimal backhaul requirements to cloud-centric approaches that leverage centralized computing resources for enhanced inference accuracy.

Aspects of the present disclosure provide apparatuses, methods, processing systems, and computer-readable mediums for non-invasively monitoring internal contents of sealed containers using external sensor elements configured to detect and analyze radio-frequency (RF) signal interactions. In various embodiments, such external sensor elements are affixed to an outer surface of the container and cooperate with RF interrogation devices to measure signal characteristics corresponding to conditions inside the container. By processing these RF responses, the system can determine parameters such as fluid level, alcohol content, and environmental changes without breaching the container or introducing any measurement probes into the enclosed space.

In one implementation, the aspects disclosed herein evolved from a desire to enable remote, accurate, and efficient tracking of aging fluids (e.g., distilled spirits) and other liquid products stored for extended periods. Traditionally, determining the condition of a liquid inside a sealed barrel involved frequent manual measurements or invasive sampling methods. Building upon improvements in RF sensing, miniaturized antennas, and advanced signal-processing techniques, aspects of the present disclosure leverage multiple externally mounted RF-responsive elements and robust analytics-thereby reducing the operational costs, contamination risks, and inaccuracies associated with prior approaches. The resulting solution not only preserves product quality but also allows real-time or scheduled data gathering and analysis across networks of containers in large-scale facilities.

At least one technical challenge addressed herein is the difficulty of accurately assessing internal container conditions (e.g., fluid level, alcohol strength, or other chemical characteristics) without physically disrupting or exposing the contents. Conventional methods relied on time-intensive sampling, physical probes that can alter flavor profiles or introduce contaminants, or bulky equipment poorly suited for rickhouse and warehouse-scale deployment. Moreover, widespread adoption of non-invasive measurement systems has historically been hindered by limitations in RF propagation through container walls, variable environmental interference, and the need to distinguish between slight changes in liquid volume versus changes in ambient conditions.

The technical solutions provided in embodiments of the present disclosure overcome these problems by integrating RF-responsive elements on external surfaces of the container, as illustrated and described herein, in conjunction with an RF interrogation apparatus and computational intelligence. These RF-responsive elements may provide signal reflections and variations corresponding to fluid levels, dielectric changes of the contained liquid, and structural conditions of the barrel or cask. By correlating these signal signatures with reference data sets or machine-learning models, the aspects described herein can infer internal fluid levels and alcohol content. In some aspects, artificial intelligence algorithms dynamically adjust interrogation parameters to mitigate signal interference or environmental noise, enabling repeated, high-fidelity measurements without dismantling or opening containers.

Advantages of these techniques include a reduced risk of contamination, since no probes intrude into the sealed container, and more consistent data collection, given the automated or semi-automated nature of RF-based measurements. Facilities benefit from near real-time monitoring of large inventories, ensuring better regulatory compliance, predictive maintenance, and timely identification of liquid losses or quality issues. By maintaining container integrity while simultaneously capturing precise analytics, the inventive system significantly enhances production oversight, supports advanced aging strategies, and offers a scalable, low-labor solution applicable across various industries.

Each of the foregoing implementations can be employed individually or in conjunction.

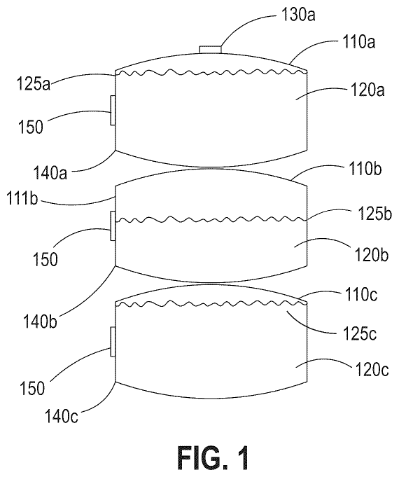

FIG. 1 illustrates a first conventional configuration for storing a plurality of barrels and the liquid contained therein.

Conventionally barrels 110a, 110b, 110c, may be filled with a liquid 120a, 120b, 120c, respectively, and stacked horizontally in racks (not shown). The implementation depicted by FIG. 1 shows the exemplary respective liquid levels 125a, 125b, 125c of liquids 120a, 120b, 120c. Access to barrels 110a, 110b, 110c, is conventional though a bung 130 individually configured in each barrel 110 (of which only bung 130a associated with barrel 110a is shown). In the depicted example the bung 130a is positioned on a side surface of the corresponding barrel 110a. Although only bung 130a associated with barrel 110a is shown, it would be recognized that bung 130 (130a, 130b, 130c) is associated with each of the illustrated barrels 110a, 110b, 110c.

Generally, the bung 130 (e.g., 130a, 130b, 130c) enables a tester (not shown) to access the liquid 120a, 120b, 120c in a corresponding one of barrels 110a, 110b, 110c. As previously discussed, the conventional manner of testing is to insert an object (e.g., a pipette,) into the bung hole 130, wherein liquid is collected in the pipette and removed from barrel 110. The liquid may then be tested to determine quality and the level of the liquid within the barrel using a graduated scale on the pipette.

However, as discussed above, the opening of the bung 130 to insert the pipette into the container 110 to test the contained liquid 120 introduces air and, possibly, other contaminants into the contained liquid. The introduced air may alter the quality of the contained liquid.

Accordingly, container monitoring system 150, disclosed herein, resolves the issues that are known to occur with the conventional means for testing the liquid level within the container. Container monitoring system 150 provides a non-invasive method for determining a level of a contained liquid 120a within barrel 110a, through its inclusion or introduction onto a face surface 140 of each of the illustrated containers or barrels 110a.

Although face surface 140a associated with barrel 110a is shown, it would be recognized that monitoring system 150 may be applied to the face surface 140 of barrels 110b, 110c to provide a non-invasive method for determining a level of a contained liquid (120b, 120c) within barrels 110b, 110c, respectively.

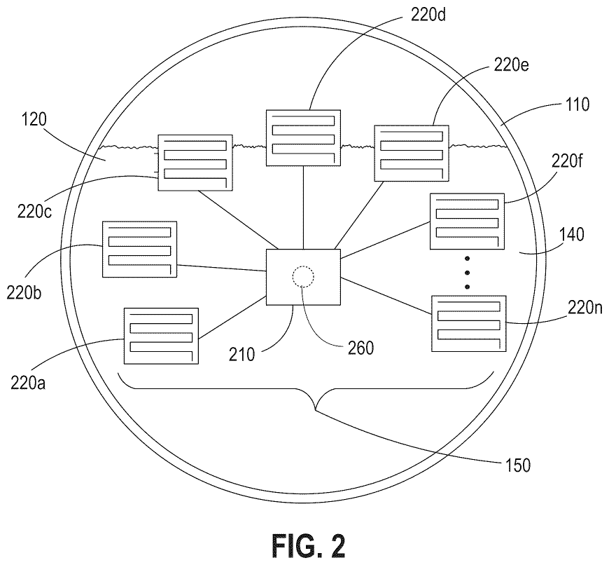

FIG. 2 illustrates a first exemplary embodiment of a monitoring system 150 in accordance with the principles of the invention.

In accordance with the principles of the invention, monitoring system 150 comprising processing section 210 and a plurality of antennas 220 (220a, 220b, 220c . . . 220n) which are positioned on a face surface 140 of a corresponding container or barrel 110.

In accordance with the illustrated aspect of the invention, monitoring system 150 is arranged circumferentially (a “wagon wheel” configuration) about the face surface 140 of barrel 110, wherein processing system 210 is at a center (or hub) of the plurality of illustrated antennas 220a, 220b, . . . 220n.

In accordance with this aspect of the invention, the position of each of the illustrated antennas 220a, 220b . . . 220n with respect to a center position 260 of face surface 140 is known and in a symmetrical relationship. For example, in this illustrated aspect, antennas 220a, 220b . . . 220n may be positioned on face 140 in a conventional “clock” formation. That is, antenna 220d is illustrated as being positioned in a 12 o'clock position with respect to center 260, antenna 220e is illustrated as being positioned at a 1 o'clock position with respect to center 260. Antenna 220f is illustrated as being positioned at a 2 o'clock position with respect to center 260 and antenna 220n may be positioned at a 4 o'clock position with respect to center 260. Similarly, antennas 220c, 220b and 220a may be positioned at 11 o'clock, 10 o'clock and 8 o'clock positions, respectively, with respect to center 260. In accordance with the principles of the invention, the positioning of the illustrated antennas establishes a relationship between a reference point (i.e., center point 260) and each of the antennas that may be used to determine a level of fluid 120 within container 110.

In another aspect of the invention, the plurality of illustrated antennas may be arranged in a physically, (i.e., non-systematical) relation, wherein antennas 220d, 220e, 220f, and 220n may be positioned as discussed above (12, 1, 2, 4 o'clock, respectively) and antennas 220c, 220b and 220a may be positioned at 11:30 o'clock, 10:30 o'clock and 8:30 o'clock positions, respectively with respect to center 260. In accordance with the principles of the invention, the positioning of the antennas 220a . . . 220n in this manner provides for a refined determination of the level of fluid 120 within container 110, as will be discussed.

In one aspect of the invention, processing system 210 provides signals to a corresponding one of the antenna 220a, . . . 220n, which operates as a transmitting antenna to transmit the signals through face 140 toward liquid 120 contained within barrel 110. The corresponding antenna 220a . . . 220n, may then operate as a receiving antenna to receive a reflection of the transmitted signal, which is caused by the interaction of the transmitted signal with the contained liquid 120.

In one aspect of the invention, antennas 220a, 220b, . . . 220n may be omni-direction antennas that emit (or transmit) signals over a wide field of view (e.g., toward and away from face 140). In another aspect of the invention, antennas 220a, 220b . . . 220n may be directional antennas that emit (or transmit) signals in a very limited field of view (e.g., toward face 140). In still another aspect of the invention antennas 220a, 220b . . . 220n may be highly directional antennas with narrow beams widths that emit (or transmit) signals in a limited and narrow field of view (e.g., toward face 140 with 1-degree beamwidth).

In one aspect of the invention, antennas 220a, 220b . . . 220n may each be configured as transmitting and receiving antenna, wherein original signals provided by processing system 210 are transmitted by antennas 220a . . . 220n and reflection signals, captured by antennas 220a . . . 220n), are provided to processing system 210. In another aspect of the invention, selected ones of the illustrated antennas 220a, 220b . . . 220n may operate as transmitting antennas to transmit signals into container 110 and selected other ones of the illustrated antennas 220a, 220b . . . 220n may operate as receiving antenna to capture reflections of the transmitted signals. The antennas designated as transmitting antennas receive signals from processing system 210 and receiving antenna provide signals to processing system 210.

In addition, antennas designated as transmitting antennas may comprise omni-directional or highly directional antenna and antennas designated as receiving antennas may be narrow beam width directional antennas.

In one aspect of the invention, a single antenna may be designated as a transmitting antenna (e.g., 220d) and the remaining of the illustrated antennas (220a, 220b, 220c, 220e . . . 220n) may be designated as receiving antenna. In this case, a single “ping” from the one transmitting antenna may be detected by a plurality of receiving antennas and the results of the detected reflections may be utilized to determine a level of fluid contained. In still another aspect of the invention, the single transmitting antenna may periodically transmit a “ping” and each of the designated receiving antenna may be selectively “turned-on” to enable the ‘turned-on’ receiving antenna to receive a reflection of the transmitted signal.

Although, monitoring system 150 is shown with processing system 210 as a central hub, it would be recognized by those skilled in the art that processing system 210 may be placed at any position on face 140 without altering the scope of the invention claimed.

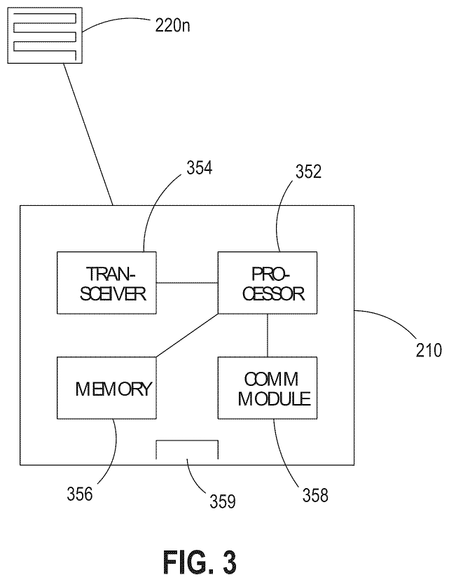

FIG. 3 illustrates a block diagram of an exemplary embodiment of a processing system for determining liquid content within a barrel in accordance with the principles of the invention.

In accordance with the principles of the invention, processing system 210 comprises a transceiving (transmitter/receiver) system 354 that is in communication with antennas 220a . . . 220n (of which only antenna 220n is shown in FIG. 3). Transceiving system 354 may include one or more switching networks (not shown) that provide signals to selectively provide signals to a corresponding one of the plurality of antennas 220a, 220b, 220c. 220n. For example, transceiving system 354 may provide through a (not shown) switching network, signals to each of the plurality of antennas shown in FIG. 2, for example, in a sequential manner such that only one antenna is transmitting and/or receiving at any given time. Alternatively, the (not shown) switching network(s) may cause more than one antenna to concurrently transmit signals and/or receive reflection signals. Alternatively, the (not shown) switching network may cause at least one of the antennas to operate as a transmitting antenna while causing at least one of the plurality of antenna to operate as a receiving antenna.

Although element 354 is referred to as a transceiving system, it would be recognized that transceiving system 354 may comprise separate receiving and transmitting system without altering the scope of the invention claimed.

Processor 352 may comprise one or more conventional processing systems (e.g., INTEL Pentium serial processors) that operates to access instructions and provides control instruction to processing system 210. PENTIUM is a registered trademark of INTEL Corporation, a Delaware, USA corporation. Alternatively, processor 352 may comprise dedicated hardware and software that may provide control instruction to processing system 210.

Memory 356 provides storage capability for instructions (software, code) that may be accessed by processor 352 to control the processing of processing system 210. Memory 356 may for example be represented as semiconductor memory, such as a combination of PROM (programmable read-only memory), wherein instructions are permanently stored or RAM (random access memory), wherein data values may be accessed and overwritten.

Communication module (i.e., transmitter/receiver) 358 represents a means to provide data collected by processor 352 to one or more external devices (not shown), which may be used to evaluate, correlate and collate the data collected. Communication module 358 may comprise a wired or a wireless communication connection to the not shown external devices. For example, communication module 358 may be in wired communication with one or more systems that may be in communication with the Internet that allows for the monitoring of the determined fluid level over a broad geographical area.

Alternatively, communication module 358 may include elements that provide information through one or more wireless communication protocols (e.g., a very short-range NFC protocol (e.g., RFID), a short-range protocol (BLUETOOTH), a longer-range protocol (Wi-Fi) and a long-range protocol (e.g., cellular)). In addition, communication module 358 may operate to receive information from an external source either through a wired communication protocol or a wireless communication protocol. Such information may, for example, comprise instructions (code) that may be stored in memory 356, information regarding the tank (e.g., volume, dimensions, a type of material comprising the tank, etc.) to which system 150 is attached, and the content of the tank. This information may include information for the reprogramming, or the pairing, of system 150 with the specific tank (or barrel) 110. In one aspect of the invention, monitoring system 150 May be “paired” with a specific barrel, such that monitoring system 150 may monitor the contents of the paired barrel 110 over multiple uses of the barrel. For example, an identification number of the container (or barrel) to which monitoring system 150 is attached, may be input into memory 354. Alternatively, barrel 110 may include an electronic identification code that may be input via a wireless communication connection into monitoring system 150 (i.e., paired) using a short-range identification communication protocol (e.g., RFID).

Power source 359 provides power (electrical energy) to the electrical/electronic components of processing system 210. In one aspect of the invention power source 359 may represent a lithium-nickel battery that provides power to monitoring system 210 for an extended period of time. In another aspect of the invention, power source 359 may be a rechargeable battery element that may be recharged by removal from processing system 210 or recharged while included within processing system 210. Alternatively, power source 359 may be an AC to DC converter that receives electrical energy from a main source of power (e.g., 120-volt outlet) and converts the received power to a direct current that is used to power the electrical/electronic components of processing system 210.

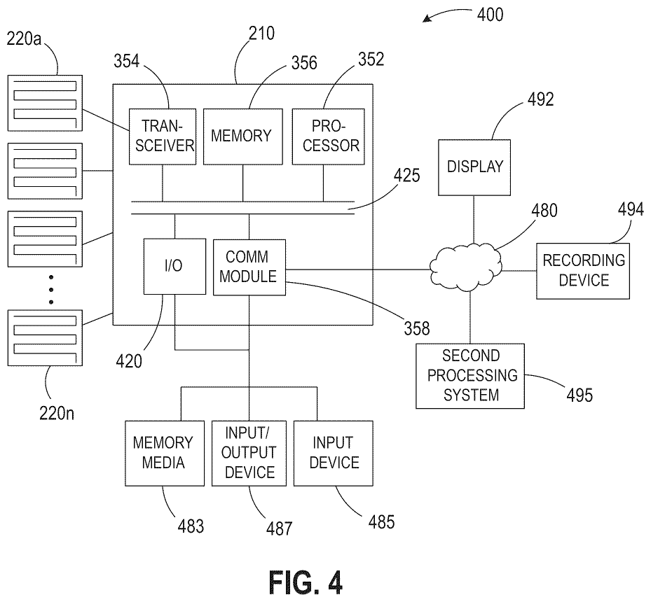

FIG. 4 illustrates a block diagram of an exemplary system for determining liquid content within a barrel in accordance with the principles of the invention.

In this exemplary system embodiment 400, input data is received from antennas (sources) 220a . . . 220n and processed in accordance with one or more programs, either software or firmware, executed by processing system 210. The results of processing system 210 may then be transmitted over network 480 for viewing on display 492, reporting device 494 and/or a second processing system 495.

In the depicted implementation processing system 210 includes one or more receiving devices 354 that receive data from the illustrated sources or devices 220a . . . 220n. The received data is then applied to processor 352, which is in communication with input/output device 420 and memory 356. Transmitting/receiving element 354, processor 352 and memory 356 may communicate over a communication medium 425, which may represent a communication network, e.g., ISA, PCI, PCMCIA bus, one or more internal connections of a circuit, circuit card or other device, as well as portions and combinations of these and other communication media.

Processor 352 may be a general processor central processing unit (CPU) or a special purpose processing unit or dedicated hardware/software, such as a PAL, ASIC, FGPA, each of which is operable to execute computer instruction code or a combination of code and logical operations. In one embodiment, processor 352 may include, or access, software or code that, when executed by processor 352, performs the operations illustrated herein. As would be understood by those skilled in the art when a general-purpose computer (e.g., a CPU) loaded with or accesses software or code to implement the processing shown herein, the execution of the code transforms the general-purpose computer into a special purpose computer. The code may be contained in memory 356 or may be read or downloaded from one or more external devices.

For example, code or software may be downloaded from a memory medium, such as a solid-state memory or similar memory devices 483, or may be provided by a manual input device 485, such as a keyboard or a keypad entry, or may be read from a magnetic or optical medium (not shown) or via downloaded from a second I/O device 487 when needed. Information items provided by external devices 483, 485, 487 may be accessible to processor 352 through input/output device 420, as shown. Further, the data received by input/output device 420 may be immediately accessible by processor 352 or may be stored in memory 356. Processor 352 may further provide the results of the processing to one or more external devices (i.e., display 492, recording device 494 or a second processing unit 495).

As one skilled in the art would recognize, the terms processor, processing system, computer or computer system may represent one or more processing units in communication with one or more memory units and other devices, e.g., peripherals, connected electronically to and communicating with the at least one processing unit. Furthermore, the devices illustrated may be electronically connected to the one or more processing units via internal busses, e.g., serial, parallel, ISA bus, Micro Channel bus, PCI bus, PCMCIA bus, USB, etc., or one or more internal connections of a circuit, circuit card or other device, as well as portions and combinations of these and other communication media, or an external network, e.g., the Internet and Intranet. In other embodiments, hardware circuitry may be used in place of, or in combination with, software instructions to implement the invention. For example, the elements illustrated herein may also be implemented as discrete hardware elements or may be integrated into a single unit (e.g., ASIC).

As would be understood, the operations illustrated may be performed sequentially or in parallel using different processors to determine specific values. Processing system 210 may also be in two-way communication with each of the sources 220a . . . 220n. Processing system 210 may further receive or transmit data over one or more network connections 480 from a server or servers over, e.g., a global computer communications network such as the Internet, Intranet, a wide area network (WAN), a metropolitan area network (MAN), a local area network (LAN), a terrestrial broadcast system, a cable network, a satellite network (cellular), and a wireless network (Wi-Fi), as well as portions or combinations of these and other types of networks. As will be appreciated, network 480 may also be internal networks or one or more internal connections of a circuit, circuit card or other device, as well as portions and combinations of these and other communication media or an external network, e.g., the Internet and Intranet.

In one aspect of the invention, external devices 483, 485, 487, 492, 494, 495 may be representative of a handheld calculator, a special purpose or general-purpose processing system, a desktop computer, a laptop computer, tablet computer, or personal digital assistant (PDA) device, etc., as well as portions or combinations of these and other devices that can perform the operations illustrated.

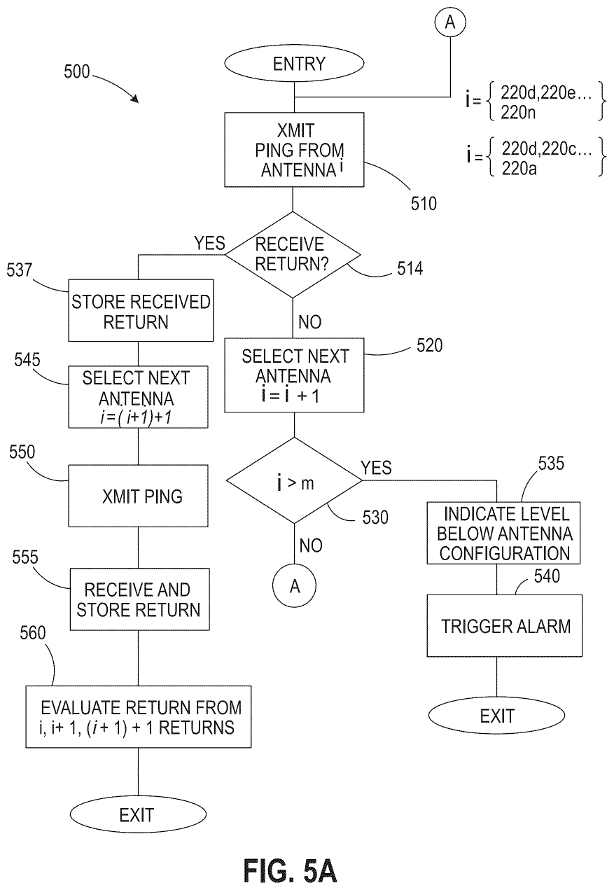

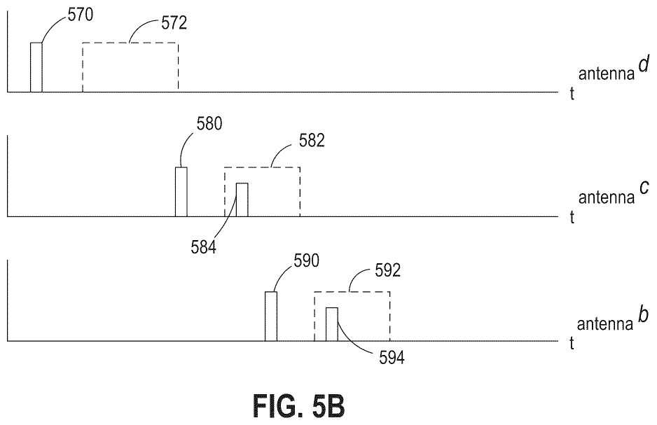

FIG. 5A illustrates a flowchart of an exemplary processing in accordance with the principles of the invention.

In this illustrated exemplary processing 500, the processing system 210 (described with reference to at least FIGS. 2-4) initiates transmission of a signal (referred to, hereinafter as “ping”) to a selected one (“i”) of the antenna 220a . . . 220n. In one aspect of the invention, the initially selected antenna may be selected as the top-most antenna (i.e., 220d, FIG. 2) as the container may be considered in an initially “full state.”

In accordance with the illustrated embodiment shown in FIG. 2, processing may operate from the highest antenna 220d positioned on face 140 to the lowest antenna positioned on face 140 (220a or 220n). In one aspect of the invention, processing may select to operate with antennas selected from a first set of antennas (i.e., 220d, 220e . . . 220n—clockwise selection). Alternatively, processing may select to operate with antennas selected from a second set of antennas (i.e., 220d, 220c . . . 220a—counterclockwise selection). In still another alternative aspect of the invention, processing may select to operate using the first set of antennas and then the second set of antennas, wherein the first and second sets of antennas may be a symmetric or a non-symmetric relation with respect to a known point (e.g., center point 260). Although examples of the selection of the one or more antenna selected to be within the set of antenna are disclosed, it would be recognized that other methods of selection of antennas within the set of antenna may be implemented without altering the scope of the invention claimed.

Processing then selects, at step 510, an initial antenna selection, referred to as “i” from which a signal or a ping is to be transmitted. At step 514, processing waits for return or reflection of the transmitted ping.

Upon not receiving a return (or reflected) signal (after a known period of time, as discussed in FIG. 5B), processing continues to step 520, where a next (“i+1”) antenna is selected from the selected clockwise or counterclockwise set of antennas. Processing then proceeds to step 530 where a check of the value (within the selected set) of the selected antenna is greater than the number of antenna (m) within the selected set of antenna. If the value of the selected antenna is greater than the number antenna within the set, then processing proceeds to step 535, wherein the returns (i.e., reflections of transmitted pings) from each of the antenna within the selected set of antenna is evaluated.

At step 535, the processing system 210 performs a test to determine if any return has been received from any antenna in the selected set. Upon determining no returns have been received from any of the antennas in the selected set, the processing system 210 sets an indication that no returns have been received from any of the antennas in the selected set and, hence, the liquid level is flagged as being “Too Low.” At step 540, the processing system 210 triggers an alarm indication to indicate the “Too Low” condition.

Returning to step 530, if the value (within the selected set) of the next selected antenna is not greater than the number of antennas within the selected set, processing proceeds to step 510 to transmit (i.e., Xmit) a ping from the selected (next) antenna.

Returning to step 514, when a return is detected, processing proceeds to step 537 where the received return is stored. At step 545, a next antenna is selected ((i+1)+1), wherein processing proceeds to step 550 to transmit a ping from the selected (next) antenna. At step 555, a return from the transmitted “ping” is received and subsequently stored.

At step 560, the returns from the i, i+1 and (i+1)+1 antenna selected are evaluated to determine a level of the contained liquid.

As the antenna selection is made from highest to lowest antenna placement on face 140 (in this illustrative processing) after two sequential returns are received, processing is halted as each of the antennas lower in position to the (I+1)+1 antenna would be in contact with the contained liquid and, thus, information from these lower antennas do not contribute any additional information to the level of the contained liquid. This limitation of the number of antennas transmitting is advantageous as it reduces the power requirements needed in obtaining a level of the contained fluid.

Although FIG. 5A refers to processing for selecting one antenna in one of a clockwise set and a counterclockwise set of antennas, it would be understood that the processing shown in FIG. 5A may be adaptable to select first one set of antennas (e.g., clockwise) and then select the other set of antennas (e.g., counterclockwise) to determine the level of the contained liquid.

In one aspect of the invention, wherein the position of the antennas within one set (e.g., clockwise) of antenna on face 140 may be spatially offset from a position of the antenna in the second set (e.g., counterclockwise) of antenna on face 140 (i.e., non-symmetrical relation), the use of information from both the first and second sets of antenna provides for a more precise determination of the liquid within container 110.

FIG. 5B illustrates an exemplary timing chart in accordance with the principles of the invention.

In this illustrated example, which corresponds to the processing shown in FIG. 5A, an initial ping or transmission 570 is made from antenna 220d (the highest antenna illustrated in FIG. 2). A return window 572 is opened. The time period the return window 572 remains open is based on the expected time of the detection of a return to ping 570.

In this illustrated example, a return is not detected within the expected time, which is flagged as a return, but a NO response. Processing proceeds to select a next antenna (e.g., antenna 220c), wherein a ping 580 is transmitted and a return window 582 is opened. In this illustrated example, return 584 is detected and window 582 is closed. A next antenna (e.g., antenna 220b) is selected from which ping 590 is transmitted and return window 592 is opened.

As illustrated, return 594 is detected and, thus, window 594 is closed.

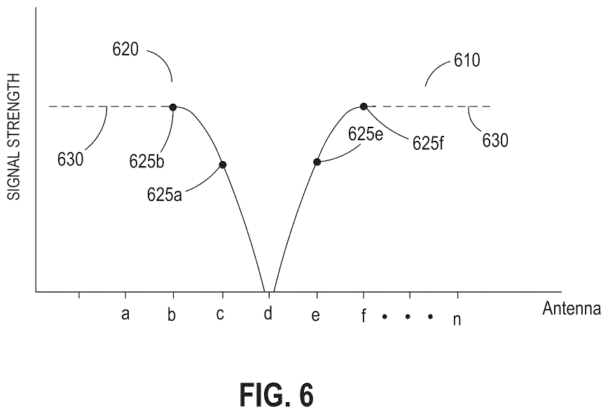

FIG. 6 illustrates a graph of an exemplary signal return chart 600 for determining liquid content within a barrel in accordance with the principles of the invention.

In this illustrated example, which is related to the timing diagram shown in FIG. 5B, the transmission of a ping from antenna 220d produces no return and, hence, no signal is shown for antenna 220d in FIG. 6. However, with the selection of antenna 220e and 220f, returns 625e and 625f detected by antenna 220 and 220f, respectively are shown on graph segment 610.

With the detection of return 625e and, a second (confirmation) return 625f, processing may be halted and a level of contained liquid may be determined.

Further illustrated are returns 625c and 625b, associated with antenna 220c and 220b, (see FIG. 2), respectively on graph segment 620.

In accordance with one aspect of the invention, returns 625b, 625c, 625e and 625f may be evaluated (e.g., signal strength) to determine a level of the contained fluid.

In accordance with another aspect of the invention, the position of antennas 220b, 220c may be spatially offset (i.e., physically displaced) from antennas 220e, 220f and, thus, the evaluation of the received returns may determine the level of the contained liquid more precisely, as previously discussed.

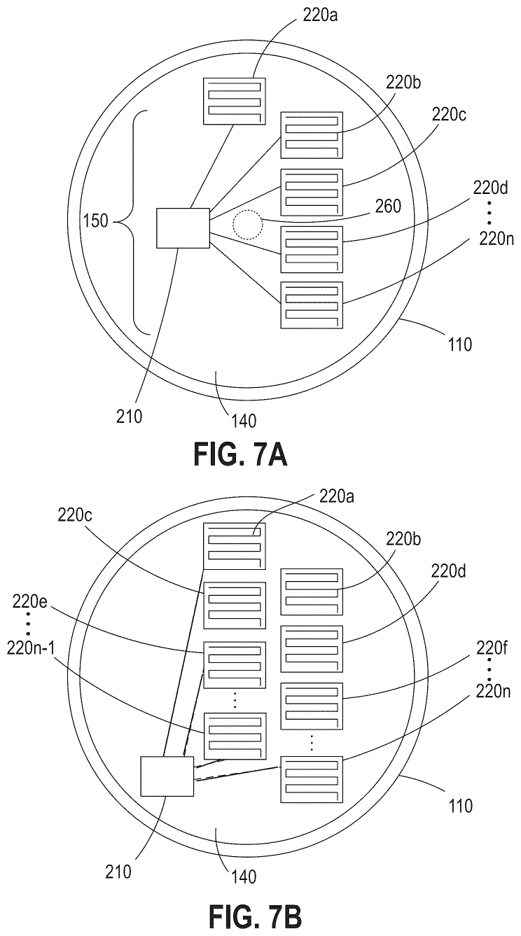

FIG. 7A illustrates a first aspect of a second exemplary embodiment of a system for determining liquid content within a barrel in accordance with the principles of the invention.

In this illustrated configuration, antenna 220a, 220b . . . 220n are arranged linearly on face 140 of barrel 110.

In this illustrated configuration, antenna 220a, 220b, 220c, . . . 220n are shown in a linear arrangement, wherein processing similar to that shown in FIGS. 5A, 5B and 6 may be performed.

FIG. 7B illustrates a second aspect of a second exemplary embodiment of a system for determining liquid content within a container (or barrel) in accordance with the principles of the invention.

In this illustrated configuration, antenna 220a, 220c . . . 220n-1 may be arranged in a first set and antenna 220b, 220d . . . 220n may be arranged in a second set of antennas that is spatially offset from the first set of antennas. As discussed with regard to FIG. 2, the positioning of the illustrated plurality of antenna in a physically non-symmetrical relation allows for a more precise determination of a level of fluid within barrel 110. In the implementation depicted by FIG. 7A the processing system 210 is disposed at a known offset distance from the center point 260 of the face 140 of the barrel 110. The implementation depicted by FIG. 7B includes but does not show the barrel 110 face 140 center point 260 that is not visible behind the depicted antenna 220e.

Accordingly, a determination of the level of a contained liquid may be made based on the receiving of reflections of transmitted pings or signals as previously discussed.

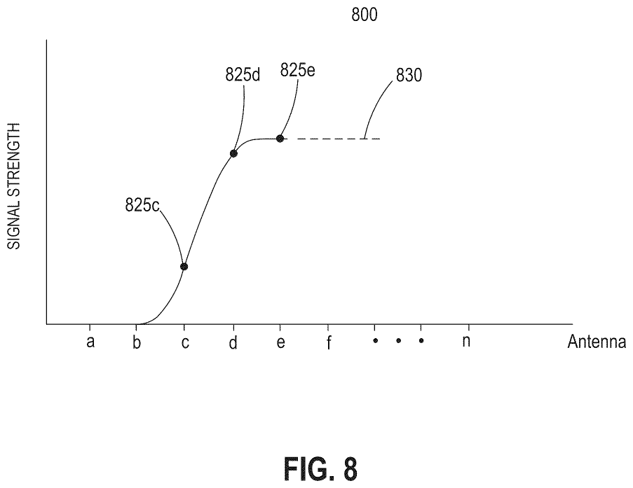

FIG. 8 illustrates a graph of an exemplary signal return chart 800 associated with the configurations shown in FIGS. 7A and 7B in accordance with the principles of the invention.

In accordance with this aspect of the invention, signals transmitted by antenna 220a, 220b (two physically highest antenna, FIGS. 7A and 7B) fail to provide a response within an expected time window (FIG. 5B) and, thus, a first return 825c is received from the transmission of a ping from antenna 220c with a subsequent return 825d received from the transmission of a ping from antenna 220d as shown on graph segment 830. As discussed previously, processing may be halted after two consecutive returns are received.

In accordance with one aspect of the invention, when the returned signal level differ by a known amount, a next transmission and return 825e may be executed to validate a previous return (e.g., 825d)

In this illustrative example, a level of the content liquid in barrel 110 may be determined as lying between the position of antenna 220b and 220c, based on the strength of return signals depicted by FIG. 8. Hence, with the knowledge of the position of each of the antenna with respect to center point 260 (FIG. 2), the level of liquid 120, and the volume content within barrel 110 may be accurately determined.

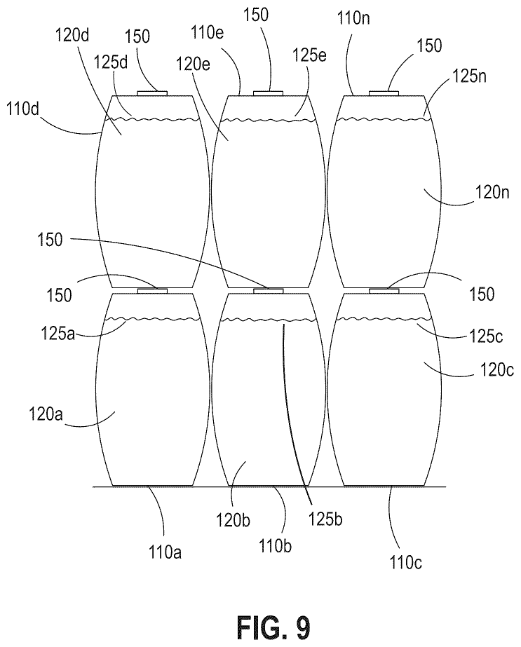

FIG. 9 illustrates a second conventional configuration for storing a plurality of barrels and the liquid contained therein.

In this second configuration of storing barrels, barrels 110a, 110b, . . . 110n are stored vertically where monitoring system 150 is attached to face 140 of each of the illustrated barrels 110. The implementation depicted by FIG. 9 shows the exemplary respective liquid levels 125d, 125e, 125n, 125a, 125b, 125c of liquids 120d, 120e, 120n, 120a, 120b, 120c.

In this illustrated configuration, it would be recognized by those skilled in the art that the level of the contained liquid with each of the barrels may be obtained from a single signal or ping, as the level of the liquid is measured from face 140.

Accordingly, monitoring system 150 may be configurated to include a single antenna configuration that may be used to monitor the vertically displaced liquid within the vertically stacked container(s) 110.

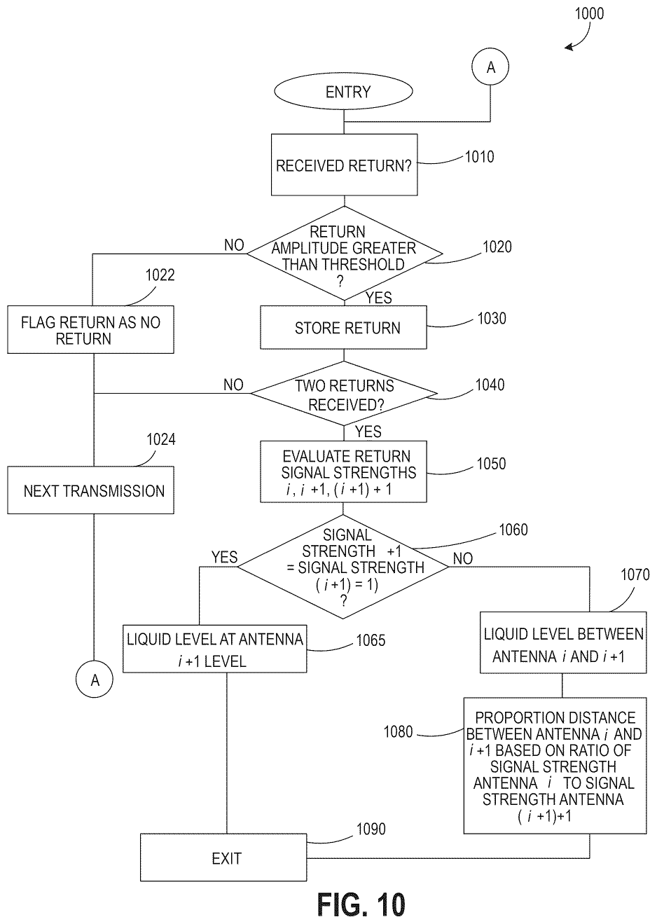

FIG. 10 illustrates a flowchart of an exemplary processing for evaluating the return signals in accordance with the principles of the invention.

In accordance with the illustrated processing 1000, a determination is made at step 1010 as to whether a return has been received. If so, a signal strength or amplitude of the received signal (i.e., the return) is evaluated with regard to a threshold level at step 1020. If the received signal strength is less than or equal to a predetermined minimum threshold level, then processing continues to step 1022, where the return is removed from the processing and an indication of NO return is associated with the transmitted ping. At step 1024, a next antenna is selected (as previously discussed) and processing continues at step 1010.

Returning to step 1020, if the return signal strength is greater than the predetermined minimum threshold, then processing proceeds to step 1030 where the return is stored.

At step 1040 a determination is made whether two consecutive returns have been received. If not, then processing proceeds to step 1024, wherein a next transmission is initiated.

However, if two consecutive returns have been received, the processing continues to step 1050 to evaluate the received signal strengths associated with the first return (i.e., antenna i+1) and the second return (i.e., antenna i+1+1).

At step 1060 a determination is made whether the received signal strengths of the two consecutive returns are approximately the same. If so then the contained liquid level is determined to be comparable to the position of the i+1 antenna at step 1065. Processing then proceeds to step 1090 where the processing is ended.

Returning to step 1060, if the signal strengths are not approximately equal, then the liquid level may be determined to be between the i^{th} and the i^{th}+1 antenna at step 1070. In one aspect of the invention, the liquid level may be determined proportionally between the i^{th} and the i^{th}+1 antenna based on the signal strength of the i^{th}+1 antenna with respect to the signal strength of the i^{th}+1+1 antenna.

Processing then proceeds to step 1090 to exit.

In accordance with the principles of the invention, the determined level of the contained liquid, based on the signal strength of at least two responses or reflections, which are greater than a threshold value, may then be transmitted to one or more of the illustrated external devices shown in FIG. 4. In one aspect of the invention, threshold value may be preset within memory 356. Alternatively, a threshold value may be downloaded into memory 356 in a manner as previously discussed. In still another aspect of the invention, the threshold value may be dynamically determined, based in part, on the characteristics of the container. For example, a size of the container, a material of the container, etc. For example, a calibration of the monitoring system 150 may occur once placed on a face 140 of a container, wherein the characteristics of the container and/or contained liquid may be entered into monitoring system 150. A series of transmissions may occur from one or more of antenna 220a . . . 220n, and the responses to the series of transmissions may be evaluated for establishing a threshold value that enables signals that may be considered valid responses to the processed.

In one aspect of the invention, a volume of the contained liquid may be obtained from at least the determined fluid level and knowledge of the physical dimensions of the container. For example, the volume of the barrel or tank may be determined as:

V(tank)=πr²L
where L is the length of the tank; and
   r is the radius of a circular segment of the tank

The filled volume of a horizontally oriented tank or barrel, for example, may be determined by first finding an area, A, of a circular segment and multiplying it by the length, L.

A partial volume calculation may next be derived as:

A=(1/2)r2(Θ−sinΘ)
where Θ=2*arccos(m/r) and
Θ is in radians.

Accordingly, a volume of a segment may be determined as:

V(segment)=(1/2)r²(Θ-sinΘ)L.

If the determined fluid level, f, is less than ½ of “d”, then the segment created from the level height and V(fill)=V(segment).

However, if the fluid level, f, is greater than ½ of “d” then, the segment that is created by the empty portion of the tank may be determined and subtracted from the total volume of the container or tank to obtain:

V(fill)=V(tank)−V(segment).

In another aspect of the invention, for vertically oriented barrels, the volume of the contained liquid may be obtained as:

V(tank)=πr²h,
where h is height of the contained fluid.

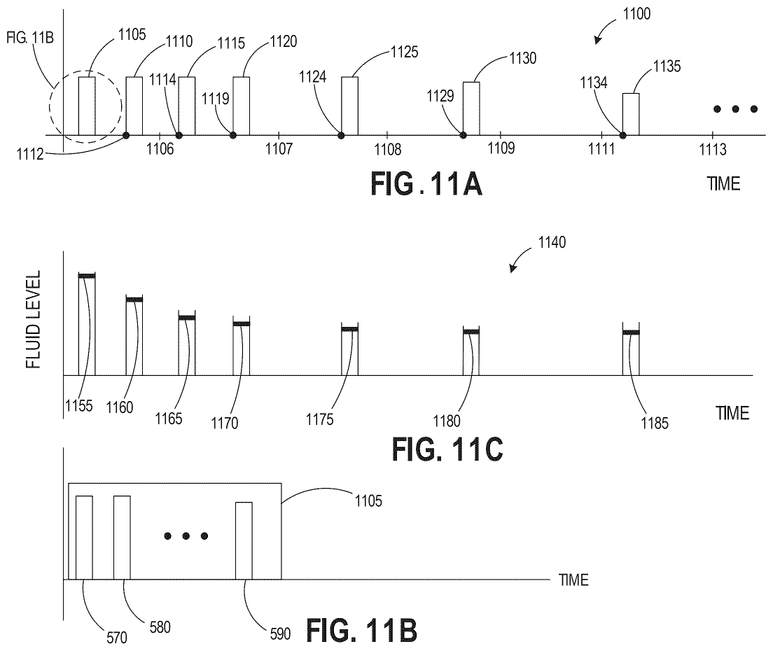

FIGS. 11A-11C illustrate exemplary signal transmission and signal return graphs as a function of time in accordance with a further aspect of the invention.

In accordance with this further aspect of the invention, the quality of a container may be determined by the long-term evaluation of the losses (leakage and/or absorption) of the liquids contained with the container. The long-term evaluation of the losses associated with a container may further be utilized to determine a rate of testing of the liquid within the container.

FIG. 11A illustrates an exemplary signal transmission graph 1100 as a function of time, wherein signal transmissions occur within bursts over an extended period of time. In accordance with the principles of the invention, the duration of the usage of monitoring system 150 is divided into a plurality of periods 1106, 1107, 1108, 1109, 111, 1113 and 1117, which are referred to in this exemplary illustration as collection time periods. Further shown are a plurality of transmission bursts 1105, 1110, 1115 . . . 1150, wherein a measurement of a fluid within a container is made.

FIG. 11B is an expanded view of burst 1105, which is identified as FIG. 11B in FIG. 11A.

In this illustrated example, a plurality of transmissions 570, 580 and 590 (which are comparable to the transmissions shown in FIG. 5B) are included within burst 1105, wherein the plurality of transmissions are associated with at least one of the illustrated antenna 220a-220n, as previously discussed. Accordingly, a collection of fluid levels may be obtained for each of the illustrated transmission bursts.

In one aspect of the invention, processing system 210 may include a timer circuit (not shown) that provides an alarm clock feature that causes processing system 210 to transmit burst 1105, containing transmissions 570, 580, 590. After processing the associated reflections from transmissions 570, 580, 590, processing system 210 may enter a sleep mode, in which little power is consumed. After burst 1105 is completed, processing system 210 may again be activated by the timer circuit (not shown) to cause the transmission of signals (i.e., 570, 580, 590) within burst 1110.

This process of sleeping after each burst is completed and activating after a known time thereafter (e.g., 1112, 1114, 1119, 1124 . . . 1149) repeats for the life of the container or barrel to which monitoring system 150 is attached.

This process of sleeping and activation is advantageous as it provides for extended usable life of a fixed, or dedicated power source.

In one aspect of the invention, the activation time may be substantially constant such that fluid measurement may be made at a known rate. For example, burst transmissions 1105 . . . 1150 may occur at a known rate (e.g., a daily basis, a weekly basis, a monthly basis, etc.). The desired rate of fluid measurement may be input into processing system 210 as previously described.

Alternatively, and as shown in FIG. 11A, the rate of fluid measurement may be made dynamically, based on changes in the fluid measurement over time.

FIG. 11C illustrates an exemplary graph 1140 of corresponding fluid levels or container volume determined based on the return signals associated with the transmission bursts.

In this exemplary graph, a fluid level or container volume value 1155 may be determined based on the signal transmissions/signal returns associated with burst 1105. Similarly, a fluid level or container volume value 1160 may be determined based on the signal transmissions/signal returns associated with burst 1110. And in accordance with the principles of the invention, fluid levels or container volumes 1165, 110, 1175, 1180 1185, 1195, etc. may be determined based on the signal transmissions/signal returns associated with corresponding transmission bursts 1115, 1120, 1125, 1130, 1135, 1150, etc.

As illustrated, the determined fluid level, or volume, initially decreases from a high value 1155 (i.e., full barrel) to a lower value 1175 and then remains substantially constant (i.e., 1175, 1180, 1185) as the losses from leakage and/or absorption decrease over time.

Accordingly, the rate of change of the fluid level or volume may, thus, be used to determine a duration of a sleep state of processing system 210. For example, when the rate of change of the fluid level is high (e.g., level 1155 to level 1160), signal transmission bursts and subsequent level measurements may be performed at a first rate (e.g., once/day). However, as the rate of change of the measured fluid level is slowing (e.g., level 1165 to level 1170) the duration of a sleep state of processing system 210 may be increased such that signal transmission bursts and measurements are performed at a second rate (e.g., once/week). In addition, as the rate of change of the measured fluid level is determined to be substantially negligible (e.g., level 1180 to level 1185) the duration of the sleep state of processing system 210 may be increased still further.

This dynamic determination of the rate of measurement is further advantageous as it further decreases the power needed to maintain system 150 for extended periods (e.g., multiple years).

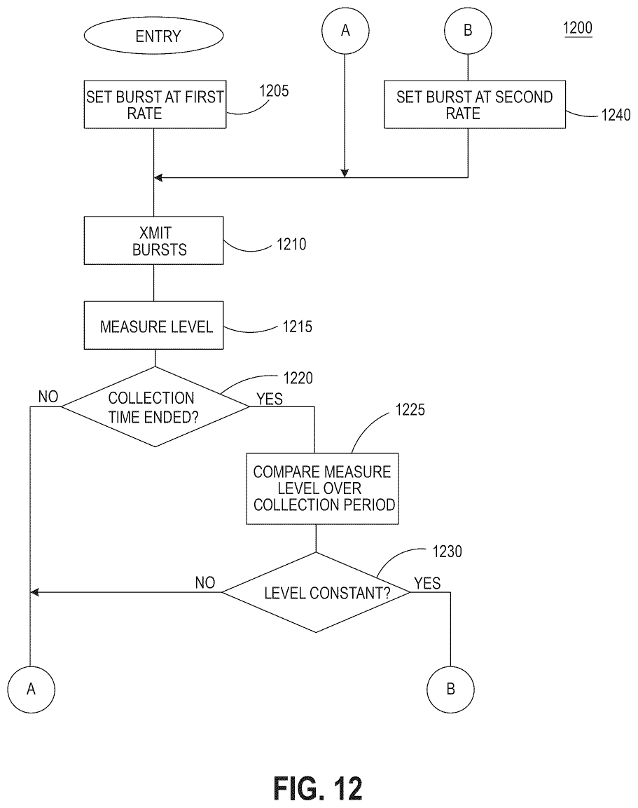

FIG. 12 illustrates an exemplary processing 1200 associated with the graphs shown in FIGS. 11A-11C.

In this illustrated process, the rate of burst transmission 1105-1150 (each containing signal transmission 570, 580 590) is set to a first rate at step 1205. At step 1210, a burst transmission (e.g., 1105) occurs wherein a fluid level (or volume) is determined at step 1215, as previously discussed. At step 1220, a determination is made whether a collection time has ended. If not, then processing proceeds to step 1210 to cause the emission of a second or next burst transmission (e.g., 1110), wherein a second measurement level is determined. At step 1220, a determination is again made as to whether a collection time has ended (e.g., 1106).

If the collection time has ended, processing proceeds to step 1225, wherein the determined fluid level (volume) (e.g., 1155, 1160) are evaluated to determine a rate of change of the determined fluid levels.

At step 1230, a determination is made whether the rate of change is small (i.e., substantially constant level). If the rate of change is not small (i.e., fluid level is not substantially constant) then processing proceeds to step 1210, wherein a next set of burst transmission (e.g., 1115, 1120) occur at the first rate.

However, if the rate of change of the fluid level is small (i.e., fluid level is determined to be substantially constant), then processing proceeds to step 1240, wherein the rate of subsequent transmission bursts is set to a second rate. As shown in FIG. 11A, the second rate is increased such that processing system 210 remains in a sleep state for a longer period and a lesser number of burst transmissions 1130, 1135 occur in an associated collection time period.

To further provide valuable information to the distillers, a measure of alcohol content of the remaining fluid may be determined from the determined evaporation/absorption of the fluid or liquid within the container.

Distilled liquids are stored in warehouses that are generally not climate controlled, and, hence, the ambient or surrounding environment affects the rate of evaporation and/or absorption of the contained liquids.

Environmental factors, such as temperature, barrel characteristics, time and geography contribute to a rate of change of an alcohol content of the fermenting liquid or fluid within a container. Local climate, which includes temperature, temperature fluctuations, and humidity, also affects the rate of evaporation. Local geography, such as altitude, seasonal variations and air quality also affects the rate of evaporation, and, consequently, the alcohol content within the barrel. In addition, the condition of the container is also a factor in the rate of production of alcohol in the container.

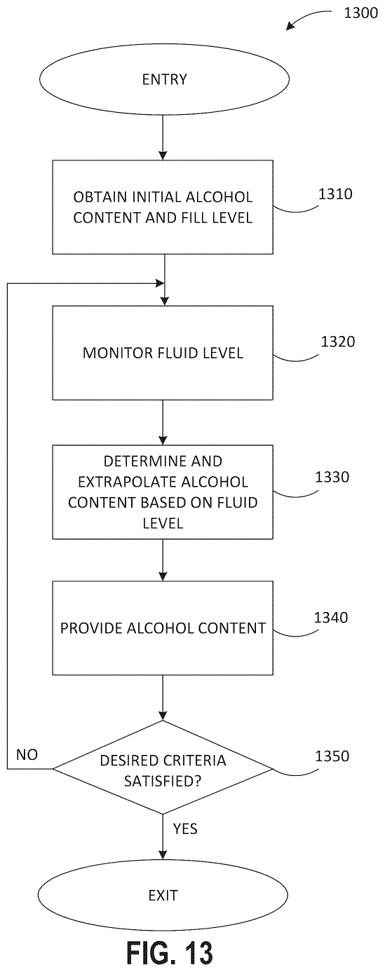

FIG. 13 illustrates an exemplary process for determining alcohol content of a fermenting fluid within a container in accordance with the principles of the invention.

In this illustrated exemplary process 1300 upon filling a barrel or container with a liquid that is to be fermented, a measure of an initial alcohol content is determined and stored at step 1310. For example, the liquid entered into the container or barrel represents a mash that has been obtained from a distillation process associated with the fermentation of a base material, such as barley, rye, corn, wheat or a combination thereof.

At step 1320, a measure of the fluid level within the container is made. The measure of fluid level may be determined continuously, periodically or intermittently, utilizing one or more of the methods previously discussed.

At step 1330, a determination of an alcohol content is performed based at least on a determined fluid level and one or more environmental factors. At step 1340, the determined alcohol content is presented to a user for evaluation.

At step 1350 a determination is made whether one or more criterion associated with a desired requirement is satisfied. For example, determined alcohol content is within a desired range and/or a minimum length of time of the aging of the liquid within the container has been exceeded.

If one or more criterion is not satisfied, processing proceeds to step 1320 for further continued monitoring of fluid level and evaluation of alcohol content. As previously discussed, the monitor of the fluid level (and evaluation of alcohol content) may be determined periodically or continuously. In one aspect of the invention, the period of sampling may be based on a duration of time the liquid is within the barrel. That is, the interval between sampling is shorter during the early stages of fermentation and longer as the period of fermentation is increased.

Otherwise, processing is ended.

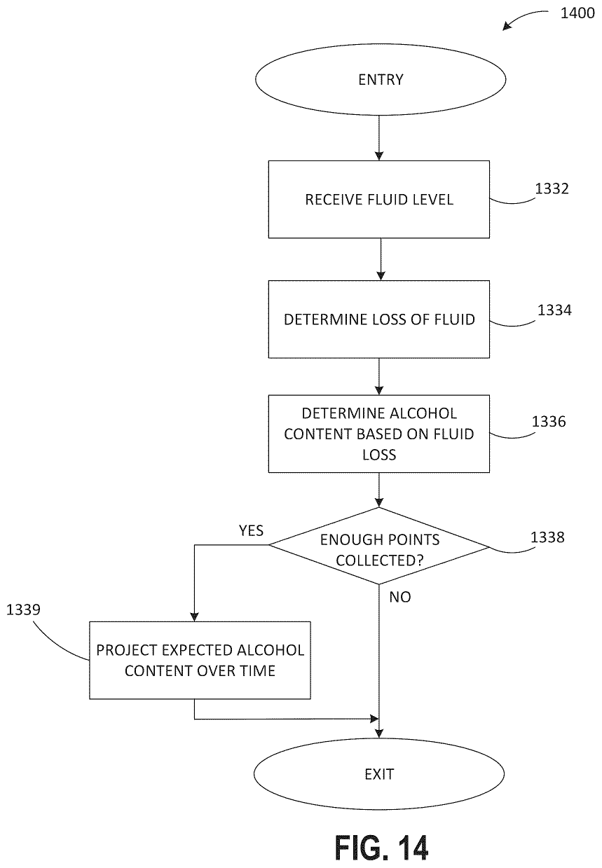

FIG. 14 illustrates a flow chart of an exemplary processing associated with step 1330 of FIG. 13 for determining and extrapolating alcohol content of a liquid within a container in accordance with the principles of the invention.

In this exemplary process 1400, processing receives at step 1332 a fluid level obtained from a monitoring system, as previously discussed. At step 1334, a determination of a loss of fluid or liquid is determined, wherein the loss of fluid may be due to evaporation of the fluid or absorption of the fluid by the container as the container remains in place over an extended period of time.

At step 1336, an alcohol content of the liquid or fluid remaining in the container is determined, wherein the alcohol content is determined based, in part, on the at least one of an initial alcohol content, and one or more environmental conditions.

At step 1338 a determination is made as to whether enough data points have been collected. If enough data points have been collected, a determination of an expected alcohol content (i.e., a projection of alcohol content) is performed at step 1339. For example, and as would be known in the art, when two data points are collected, a straight-line approximation of the alcohol content may be obtained. In an illustrative example, when three data points are collected, a curved line, passing through the collected points, may be formulated that provides for an approximation of the expected alcohol content. When additional sample points are collected a more accurate approximation of the expected alcohol content may be obtained. In one aspect of the invention, at least three data or samples points are to be collected to obtain a first order approximation of the expected alcohol content. In another aspect of the invention, the process of determining an approximation of the expected alcohol content is performed as a selected number (e.g., a specified subset, or all) of the data points collected so as to obtain a more accurate approximation of the expected alcohol content.

Otherwise, processing exits.

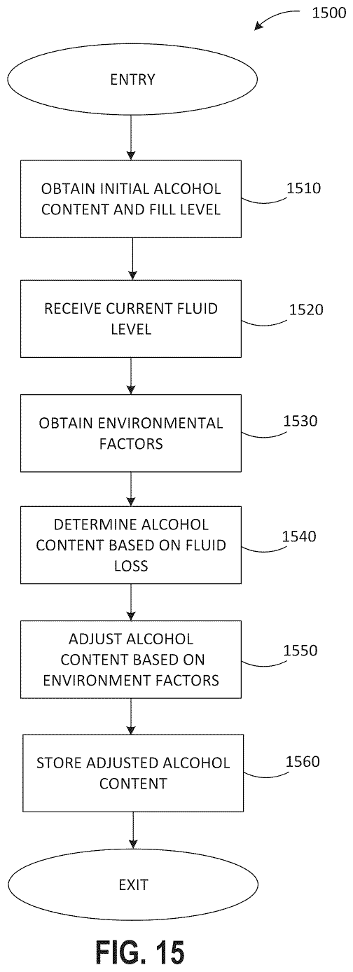

FIG. 15 illustrates a flowchart of an exemplary process associated with step 1336 of FIG. 14 for determining alcohol content of a liquid within a container in accordance with the principles of the invention.

In the illustrated process, an initial alcohol content and fill level are obtained at step 1510. At step 1520, a determination of a loss in liquid or fluid level is based on the initial fill level and the determined current fill level. At step 1530, environmental factors surrounding the container are obtained. These factors may include information regarding temperature, humidity, seasonal variations, etc.

At step 1540 a determination of a current alcohol content is determined from the determined loss, wherein a nominal alcohol content decrease (or increase) is utilized to determine the current alcohol content. In one aspect of the invention, the nominal alcohol content decrease or increase is substantially constant over time. In another aspect of the invention, the nominal alcohol content increase or decrease may be variable, wherein the nominal alcohol content increase or decrease varies over time. In one aspect of the invention, the alcohol content may be determined based on a model of alcohol content over time, wherein the model may be developed by a series of actual measurements obtained over a known time period.

At step 1550 the determined alcohol content is subjected to a process for adjusting the determined alcohol content based on environment factors. And at step 1560, the adjusted alcohol content is stored for subsequent processing.

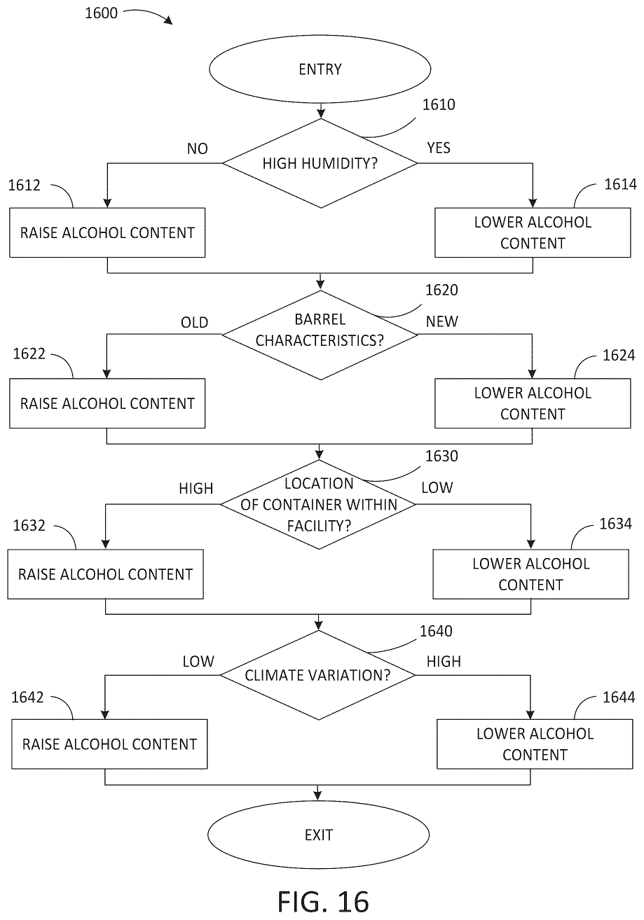

FIG. 16 illustrates a flowchart of an exemplary process associated with adjusting alcohol content presented in step 1550 based on one or more environmental factors or considerations.

In accordance with this exemplary processing 1600, at step 1610, a determination is made regarding the humidity level in the surrounding environment. If it is determined that the humidity level is high, then the alcohol content is lowered, at step 1614, as alcohol evaporates more quickly in high humidity conditions than water. In an illustrative example, a threshold humidity level of fifty percent may be considered as a high humidity level. The threshold humidity level may be adjusted in accordance with what would be known to one of ordinary skill. Otherwise, the alcohol content is raised at step 1612. The degree of raising or lowering the alcohol content may be constant. Alternatively, the degree of raising or lowering the alcohol content may be varied based on the level of humidity. In still another aspect, the degree of raising or lowering the alcohol content may be varied based on a length of time the contained fluid or liquid has been subjected to the humidity conditions.

At step 1620, a determination is made regarding the condition of the container. If it is determined that the container is essentially new, or has been used a few times, then processing proceeds to step 1624 where the alcohol content is lowered, as a newer container affords greater absorption of the contained fluid. Otherwise, at step 1622 the alcohol content is raised, as there is less absorption of the contained fluid.

At step 1630, a determination is made regarding a location of the container. For example, a determination of a high position within a stack of containers may require a raising of the alcohol content, at step 1632 as the higher position may cause greater temperature fluctuations around the container. Otherwise, the alcohol content may be lowered at step 1634 as it is expected that less temperature fluctuation exists around the lower positioned container.

At step 1640, a determination is made regarding climate variations, wherein high climate (e.g., high altitude, northern geographical location, etc.) may require a lowering of the alcohol content at step 1644, while a lower climate (e.g., lower altitude, more southern geographical location, etc.), may require the alcohol content to be retained the same or raised at step 1642.

Processing then exits with an adjusted alcohol content level.

Although processing 1600 refers to a limited number of factors that may be considered in adjusting a determined alcohol content, it would be within the knowledge of those skilled in the art to include additional factors that may affect the alcohol content of a contained liquid over time. For example, such additional factors may include but not be limited to the condition of the container (the condition of the container may include further elements of the nature of the inner surface (e.g., charred, not charred) of the container), the length of time the container has been in service, the number of times or cycles that the container has been utilized, and the like.

Such additional factors have been contemplated by the inventors and are included within the scope of the invention claimed.

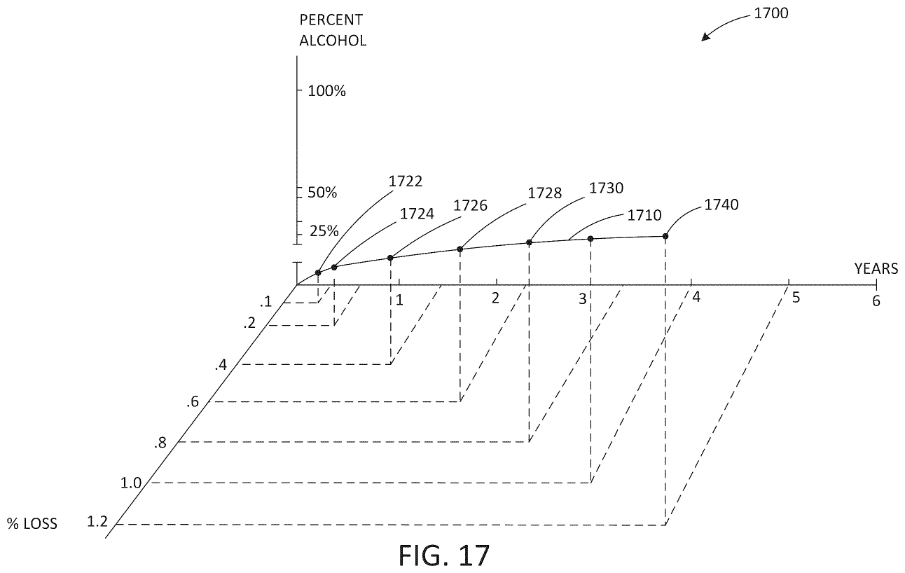

FIG. 17 illustrates an exemplary plot of determined alcohol content and extrapolating alcohol content in accordance with the principles of the invention.

In this illustrated example, chart 1700 comprises a horizontal axis on to which a number of years of storage of a container is plotted and a vertical axis onto which is plotted a percentage of alcohol content of the contained fluid. In addition, on a third coordinate, is shown that represents a percentage of loss of liquid within the container. Accordingly, a three-dimensional formulation or model of alcohol content versus percentage of fluid loss versus a period of time may be determined, wherein an expected or typical alcohol content may be determined from a determination of a loss of fluid.

Plot line 1710 represents an approximation of a change in alcohol content versus fluid loss, wherein plot line 1710 represents an idealized representation of the development or production of alcohol of the fermenting liquid within the container that may be obtained using mathematical formulation. Alternatively, plot line 1710 may correspond to a series of actual measurements of alcohol content made using conventional methods. For example, measurements or data points 1722, 1724, 1726, 1728, 1730 . . . 1740 may represent one or more measurements of both fluid loss and alcohol content of the fermenting liquid at known periods of time. For example, measurements 1722, 1724 . . . 1740 may be taken periodically (i.e., monthly) or randomly,

Based on the measurements or data points 1722-1740, plot line 1710 may then be formulated using statistical methods (e.g., “line of best fit,” “least squares,” etc.) to produce an approximation of the change in alcohol content that best represents the measured points. Although a “line of best fit” or “least squares” method are discussed, it would be recognized by those skilled in the art that other statistical methods may be utilized to formulate plot line 1710 without altering the scope of the invention claimed.

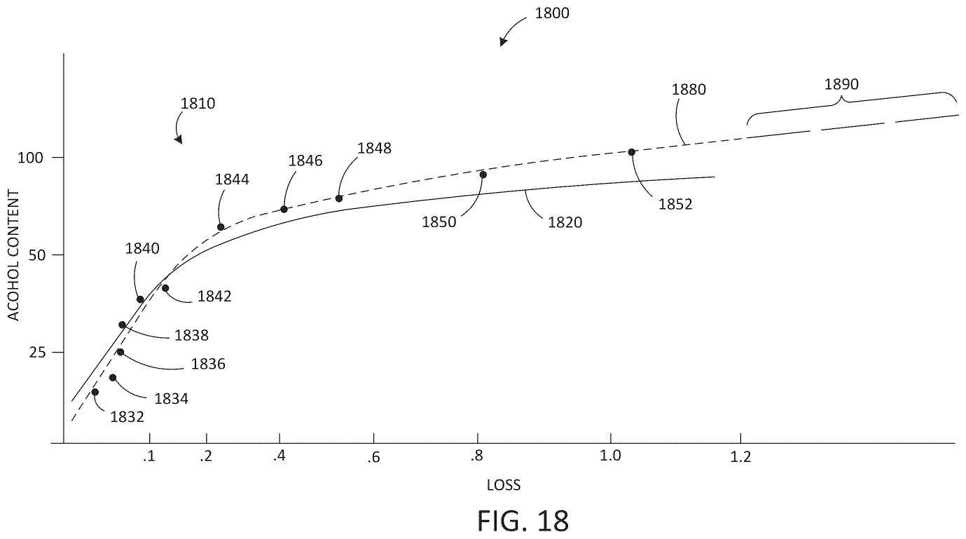

FIG. 18 illustrates an exemplary graph of alcohol content determination in accordance with the principles of the invention.

FIG. 18 represents a projection 1820 of the idealized or model plot line 1710 onto the two-dimensional plane 1810 of alcohol content verses percent of loss shown in FIG. 17.

In accordance with the principles of the invention, utilizing the idealized projection 1820, an estimate, (i.e., a first order) measure of alcohol content may be determined based on a determined fluid loss. That is, preliminary (i.e., first order) alcohol content measurements may be determined for each of the measurement points based on the determined fluid loss taken at different times. A more refined alcohol measurement may be determined from the preliminary alcohol content at each of the measured (i.e., collected) data points by adjusting or modifying the preliminary alcohol content by one or more environmental factors, as shown and discussed with regard to FIG. 16. Measurements 1832,1834, 1836, 1838 . . . 1852, represent the more refined measurements of alcohol content after considering one or more environmental factors for corresponding ones of the expected alcohol content based on the model shown in FIG. 17.

In accordance with the principles of the invention, the measurements of fluid loss (and determination of alcohol content) may be taken at a first rate during a first period of time while subsequent measurements of fluid loss may be taken at a second rate during a second period of time, wherein the second rate is longer than the first rate. That is, the periodicity of the measurement rate (i.e., first rate and second rate) increases over time.

For example, during a first year, when alcohol production and fluid loss is greatest, fluid loss measurements may be taken at a first rate (e.g., 1832 . . . 1842) and when alcohol production and fluid loss is less, fluid loss measurements may be taken at a second rate (e.g., 1848, 1850, 1852), wherein the first rate is higher (i.e., measurements performed more often) than the second rate.

Further illustrated is a statistical formulation of the measured samples 1832, 1834 . . . 1852, as represented by dashed plot line 1880. Similar to the formulation discussed with regard to FIG. 17, dashed plot line 1880 represents a model that may be provided to refine the model shown in FIG. 17. For example, a plurality of plot lines 1880, taken from a corresponding measurements of a plurality of containers that have similar characteristics (i.e., geographical location) may be accumulated and included in model 1710.

In one aspect of the invention, plot line or model 1880 may be utilized to determine a projection 1890 of an expected alcohol content that when projected onto the plane of alcohol v. years, shown in FIG. 17, may provide information of alcohol production for subsequent years.

Various implementations have been disclosed with reference to the Drawings. However, other implementations are possible. For example, an exemplary method may comprise receiving information regarding an initial state of a liquid in a container, the initial state comprising at least one of: an alcohol measure and a level of the liquid within the container; monitoring a level of the liquid within the container, wherein the monitoring is performed external to the container; determining an amount of loss of the liquid within the container based on the initial level and the monitored level; and estimating the alcohol measure of liquid remaining within the container based on the determined amount of loss of the liquid.

The method may further comprise storing a plurality of estimated alcohol measures.

The method may further comprise extrapolating an expected alcohol measure based on the stored plurality of estimated alcohol measures.

Estimating the alcohol measure may further comprise adjusting the estimated alcohol measure based on at least one environmental condition.

The at least one environmental condition may be selected from a group consisting of: temperature, location within a facility, a geographic location of the facility, and container condition.

The determination of estimating the alcohol measure may be performed periodically.

The rate of periodicity estimating the alcohol measure may be adjustable.

The rate of periodicity estimating the alcohol measure may be increased as a function of time.

An exemplary system may comprise a plurality of antennas positioned on an exterior surface of a barrel, wherein the plurality of antennas are configured to capture signals reflected by a liquid within the barrel; and a processor configured to: receive the reflected signals; and determine a level of the liquid within the barrel, wherein based on the determined level of the liquid within the barrel, the processor is further configured to: determine a loss of liquid within the barrel based on the determined level of fluid and an initial level of fluid; and estimate an alcohol content of the liquid within the barrel based on the determined loss of liquid.

The system may further comprise the processor may be configured to store the estimated alcohol content.

The system may further comprise the processor may be configured to extrapolate an expected alcohol content based on a stored plurality of estimated alcohol measures.

The system may further comprise the processor may be configured to adjust the estimated alcohol content based on at least one environmental condition.

The at least one environmental condition may be selected from a group consisting of: temperature, location within a facility, a geographic location of the facility, and container condition.

The determination of estimating the alcohol content may be performed periodically.

The rate of periodicity estimating the alcohol content may be adjustable.

The rate of periodicity estimating the alcohol content may be adjustable as a function of time.

An exemplary method may comprise: determining, by a monitoring system external to a container, a level of a liquid within the container; and estimating an alcohol content of the liquid within the container based on the determining level of the contained liquid, wherein the estimation comprises: determining an amount of loss of liquid based on the determined level of liquid; obtaining a first order alcohol content based on a model expectation of alcohol content; and determining the alcohol content based on adjusting the first order alcohol content based on at least one environmental condition.

The at least one environmental condition may comprise at least one of: a temperature, a location within a facility, a geographic location of the facility, and a container condition.

The method may further comprise projecting an estimated alcohol content based on a plurality of the determined alcohol content.

The method may further comprise determining the level of liquid within the container periodically, wherein measurements of the level of liquid is performed at a first rate during a first period of time and at a second rate during a second period of time, the first rate being faster than the second rate.

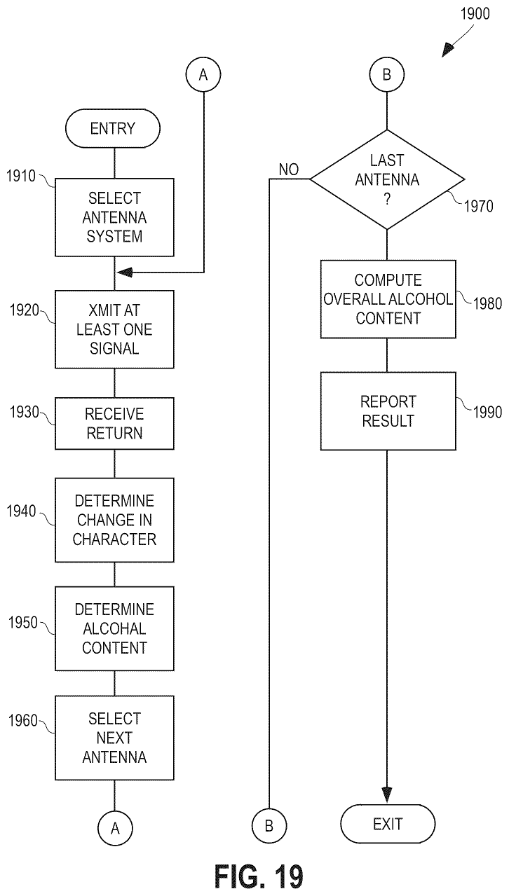

FIG. 19 illustrates a flowchart of a second exemplary process for determining alcohol content of a fluid within a container in accordance with the principles of the invention.

In a further aspect of the invention claimed, the system configurations shown in FIGS. 2, 7A, 7B may be further utilized to determine alcohol content directly. Thus, while alcohol content determination has been discussed with regard to determination of fluid loss, the system may employ a second algorithm in conjunction with or independent of the processing previously disclosed.

In this aspect of the invention, system 150 may implement the illustrated exemplary process 1900, wherein a first antenna from a set of antenna selected from a plurality of antenna associated with the externally mounted antennas shown in FIGS. 2, 7A, 7B is selected at step 1310. In one aspect of the invention, the first antenna among the set of antenna selected may be associated with the lowest position (e.g., 220n) among the illustrated plurality of antenna 220a-220n. Alternatively, the first antenna among the set of antenna may be selected based on a determined level of fluid within the container, wherein the first antenna selected is that antenna that is associated with the highest level of fluid (e.g., the top-most antenna initially). In still a further alternative embodiment, the set of antenna may be selected as a single antenna. For example, the physically lowest positioned antenna may be selected as being included as the sole selected antenna within the set of antenna. In still a further aspect, the sole selected antenna within the set of antenna may be selected as that antenna located physically positioned at or just below the fluid level of the fluid within the barrel. In still a further aspect, the sole selected antenna within the set of antenna may be selected as that antenna physically positioned between the lowest positioned antenna and the antenna positioned at or just below the level of fluid. Although examples of the selection of the one or more antenna selected to be within the set of antenna are disclosed, it would be recognized that other methods of selection of antennas within the set of antenna may be implemented without altering the scope of the invention claimed.

In one aspect of the invention upon filling a barrel or container with a liquid that is to be fermented, a measure of an initial alcohol content may be determined and stored at step 1910. For example, the liquid entered into the container or barrel may represent a mash that has been obtained from a distillation process associated with the fermentation of a base material, such as barley, rye, corn, wheat or a combination thereof.

At step 1920, at least one signal may be transmitted in at least one frequency band from the selected antenna into the contained fluid. A return (i.e., return or reflected signal) associated with each of the transmitted at least one signal is captured at step 1930. At step 1940 a determination of a difference in at least one characteristic (e.g., signal strength, frequency, phase, distance and/or time traveled) between the transmitted signal and the return or reflected signal is made.

In one aspect of the invention, processing system 210 may include a frequency shifting measurement circuit that allows for the determination of a difference between a frequency of the transmitted signal and a frequency of the associated return signal. Alternatively, processing system 210 may include phase shifting measurement circuitry that allows for the determination of a difference between a phase of the transmitted signal and a phase of the associated return signal.

At step 1950, an alcohol content associated with the selected antenna of the fluid within the container may be obtained based on a change in the characteristic (e.g., signal strength, frequency, phase, distance and/or time traveled) of the returned signal.

In one aspect of the invention, the selected antenna may transmit at least one signal (or plurality of signals) at a same frequencies with different phases into the contained fluid, wherein the difference in phase between each of the at least one (plurality of) transmitted signals and the associated returned signal may be determined. In one aspect of the invention, the at least one (plurality of) phase differences may be accumulated and averaged, for example, to obtain an average phase difference. An alcohol content, associated with the selected antenna, may be determined, for example, based on the obtained average phase difference. In another aspect of the invention, the selected antenna may transmit at least one signal (or a plurality of signals) at different frequencies with a same phase into the contained fluid, wherein a difference in frequency between each of the at least one (plurality of) transmitted signal(s) and the associated return may be determined. In one aspect of the invention, the at least one (plurality of) frequency differences may be accumulated and averaged, for example, to obtain an average frequency difference. An alcohol content, associated with the selected antenna may be determined, based on the obtained frequency difference. In still another aspect of the invention, the selected antenna may transmit a plurality of signals at different frequencies and at different phases. Differences in frequency and phase between the transmitted signals and the return signals may be determined, accumulated and averaged to obtain an average frequency and phase values. An alcohol content may be determined based on the averaged frequency and phase values.

At step 1960, a next antenna from the set of antenna of the plurality of antenna is selected. At step 1970 a determination is made whether a last antenna has been selected. In one aspect of the invention, the last antenna may be selected as the last antenna among the plurality of antenna. In another aspect of the invention, the last antenna may be selected as the last antenna associated with a fluid level within the container.

If the last antenna is not selected, processing proceeds to step 1920 wherein at least one signal is transmitted by the selected antenna and the processing illustrated by at least steps 1930 to 1950 for obtaining alcohol content associated with the selected antenna is performed.

However, if the last antenna of the set of antenna has been selected, then processing proceeds to step 1980, wherein an average (or a median) alcohol content may be determined based on the previously determined alcohol content associated with each of the selected antenna. At step 1990, a report of the determined alcohol content may be provided.

In one aspect of the invention, the average (or median) alcohol content may be determined based on a filtering of the alcohol content associated with each of the selected antenna. For example, the average (or median) alcohol content may be obtained by removing a high alcohol content and a low alcohol content from the collected set of alcohol content in order to remove singular values. Alternatively, the average (or median) alcohol content may be obtained by first removing the alcohol content associated with the last selected antenna and averaging or accumulating the remining values. In this manner, the determined average (or median) alcohol content obtained is not influenced by an alcohol content at the fluid/air boundary.

Although, the process shown in FIG. 19 contemplates determining an alcohol content from the determined alcohol content associated with each of the selected antenna, it would be recognized by those skilled in the art that resultant characteristics (e.g., signal strength change, frequency shift, phase shift, change in distance and/or time traveled, etc.) may be obtained for each of the selected antenna, and an alcohol content may be obtained based on a resultant characteristic obtained over all the selected antenna within the set of antenna.

In accordance with the principles of the invention, the alcohol content obtained utilizing the processing shown in FIG. 19 may be further adjusted in a manner similar to that described with regard to FIGS. 15 and 16.

In still another aspect of the invention, the alcohol content obtained utilizing the processing shown in FIG. 19, may be correlated with the alcohol content obtained utilizing the processing shown in FIG. 14. Alternatively, the alcohol content obtained utilizing the processing shown in FIG. 19, may supplement the alcohol content obtained utilizing the processing shown in FIG. 14 to improve the model as shown in FIGS. 17 and 18.

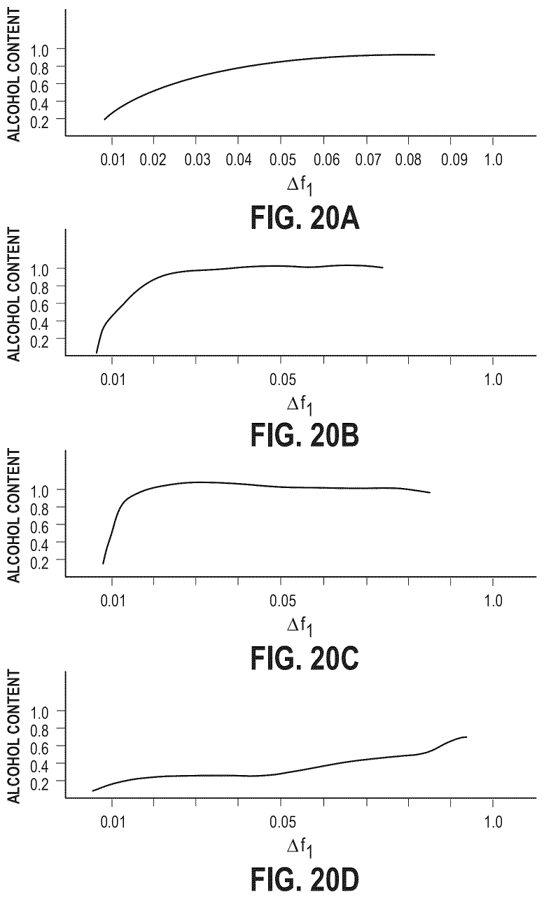

FIGS. 20A-D illustrate exemplary charts of alcohol content of a liquid within a container for different transmitted frequencies.

FIG. 20A illustrates an exemplary chart (or mapping) of an alcohol content of a liquid within a container over a transmitted frequency wherein the transmitted signal frequency is represented as f₁. In this exemplary chart alcohol content is represented on a vertical axis and a frequency difference (Δf₁) between a transmitted signal and a return signal is shown on a horizontal axis. As shown, as the alcohol content increases, the expected difference in frequency between the transmitted signal and the return signal increases. Accordingly, a measurement of the frequency difference provides a means for determining an alcohol content of a fluid or liquid within the container.

Accordingly, a measurement of the frequency difference provides a means for determining an alcohol content of a fluid or liquid within the container.

FIGS. 20B-20D represent charts, similar to the chart shown in FIG. 20A, representing a measure of alcohol content associated with a fluid or liquid for different transmitted frequencies (represented as f₂, f₃, f₄ respectively).

Accordingly, an alcohol content may be determined for each of a plurality of measured frequency returns for each of the selected antenna configurations. Accordingly, an overall alcohol content may be determined based on the collection of one or more alcohol content taken over one or more frequency measurements over one or more antenna configurations.

Although only four (4) frequencies are illustrated, it would be within the knowledge of those skilled in the art to create additional charts showing frequency shift as a function of alcohol content without undue experimentation. As the number of charts similar to those shown in FIGS. 20A-20D is expanded, the accuracy of the measurement of alcohol content would increase as the number in the transmitted frequencies increases.

Although FIGS. 20A-20D illustrate exemplary charts of alcohol content as a function of change in frequency, it would be recognized by those skilled in the art that a similar set of exemplary charts may be obtained as a function of phase change, signal strength change, change in distance and/or time traveled or other similar characteristic associated with the transmitted signal, without altering the scope of the invention claimed.

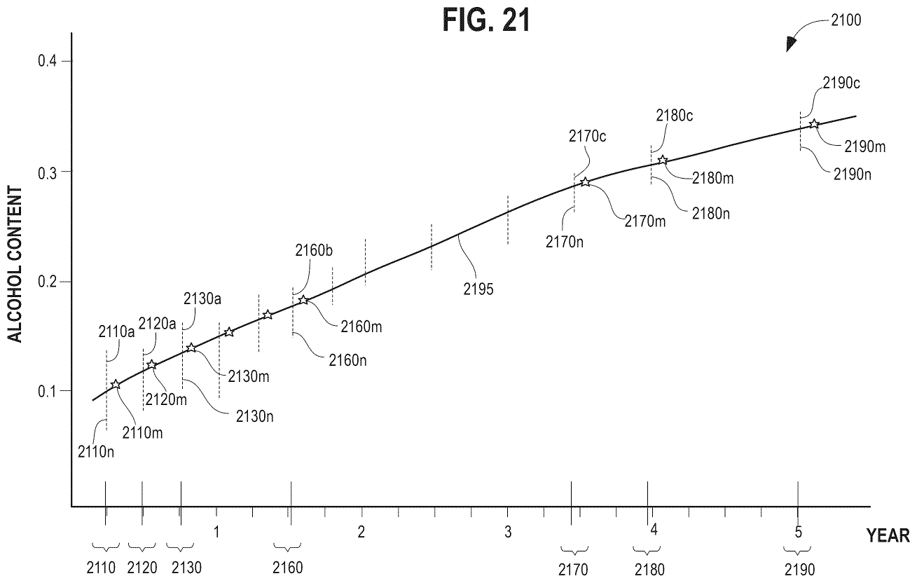

FIG. 21 illustrates an exemplary chart of determined alcohol content as a function of time in accordance with the principles of the invention.

In this illustrated example, chart 1500 represents a plurality of measurement sets 2110, 2120, 2130, 2140, 2150, 2160, 2170, 2180 and 2190 that are taken at intervals over a period of time. For example, the measurements 2110a-2110n associated with measurement set 2110 represent a measured alcohol content for each of a plurality of antenna 220a-220n (similar to the configurations shown in FIGS. 2, 7A and 7B). In this illustrated example, the value of “n” is selected as eight (8) to illustrate the principles of the invention. Similarly, the measured data points are shown as individual data points linearly spaced apart to show the individual measurements. Generally, it would be expected that one or more measurements or measurement points a-n of any measurement set 2110, 2120, 2130, 2140, 2150, 2160, 2170, 2180 and 2190 may be the same or substantially the same.

In addition, each of the illustrated measurement points 2110a-2110n may represent an accumulated value taken over multiple transmission frequencies (and/or phase) measurements (e.g., frequencies f₁, f₂, f₃, f₄, as shown in FIGS. 14A-14D). For example, measurement set 2120 and 2130 represent similar measurements taken, in this illustrated case, at a first known rate (e.g., quarterly) during a first or initial period (i.e., first year).

In accordance with the principles of the invention, an average or median value (i.e., 2110m) associated with measurement points 2110a-2110n of measurement set 2110 may be calculated to determine an alcohol content associated with measurement set 2110. Similarly, median values 2120m, 2130m associated with measurements 2120a-2120n, 2130a-2130n, respectively, may be calculated to represent an overall alcohol content for measurement sets 2120, 2130, respectively. Similar average or median values may be determined for each of the measurement sets 2140-2190.

Accordingly, a median alcohol content for each of the illustrated measurement sets 2110-2190 may be determined and utilized to determine a progression of alcohol content of the liquid or fluid within a container without the need to interrupt the process by taking conventional measurements.

Further illustrated, with regard to measurement set 2160 are measurements 2160b-2160n associated with antenna 220b-220n. In this illustrated example, as evaporation or absorption of the fluid within the container occurs, it may be determined that a signal transmitted by antenna 220a provides no useful information and, thus, a signal from antenna 220a is not transmitted nor included within the calculations of an overall alcohol content (i.e., 2160m). A similar selection of antenna is shown by measurement sets 2170-2190 where signals associated with antenna 220b are neither transmitted nor included within the calculation of overall alcohol content (e.g., 2170m-2190m).

In accordance with the principles of the invention, the measurements of fluid loss (and determination of alcohol content) may be taken concurrently at a first rate (e.g., quarterly) during a first period of time (e.g., first and second year) while subsequent measurements of fluid loss and alcohol content may be taken concurrently at a second rate (semi-annually, annually) during a second (e.g., 2^{nd} and 3^{rd} year, 4^{th} and 5^{th} year) period of time, wherein the second rate is longer than the first rate. That is, the periodicity of the measurement rate (i.e., first rate and second rate) may increase over time.

For example, during a first year, when alcohol production is greatest, measurements may be taken at a first rate (e.g., measurement sets 2110, 2120 . . . 2160) and when alcohol production is less, measurements may be taken at a second rate (e.g., 2170, 2180, 2190), wherein the first rate is higher (i.e., measurements performed more often) than the second rate.

Alternatively, the measurement of fluid loss and alcohol content progression may be taken asynchronously wherein fluid loss may be determined at a fluid loss first and second rate as previously discussed and alcohol content progression may be determined at an alcohol progression first and second rate, wherein the fluid loss rates and the alcohol progression rates are different.

Further illustrated is a statistical formulation of the measured sets 2110m-2190m as represented by dashed plot line 2195. Dashed plot line 2195 represents a model that may be utilized to refine the models shown in FIG. 20A-20D. For example, a plurality of plot lines 2195, taken from a corresponding measurements of a plurality of containers that have similar characteristics may be accumulated and included in the model shown in FIGS. 20A-20D.

FIG. 21, similar to FIG. 18, illustrates the variation in the time period between measurements that may occur for the measurement of an alcohol content associated with the processing shown in FIG. 19. As previously discussed, the time period of measurement for alcohol content may occur concurrently with the time period of measurement for fluid level shown in FIG. 18. However, it would be recognized that the time period for alcohol content determination may be different than the time period of measurement for fluid level. Thus, the two processes shown may operate concurrently or independently without altering the scope of the invention claimed.

In accordance with one aspect of the invention, the transmitted signal discussed may comprise a Frequency Modulated (FM) signal, in which the frequency of the transmitted signal is varied (i.e., modulated) during the duration of the transmitted signal. In this manner, the fluid loss, as discussed, may more accurately be determined, as the alteration or change of the transmitted frequency during a specific period provides for different time of returns of the transmitted frequency. Thus, the signals during each of the transmissions 570, 580, 590, etc., shown in FIG. 5B, may comprise a plurality of frequencies (i.e., frequency modulate), wherein the corresponding return windows may be varied based on the transmitted frequency. For example, the transmission signal within a transmission may be continuously varied from a first frequency to a second frequency Alternatively, the transmission signal with a transmission may be discretely altered in a known pattern (e.g., f1, f1+x, f1+2x, etc.) in still another aspect the frequencies within a transmission may be varied (i.e., modulated) in a pseudo-random pattern (e.g., f1, f1+x, f1−2x, f1−x, . . . , etc.). In this manner the time of the return signals associated with each of the transmitted signals may be varied (i.e., modulated) based on the transmitted frequency. In one aspect of the invention, the transmitted signals may be transmitted as Frequency Modulated Continuous Wave (FMCW) signals. In another aspect of the invention, the transmitted signals may be transmitted as pulse signals within a frequency of each of the pulses within a transmission is modulated. In still another aspect, each of the pulses within a transmission may comprise a “chirp” signal, wherein the frequency of a transmitted signal is varied during the transmission of a pulse signal.

In one aspect of the invention, the range of frequency variation may vary based on a determined distance, as will be discussed. In another aspect of the invention, the starting frequency value for each of the signal transmissions 1105, 1115 (FIG. 11A) or the transmissions 570, 580 . . . within each of the transmissions 1105, 1115 . . . (FIG. 11B) may be varied. Alternatively, the pattern of frequency modulation with signal transmissions 1105, 1115 . . . or transmissions 570, 580 with transmission 1105, 1115 . . . may be varied.

Although FIGS. 11A and 11B illustrate time variable transmissions, it would be recognized that the interval being transmissions may be increased, decreased or remain steady over time.

In one aspect of the invention, an average value of the time of the return signals may be determined to determine an overall time that may be used to determine at least one of a fluid level, a mash level and a solid material level. Alternatively, a filtering of the return times may be made to remove those times that are not representative of returns. For example, the highest return time and the lowest return time may be removed from any calculation of an overall time based on the remaining return times.

In accordance with the principles of the invention, a measure of different content of a container may be determined based on different returns of the modulated signal transmission. For example, in a content comprising both a fluid, a mash (i.e., fluid plus solid content) and a solid content, the signals returns associated with the different content may vary based on the transmitted frequency. For example, selected frequency transmissions may result in returns that are reflected from the fluid, selected other frequency transmission may result in returns that are reflected from the mash and selected other frequency transmissions may result in returns that are reflected from the content.

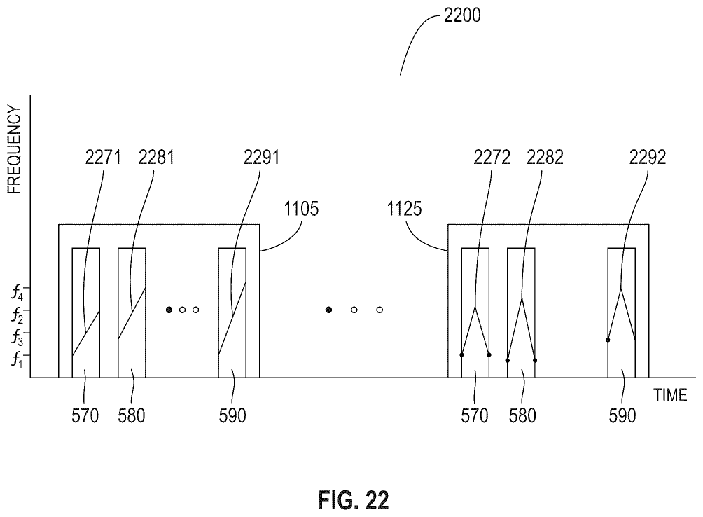

FIG. 22 illustrates an exemplary example of a transmission sequence in accordance with the principles of the invention.

In the illustrated exemplary transmission sequence 2200, which is similar to the sequence shown in FIGS. 11A-11C, transmissions are shown as a function of time (X-axis) and frequency (Y-axis). As shown in FIG. 22, transmissions 570, 580 . . . 590 within a single burst transmission 1105 may comprise one or more transmission frequency changes, wherein the transmission frequency is varied. For example, during transmission 570 during interval 1105 the signals emitted vary between frequency f1 and f2. Similarly, during transmission 580 of interval 105, the signals transmitted or emitted vary between f3 and f4. Similar frequency variation occur for transmission 590. In the illustrated examples, the frequencies are transmitted, within a single transmission, in a continuous pattern (e.g., sawtooth).

In another example, shown with regard to burst transmission 1125, the frequencies within a single transmission 570, 580, . . . 590 may be modulated, which in this case is shown as sinusoidally or triangularly modulated (e.g., upward and downward).

Further illustrated is the starting and stopping frequencies within each of the single transmission 570, 580, . . . 590 within a corresponding burst transmission 1105, 1125 may be different in the selection of the different starting frequencies (e.g., f1, f3, etc.), the rate of frequency modulation and the type of frequency modulation (e.g., sawtooth, triangular, pseudo-random etc.) may be determined, in part, based on the type of material (i.e., fluid, mash, solid) that is being evaluated. For example, for a container containing only fluid (e.g., water tower), a single same starting frequency may be selected wherein the modulation may be one of unmodulated or modulated using a sawtooth pattern. In another example, wherein both fluid and mash are expected with a container, a plurality of frequencies may be selected, wherein the starting frequency and modulation type may be varied to provide diversity to the timing of the return frequencies may be evaluated to obtain a picture of the container (i.e., level of mash and level of fluid atop the mash).

As discussed with regard to FIG. 5B, a return window may be established for each of the transmitted frequencies, wherein the timing of the return window may increase, or decrease based on the transmitted frequency and the expected content.

As would be appreciated by those skilled in the art, the return window may be initially set as a large number (to accommodate an empty tank) and decreased as the tank fills with one or more of a fluid, a mash or a solid content, as the level of the contained content (e.g., solid content) would not decrease (only increase) over time. Hence, the expected return window time may be decreased as the return signal is expected is a shorter time.

In one aspect of the invention, the system shown in FIGS. 7A and 7B may comprise a single transmission antenna to transmit a plurality of signals at different frequencies and frequency ranges (or a plurality of antennae if different frequency ranges require different antenna capabilities) and a receiving antenna to receive a plurality of signals at different frequencies and frequency ranges (or a plurality of antennae if different frequency ranges require different antenna capabilities). The receiving antenna and the transmitting antenna may be separated wherein the transmitting antenna transmits a signal into the container at an angle wherein the reflection is returned to the receiving antenna at substantially the same angle. The angle of transmission may further be altered as the level of the content within the container increases. As the steepness of the angle of transmission decreases (i.e., from an axis perpendicular to the monitoring system), a limit factor may be established such that an alarm is generated when the steepness of the transmission angle falls below a threshold. Thus, as the angle of transmission decreases from 70 degrees to 20 degrees with regard to an axis perpendicular to the monitoring system, an alarm may be trigged to indicate the level of fill of one or more of the content within the container.

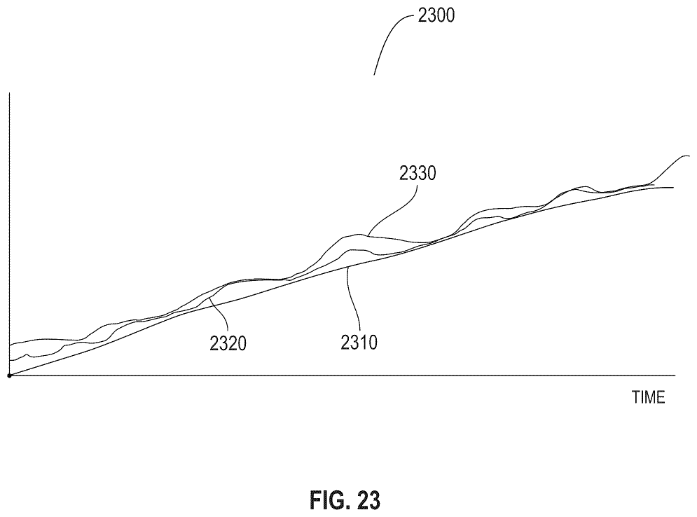

FIG. 23 illustrates an exemplary chart of the collection of one or more materials within a container monitored by the external monitoring system disclosed herein.

In this exemplary chart 2300, solid content 2310 accumulates over time from a zero value to a greater value. The solid material generally increases over time as there is no means for removing the material during the monitoring period. The amount of a mash material, i.e., a mixture of fluid and solid material 2320 similarly over time. However, the amount of mash material increases randomly over time as the fluid within the mash is dispensed, while the remainder of the mash material becomes of a more solid content as the mash settles on-top of the solid material and the fluid is removed. The amount of fluid material similarly increases over time. However, because means for removing the fluid material from the container may exist, the amount of fluid also decreases over time such that the fluid first mixes with the mash material and is later dispensed.

In one aspect of the invention, the frequencies of transmission shown in FIG. 22, may be based, in part, on the content of the container. For example, in a new configuration transmission signals may comprise a single frequency or range of frequencies as the expected content is limited to fluid, whereas the number of frequencies and/or ranges (and the variations of frequencies and ranges) may increase as different types of content are detected within the container. Thus, as solid content increases, a frequency and/or frequency range applicable to solid content may be introduced into the transmission sequence. Returning to FIG. 22, transmissions 570, 580 . . . 590 within burst transmission 1105 may include only a single frequency (or frequency range) transmission whereas the transmissions 570, 580, 590 within burst transmission 1125 may include a plurality of frequency (and/or frequency ranges). The starting frequencies of the plurality of transmitted signals may be selected to be the same or different.

Although FIG. 22 illustrates a plurality of transmitted signals 570, 580, . . . 590, if would be recognized that in aspects of the invention, wherein only a signal transmitting antenna is utilized (see FIG. 1, for example), then only a signal transmitting may occur during each interval. Alternatively, a plurality of transmitted signals may occur during each interval wherein the transmitted signal is the same. The number of transmitted signals and the frequency of each interval may be adjusted to conserve power (in a battery-operated configuration).

The level of solid content and/or the fill rate may be monitored such that a condition of the tank or container may be determined and potential issues regarding overflow, for example, may be averted. In one aspect of the invention, the information collected from the level of the content within the tank or container may, as previously discussed, be provided to one or more external or remote sites such that the information is provided to appropriate personnel.

Hence, in the application of the external monitoring systems disclosed in FIGS. 7A and 7B, which has been discussed with regard to fluid measurement and alcohol content within a barrel or container, it would be recognized that the configuration shown in FIG. 1, for example, would be application to other systems that are arranged in this manner.

For example, a conventional septic system, wherein in a measurement of a solid content within the septic system may be determined. In another application of waste management, wherein large amounts of waste product are contained in containers (or pools), a measurement of the solid content within the container may be used to determine when appropriate dredging operations are to occur. In addition, the external monitoring system disclosed may further provide information regarding a rate of fill or collection of solid materials. In addition, the information collected by the external monitoring system disclosed may provide an indication that the health of a collection system, when the collection system is performing not performing properly. For example, a determined level of fill of solid content may trigger a message that the system is near capacity and cleaning and/or removal of the solid material is needed.

In another aspect, the maintenance of fluid within the system may be an indication of the improper egress of the fluid from the system.

In one aspect of the invention, and as previously discussed, information regarding the level of solid content, the level of fluid level and/or the health of the system may be transmitted to one or more external devices to provide information to those persons needing such information to manage the system (e.g., homeowner, site managers, etc.).

In one aspect of the invention, and as discussed previously, the measurement of content (i.e., fluid and/or solid material) can be recorded and provided to external (or remote) monitoring systems over time such that a projection of fill rate may be determined. The projection, which may be adjusted and/or modified, over time provides a means for determining potential failures. In addition, the collected recorded data may be used to provide information regarding regulatory compliance with required environmental monitoring. The providing of the determined data may be transferred to one or more external systems through one or more of: a wired connection and a wireless connection (e.g., Wi-Fi, BLUETOOTH, etc.). In still another aspect, the collected data may be retained locally and downloaded onto tangible medium (e.g., USB drive) for subsequent processing by one or more external or remote devices.

Although the example provided with regard to employing the external monitoring system for the monitoring of content (e.g., fluid, mash, solid waste) of a septic system, it would be understood that the system disclosed would be applicable to other systems. For example, in a commercial wastewater management systems (or underground storm drains, retention pond water levels, etc.) measurements made using the disclosed system may provide information to be used in determining with required regulatory compliance standards and sustainability. The external monitoring system disclosed would also be applicable to fluid storage systems (e.g., storing oil, fuel, chemical and water tanks). Or to agricultural systems such as irrigation reservoirs, manure pits or livestock waste.

In accordance with the principles of the invention, the non-invasive monitoring system provides a modern, reliable and low maintenance solution for tracking fluid/waste content, which replaces outdated invasive methods or devices (e.g., float sensors, manual inspection). The non-invasive system provides a more reliable, lower cost solution to the content management that ensures detection of early problems, reduced maintenance costs and improved environmental compliance.

Although the monitoring systems disclosed, herein, have made reference to the use of radio frequency transmission, it would be recognized by those skilled in the art that the signals transmitted may be in the ultra-sonic range. Transmission of ultra-sonic signals may be suitable for systems that are an area in which radio frequency transmission is not suitable or allowed. Signals transmitted in an ultra-sonic frequency range may be transmitted as discussed using steady transmission or frequency modulated transmissions.

Various implementations have been disclosed with reference to the Drawings. However, other implementations are possible.

Implementation 1. A method for determining an alcohol content of a fluid within a barrel, the method comprising the steps of: transmitting at least one signal from each antenna within a set of antenna selected from among a plurality of antenna positioned externally to the barrel into the barrel; receiving a response associated with the transmitted at least one signal, determining a change in at least one characteristic between corresponding transmitted at least one signal and the received response; determine a change of a characteristic between the at least one transmitted signal and a corresponding received response; and determine from the determined change in the characteristic an alcohol content of the fluid within the barrel based on a mapping of an alcohol content with respect to the change in characteristic.

Implementation 2. The method of implementation 1, wherein the step of determining a change in characteristic comprises the steps of: accumulating the change in characteristic between each of the at least one transmitted signal and the corresponding received response; and setting the determined change in characteristic as the accumulated change in characteristics.

Implementation 3. The method of implementation 2, wherein the step of determining a change in characteristic comprises the steps of: averaging the accumulated change in characteristic between each of the at least one transmitted signal and the corresponding received response; and setting the determined change in characteristic as the average accumulated change in characteristic.

Implementation 4. The method of implementation 1, wherein the characteristics is selected from at least one of: a signal strength, a frequency shift, and a phase shift.

Implementation 5. The method of implementation 1, wherein the step of determining a change in characteristic comprises the steps of: determining an average value of the change in characteristic for each of the at least one signal for each of the antenna within the set of antenna; determining an alcohol content for each of the average values; and determining an overall alcohol content based on the determined alcohol content for each of the average values.

Implementation 6. The method of implementation 1, comprising: identifying among the plurality of antenna, an antenna positioned within a range of the fluid within the barrel; and selecting the identified antenna as being within the set of antenna.

Implementation 7. The method of implementation 6, wherein the step of identifying comprises the steps of: transmitting from the plurality of antenna, a measurement signal into the barrel; receiving a return signal of the transmitted measurement signal; determining a signal strength of the received return signal, and identifying an antenna associated with a signal strength greater than a threshold value as being within the range of the fluid.

Implementation 8. The method of implementation 1, transmitting at least one signal from each antenna comprises the steps of: transmitting at a first rate during a first period of time; and transmitting at a second rate during a second period of time.

Implementation 9. The method of implementation 8, wherein the first rate is greater than the second rate.

Implementation 10. A system for determination of an alcohol content of a fluid within a barrel, the system comprising: a plurality of antenna positioned external to the barrel; and a transmission/reception system configured to: transmit at least one transmission signal within at least one frequency band from a set of antenna selected from the plurality of antenna; receive a return signal associated with′ the transmitted at least one transmission signal; and determine a change in at least one characteristic between the at least one transmission signal and a corresponding return signal; and determine from the determined change in the at least one characteristic an alcohol content of the fluid based on a mapping of alcohol content with respect to the change in the at least one characteristic.

Implementation 11. The system of implementation 10, wherein the change in the at least one characteristic comprises at least one of: a signal strength change, a frequency change or a phase change.

Implementation 12. The system of implementation 10, wherein the transmission/reception system is configured to: determine the change in the at least one characteristic as an average of the change in the at least one characteristic over each determined change in the at least one characteristic.

Implementation 13. The system of implementation 10, wherein the transmission/reception system is configured to: determine an average change in the at least one characteristic as: an average value of the change in the at least one characteristic over each of the at least one determined change in the at least one characteristic for corresponding ones of the antenna within the set of antenna; and an average of the average values.

Implementation 14. The system of implementation 10, wherein set of antenna comprises at least one antenna associated with a determined level of fluid within the barrel.

Implementation 15. The system of implementation 14, wherein the system is configured to: transmit a measurement signal into the barrel from an antenna selected from the plurality of antenna; receive a return signal associated with the measurement signal; determine a signal strength of the return signal; and assign, when the signal strength is greater than a threshold value, a corresponding antenna selected from the set of antenna.

Implementation 16. A system for determining an alcohol content of a fluid within a barrel, the system comprising: a plurality of antenna arranged at known locations on a face of the barrel; and a processing system comprising: a transmitting system and a receiving system in communication with each of the plurality of antenna; and a processing system configured to: receive, from the receiving system, information regarding a signal transmitted into the barrel by the transmitting system, wherein the information is associated with the signal transmitted to determine a level of fluid within the barrel and an alcohol content of the fluid within the barrel, wherein a determination of the level of fluid comprises: causing transmission of a measurement signal from each of the plurality of antenna; receiving a response associated with the transmitted measurement signal; and determining at least one factor associated with the received response; and assigning, when the at least one factor is greater than a threshold value, a corresponding antenna to a set of antenna; and wherein the determination of the alcohol content comprises: causing the transmission of at least one signal from each of the antenna within the set of antenna; receiving at least one return signal in response to the transmission of the at least one signal transmission; determining a change in a characteristic between the at least one signal transmission and a corresponding return signal; and determining an alcohol content associated with the fluid within the barrel as a function of a mapping of alcohol content with respect to the change in the characteristic.

Implementation 17. The system of implementation 16, wherein the processing system is further configured to periodically perform the determination of fluid level and alcohol content, and wherein a rate of performance of the determination of fluid level and alcohol content may be the same or different.

Implementation 18. The system of implementation 16, wherein the mapping of alcohol content with respect to change in characteristic is established on an individual transmission frequency basis.

Implementation 19. The system of implementation 16, wherein performance of the determination of alcohol content is performed starting with a lowest positioned antenna within the set of antenna.

Implementation 20. The system of implementation 16, wherein performance of the determination of alcohol content is performed starting with a highest positioned antenna within the set of antenna.

For example, an exemplary method may comprise receiving information regarding an initial state of a liquid in a container, the initial state comprising at least one of: an alcohol measure and a level of the liquid within the container; monitoring a level of the liquid within the container, wherein the monitoring is performed external to the container; determining an alcohol content of the liquid within the container based on the initial level; and estimating the alcohol measure of liquid remaining within the container. In addition, a measure of the loss of fluid in the container may be used to limit the measurements taken to only those configurations that would provide useful information in determining the alcohol content.

The method may further comprise storing a plurality of estimated alcohol measures.

The method may further comprise extrapolating an expected alcohol measure based on the stored plurality of estimated alcohol measures.

Estimating the alcohol measure may further comprise adjusting the estimated alcohol measure based on at least one environmental condition.

The at least one environmental condition may be selected from a group consisting of: temperature, location within a facility, a geographic location of the facility, and container condition.

The determination of estimating the alcohol measure may be performed periodically.

The rate of periodicity estimating the alcohol measure may be adjustable.

The rate of periodicity estimating the alcohol measure may be increased as a function of time.

An exemplary system may comprise a plurality of antennas positioned on an exterior surface of a barrel, wherein the plurality of antennas are configured to capture signals reflected by a liquid within the barrel; and a processor configured to: receive the reflected signals; and determine a change in at least one characteristic of the return signal, wherein based on the determined change in the at least one characteristic an alcohol content may be determined. The processor may be further configured to: determine a loss of liquid within the barrel based on the determined level of fluid and an initial level of fluid; and limit the signal transmission from those antenna that would provide useful information in the estimation of an alcohol content of the liquid within the barrel.

The system may further comprise the processor may be configured to store the estimated alcohol content.

The system may further comprise the processor may be configured to extrapolate an expected alcohol content based on a stored plurality of estimated alcohol measures.

The system may further comprise the processor may be configured to adjust the estimated alcohol content based on at least one environmental condition.

The at least one environmental condition may be selected from a group consisting of: temperature, location within a facility, a geographic location of the facility, and container condition.

The determination of estimating the alcohol content may be performed periodically.

The rate of periodicity estimating the alcohol content may be adjustable.

The rate of periodicity estimating the alcohol content may be adjustable as a function of time.

An exemplary method may comprise: determining, by a monitoring system external to a container, an estimation of an alcohol content of the liquid within the container based on the a determination of one or more characteristic associated with one or more signals transmitted within the barrel, wherein the estimation comprises: determining a change in at least one characteristic at a known frequency (or phase); obtaining a first order alcohol content based on a model expectation of alcohol content at the known frequency (or phase); and determining the alcohol content based on adjusting the first order alcohol content based accumulating a plurality of first order alcohol content associated with different locations of fluid within the barrel or container.

The method may further comprise projecting an estimated alcohol content based on a plurality of the determined alcohol content.

The method may further comprise determining the alcohol content of the liquid within the container periodically, wherein measurements of the alcohol content are performed at a first rate during a first period of time and at a second rate during a second period of time, the first rate being faster than the second rate. The first rate and the second rate of the transmission of signals for the determination of alcohol content may be based, at least in part, as previously discussed with regard to the transmission of signals for the determination of fluid level. For example, the first rate may occur during a first period of time and the second rate may occur during a second period of time. The first rate may be greater than the second rate. For example, during a period of expected rapid change in alcohol content measurement associated with the determination of alcohol content, measurements may occur once/week, whereas during a period of expected slowing of the change in alcohol content, the determination of alcohol content may occur once/month, semi-annually, etc.

It would be understood and recognized that the first and second rates associated with fluid level measurement and alcohol content may be same or different. In addition, it would be recognized that the measurement of fluid level and alcohol content may be performed periodically wherein the period of measurement of fluid level and the period of determination of alcohol content may be the same or different. That is, fluid level measurement and alcohol content measurement may be performed at the same time and the same rates. Alternatively, fluid level measurement and alcohol content measurement may be performed at different times and at different rates.

In summary, the presented invention, provides for the determination of alcohol production progression during the distilling of an alcohol based liquid within a container without causing any interference with the alcohol production by the need to physically test the liquid, wherein the measure of alcohol with the container is based on a system that may be attached to a face of a container, that causes the transmission of one or more signals in at least one frequency range into the container, where the transmitted signals that are reflected off the fluid or liquid contained within the container are captured and evaluated to determine a level of the fluid or liquid within the tank. A measure of the alcohol content is then based on the determination of the loss of fluid or liquid.

The system disclosed achieves technical advantages over the prior art as the invention disclosed remains external to the enclosed system (barrel, etc.) and does not affect the internal ecosystem or contents of the barrel.

In addition, a method associated with the present invention is disclosed, wherein the method comprises the steps of: transmitting at least one signal into said tank; receiving a response associated with selected ones of said transmitted at least one signal; and evaluating said received response associated with selected ones of said transmitted at least one signal, wherein said evaluation comprises: determining a signal strength of each of said received response; selecting at least two of said received responses, wherein said selected responses are associated with a highest signal strength; and determining said fluid level based on a relationship between said selected at least two of said received responses.

In addition, a method associated with the present invention is disclosed wherein the method comprises the steps of: obtaining an initial alcohol content and level of a fluid within a container and obtaining measurements of the fluid level over time to evaluate and determine a loss of fluid due to one of evaporation and absorption, computing an expected alcohol content based on the initial alcohol content and the loss of fluid and further adjusting the expected alcohol content by one or more environment factors associated with at least the conditions surrounding the storage of the fluid.

Although various features have been described with reference to the Figures, other features are possible. For example, a device implementation in accordance with the present disclosure may comprise modular units with a varying thickness print flex antenna across a barrel face. The device may be implemented with a custom-designed PCB motherboard configured to be mounted in the middle of the barrel face. The device may comprise radar and radio frequency chips and a separate data transceiver module. The data transceiver module may be configured to operate using BLUETOOTH, LORAWAN or another band protocol. The device may be configured with a defined power source, for example a C1, D2 certified single core battery. The device may be attached to the face of an enclosed system (e.g., a whiskey barrel) with the printed antenna arrays located with reference to a defined position of a watch/barrel face. The antenna arrays may be located with reference to the center point of the watch/barrel face. The devices may be adhered or attached to the barrel face with an adhesive or attached with composite fasteners (screw/nail/staples, or the like).

The device may be configured to use a combination of Millimeter Wave (MM Wave) and or Radio Wave (RF), and/or other direct analog measurement methodologies to determine the liquid substrate level behind a barrel face. Liquid-level measurements may be relayed to multiple central communications hubs via BLUETOOTH, LORAWAN or any other communications technology, depending on the distance from the barrel to central device. From the central device, measurement data may be exported out of the rickhouse via satellite, cellular, or fiber connection to the cloud or a handheld device. A device implementation deployed on a barrel may be configured to broadcast measurement data packets from the barrel to the central device and from there exported out of the rickhouse via satellite, cellular, or fiber connection to the cloud or a handheld device configured to collect the measurement data packets exported from the central receiving device.

The device implementation may be configured to account for the introduction of foreign bodies or materials such as wooden staves, woods chips, or anything else that would displace the liquid level. For example, software may be configured to account for the displacement measurement and the displacement differential of any object inserted into the liquid to maintain an accurate measurement. In an illustrative example, the displacement and/or differential measurement software implementation may have a foreign body displacement measurement mode that determines displacement differential between liquid levels measured at different points in time, that is, before and after a foreign body is introduced to the container. The device implementation may incorporate the use of RFID to connect the device to software to track the device/barrel location in a “rickhouse.”

The device implementation may use MM Wave, RF Wave, or another lower frequency or band as needed. This radar may be a low enough frequency to ensure penetration of the wood or the material associated with the container. The signal that is transmitted into the barrel by the antenna would be reflected back at levels where the liquid is present, in contrast with no reflections from levels where the liquid is not present. This group of reflections and non-reflections produces a total measured signal that is processed by the device to determine an estimate of the height of the liquid-air interface.

In an illustrative example, a device implementation may be configured to determine liquid level measurements in a horizontal rick storage mode. For example, a horizontal rick storage mode implementation may be configured to measure the liquid level over time as it relates to where any substrate is in contact with the barrel face as well as the liquid-air interface. Such an implementation will be able to determine fluid volume at any given period. Distillers are required by law to log exactly how many proof gallons they put into any barrel at any time. The device implementation may be calibrated by inputting the exact amount of whiskey/tequila/spirits/etc. (substrate) reported to all required international governmental agencies on to the device, permitting the device to measure the differential of evaporation over time (AKA “The Angels Share”). In an illustrative example, the device implementation can then determine loss over time based on how antennas read the liquid-air interface behind each antenna. In this example implementation, the device is directly measuring the difference in liquid level between points of a varying printed antenna design as well as any liquid-air gaps in the antenna array which may vary in size and orientation.

In another illustrative example a device implementation may be configured to determine liquid level measurements in a vertical palletized storage mode. For example, a vertical palletized storage mode implementation may be configured to measure the reflection between the waves as it pertains to liquid content of an aging barrel. In this mode one or more antennas will reflect waves downward through the barrel face and measure the reflection time between device and barrel, device and substrate, device and barrel bottom as well as any materials inserted or placed in the barrel. This measurement may calculate the distance and relative length of the wave and convert that measure into an accurate measure of substrate. Some waves will go through the barrel and never return and will be disregarded. The device may be configured to only interpret what the device knows as operative space and measure total volume.

The device implementation may be a combination of a peel and stick design and/or with a potential non-metal/composite screw/staple/nail or fastening device that would allow distillers to adhere/attach the device to the barrel face at the time of barrel fill.

During barrel fill distillers may be required by law to exactly track and log the amount of liquid put in the barrel as stated above. All barrels may not be filled to the same fill level or amount. Accordingly, one or more calibration steps may be performed, as described herein. Connecting the device to the barrel and the system may benefit from calibration to ensure correct and accurate measurements. In an illustrative example a software application may be configured to uniquely associate the barrel to the device for the barrel's primary lifespan (these could be sent to a secondary market). For example, a unique hardware identifier for a barrel may be associated in a database with a unique identifier for an instance of the measurement device disclosed herein. In such an example, particular calibration data determined for the barrel/measurement device pair may be uniquely associated with the measurement device in the database, permitting the calibration and measurement performance of the device to be tracked over time.

As these “rickhouse” environments are quite harsh, a very strong adhesive or other fastening device may be used to adhere/attach the device to the barrel face in both horizontal (traditional rick storage) and vertical (palletized) storage options. We may also encase the designed PCB board and all of the components in a strong epoxy resin potting material or other hard casing to protect all electronics from any potential damage. Damage could be from forces like bumps, scrapes, dings to whiskey leaking on top and heat and/or humidity.

Once the barrel is filled and calibrated, the device is capable of providing a near absolute liquid level measurement. Barrels may range in total volume (the industry average is a 53-gallon barrel which will vary in finished size.) Barrels can be filled above 53 gallons. In an illustrative example the device may adhere/attach to the barrel face in the same fashion regardless of barrel size or storage options such as horizontal rick storage and palletized storage. Antenna arrays can vary in size and orientation based on the size of the barrel face as the barrels vary in total surface volume.

After filling, barrels may be moved to their storage locations where they will sit for varying periods of time. Because of this the device design may comprise a single core ATEX certified battery system which will give a potential life span between 6-10 years. In an illustrative example the device may be configured to satisfy a fire safety class 1 div 2 classifications according to DISCUS, NEC, and as well as ATEX class 2. Keeping fire safety in mind, the single core battery may be used because the single core battery traditionally has a slower discharge rate than reusable or rechargeable batteries. The device may be configured to ping only once a month, every month for the life span of the device or barrel, to conserve battery energy.

The device may be configured to be in communication with a central receiver. The central receiver may be configured in communication with other sensors such as ambient temperature and humidity. Once the device is pinged from the central receiver, the device will activate; once activated the device programming will cause the device to follow distinct operation sequences for horizontal storage and palletized storage device implementations.

In an illustrative example a device implementation designed for a horizontal storage mode in a traditional rickhouse may be configured to perform operations comprising: the device will activate an RF signal which goes across the antenna array; the device will measure exactly the differential of what is behind the barrel head and any relation to the space liquid-air differential between antennas across the clock face of the barrel and the device; as well as the relation of what's behind the wood to our antenna array will allow for volume measurement.

In an illustrative example a device implementation designed for a palletized storage mode may be configured to perform operations comprising: the device will activate in a similar manner as the horizontal storage mode implementation but rather with an MM signal. The device will fire downward and register the wavelength and reflection between device, barrel face, liquid, barrel bottom, and any particulate inside the barrel; the device will then interpret the total space of liquid contained and a measurement will be calculated.

All measurements will be saved in platform for the distiller or end user to make both qualitative and quantitative inferences. These qualitative and quantitative inferences may be used to calculate predictions for Barrel Yield, Tax Planning, Barrel Provenance, and Supply Chain planning.

If a distiller can understand exactly where their total run volume stands more accurately than current industry models of 2-4% loss per year they can make better decisions and inferences on metrics such as barrel performance as it relates to the quality of a cooperage (barrel maker), how any potential variable may affect a barrel such as heat, humidity, any coating material or R&D experiment. Knowing the volume of barrel can allow distillers to make many decisions to both increase efficiency and reduce industrial waste.

Another value add is that with the accurate volume, distillers can work with their insurance provider to reduce potential premiums as well as make sure that they are neither under-insured nor over-insured. They would just be adequately insured for loss.

Potential Yield: In the pursuit of optimizing production, distilleries need to and want to accurately gauge the volume of whiskey in each barrel. This not only helps in maximizing the yield from each batch but also in efficiently utilizing resources. Precise measurements allow for better supply chain forecasting and planning, ensuring that each step of the distillation and aging process is conducted with the utmost efficiency. Also helping with yield as it pertains to number of bottles and cases for their distributors.

Tax Planning: The taxation on distilled spirits can be complex, and it's based, in part, on the volume of product produced and stored. Accurate barrel measurements are essential for distilleries to comply with tax regulations accurately. This precision helps avoid over or underpayment of taxes, which can have significant financial implications. By knowing exactly how much whiskey is in each barrel, distilleries can file more accurate tax returns, thus avoiding potential legal and financial issues. There are major benefits to knowing your PGs (proof gallons) as tax rates do change from around $2.85 and $13.25 once a distillery crossed a set limit (100,000 PGs or roughly 1886 barrel) taxes increase.

Provenance: from its distillation to its aging-knowing volume and history adds to the product's allure and value consumers will pay. Precise barrel measurement contributes to the detailed tracking of each batch's journey, ensuring that the provenance is well-documented and authentic. This level of detail enriches the narrative of the whiskey, providing whiskey enthusiasts with a deeper appreciation of its heritage and quality.

The device may be implemented with a flex tail antenna array that will cover the clockface or in a wagon wheel design of a whiskey barrel that is adhered by a durable adhesive or composite fastener. RF and MM wave chips may be used to determine the liquid levels. The device may include a fire safety approved battery. The device may be configured with multiple interfaces to push data both into and out of the device. The device may be encased in a hard epoxy potting or protective casing. The device may be configured with BLUETOOTH, LORAWAN or another communication band to carry data in and out of the device.

The device remains external to the barrel and does not impede the aging process. The device lowers labor cost over handheld devices and is more accurate. The device is also on the face of the barrel; thus, the barrel can be rolled without the device having to be removed.

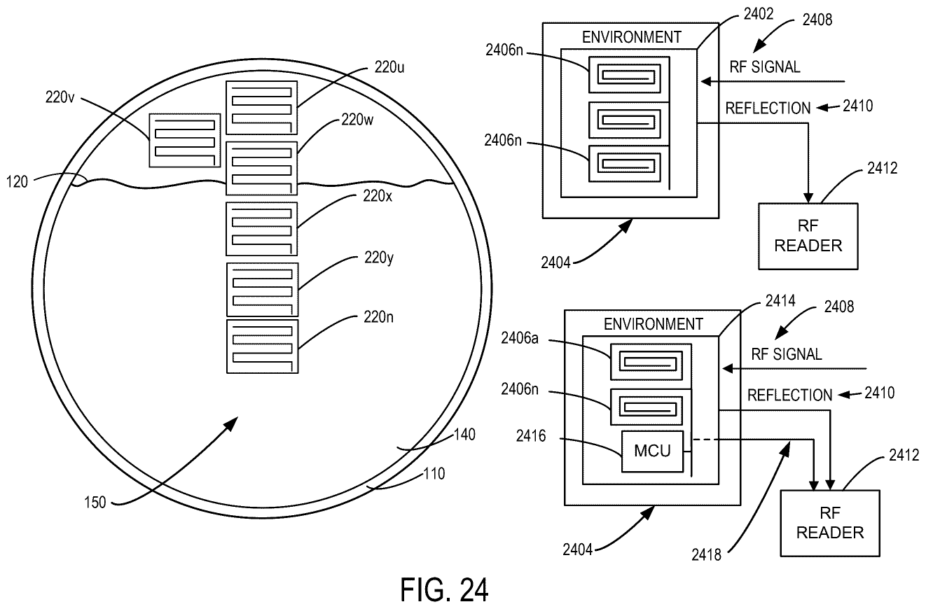

Referring to FIG. 24, a system for obtaining internal conditions of a container is illustrated in accordance with examples of the present disclosure. In some aspects, FIG. 24 depicts a container 140, which may be a wooden cask, barrel, or other sealed liquid-containing vessel having an exterior wall 110. The container 140 holds a liquid 120, such as a distilled spirit, wine, or other liquid product. The liquid 120 occupies a portion of the interior volume of container 140, creating a liquid-air interface or liquid level at a specific height within the container.

In some aspects, a plurality of radio frequency (RF) responsive elements 220u, 220v, 220w, 220x, 220y, and 220n are disposed externally on the wall 110 of container 140. These RF-responsive elements 220u-220n may be arranged in a substantially vertical array along the exterior surface of the container, with each element positioned at a different height relative to a bottom portion of the container 140. The RF-responsive elements 220u-220n may include passive RFID tags, semi-passive tags, metasurface structures, resonant circuits, or other RF-interactive devices configured to interact with incident RF signals in a manner that is influenced by the adjacent internal environment within the container.

In some aspects, each RF-responsive element 220u-220n includes an antenna structure configured to receive RF signals and to reflect, backscatter, or otherwise interact with these signals. The RF-responsive elements 220u-220n may be configured to respond to signals within various frequency bands, such as ultra-high frequency (UHF) bands commonly used for RFID applications, sub-6 GHz frequencies used in cellular or Wi-Fi communications, or other suitable RF bands. The design of the antenna associated with each RF-responsive element 220u-220n may be optimized to enhance sensitivity to dielectric variations on the opposite side of the container wall.

In some aspects, the RF-responsive elements 220u-220n are strategically positioned such that some elements may be adjacent to portions of the container containing liquid 120, while other RF-responsive elements 220u-220n may be adjacent to portions containing only air and/or vapor. As the liquid level changes over time due to processes such as evaporation, absorption, or consumption, different RF-responsive elements 220u-220n become aligned with the liquid-air interface.

To the right side of FIG. 24, a schematic representation illustrates one or more operational principles of an RF-responsive element 220u-220n. In some aspects, the RF-responsive element 2402 represents an example RF-responsive element similar to elements 220u-220n mounted on the container. The RF-responsive element 2402 is shown in relation to its environment 2404, which represents the surrounding conditions that influence the RF signal interactions. Within the RF-responsive element 2402, the internal components 2406a-2406n (such as antennas, resonators, or other RF-interactive structures) interact with incoming RF signals. These components may be configured in various arrangements to optimize their response to environmental changes. In some aspects, each RF-responsive element 2402 includes internal components 2406a-2406n, which may comprise antenna structures, loops, coils, inductors, resonators, metamaterials, or other RF-interactive components designed to receive and interact with RF signals. The configuration and composition of these internal components 2406a-2406n may determine how the RF-responsive element 2402 interact with incident RF signals and how they are affected by the adjacent environment 2404. The internal components may be tuned to specific frequencies or designed with particular geometries to enhance their sensitivity to environmental changes on the opposite side of the container wall.

In some aspects, an RF signal 2408 is transmitted toward the RF-responsive element 2402 from an external source. The RF signal 2408 may be generated by an RF reader 2412 or other transmitting device. The RF signal 2408 may comprise continuous waves, modulated signals, or pulsed transmissions configured to interact with the internal components 2406a-2406n of the RF-responsive element. When the RF signal 2408 encounters the RF-responsive element 2402 and its internal components 2406a-2406n, a portion of the signal is reflected, backscattered, or reradiated, producing a reflected signal 2410 that returns to the RF reader 2412. The characteristics of this reflection signal 2410, such as its amplitude (received signal strength indicator or RSSI), phase, resonant frequency, or channel state information (CSI), may be influenced by the environment 2404 adjacent to the RF-responsive element.

The RF reader 2412 receives and processes the reflection signal 2410 to extract information about the internal condition of the container. By analyzing the collective response of the vertically arranged RF-responsive elements 220u through 220n and their respective internal components 2406a-2406n, the system can determine where the transition occurs between liquid-influenced and air-influenced responses, thereby identifying the liquid level 120 within the container 140. That is, the system operates on the principle that an RF-responsive element 2402 positioned adjacent to a liquid-backed portion of the container wall will interact with RF signals differently than an RF-responsive element 2402 that may be located adjacent to an air-backed portion, due to the different dielectric properties affecting the internal components 2406a-2406n of each element. This non-invasive monitoring approach allows for continuous or periodic measurement of liquid levels without breaching the integrity of the container or disturbing its contents. The system represented in FIG. 24 provides a means for monitoring the internal contents of sealed containers by leveraging the interactions between RF signals, the specially designed internal components of the RF-responsive elements, and the varying environments they encounter.

FIG. 24 additionally depicts a variation of the RF-responsive element 2414, which includes an integrated microcontroller unit (MCU) 2416 configured to locally process signal information and generate characteristics 2418 corresponding to one or more properties of the received RF signal. In some aspects, the RF-responsive element 2414 is not merely passive but may exhibit active or semi-passive capabilities, such as sensing, signal processing, or data logging.

In operation, an RF signal 2408 (which may be an RF interrogation signal) is transmitted toward the environment 2402, which may include a liquid-filled container 110 having a surface 120 and outer wall 140. As the RF signal 2408 interacts with the environment, a reflected signal 2410 is returned to the RF reader 2412. Simultaneously, RF-responsive element 2414 receives the RF signal and internally evaluates signal features such as RSSI, signal phase, frequency offset, CSI, or channel quality metrics using its onboard components, including MCU 2416. In some aspects, the MCU 2416 processes these internally measured signal characteristics and generates corresponding data values 2418, which may be stored locally or encoded into a backscattered response. These values 2418 may represent, for example, the RSSI as measured at the RF-responsive element 2414, or signal integrity parameters indicative of the propagation conditions through the surrounding medium (e.g., air, vapor, or liquid). In some implementations, the RF-responsive element 2414 may also sense temperature, humidity, or vibration, and include such data in the measured characteristics 2418.

In some aspects, the characteristics of the reflected signal 2410—as measured by the RF reader 2412—may be compared with the internally sensed characteristics 2418 measured by RF-responsive element 2414. By analyzing differences or correlations between these two sets of signal data, the system can improve diagnostic accuracy, validate environmental readings, or identify anomalies such as signal interference, tag detuning, material seepage, or unexpected liquid ingress. For example, if the RSSI measured by the RF-responsive element 2414 significantly deviates from that observed at the RF reader 2412, the system may infer that signal absorption or multi-path distortion is occurring in the monitored environment. Such discrepancies can be indicative of evolving conditions, such as fluid level changes, material degradation, or external contamination.

In some implementations, MCU 2416 may support additional logic or learning algorithms that allow RF-responsive element 2414 to autonomously detect patterns or generate alerts based on predefined thresholds or historical trends. These features enable improved sensing architectures, particularly for applications involving long-term container monitoring, such as barrel aging, chemical storage, or environmental exposure analysis.

In certain embodiments, the RF-responsive element 2414 may refer to a sensing component that is configured to generate a signal indicative of at least one characteristic of an environment, substance, or system under observation. As used herein, “indicative of” generally encompasses situations in which the generated signal reflects, represents, or is correlated to a given property without necessarily providing a direct, one-to-one measurement. For example, the sensing component may output an analog signal that undergoes post-processing or interpretation, where even a subtle change in resonant frequency or waveform amplitude can reveal underlying information about a fluid's dielectric constant, temperature, or other relevant parameters. In this sense, a frequency shift, phase variation, or coded output may each qualify as “indicative of” the property in question, so long as the signal conveys information that may be interpreted or mapped to the characteristic.

Moreover, in various aspects, the phrase “at least one characteristic” may not limit the system to measuring only a single property. A single sensing component or an array of sensing components may detect multiple attributes—such as fluid level, density, composition, or dielectric constant—and still satisfy scenarios in which only one selected property is processed or analyzed. That is, additional measured attributes may be incorporated, thereby supporting expanded functionality without departing from the fundamental principle that a generated signal need only correlate with one or more properties of interest. Whether the signal is a digital bitstream, an analog waveform, or a frequency response curve, the signal may be transformed, filtered, or otherwise analyzed to derive meaningful insights regarding the measured environment or substance.

In certain implementations, a sensing component is configured to generate a signal indicative of at least one dielectric property associated with a fluid stored within the container. By “associated with the fluid,” it is understood that the signal may reflect how the fluid's presence, composition, or level influences the electromagnetic or electrostatic environment in proximity to the sensing module. For instance, if the fluid partially fills the container, the interface between the fluid and the surrounding headspace can induce a measurable change in resonant frequency, impedance, or signal strength, thereby serving as an indirect measure of fluid level. Conversely, when the fluid's composition or concentration shifts—such as through evaporation, absorption, or chemical exchange with the container material—these alterations produce corresponding variations in the fluid's dielectric response, which the sensing component can capture and convey. Thus, a system may generate a signal (e.g., an active signal, a reflected signal, etc.) that is indicative of at least one dielectric property associated with the fluid, such that a system can conduct at least one of a fluid-level detection (where the fluid boundary modulates the measured property) or a composition-based sensing (where changing fluid characteristics modify the dielectric constants being measured).

In some aspects, the fluid contained in a container is subject to composition-based or environment-driven changes resulting from interactions with the container itself. These changes may arise, for example, as the fluid undergoes chemical exchange with the inner surface of the container, experiences variations in temperature or pressure, or is exposed to differing levels of humidity or ambient gases. In certain scenarios, the fluid's concentration, chemical composition, or physical properties (e.g., viscosity, dielectric constant, or pH) can shift over time due to absorption, evaporation, oxidation, or other reactions facilitated by the container's material.

Moreover, these environment-driven or composition-based alterations may occur progressively, even without direct human intervention, as a function of storage duration, atmospheric conditions, or intrinsic material properties of the container. As an illustrative example, a wooden barrel used for aging spirits or wine may impart flavor compounds or cause water-ethanol exchange that changes the fluid's flavor profile, alcohol concentration, and coloration. In other instances, a polymeric or metallic container might introduce subtle chemical interactions affecting the fluid's longevity or stability. Regardless of the specific mechanism, these changes collectively contribute to time-varying characteristics of the fluid, offering opportunities for monitoring, analysis, or adaptive control.

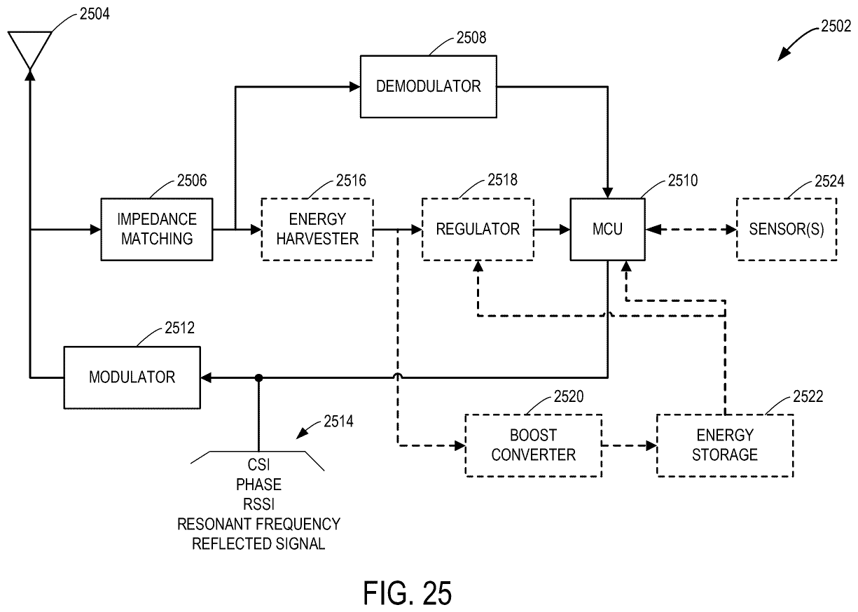

FIG. 25 illustrates a block diagram of an exemplary architecture 2502 of a radio-frequency (RF) sensing device that may be implemented in various embodiments of a non-invasive sensing system for monitoring internal conditions of containers in accordance with examples of the present disclosure. The RF sensing device may be the same as or similar to the RF-responsive element 220u-220n and/or RF-responsive element 2402 of FIG. 24. The architecture 2502 may be configured to operate in passive, semi-passive, or active modes depending on the specific implementation and power requirements of the sensing application.

An RF sensing device may include an antenna 2504 configured to receive and/or transmit RF signals in communication with external reading devices. The antenna 2504 may comprise a suitable antenna structure including, but not limited to, dipole configurations, patch arrangements, loop structures, microstrip designs, fractal patterns, or other antenna geometries selected based on operating frequency, spatial constraints, gain requirements, and directional characteristics. In some aspects, the antenna 2504 may be designed as a conformal structure that can be adhered to curved surfaces such as barrel staves or container walls. The antenna 2504 may be tuned to operate in various frequency bands including ultra-high frequency (UHF) ranges commonly used in RFID applications (e.g., 860-960 MHz), industrial, scientific and medical (ISM) bands (e.g., 2.4 GHz or 5.8 GHz), or other frequency ranges suitable for penetrating container materials and/or interacting with contained liquids. In some aspects, the antenna 2504 may serve dual functions in many implementations: receiving interrogation signals from external reading devices and/or reflecting or transmitting response signals that carry sensed information.

In some aspects, the antenna 2504 may be coupled to an impedance matching network 2506, which may optimize power transfer between the antenna 2504 and the device's internal circuitry. The impedance matching network 2506 may comprise passive components such as capacitors, inductors, transformers, transmission line sections, or active components that dynamically adjust matching parameters based on operating conditions. In some aspects, the impedance matching network 2506 may minimize reflection losses by providing a conjugate match to the RF input impedance of downstream components, thereby maximizing the power transferred from the antenna to the processing circuitry or, conversely, from the device back to the external reader. In some aspects, the impedance matching network 2506 may be designed with a variable or switchable configuration to accommodate different operational modes or frequency bands.

In some aspects, a demodulator 2508 may be coupled to the impedance matching network 2506 and may extract baseband information from the incident RF carrier signal. The demodulator 2508 may implement various demodulation techniques including amplitude shift keying (ASK), phase shift keying (PSK), frequency shift keying (FSK), or more complex schemes such as quadrature amplitude modulation (QAM) depending on the communication protocol employed. The demodulator 2508 processes the received RF signal to recover command and control data that may include timing instructions, sensor polling requests, configuration parameters, or other information transmitted from an external reader. In some aspects, the demodulator 2508 may incorporate signal conditioning components such as filters, amplifiers, or level shifters to improve signal quality before demodulation.

In some aspects, the demodulated signal from the demodulator 2508 may be provided to a microcontroller unit (MCU) 2510, which may operate as a central processing element of the RF sensing device. The MCU 2510 may be implemented using a microcontroller (typically low-power), processor, application-specific integrated circuit (ASIC), field-programmable gate array (FPGA), system-on-chip (SoC), or other suitable processing element configured to coordinate the operation of the sensing and communication components. The MCU 2510 executes firmware or software instructions to manage power consumption, process sensor readings, control modulation parameters, and implement application-specific algorithms for environmental sensing. In some aspects, the MCU 2510 may incorporate sleep modes, wake-up timers, or event-triggered processing to minimize power consumption during periods of inactivity.

In some aspects, the RF sensing device may include a modulator 2512 coupled to the MCU 2510 and the antenna 2504, which may be configured to encode information in the reflected or transmitted signal. The modulator 2512 may operate by varying the impedance of the antenna 2504 in response to control signals from the MCU 2510, thereby modulating the backscattered signal in a process known as load modulation. Alternatively, or in addition, in active implementations, the modulator 2512 may generate an actively transmitted waveform carrying the encoded information. The modulation may encode identification data, sensor readings, signal metrics, or other information derived from the RF interaction or sensor measurements. The modulator 2512 may implement various modulation schemes including amplitude modulation, phase modulation, frequency modulation, or combinations thereof to efficiently encode information while maintaining compatibility with reader systems.

Example information 2514 indicates various signal characteristics that may be sensed, modulated, or encoded by the RF sensing device. These characteristics include, but are not limited to, channel state information (CSI), which provides a fine-grained frequency-domain profile of the RF channel including amplitude and phase information across multiple subcarriers; signal phase information capturing the relative carrier phase between transmitted and received signals, which is sensitive to propagation delay; received signal strength indicator (RSSI), which provides an indication of power level received or reflected; resonant frequency measurements, which may shift based on changes in nearby dielectric loading such as liquid level behind the tag, and the reflected signal. These signal characteristics may be processed by the MCU 2510 to infer information about the environment surrounding the RF sensing device or may be transmitted to an external reader for further analysis.

In some aspects, an energy harvester 2516 may be coupled to the impedance matching network 2506 and functions to convert a portion of the received RF energy into DC power for operating the device. The energy harvester 2516 may include rectifier circuits (e.g., using Schottky diodes, CMOS transistors, or other semiconductor devices), voltage multipliers (e.g., Dickson, Villard, or Cockcroft-Walton configurations), and impedance-matching networks optimized for power conversion rather than signal integrity. In some aspects, the energy harvester 2516 may be designed to operate efficiently across multiple frequency bands or to extract energy from ambient RF sources other than the primary interrogation signal. The efficiency of the energy harvester 2516 may vary based on factors such as input power level, frequency, polarization, and impedance matching accuracy. The harvested energy from the energy harvester 2516 may be provided to a regulator 2518, which conditions the power for use by the device's active components. The regulator 2518 may include voltage stabilization circuits, current limiting protection, and noise filtering to provide a stable DC supply voltage despite variations in harvested power. In some implementations, the regulator 2518 may incorporate multiple output voltages to serve different components with different power requirements or may implement dynamic voltage scaling to balance performance and power consumption based on available energy.

In some aspects, the architecture 2502 may include a boost converter 2520 coupled to the regulator 2518 or directly to the energy harvester 2516. The boost converter 2520 steps up the voltage level from the harvested energy to meet the requirements of downstream components or to efficiently charge an energy storage element. The boost converter 2520 may implement inductor-based, capacitor-based (charge pump), or transformer-based topologies depending on efficiency requirements, space constraints, and voltage conversion ratios. An energy storage element 2522 may be coupled to the boost converter 2520 or directly to the regulator 2518, providing temporary energy storage to support operation during periods without sufficient incoming RF power. The energy storage element 2522 may comprise capacitors, supercapacitors, thin-film batteries, or other rechargeable storage technologies selected based on capacity requirements, leakage characteristics, cycle life, and physical constraints. In semi-passive implementations, the energy storage element 2522 enables the device to perform sensing or processing operations independent of the availability of an interrogation signal, thereby extending the functionality of the device.

The architecture 2502 may incorporate one or more sensors 2524 coupled to the MCU 2510, which gather data about the environment adjacent to the RF sensing device. These sensors 2524 may include temperature sensors (e.g., thermistors, resistance temperature detectors, digital temperature sensors), humidity sensors, pressure sensors, ethanol vapor detectors, moisture sensors, accelerometers, or other environmental sensors relevant to the application. In some aspects, the RF characteristics themselves may serve as implicit sensors, with the MCU 2510 analyzing parameters such as antenna resonance, impedance, or signal propagation to infer environmental conditions without dedicated sensor components. The sensors 2524 may operate on various principles including resistive, capacitive, piezoelectric, optical, or electrochemical sensing mechanisms, selected based on the parameter being measured and power constraints.

In operation, the architecture 2502 enables dual-mode RF sensing whereby the device receives a downlink RF signal, measures its characteristics, and either backscatters or actively transmits a return signal to a reader. Changes in the environment, such as but not limited to variations in liquid level, vapor concentration, or container wall moisture, modulate both the received signal characteristics and the reflected signal, enabling inference of internal conditions based on comparative analysis. This dual-path sensing approach provides enhanced sensitivity and reliability compared to single-parameter measurement methods, particularly in challenging environments such as wooden barrels or other containers with variable material properties.

The architecture 2502 may support various operating modes, including continuous monitoring, periodic sampling, or event-triggered operation, depending on power availability and application requirements. In passive implementations, the device operates solely from harvested RF energy during reader interrogation. In semi-passive configurations, the energy storage element 2522 provides power for sensing and processing between reader interrogations while still using backscatter for communication. In active implementations, the device may transmit signals independently using harvested or stored energy. The architecture 2502 may be implemented using discrete components, integrated circuits, system-in-package technologies, or custom ASICs depending on production volume, size constraints, and performance requirements. The physical implementation may be optimized for conformal mounting on container surfaces, with considerations for environmental protection, adhesion methods, and minimizing impact on container aesthetics or functionality.

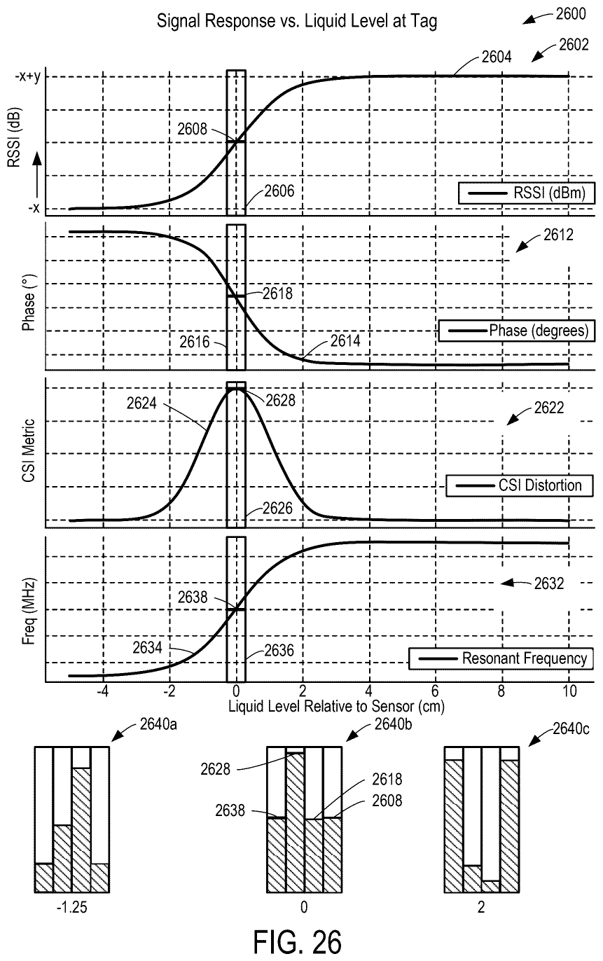

Referring now to FIG. 26, a signal response profile diagram 2600 illustrating various radio frequency (RF) signal characteristics as functions of liquid level relative to an RF-responsive element is depicted according to embodiments of the present disclosure. The diagram 2600 comprises multiple subgraphs arranged vertically, each representing a different signal metric that may be measured or derived when an RF signal interacts with an RF-responsive element positioned externally on a container surface and/or wall.

The top subgraph 2602 illustrates the Received Signal Strength Indicator (RSSI) response curve 2604 plotted as a function of liquid level relative to the RF-responsive element position (measured in centimeters). The RSSI response curve 2604 demonstrates a transition when the liquid level crosses the position of the RF-responsive element (at the 0 cm position). When the liquid level is above the sensor (negative values on the x-axis), the RSSI value remains relatively low as indicated by the substantially flat portion of curve 2604 to the left of position 2606. As the liquid level approaches and passes the sensor position at point 2606 (0 cm mark), the RSSI value increases substantially, as shown by the steep transition region of curve 2604. The RSSI value then stabilizes at a higher level when the liquid level is below the position of the RF-responsive element, as indicated by the substantially flat portion of curve 2604 to the right of position 2608. The characteristic S-curve arises from the transition between low-dielectric air and high-dielectric liquid, which alters the RF signal propagation and reflection, typically leading to changes in RSSI and phase due to increased dielectric loading and absorption. That is, the change in dielectric environment when liquid replaces air behind the RF-responsive element may modify the reflection and absorption characteristics of the signal due to the higher dielectric constant and loss tangent of the liquid, which can lead to measurable changes in signal strength (RSSI), phase, and/or resonance characteristics.

The second subgraph 2612 depicts the phase response curve 2614 as a function of liquid level relative to the position of the RF-responsive element. The phase response exhibits an inverse relationship compared to the RSSI curve. When the liquid level is below the sensor (negative values on the x-axis), the phase value remains relatively high. As the liquid level approaches and crosses the position of the RF-responsive element at point (0 cm mark), the phase value decreases, as shown by the steep transition region of curve 2614. Position 2618 marks a point on the phase response curve where the liquid level is above the position of the RF-responsive element, and the phase has stabilized at a lower value. This phase transition occurs because the propagation characteristics of the RF signal change when interacting with liquid versus air, resulting in a measurable phase shift that can be detected by an RF reader system.

The third subgraph 2622 illustrates the Channel State Information (CSI) metric represented by curve 2624. Unlike the monotonic transitions seen in RSSI and phase responses, the CSI metric exhibits a peak-like behavior centered around the liquid level transition point. At position 2626 (0 cm mark), where the liquid level aligns with the position of the RF-responsive element, the CSI distortion may reach a maximum value as indicated by the peak of curve 2624. Points 2628 represent measurements at the peak where the liquid level at the position of the RF-responsive element. The CSI metric may provide a highly sensitive indicator of the exact liquid-air interface position, as it captures frequency-domain characteristics of the RF channel that are particularly responsive to dielectric boundaries.

The fourth subgraph 2632 shows the resonant frequency response curve 2634 as a function of liquid level. When the liquid level is below the position of the RF-responsive element (negative values on the x-axis), the resonant frequency remains at a baseline value. As the liquid level approaches and crosses the position of the RF-responsive element at 2636 (0 cm mark), the resonant frequency may shift to a different value as shown by the transition region of curve 2634. Position 2638 marks a point where the liquid level is at the position of the RF-responsive element, and the resonant frequency has stabilized at a new value. This frequency shift occurs because the resonance characteristics of the RF-responsive element are influenced by the dielectric properties of the adjacent materials, with liquid causing a measurable shift in the resonant frequency compared to air.

The lower portion of FIG. 26 includes three diagrams 2640a, 2640b, and 2640c, each representing a different liquid level scenario corresponding to specific points on the x-axis of the signal response graphs. The left diagram 2540a shows a liquid level at position −1.25 cm relative to the position of the RF-responsive element. The center diagram 2540b shows a liquid level at position 0 cm, aligned with the position of the RF-responsive element. The right diagram 2640c shows a liquid level at position +2 cm relative to the position of the RF-responsive element.

Together, these diagrams illustrate profile readings based on one or more scenarios corresponding to the signal responses shown in the graphs above. Thus, the signal response profile illustrated in diagrams 2640a, 2640b, and 2640c depicts how multiple RF signal characteristics can be measured simultaneously to provide a determination of liquid level within a container using externally mounted RF-responsive elements. By analyzing combinations of these signal metrics, the system can achieve accuracy and reliability in non-invasive liquid level sensing applications, particularly for wooden casks, barrels, or other containers where traditional invasive sensors would be undesirable. Although the signal responses illustrated from left to right in FIG. 26 reflect a rising liquid scenario, it will be understood that in certain applications, such as aging or evaporation monitoring, the liquid level may initially begin above the sensor and subsequently drop below it. In such cases, the signal response profiles may exhibit inverse transitions. In some aspects, an RF-responsive element may be positioned in the bottom half of a container such that variations in liquid level above the RF-responsive element do not materially affect its signal response. In such configurations, observed changes in signal characteristics may instead be attributable to evolving properties of the liquid itself, such as alcohol by volume (% ABV), color, viscosity, or other dielectric-affecting parameters.

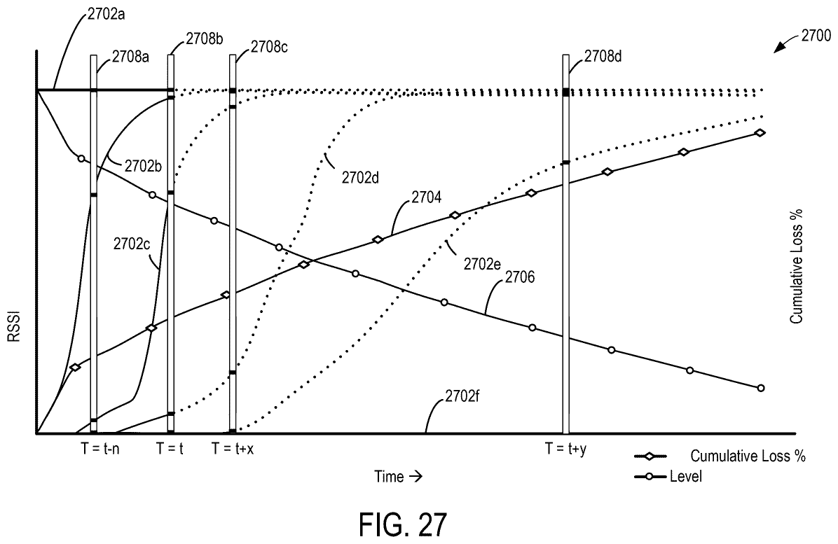

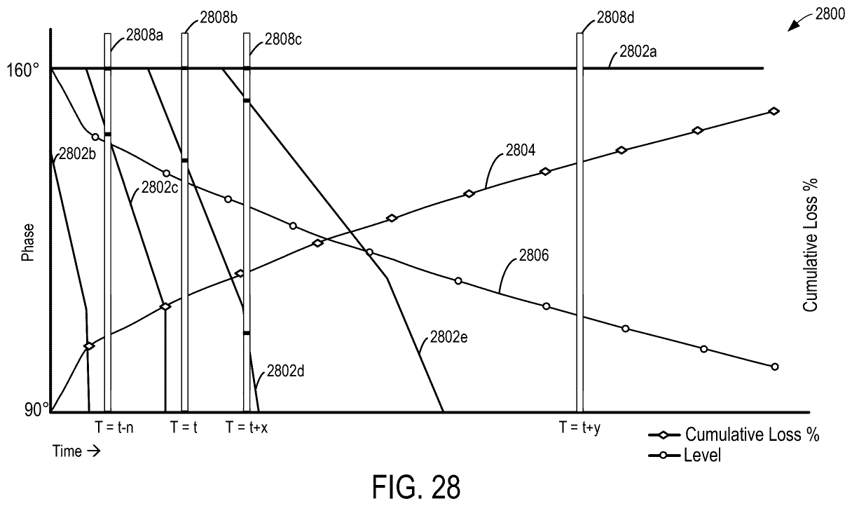

Referring now to FIG. 27, illustrated is a composite time-domain chart 2700 showing signal behavior and environmental change over time for a container monitoring system according to at least one aspect of the present disclosure. The chart 2700 depicts a multi-parameter visualization wherein different signal characteristics and derived metrics are plotted as functions of time, enabling correlation between RF signal responses and physical changes occurring within the monitored container.

The horizontal axis of chart 2700 represents time, advancing from left to right across defined time intervals or measurement events, including timestamps T=t−n, T=t, T=t+x, and T=t+y. These timestamps correspond to successive measurement or sampling points at which the RF-responsive elements interact with an interrogation signal from a reader or base station. The vertical axis on the left side represents RSSI values, while the vertical axis on the right side represents cumulative loss percentage of the liquid within the container. The chart 2700 includes multiple plotted curves 2702a, 2702b, 2702c, 2702d, 2702e, and 2702f (collectively referred to as curves 2702), each representing an RSSI trend associated with an individual RF-responsive element positioned at a different height on the exterior wall/surface of a container. The RF-responsive elements may comprise passive RFID tags, semi-passive sensing transceivers, reconfigurable intelligent surfaces, or other RF-interactive structures configured to modulate, reflect, or backscatter an incident RF signal. These RF-responsive elements may be arranged in a substantially vertical array along the container's exterior surface, with each element having a known position relative to the container's bottom or reference point.

Each curve 2702 exhibits characteristic behavior indicative of the interaction between the corresponding RF-responsive element and its local environment. For instance, the signal strength reflected from an RF-responsive element may change based on whether the element is adjacent to a portion of the container containing liquid, vapor, or air. This change occurs because the dielectric properties of these different media affect the electromagnetic coupling between the RF-responsive element and the incident RF signal. Liquid typically has a higher dielectric constant than air, which can alter the resonance, impedance, or backscatter characteristics of the RF-responsive element.

For example, curve 2702a corresponds to an RF-responsive element positioned near the top of a container. Its relatively flat and high RSSI indicates that the tag remains adjacent to air throughout the measurement period, confirming that the liquid level never reaches it. In contrast, curve 2702b demonstrates a pronounced increase in RSSI over time, starting at a lower signal level (indicating that the RF-responsive element is adjacent to liquid) and rising sharply as the liquid level drops and the tag transitions to being adjacent to air and/or vapor. Similarly, curve 2702c, curve 2702d, and curve 2702e demonstrate a pronounced increase in RSSI over time, starting at a lower signal level (indicating that the RF-responsive element is adjacent to liquid) and rising sharply as the liquid level drops and the tag transitions to being adjacent to air and/or vapor. The dotted lines in FIG. 27 represent a future RSSI reading.

Curve 2704 corresponds to the cumulative loss percentage, which quantifies the volume of liquid that has evaporated, leaked, or been absorbed by the container walls over time. This metric may be calculated based on the change in liquid level (curve 2706) and knowledge of the container's internal geometry. The cumulative loss percentage curve 2704 enables tracking of long-term trends in liquid depletion, which may be particularly valuable for aging processes like spirit maturation or quality control in storage applications.

Curve 2706 represents the inferred liquid level within the vessel over time, which may be derived from the RSSI data of the multiple RF-responsive elements. This liquid level curve 2706 may be computed through various methods, such as threshold detection (identifying which tags exhibit RSSI values above a predetermined threshold), interpolation between adjacent RF-responsive elements, or more complex signal processing algorithms that consider the relative RSSI changes across multiple elements. The liquid level curve 2706 provides a continuous or discrete estimate of where the liquid-air interface is located within the container at any given time.

Curve 2704 corresponds to the cumulative loss percentage, which quantifies the volume of liquid that has evaporated, leaked, or been absorbed by the container walls over time. This metric may be calculated based on the change in liquid level (curve 2706) and knowledge of the container's internal geometry. The cumulative loss percentage curve 2704 enables tracking of long-term trends in liquid depletion, which may be particularly valuable for aging processes like spirit maturation or quality control in storage applications.

Vertical lines 2708a, 2708b, 2708c, and 2708d (collectively referred to as lines 2708) mark specific timestamps or events in the monitoring timeline. Line 2708a may indicate the initiation of monitoring or a calibration event. Line 2708b might correspond to a moment when the RSSI from a particular RF-responsive element (e.g., 2702b) reached a transition value, suggesting alignment with the liquid-air interface. Line 2708c could correspond to a moment when the RSSI from a particular RF-responsive element (e.g., 2702c) reached a transition value, suggesting alignment with the liquid-air interface and the RF-responsive element associated with the line curve 2702c. Line 2708d might correspond to a moment when the RSSI from a particular RF-responsive element (e.g., 2702d) reaches a transition value, suggesting alignment with the liquid-air interface and the RF-responsive element associated with the line curve 2702c.

In some aspects, the temporal relationships between the various signal responses may be used to infer both instantaneous liquid levels and longer-term dynamics such as evaporation rates. For instance, the time delay between inflection points in curves 2702c and 2702d may correspond to the time taken for the liquid level to descend between the respective positions of the corresponding RF-responsive elements. By analyzing these temporal patterns and correlating them with environmental data, the system can estimate various parameters of interest, such as liquid loss rate, vapor-phase development, or compositional changes in the container contents. In some implementations, computational logic or algorithms that track the evolution of RSSI values over time, detect signal features such as peaks, valleys, inflection points, or slope changes, and compute derived metrics like the liquid level (curve 2704) or cumulative loss percentage (curve 2706) may be used. These algorithms may employ statistical techniques, machine learning models, or physics-based simulations to improve the accuracy and reliability of the inferred parameters.

The approach exemplified by chart 2700 can support various types of RF-responsive elements, including passive RF-responsive elements that rely solely on the energy of an incident interrogation signal, semi-passive RF-responsive elements that include a battery or energy harvester to power some functions, or active devices that can transmit signals independently. The RF communication may operate in various frequency bands, such as UHF RFID (860-960 MHz), Wi-Fi (2.4 GHz or 5 GHZ), or cellular bands (e.g., 5G sub-6 GHz or mmWave), depending on the specific requirements of the application. By integrating temporal information with spatial signal patterns, a system can provide comprehensive insights into the internal conditions of a sealed container without requiring direct access to its contents. This non-invasive monitoring approach may be particularly valuable for processes where maintaining container integrity is critical, such as aging of spirits, long-term storage of sensitive chemicals, or quality control in food processing.

In operation, the chart 2700 may be generated either in real-time as new measurements are collected or retrospectively from stored data. The visualization provided by chart 2700 enables operators to observe trends, identify anomalies, and make informed decisions about the monitored container. For example, an unexpected rapid change in the RSSI of one or more RF-responsive elements might indicate a leak or accelerated evaporation, prompting investigation or intervention. Similarly, a gradual flattening of the cumulative loss curve 2706 might suggest that the container has reached equilibrium with its environment, potentially signaling the completion of a maturation process. The monitoring approach illustrated by chart 2700 may be extended to include additional signal characteristics beyond RSSI, such as phase shift, resonant frequency, or channel state information, which may provide complementary information about the container contents. The approach may also be scaled to monitor multiple containers simultaneously, enabling comparative analysis across a set of containers under similar or different conditions.

Referring now to FIG. 28, illustrated is a composite phase-domain chart 2800 showing signal behavior and environmental change over time for a container monitoring system according to at least one aspects of the present disclosure. The chart 2800 depicts a multi-parameter visualization wherein different signal characteristics and derived metrics are plotted as functions of time, enabling correlation between RF phase responses and physical changes occurring within the monitored container.

The horizontal axis of chart 2800 represents time, advancing from left to right across defined time intervals or measurement events, including timestamps T=t−n, T=t, T=t+x, and T=t+y. These timestamps correspond to successive measurement or sampling points at which the RF-responsive elements interact with an interrogation signal from a reader or base station. The vertical axis on the left side represents phase values in degrees, while the vertical axis on the right side represents cumulative loss percentage of the liquid within the container. The chart 2800 includes multiple plotted curves 2802a, 2802b, 2802c, 2802d, and 2802e (collectively referred to as curves 2802), each representing a phase response trend associated with an individual RF-responsive element positioned at a different height on the exterior wall/surface of a container. The RF-responsive elements may comprise passive RFID tags, semi-passive sensing transceivers, reconfigurable intelligent surfaces, or other RF-interactive structures configured to modulate, reflect, or backscatter an incident RF signal with characteristic phase shifts. These RF-responsive elements may be arranged in a substantially vertical array along the container's exterior surface, with each element having a known position relative to the container's reference point.

Each curve 2802 exhibits characteristic behavior indicative of the interaction between the corresponding RF-responsive element and its local environment. Unlike RSSI measurements, phase responses typically decrease when an RF-responsive element transitions from liquid-backed to air-backed states. This phase shift occurs because the propagation characteristics and effective electrical path length change based on the dielectric properties of the adjacent media.

For example, curve 2802a corresponds to an RF-responsive element positioned near the top of a container. Its relatively flat and high phase value (approximately) 160° indicates that the tag remains adjacent to air throughout the measurement period, confirming that the liquid level never reaches this height. In contrast, curve 2802b demonstrates a pronounced decrease in phase over time, starting at a higher phase value (indicating that the RF-responsive element is initially adjacent to air) and dropping sharply as liquid evaporates and the sensor gradually transitions to being adjacent to vapor or air instead of liquid. Similarly, curves 2802c, 2802d, and 2802e demonstrate characteristic phase transitions at their respective heights as the liquid level decreases over time.

Curve 2804 corresponds to the cumulative loss percentage, which quantifies the volume of liquid that has evaporated, leaked, or been absorbed by the container walls over time. This metric increases steadily throughout the monitoring period, reflecting the ongoing evaporation or “angel's share” typical in aging processes like spirit maturation. The cumulative loss percentage enables tracking of long-term trends in liquid depletion, which may be particularly valuable for quality control and inventory management.

Curve 2806 represents the inferred liquid level within the vessel over time, which may be derived from the phase data of the multiple RF-responsive elements. This liquid level curve 2806 may be computed through various methods, such as threshold detection (identifying which tags exhibit phase values below a predetermined threshold), interpolation between adjacent RF-responsive elements with transitioning phase values, or more complex signal processing algorithms that consider the relative phase changes across multiple elements. The liquid level curve 2806 decreases over time, consistent with expected evaporation in a sealed container.

Vertical lines 2808a, 2808b, 2808c, and 2808d (collectively referred to as lines 2808) mark specific timestamps or events in the monitoring timeline. Line 2808a may indicate the initiation of monitoring or a calibration event. Line 2808b might correspond to a moment when the phase response from RF-responsive element 2802b reached a transition value (approximately) 120°, suggesting alignment with the liquid-air interface. Line 2808c could correspond to a moment when the phase response from RF-responsive element 2802c crossed a similar transition threshold. Line 2808d might mark a significant milestone in the aging process, such as a scheduled quality check or sampling event.

In some aspects, the temporal relationships between the various phase signal responses may be used to infer both instantaneous liquid levels and longer-term dynamics such as evaporation rates. For instance, the time delay between inflection points in curves 2802c and 2802d may correspond to the time taken for the liquid level to descend between the respective positions of the corresponding RF-responsive elements. By analyzing these temporal patterns and correlating them with environmental data, the system can estimate various parameters of interest, such as liquid loss rate, vapor-phase development, or compositional changes in the container contents.

In some implementations, a system may incorporate computational logic or algorithms that track the evolution of phase values over time, detect signal features such as transitions, inflection points, or slope changes, and compute derived metrics like the liquid level (curve 2806) or cumulative loss percentage (curve 2804). These algorithms may employ statistical techniques, machine learning models, or physics-based simulations to improve the accuracy and reliability of the inferred parameters.

The approach exemplified by chart 2800 can support various types of RF-responsive elements, including passive RF-responsive elements that rely solely on the energy of an incident interrogation signal, semi-passive RF-responsive elements that include a battery or energy harvester to power some functions, or active devices that can transmit signals independently. The RF communication may operate in various frequency bands, such as UHF RFID (860-960 MHz), Wi-Fi (2.4 GHz or 5 GHZ), or cellular bands (e.g., 5G sub-6 GHz or mmWave), depending on the specific requirements of the application. By integrating temporal information with spatial phase patterns, a system can provide comprehensive insights into the internal conditions of a sealed container without requiring direct access to its contents. This non-invasive monitoring approach may be particularly valuable for processes where maintaining container integrity is critical, such as aging of spirits, long-term storage of sensitive chemicals, or quality control in food processing.

In operation, the chart 2800 may be generated either in real-time as new measurements are collected or retrospectively from stored data. The visualization provided by chart 2800 enables operators to observe trends, identify anomalies, and make informed decisions about the monitored container. For example, an unexpected rapid change in the phase response of one or more RF-responsive elements might indicate a leak or accelerated evaporation, prompting investigation or intervention. Similarly, a gradual flattening of the cumulative loss curve 2804 might suggest that the container has reached equilibrium with its environment, potentially signaling the completion of a maturation process.

The monitoring approach illustrated by chart 2800 demonstrates how phase measurement complements RSSI data, providing an additional dimension of signal characteristics that can enhance detection sensitivity. This approach may be further extended to include other parameters such as resonant frequency or channel state information, which may provide complementary information about the container contents. The approach may also be scaled to monitor multiple containers simultaneously, enabling comparative analysis across a set of containers under similar or different conditions.

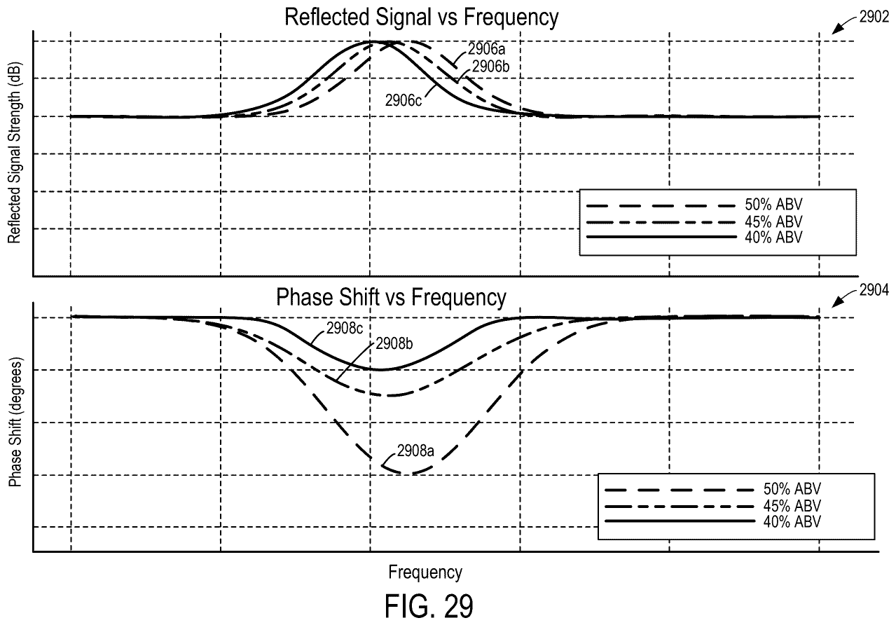

Referring now to FIG. 29, illustrated are frequency-domain plots showing how reflected radio frequency (RF) signal characteristics vary as a function of alcohol content within a liquid adjacent to an externally affixed RF-responsive element in accordance with aspects of the present disclosure. A system may implement frequency-dependent reflection and phase behavior to determine or estimate the alcohol content of liquid contained within a vessel such as a cask, barrel, or tank, without requiring direct contact with the liquid. FIG. 29 comprises two separate graphs arranged in vertical alignment. The upper graph 2902 depicts reflected signal strength as a function of frequency, while the lower graph 2904 shows phase shift as a function of frequency for corresponding measurements.

In the upper graph 2902, the vertical axis represents reflected signal strength measured in decibels (dB), and the horizontal axis represents frequency. The graph 2902 illustrates how the amplitude of reflected RF signals exhibits characteristic frequency-dependent behavior that correlates with the alcohol content of the liquid within the container. Three distinct curves 2906a, 2906b, and 2906c are shown, each corresponding to different alcohol by volume (ABV) percentages. Specifically, curve 2906a represents the reflected signal amplitude profile associated with liquid having approximately 50% ABV, curve 2906b represents the reflected signal amplitude profile associated with liquid having approximately 45% ABV, and curve 2906c represents the reflected signal amplitude profile associated with liquid having approximately 40% ABV.

Each of the curves 2906a, 2906b, and 2906c exhibits a resonant response characterized by a peak amplitude at a specific frequency. The resonant frequency at which this peak occurs shifts systematically as a function of the liquid's alcohol content. This frequency shift phenomenon occurs because the dielectric properties of the liquid vary substantially with ethanol concentration. For instance, curve 2906a (corresponding to 50% ABV) shows a resonant peak at a higher frequency compared to curve 2906c (corresponding to 40% ABV). This relationship between resonant frequency and alcohol content provides a basis for non-invasive determination of the liquid's alcohol concentration through analysis of the frequency-domain characteristics of reflected RF signals.

The lower plot 2904 illustrates corresponding phase shift measurements across the same frequency range. The vertical axis represents phase shift measured in degrees, while the horizontal axis again represents frequency. Similar to the amplitude plot 2902, the phase plot 2904 includes three distinct curves 2908a, 2908b, and 2908c, corresponding respectively to liquids with 50% ABV, 45% ABV, and 40% ABV. Each of the phase curves 2908a, 2908b, and 2908c demonstrates a characteristic phase transition or “dip” centered approximately at the resonant frequency identified in the amplitude plot 2902. The depth and sharpness of these phase transitions correlate with the ethanol concentration of the liquid. For example, curve 2908a (50% ABV) exhibits a deeper and more pronounced phase dip compared to curve 2908c (40% ABV). Additionally, the frequency position at which the phase dip occurs shifts in a manner consistent with the shifts observed in the amplitude resonance peaks, providing complementary information that may enhance measurement reliability or precision.

The frequency-domain measurements depicted in FIG. 29 may be obtained using various RF sensing architectures. In some embodiments, the system may utilize passive or chipless RF-responsive elements (e.g., RFID tags), resonant backscatter devices, or frequency-selective surfaces mounted externally on the container surface. In other embodiments, the system may employ active transceiver modules capable of swept-frequency measurements or orthogonal frequency division multiplexing (OFDM) techniques to capture the full complex-valued frequency response of the liquid-container system. The characteristic frequency-dependent behaviors shown in FIG. 29 enable non-invasive compositional analysis of the container contents. By analyzing the precise frequency position of resonant peaks in the amplitude response (curves 2906a, 2906b, 2906c) or the location and depth of phase transitions (curves 2908a, 2908b, 2908c), a system can determine alcohol concentration without requiring physical samples to be extracted from the container. This approach preserves the integrity of the aging or storage process, which may be particularly valuable in applications such as spirit maturation where container breaching is undesirable.

In some implementations, the frequency-domain signatures may be processed using reference-based comparison, wherein measured frequency responses are matched against a calibrated database of known alcohol concentrations. In other implementations, machine learning algorithms may be trained to recognize the spectral features corresponding to specific ABV levels from the full complex-valued frequency response. Such approaches may compensate for variations in container materials, geometry, or environmental conditions that might otherwise affect measurement accuracy. The frequency-domain analysis depicted in FIG. 29 may be performed at discrete time intervals to track changes in alcohol concentration over extended periods. Such temporal monitoring may enable observation of evaporation dynamics, ethanol-to-water ratio shifts, or loss of volatile compounds over time in aging spirits or other liquids, providing valuable insights into maturation processes without disturbing the container contents.

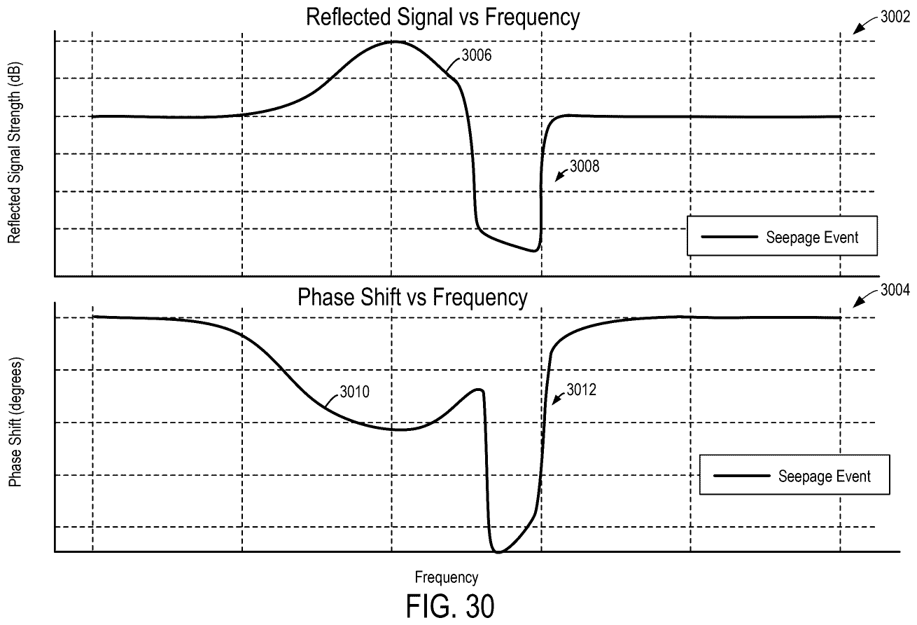

Referring now to FIG. 30, illustrated are frequency-domain plots showing how reflected radio frequency (RF) signal characteristics vary as a function of moisture or seepage conditions detected by an externally affixed RF-responsive element in accordance with aspects of the present disclosure. FIG. 30 comprises two separate graphs arranged in vertical alignment, showing complementary signal metrics that may be analyzed to detect anomalous conditions within a container. The upper graph 3002 depicts reflected signal strength as a function of frequency. The vertical axis represents reflected signal strength measured in decibels (dB), and the horizontal axis represents frequency. The graph 3002 illustrates how the amplitude of reflected RF signals exhibits characteristic frequency-dependent behavior that correlates with the presence of seepage or moisture migration within the container wall or at the exterior-interior boundary. The solid line represents a seepage event signature, characterized by a resonant response with a peak amplitude 3006 at a specific frequency followed by a pronounced signal depression 3008. This resonant profile differs substantially from normal operating conditions, where the reflected signal may exhibit more uniform amplitude across the measured frequency range.

The specific shape and characteristics of the reflected signal profile can provide information about the nature and extent of the seepage event. For instance, the height of peak 3006 relative to the baseline signal level, the width of the resonant peak, the depth of depression 3008, and the sharpness of transitions between these features may correlate with factors such as the extent of moisture penetration, the composition of the leaking fluid, or the progression of the seepage event over time. In some implementations, these characteristics may be quantified through parameters such as quality factor (Q-factor), peak-to-valley ratio, or spectral moments to enable automated detection and classification of seepage events.

The lower graph 3004 illustrates corresponding phase shift measurements across the same frequency range. The vertical axis represents phase shift measured in degrees, while the horizontal axis again represents frequency. Similar to the amplitude plot 3002, the phase plot 3004 depicts the characteristic signature of a seepage event. The phase response exhibits a gradual transition 3010 followed by a sharp discontinuity 3012, creating a distinctive profile that can be detected through phase-sensitive measurements.

The RF-responsive elements that generate these signal characteristics may be implemented using various technologies. For example, they may comprise passive RF-responsive elements (e.g., RFID tags), resonant circuits, metasurfaces, or other RF-interactive structures capable of reflecting incident RF signals with characteristic modulation of amplitude and phase. These elements may be designed with specific frequency responses that maximize sensitivity to the dielectric changes associated with moisture penetration or liquid seepage through container walls. The RF-responsive elements may be affixed to the exterior surface of the container in locations likely to experience seepage or moisture accumulation, such as near joints, seams, or areas subject to mechanical stress.

The frequency-domain measurements depicted in FIG. 30 may be particularly valuable for early detection of container integrity issues before they progress to observable liquid leakage. By analyzing the precise frequency position, magnitude, and shape of resonant features in both amplitude and phase responses, a monitoring system can identify subtle changes in the container wall's moisture content or the development of microfractures that might eventually lead to leakage. This approach enables preventive maintenance or intervention before significant product loss occurs. In some implementations, the frequency-domain signatures may be processed using pattern recognition algorithms, reference-based comparison, or machine learning techniques to distinguish normal variations in signal characteristics from the specific patterns indicative of seepage events. These computational approaches may compensate for environmental factors, material aging, or other non-fault conditions that might otherwise trigger false alarms. The frequency-domain analysis approach depicted in FIG. 30 may be performed continuously, periodically, or on-demand to monitor container integrity over extended periods. Such monitoring may be particularly valuable in applications involving high-value contents, hazardous materials, or aging processes where container breaches could compromise product quality or safety.

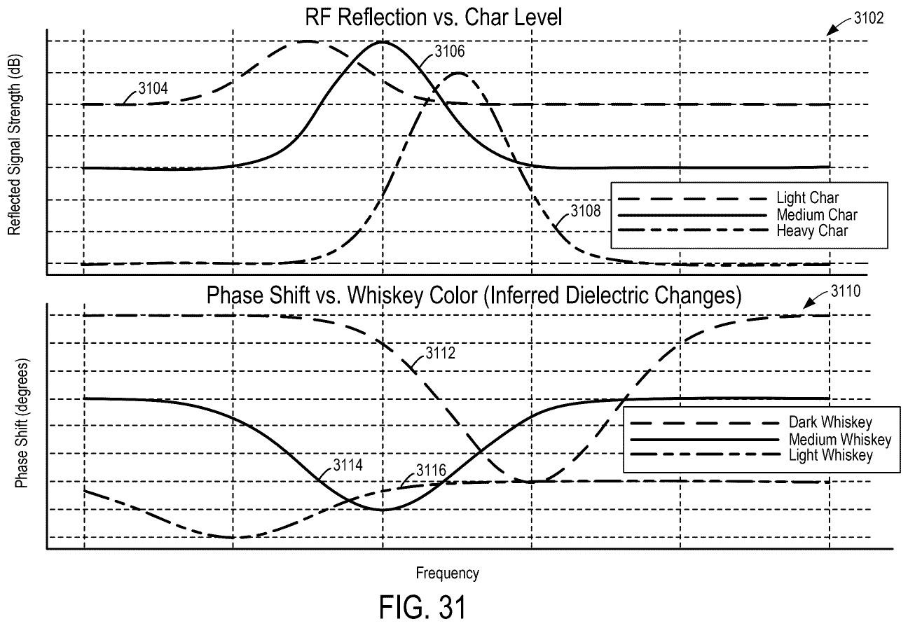

Referring now to FIG. 31, illustrated is a set of frequency-domain signal plots 3102 showing radio frequency (RF) signal characteristics as affected by both char level of a container's interior wall and the color/maturity of a contained liquid such as whiskey in accordance with aspects of the present disclosure. The signal plots 3102 demonstrate the relationship between these physical characteristics and measurable RF signal parameters due to variations in dielectric and electromagnetic properties of the container materials and contained liquid.

The upper plot 3102 presents reflected signal strength as a function of frequency. The vertical axis represents reflected signal strength measured in decibels (dB), while the horizontal axis represents RF frequency. The upper plot 3102 includes three distinct response curves 3104, 3106, and 3108, each corresponding to different char levels applied to the interior surface of the container. Specifically, curve 3104 corresponds to light char, curve 3106 corresponds to medium char, and curve 3108 corresponds to heavy char. The char level, which may be established during container fabrication or reconditioning, influences the electromagnetic response characteristics as depicted by the different reflection profiles. The light char curve 3104 exhibits a relatively flat, broadband reflection pattern, indicating minimal interaction with the RF signal. The medium char curve 3106 demonstrates a well-defined resonant peak, which may result from moderately enhanced surface conductivity and dielectric transition properties associated with the carbonized layer. The heavy char curve 3108 shows a dampened peak with a frequency shift relative to the medium char response, which may be attributed to increased thickness of the carbonized layer, greater absorption characteristics, and enhanced porosity of the heavily charred surface.

The lower plot 3110 depicts phase shift versus frequency behavior under varying contained liquid conditions. In this plot, the vertical axis represents phase shift measured in degrees, while the horizontal axis again represents frequency. The three curves 3112, 3114, and 3116 correspond to different whiskey color profiles, which serve as indicators of chemical composition, aging progression, and interaction between the liquid and the container wall. Specifically, curve 3112 represents dark whiskey, curve 3114 represents medium whiskey, and curve 3116 represents light whiskey. The phase curves demonstrate that changes in the liquid composition-including factors such as ethanol concentration, dissolved tannins, esters, and lignin-derived compounds-result in measurable variations in RF phase response. The dark whiskey curve 3112 exhibits a broader phase transition region and deeper phase roll-off, which may correspond to greater interaction with the container wall and extended aging periods. The medium whiskey curve 3114 shows intermediate phase characteristics. The light whiskey curve 3116 demonstrates sharper phase transitions occurring at comparatively higher frequencies, which may indicate lower dielectric loss and predominance of ethanol in the composition.

The frequency-domain signatures illustrated in FIG. 31 may be utilized by an RF sensing system to determine multiple characteristics of the container and its contents without requiring direct physical access to the interior. For example, the system may infer the container's char level by analyzing the resonance shape and reflection magnitude patterns. Similarly, the system may determine liquid maturity or chemical concentration by evaluating phase shift characteristics. Additionally, the system may track changes over time related to aging processes, refill cycles, or reconditioning treatments by monitoring shifts in these RF response patterns. In various implementations, the RF response profiles shown in FIG. 31 may be processed using spectral analysis techniques, pattern recognition algorithms, machine learning models, or parameterized physical models to quantify char level, estimate maturation progress, or identify container conditioning stages. The actual measurement may be performed using various RF sensing approaches, which may include RF-responsive elements (e.g., passive chipless tags), backscatter radio-frequency identification (RFID) devices, or broadband RF transceivers mounted externally on the container surface. The RF sensing approach illustrated by FIG. 31 provides a non-invasive method for monitoring both container characteristics and liquid properties, which may be particularly valuable in applications where maintaining container integrity is essential, such as in spirit aging, chemical storage, or specialized fluid processing operations.

In certain embodiments, the RF-responsive element may be arranged in the bottom half of the container such that variations in liquid level above the element do not materially affect its signal response. This configuration enables the system to focus specifically on changes in the contained liquid's properties (such as alcohol content, color, or chemical composition) without the confounding variable of liquid level fluctuations, as the element remains consistently adjacent to a liquid below the anticipated evaporation zone throughout the monitoring period.

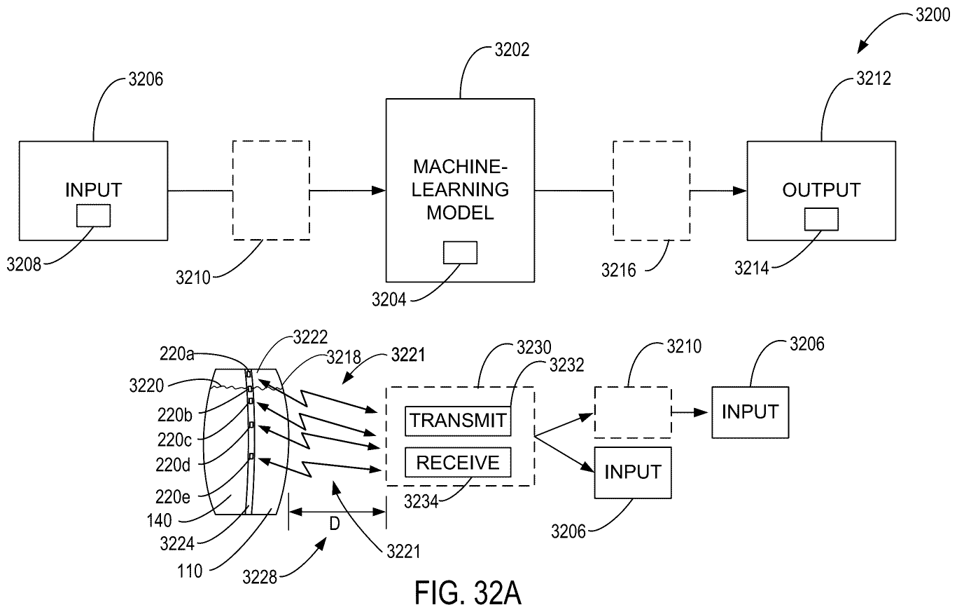

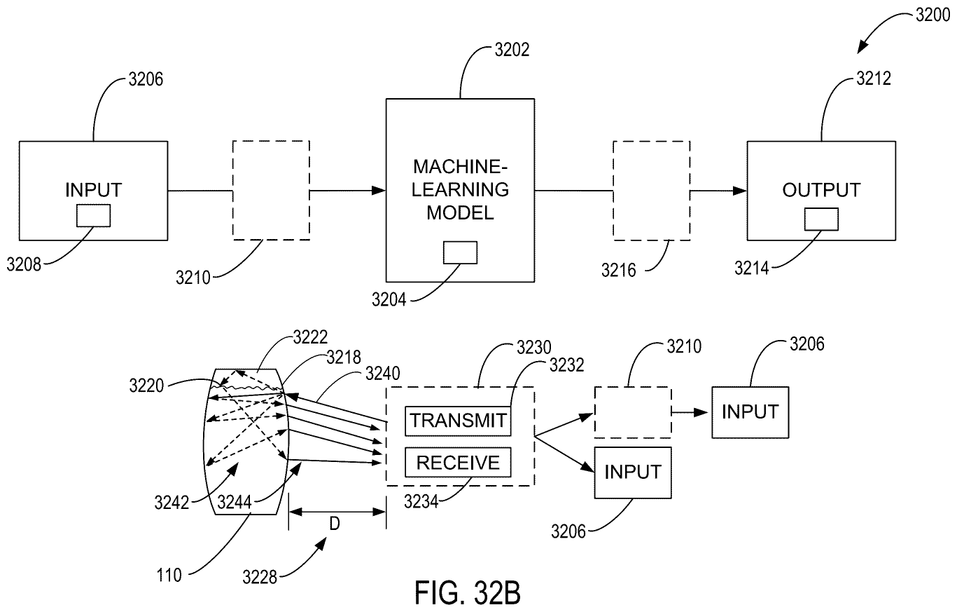

Referring now to FIG. 32A, illustrated is an embodiment of an AI-driven RF sensing system 3200 configured for analyzing environmental signal characteristics associated with a liquid-filled container (e.g., a barrel or cask) in accordance with aspects of the present disclosure. The system 3200 is configured to collect RF signal response data from one or more external sensing structures mounted on a container 140 and to process that data using a machine-learning model 3202 to infer internal conditions of interest, such as but not limited to liquid level, evaporation rate, alcohol concentration, char level, or seepage presence.

The AI-driven RF sensing system 3200 represents an integrated solution for non-invasive monitoring of containers through radio frequency interaction analysis. The system 3200 may include hardware components such as RF transmission and reception modules, along with software components that implement signal processing algorithms and machine learning inference capabilities. In certain aspects, the system 3200 may be implemented as a distributed architecture wherein sensing components are positioned proximally to containers while processing components may be located either locally or remotely. The system 3200 can support various operational modes including real-time monitoring, scheduled polling, or event-triggered analysis of container contents. Unlike traditional invasive measurement techniques that require opening containers or inserting probes, system 3200 enables continuous monitoring without disturbing the aging process, potentially preserving product quality while providing enhanced data collection capabilities. The system 3200 may be configured to monitor individual containers or simultaneously track multiple containers throughout a storage facility, with scalability accommodated through distributed sensing nodes and centralized processing. In some implementations, the system 3200 may operate with minimal power requirements, potentially using energy harvesting or long-life batteries to support extended deployment periods matching typical aging durations of several years.

The machine-learning model 3202 may be configured to analyze input data 3206 derived from RF signal interactions with an RF-responsive element, container, and/or the content of the container. The machine-learning model 3202 may comprise various computational architectures such as neural networks, random forests, support vector machines, or ensemble methods that transform signal characteristics into inferences about container conditions. The machine-learning model 3202 may be trained using supervised learning techniques wherein labeled datasets pair known container states (e.g., verified liquid levels or measured alcohol contents) with corresponding RF signal patterns. Alternatively, the machine-learning model 3202 may employ unsupervised or semi-supervised learning to identify patterns and anomalies without extensive labeled data. In certain implementations, the machine-learning model 3202 may incorporate transfer learning principles, allowing knowledge gained from one container type to be applied to others with appropriate calibration adjustments. The machine-learning model 3202 may execute on various computational platforms ranging from embedded processors within sensing units to cloud-based computing environments accessing data through wireless transmission. Through continual refinement, the machine-learning model 3202 can adapt to evolving container conditions, potentially improving accuracy over time as additional data is collected and incorporated into training datasets.

The neural network 3204 may be included within or represent an implementation of the machine-learning model 3202, providing a specific architectural approach to processing RF signal data. The neural network 3204 may comprise multiple layers of interconnected nodes or neurons that transform input features into increasingly abstract representations before generating output predictions. For RF signal processing applications, the neural network 3204 may include convolutional layers suitable for identifying spatial patterns in signal data, recurrent structures for analyzing temporal sequences, or attention mechanisms for focusing on relevant signal components. The network architecture may be tailored to the specific sensing modality employed, such as specialized layers for processing phase information, RSSI variations, or channel state information matrices. During operation, the neural network 3204 applies learned weights and biases to input data 3206, propagating transformed values through activation functions to generate inferences about container contents. These inferences may include continuous values (e.g., precise liquid level measurements), categorical classifications (e.g., presence of seepage), or temporal predictions (e.g., estimated remaining aging duration). Implementations of the neural network 3204 may vary in complexity from lightweight models suitable for edge deployment to deep architectures requiring substantial computational resources.

Input data 3206 encompasses the signal-derived information fed into the machine-learning model 3202 for analysis. This data may include raw or processed measurements obtained from RF interactions with the container 140, the RF-responsive element, and/or the contents of the container. The input data 3206 may comprise multiple signal characteristics including received signal strength indicators (RSSI), phase shifts, channel state information (CSI), resonant frequency variations, or other electromagnetic parameters that vary based on material properties and geometric arrangements. Depending on the implementation, input data 3206 may incorporate time-series information showing how signals change over measurement intervals, potentially revealing trends in evaporation rates or compositional changes. The data may include metadata such as timestamps, container identifiers, environmental readings, or calibration references that provide context for the signal measurements. In some embodiments, input data 3206 may represent differential measurements comparing current readings against baseline values, highlighting changes rather than absolute states. The format, resolution, and dimensionality of input data 3206 may vary based on the specific sensing technology employed and the machine learning architecture, ranging from simple scalar values to complex multi-dimensional tensors representing spatial and spectral distributions of RF responses. In some aspects, the input data may include one or more profile readings based on one or more scenarios corresponding to the signal responses shown in the graphs of FIG. 26. Thus, the signal response profile illustrated in diagrams 2640a, 2640b, and 2640c may include multiple RF signal characteristics measured simultaneously to provide data to make a determination of liquid level within a container using externally mounted RF-responsive elements.

Optional pre-processing 3210 represents signal conditioning or feature extraction operations performed on raw measurements before they are provided to the machine-learning model 3202. This processing stage may include noise reduction techniques such as filtering, smoothing, or outlier removal to enhance signal quality. Feature extraction algorithms may identify relevant characteristics from raw waveforms, potentially calculating statistical moments, spectral components, or correlation metrics that facilitate subsequent analysis. Normalization or standardization procedures may scale input values to ranges suitable for the neural network 3204, while dimensionality reduction techniques might compress redundant information for computational efficiency. In some implementations, optional pre-processing 3210 may incorporate domain-specific transformations that emphasize container-relevant signal characteristics, such as emphasizing frequency bands known to interact strongly with liquid-air interfaces. The specific pre-processing operations may be selected based on empirical performance evaluations or theoretical electromagnetic models of container interactions. While illustrated as a distinct block in FIG. 32A, the optional pre-processing 3210 functionality may be integrated within sensor firmware, implemented in dedicated signal processing hardware, or executed as part of the machine learning pipeline.

Output data 3212 represents the processed results generated by the machine-learning model 3202 after analyzing input data 3206. The output data 3212 may provide quantitative measurements, qualitative assessments, predictions, or alerts related to container conditions. Depending on the implementation, output data 3212 may be formatted as numerical values (e.g., liquid level in centimeters, alcohol content percentage), categorical classifications (e.g., normal condition vs. leak detected), trend projections (e.g., estimated time to reach target maturation), or multidimensional representations of container state. The output data 3212 may include confidence metrics or uncertainty estimations that indicate the reliability of inferences based on signal quality or model certainty. In networked implementations, output data 3212 may be transmitted to monitoring systems, database servers, or user interfaces for visualization and decision support. The format and content of output data 3212 may be tailored to specific use cases, such as compliance reporting for regulatory authorities, quality control metrics for production managers, or simplified status indicators for operational staff. The temporal resolution of output data 3212 may vary from real-time continuous monitoring to periodic batch updates depending on application requirements and power constraints.

The inferred characteristics 3214 represent specific container and/or liquid conditions or properties determined by the machine-learning model 3202 as components of the output data 3212. These characteristics may include liquid level, alcohol content (ABV), evaporation rate, leak detection, char layer classification, or predictive aging indicators. Each characteristic may be derived from different aspects of the RF signal response, potentially requiring specialized feature extraction or model components. The characteristics 3214 may include both current state information and trend analysis showing how conditions have changed over time. For example, an evaporation trend might be calculated by analyzing sequential liquid level measurements and environmental factors. In some implementations, the inferred characteristics 3214 may include actionable insights such as optimal rotation scheduling for barrels experiencing uneven aging conditions or alerts when measurements deviate from expected patterns. The precision and accuracy of these characteristics may vary based on sensing technology, signal quality, and model training, with certain implementations providing uncertainty bounds or confidence intervals alongside point estimates. The specific characteristics included in system output may be configurable based on operational requirements, with additional characteristics potentially added through model retraining or extended feature extraction.

Optional post-processing 3216 encompasses additional analysis or transformation operations performed on the machine-learning model's immediate outputs before presenting final results. This processing stage may include calibration adjustments that account for systematic biases or sensor-specific variations, potentially referencing historical measurements or calibration constants. The post-processing operations might apply business rules or compliance thresholds that convert raw measurements into actionable statuses, such as flagging containers that approach regulatory limits or quality thresholds. Temporal analysis algorithms may process sequential measurements to calculate derived metrics like rates of change or to perform anomaly detection by comparing measurements against expected trends. In some implementations, optional post-processing 3216 may generate visualizations, summary statistics, or reports that contextualize measurements for human operators. While shown as a distinct component in FIG. 32A, the post-processing functionality may be integrated into reporting systems, implemented within user interfaces, or executed as part of a broader analytics pipeline. The specific post-processing operations may be customized based on industry requirements, operational preferences, or regulatory frameworks applicable to the contained substances.

In some aspects, a set 3224 of passive or semi-passive RF-responsive elements 220u-220n is affixed to the external surface of the container 140. These RF-responsive elements may be configured as specially designed antennas, resonators, or backscatter devices that interact with incident radio frequency signals. Each element 220u-220n may be positioned at a different height or location on the container to enable spatial monitoring of internal conditions. The RF-responsive elements 220u-220n may operate without internal power sources in passive implementations, deriving energy from incident RF signals to modulate and reflect responses. In semi-passive implementations, the elements may include energy storage components such as batteries or capacitors that power signal processing or sensing functions while still using backscatter for communication. The physical design of these elements may be optimized for specific frequency bands, polarizations, or reflection characteristics suited to detecting liquid interfaces, vapor density, vapor density in head space 3222, or material properties 3218 through the container wall. The RF-responsive elements 220u-220n may include tuned circuits, impedance-controlled structures, or frequency-selective surfaces that produce measurable signal variations when their electromagnetic environment changes due to shifts in adjacent materials. In some embodiments, these elements may include additional sensing components such as temperature sensors, strain gauges, or humidity detectors that provide complementary measurements alongside RF interactions.

The RF signals 3221 represent electromagnetic waves transmitted toward the container 140 and the RF-responsive elements 220u-220n by the transmit module 3232. These signals may operate in various frequency bands, potentially including ultra-high frequency (UHF), microwave, or millimeter-wave ranges selected based on penetration characteristics through container materials and sensitivity to internal conditions. The RF signals 3221 may be configured with specific modulation patterns, frequency sweeps, or pulse characteristics optimized for detecting liquid interfaces or compositional properties. In some implementations, the signals may employ spatial diversity through multiple transmission antennas or beamforming techniques that focus energy toward specific container regions. The signals 3221 may be transmitted continuously for real-time monitoring, periodically to conserve power, or on-demand when triggered by schedule or external request. The signal properties including frequency, power level, polarization, and modulation scheme may be dynamically adjusted based on environmental conditions or specific monitoring objectives. In certain embodiments, the RF signals 3221 may incorporate frequency-hopping or spread-spectrum techniques to mitigate interference in environments with multiple containers or RF systems.

The reflected signals 3223 represent electromagnetic waves that return from the RF-responsive elements 220u-220n after interaction with the container 140, the RF-responsive element, and/or the content of the container. In some aspects, these signals may carry information as a reflected transmitted signal 3223. In some aspects, these signals may carry information about the container's internal state encoded in various signal characteristics such as amplitude, phase, frequency shift, or time delay. The reflected signals 3223 may exhibit different properties depending on whether the corresponding RF-responsive element is adjacent to liquid, vapor, or air within the container, due to the varying electromagnetic properties of these materials. In implementations using passive backscatter communication, the reflected signals 3223 may be modulated by the RF-responsive elements through load modulation, where impedance variations create distinctive reflection patterns. The reflected signals 3223 may travel in multiple paths due to scattering from container structures, potentially carrying additional information about container geometry or material conditions. Signal strength, coherence, and quality may vary based on factors such as distance, orientation, and environmental conditions. Reception techniques including diversity combining, coherent detection, and/or statistical signal processing may be employed to extract maximum information from these reflections despite potential signal degradation or interference.

Container 140 represents a vessel designed to hold liquid, such as a whiskey barrel, wine cask, or other storage receptacle being monitored by the RF sensing system. The container 140 may be constructed from various materials including wood, metal, plastic, or composite materials, with specific electromagnetic properties that influence RF signal propagation. The container 140 may have a defined geometry such as a cylindrical barrel with curved staves and metal hoops, with dimensions and proportions that affect signal paths and reflections. Internal contents of container 140 may include liquids, vapors, solids, or combinations thereof, potentially stratified or mixed depending on the specific application and stage of processing. The container 140 may undergo various physical changes during normal operation, such as expansion, contraction, or moisture absorption, which might affect signal propagation characteristics over time. In aging applications, the container material may interact with contents through processes such as absorption, extraction, or oxidation, potentially changing both the contents and the container properties. The RF-responsive elements 220u-220n may be affixed to the exterior surface of container 140 without penetrating the container wall, thereby preserving the integrity of the aging environment while enabling non-invasive monitoring of internal conditions.

The liquid 120 represents the fluid contents within container 140 being monitored by the RF sensing system. This liquid may be a distilled spirit, wine, beer, or other substance with specific electromagnetic properties that affect RF signal interaction. The liquid 120 may have a defined surface level 3220 that creates a liquid-air interface within the container, potentially changing over time due to evaporation, absorption, consumption, or leakage. The composition of liquid 120 may evolve during aging processes through oxidation, extraction of compounds from container walls, or chemical reactions, potentially affecting its dielectric properties and RF signal interactions. The liquid 120 may have varying dielectric constant, conductivity, or loss tangent depending on its composition, with properties such as alcohol content potentially influencing these electromagnetic characteristics in measurable ways. Temperature gradients or stratification may exist within the liquid, creating spatial variations in properties that might be detectable through appropriate sensing techniques. The level, volume, and composition of liquid 120 represent primary monitoring targets for the RF sensing system, with changes in these parameters potentially correlated with quality indicators, process status, or compliance requirements depending on the specific application context.

The transmit module 3232 represents the hardware and control components responsible for generating and emitting RF signals 3221 toward the container 140 and its attached RF-responsive elements 220u-220n. This module may include RF signal generators, power amplifiers, and antenna systems configured to produce signals with suitable frequency, power, modulation, and directional characteristics for container monitoring applications. The transmit module 3232 may support various operational modes including, but not limited to, continuous wave transmission, frequency-modulated continuous wave (FMCW), pulsed operation, impulse radar, guided wave radar (GWR), continuous-wave Doppler radar (CW Doppler), Time-Of-Flight (TOF) radar, Phase-Based Radar, Millimeter-Wave Radar, or more complex waveform generation tailored to specific sensing objectives. In some implementations, the module may incorporate beamforming capabilities through phased arrays or multiple antennas to direct energy toward specific container regions or to simultaneously monitor multiple containers. The transmit module 3232 may include control logic that manages transmission timing, power levels, or frequency selection based on application requirements or environmental conditions. Power management features may optimize energy consumption for battery-powered implementations, potentially including sleep modes, duty cycling, or adaptive power control based on distance or signal quality requirements. The physical configuration of transmit module 3232 may range from integrated units mounted near containers to distributed systems with centralized generation and remote antenna placement depending on facility layout and monitoring architecture.

The receive module 3234 encompasses the hardware and signal processing components that capture, condition, and interpret the reflected signals 3223 returning from the RF-responsive elements 220u-220n. This module may include receive antennas, low-noise amplifiers, filtering circuits, and demodulation systems designed to extract relevant information from potentially weak or distorted reflections. The receive module 3234 may implement various detection methods including coherent reception, envelope detection, or I/Q demodulation depending on the specific signal characteristics being measured. Signal processing capabilities within the module may include amplification, filtering, digitization, and preliminary analysis of received waveforms before forwarding processed data to subsequent analysis stages. In some implementations, the receive module 3234 may incorporate diversity reception through multiple antennas or channels to improve signal quality in challenging environments. The receive sensitivity, bandwidth, and dynamic range may be optimized for the expected signal characteristics and operational conditions, potentially including adaptive gain control to accommodate varying signal strengths. Depending on the system architecture, the receive module 3234 may be physically integrated with the transmit module 3232 in a single transceiver unit 3230 or implemented as a separate component positioned for optimal reception of reflected signals. The module may include calibration capabilities that compensate for environmental variations or component aging to maintain measurement accuracy over extended deployment periods. In some aspects, the transceiver unit 3230 may be the same as or similar to the RF reader 2412 of FIG. 24.

The spacing D 3228 represents the distance between the RF sensing system components (particularly the transmit module 3232 and receive module 3234) and the container 140 with its attached RF-responsive elements 220u-220n. This spacing may vary depending on the specific implementation, ranging from direct contact mounting to standoff distances of several meters. The spacing D 3228 may influence signal propagation characteristics, power requirements, and measurement accuracy, with different sensing modalities potentially operating optimally at different distances. In some applications, minimizing spacing D may improve signal strength and measurement precision by reducing path loss, while in other scenarios, increased spacing may provide broader coverage or accommodate operational constraints such as access requirements or thermal isolation. The spacing D may be fixed in permanent installations or variable in mobile monitoring systems that approach containers temporarily for periodic measurements. Signal processing algorithms may incorporate knowledge of spacing D 3228 to compensate for path loss, propagation delays, or beam spreading effects in measurement calculations. In multi-container monitoring environments, the spacing D may differ between containers based on physical arrangement, potentially requiring calibration adjustments or adaptive transmission parameters to maintain consistent performance across varying distances.

Referring now to FIG. 32B, illustrated is another example of an RF-based sensing architecture for analyzing contents within a container 140, wherein a transmitted signal 3240 is directed toward the container and is reflected back as signal 3244. In contrast to RF-responsive element-based sensing, the configuration shown in FIG. 32B allows direct interrogation of the container using signal reflections, without requiring contact sensors or affixed components on the vessel itself. In some aspects, radar principles can be utilized to infer characteristics of the internal environment of the container 140 based on signal behavior as it reflects from liquid boundaries, air-liquid interfaces, or structural features of the container interior.

The reflected signal 3244 may follow one or more propagation paths 3242 due to partial reflections, scattering, and transmission through the medium within the container. These multiple paths 3242 may include surface reflections, internal bounces, or wall interactions that provide rich spatial and spectral features. Such reflections may be processed by the system using signal processing techniques and fed into a machine-learning inference engine to identify or track characteristics including, but not limited to, liquid level, volume, structural anomalies (e.g., cracks or bulges), surface disturbances, or seepage. By analyzing both time-of-arrival and phase response from the signal paths 3242, the system may isolate specific spatial zones inside the container where variations in electromagnetic properties occur, indicating a change in contents or condition. To enable such functionality, the system may implement one or more radar modalities, each providing distinct advantages for different environmental or application scenarios. In some embodiments, the system may switch dynamically between modalities or operate them in parallel to enhance accuracy and robustness.

The radar modalities that may be utilized in the system may include FMCW radar that transmits a continuous signal whose frequency is linearly modulated over time. By measuring the frequency shift between transmitted and received signals, highly accurate distance and level measurements can be achieved. This technique is particularly suitable for continuous monitoring of fluid levels and can resolve sub-millimeter changes within the container. The radar modalities that may be utilized in the system may include impulse radar that transmits short-duration, high-bandwidth pulses that offer impactful temporal and spatial resolution. The high penetration capabilities of UWB signals make them suitable for sensing through container walls (e.g., wooden staves or metal jackets), while their fine resolution allows for detailed profiling of fluid layers and interfaces, including foam or vapor regions. The radar modalities that may be utilized in the system may include Guided Wave Radar (GWR) that utilizes electromagnetic pulses transmitted along a guided medium (e.g., a probe or rod) inserted into or near the container. The reflections from material interfaces along the probe are used to determine the liquid level or detect phase changes. GWR is particularly effective in containers with complex internal structures or turbulent/foamy contents. The radar modalities that may be utilized in the system may include Continuous-Wave Doppler Radar (CW Doppler) that uses continuous transmission and measures the frequency shift caused by moving targets-such as ripples or level changes-within the container. This modality can detect micro-movements of the fluid surface and may be used to infer theft, tampering, or agitation within the container during transport. The radar modalities that may be utilized in the system may include Time-of-Flight (ToF) Radar that emits short RF pulses and calculates the time taken for the pulse to reflect back from the internal liquid surface. The precise timing provides accurate distance measurements, suitable for use in containers requiring frequent, reliable updates on liquid status. The radar modalities that may be utilized in the system may include Phase-Based Radar that analyzes phase changes in reflected RF waves to determine sub-wavelength shifts in distance or material composition. Because phase changes are highly sensitive to minor environmental variations, this radar type excels in detecting gradual evaporation or small-scale compositional changes in the liquid. The radar modalities that may be utilized in the system may include Millimeter-Wave Radar that operates at frequencies above 24 GHz (often 60 GHz or 77 GHz), and provides fine resolution, high sensitivity to dielectric transitions, and compact form factors. This modality is particularly beneficial for integration into space-constrained environments or for use with small-scale barrels and sample vessels.

In some embodiments, the reflected signal 3244 may be analyzed by a local or remote processing system comprising a machine-learning model similar to the model 3202 described in FIG. 32A. The model may analyze time-domain, frequency-domain, and/or phase information from the reflected signal to infer the liquid level or other relevant attributes of the container contents. In multi-path environments, algorithms may separate overlapping reflections to estimate distances to multiple internal surfaces, stratified fluid layers, or structural obstructions. The signal behavior over time may also allow the system to detect dynamic changes such as active seepage, foam buildup, fermentation activity, or thermal layering. Advantageously, the example shown in FIG. 32B enables non-contact RF-based inspection and monitoring of a container using only external radar sensing components. This configuration may be beneficial for sealed barrels, aged spirits, or environments requiring strict hygiene, where opening containers or applying contact sensors is undesirable or impractical. Additionally, the diversity of supported radar modalities ensures that the system can be adapted to a wide range of materials, fluid types, environmental conditions, and monitoring requirements.

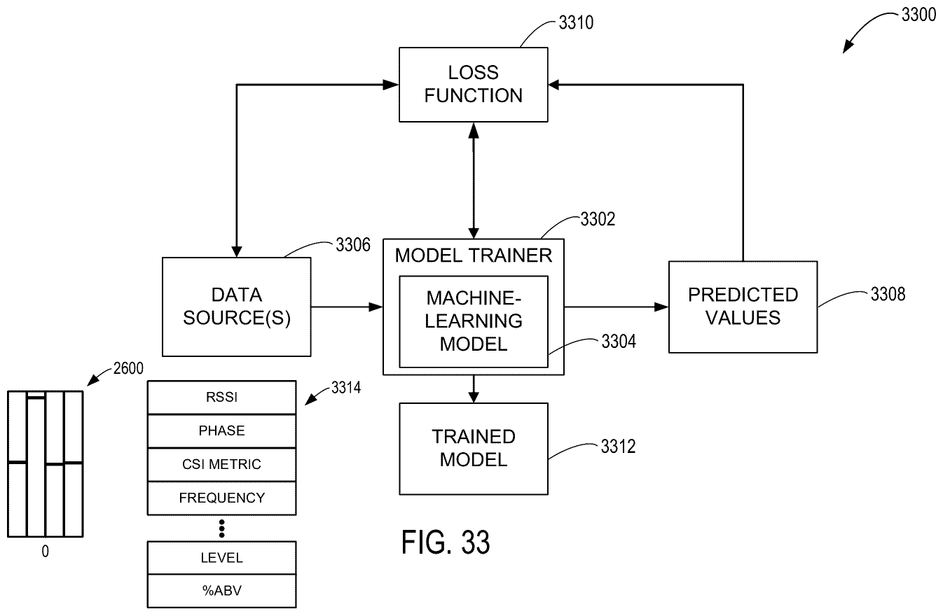

FIG. 33 illustrates a training system 3300 for generating a machine-learning model capable of inferring internal conditions of a container using radio-frequency (RF) signal features. The training system 3300 includes several interacting components that work together to develop a model that can predict parameters such as liquid level or alcohol content based on RF signal characteristics obtained from external sensing elements. The training system 3300 comprises a model trainer 3302, which coordinates the training process and includes a machine-learning model 3304. The system further incorporates data source(s) 3306 that provide input features for training, a predicted values module 3308 that captures model outputs, a loss function module 3310 that evaluates prediction accuracy, and a trained model 3312 that represents the finalized model after training completion.

The data source(s) 3306 supply training data including various RF signal features 3314 such as RSSI, phase measurements, CSI metrics, and frequency domain characteristics. These features may be derived from measurements taken by RF-responsive elements affixed to the exterior of containers as previously described. The data sources 3306 may additionally include ground truth information about container contents, such as actual liquid level measurements and alcohol by volume (% ABV) values, which serve as labels for supervised learning. The model trainer 3302 orchestrates the training process by feeding input features from data source(s) 3306 into the machine-learning model 3304. The machine-learning model 3304 processes these inputs and generates predicted values 3308 representing estimates of internal container conditions. These predicted values 3308 are compared against corresponding ground truth values using the loss function 3310, which calculates discrepancies and provides feedback to the model trainer 3302. Based on the feedback from the loss function 3310, the model trainer 3302 adjusts parameters within the machine-learning model 3304 to minimize prediction errors. This iterative process continues until the model achieves satisfactory performance, at which point it is saved as trained model 3312 for deployment in operational RF sensing systems.

As previously described, the model trainer 3302 functions as the central orchestration component within training system 3300, managing the iterative refinement of the machine-learning model 3304. The model trainer 3302 may implement various optimization algorithms that systematically adjust model parameters to improve prediction accuracy. The model trainer 3302 may employ techniques such as stochastic gradient descent, Adam optimization, or other specialized methods appropriate for the specific machine learning architecture being developed. In operation, the model trainer 3302 first initializes the machine-learning model 3304 with starting parameters, which may be randomly assigned or transferred from a previously trained model in a transfer learning approach. During each training iteration, the model trainer 3302 selects batches of training examples from data source(s) 3306, forwards them through the machine-learning model 3304, and retrieves the resulting predicted values 3308. After the loss function 3310 calculates the prediction error, the model trainer 3302 computes parameter gradients and applies appropriate updates to the machine-learning model 3304. The model trainer 3302 may implement various training regimes, including early stopping when validation performance plateaus, learning rate scheduling to adjust optimization step sizes, or regularization techniques to prevent overfitting. For complex RF sensing applications, the model trainer 3302 may also implement cross-validation across different container types or environmental conditions to ensure the resulting model generalizes effectively to diverse operational scenarios.

Additionally, the model trainer 3302 may manage computational resources, potentially distributing training across multiple processing units or adjusting batch sizes to optimize memory utilization. The model trainer 3302 may also log training progress, capturing performance metrics, parameter statistics, and example predictions for later analysis or comparison between model versions. When training converges according to predefined criteria—such as reaching a target loss threshold or completing a maximum number of epochs—the model trainer 3302 finalizes the machine-learning model 3304 and serializes it as trained model 3312, making it ready for deployment in production environments.

The machine-learning model 3304 represents the computational architecture that transforms RF signal features into predictions about container internals. This model may be implemented using various approaches depending on the complexity of the RF sensing task and the characteristics of available training data. For RF sensing applications in container monitoring, the machine-learning model 3304 may be structured as a neural network with specialized layers suited to processing spatial, temporal, or spectral signal patterns. Such architectures might include convolutional layers for extracting spatial features from multi-tag arrangements, recurrent or transformer layers for analyzing temporal signal evolution, or combinations thereof in a hybrid design. Alternatively, the machine-learning model 3304 could employ ensemble methods like random forests or gradient-boosted trees, which may offer interpretability advantages in regulatory contexts where prediction rationales are important.

The input layer of machine-learning model 3304 is dimensioned to accommodate the feature vectors provided by data source(s) 3306, which may include multiple signal characteristics (RSSI, phase, etc.) potentially across multiple measurement positions or time points. Internal layers transform these inputs through linear and non-linear operations, gradually mapping the raw signal features to more abstract representations that correlate with container properties.

The output layer of machine-learning model 3304 is configured to produce the specific container parameters being predicted, such as liquid level height, alcohol content percentage, or categorical assessments of container integrity. For regression tasks like level prediction, the output might be a single continuous value, while for multi-parameter inference, the output could comprise multiple nodes representing different aspects of the container state. During forward propagation, machine-learning model 3304 computes activations through its layers, applying weights, biases, and activation functions to transform the input features into predicted values 3308. During backward propagation guided by model trainer 3302, these weights and biases are adjusted to reduce the discrepancy between predictions and ground truth as measured by loss function 3310. The machine-learning model 3304 may also incorporate architectural features specific to RF sensing challenges, such as attention mechanisms that focus on particularly informative frequency bands or tag positions, or specialized layers that incorporate electromagnetic propagation principles as inductive biases.

The data source(s) 3306 component represents the repositories and pipelines that provide training examples to the machine-learning model 3304. These sources supply both input features derived from RF signal measurements and the corresponding ground truth values for supervised learning. In the context of RF-based container monitoring, data source(s) 3306 may include databases of historical measurements collected from containers with known internal states, simulated datasets generated through electromagnetic modeling of RF-material interactions, or combinations of real and synthetic data used for model pre-training or augmentation. The RF signal features 3314 provided by data source(s) 3306 may include various signal characteristics such as RSSI, which measures the power level of received signals and varies based on the dielectric properties of materials between and around the RF elements; phase measurements, which capture timing relationships between transmitted and received signals that can be affected by propagation paths; CSI metrics, which provide detailed frequency-domain profiles of the RF channel including amplitude and phase information across multiple subcarriers; and frequency-domain patterns that characterize resonant behavior of the RF elements as influenced by nearby materials. These signal features may be collected from passive or semi-passive RF-responsive elements attached externally on containers, without requiring direct access to the container contents. The features may be preprocessed before serving as inputs to the machine-learning model 3304, with operations such as normalization, dimensionality reduction, or feature engineering applied to enhance learning efficiency. In some aspects, the data sources(s) 3306 may include one or more profile readings based on one or more scenarios corresponding to the signal responses shown in the graphs of FIG. 26. Thus, the signal response profile illustrated in diagrams 2640a, 2640b, and 2640c may include multiple RF signal characteristics measured simultaneously to provide data to make a determination of liquid level within a container using externally mounted RF-responsive elements.

The ground truth labels paired with these features may represent the actual internal conditions of the containers at the time of measurement, such as liquid levels, alcohol percentages, or other parameters of interest. These labels may be obtained through traditional measurement methods during controlled experiments, certified measuring instruments, or other reliable reference sources. Data source(s) 3306 may implement various sampling strategies to ensure balanced representation across different container types, fill levels, or environmental conditions, potentially oversampling rare conditions to improve model robustness. The component may also manage data versioning, validation, and partitioning into training, validation, and test sets to support rigorous model development and evaluation.

The predicted values 3308 represent the outputs generated by the machine-learning model 3304 when processing input features from data source(s) 3306. These values constitute the model's estimates of container internal conditions based on RF signal characteristics, and serve as the basis for performance evaluation during training. In RF-based container monitoring applications, predicted values 3308 may include continuous estimates such as liquid level height in centimeters, alcohol concentration as a percentage, or evaporation rate over time. Alternatively, they might represent categorical assessments such as char level classification, seepage detection results, or quality categorizations. For multi-task models, predicted values 3308 could comprise vectors of multiple parameters simultaneously inferred from the same RF measurements. During training, these predicted values 3308 are generated through forward propagation within the machine-learning model 3304, with the model's current parameters determining how input features are transformed into output estimates. The predicted values 3308 are then passed to the loss function 3310, which quantifies their deviation from known ground truth values to guide model refinement. The format and interpretation of predicted values 3308 depend on the specific architecture of the machine-learning model 3304. For regression tasks, they might be direct scalar outputs from the model's final layer, while for classification tasks, they could be probability distributions across possible categories. Some model designs might produce both point estimates and uncertainty measures, enabling risk-aware decision-making in operational settings.

The precision, accuracy, and reliability of predicted values 3308 typically improve over the course of training as the model trainer 3302 adjusts model parameters to minimize prediction errors. By tracking the evolution of predicted values 3308 across training iterations, developers can assess learning progress, identify challenging prediction cases, and diagnose potential issues such as overfitting or underfitting. After deployment of the trained model 3312, the equivalent of these predicted values becomes the operational output of the RF sensing system, potentially feeding into monitoring dashboards, automated control systems, or compliance reporting tools in distillery or warehouse environments.

The loss function 3310 quantifies the discrepancy between predicted values 3308 generated by the machine-learning model 3304 and the corresponding ground truth values provided by data source(s) 3306. This component plays a critical role in the training process by providing the optimization signal that guides parameter updates within the model. For RF sensing applications in container monitoring, the loss function 3310 may be selected based on the specific prediction tasks and their operational importance. Common implementations include mean squared error (MSE) for continuous parameters like liquid level, cross-entropy loss for categorical predictions like seepage classification, or custom formulations that combine multiple error terms with task-specific weighting factors. In some implementations, loss function 3310 may incorporate domain knowledge about RF sensing physics or container monitoring priorities. For example, it might apply higher penalties to errors in specific operating ranges where precision is particularly important, such as near regulatory thresholds for alcohol content. The loss function may also include terms that promote physically plausible predictions, such as monotonicity constraints for liquid level estimates or temporal consistency for sequential measurements.

During each training iteration, loss function 3310 receives the current batch of predicted values 3308 along with their corresponding ground truth values, computes the appropriate error metric, and returns this value to model trainer 3302. The model trainer then uses this error signal to compute gradients and update model parameters in a direction that reduces the loss. For container monitoring systems that must balance multiple objectives—such as accuracy across different content types, robustness to environmental variations, and computational efficiency—loss function 3310 may implement a weighted combination of terms that collectively guide the model toward an optimal trade-off. The relative weights of these terms may be fixed in advance based on application requirements or dynamically adjusted during training. Beyond its role in parameter optimization, loss function 3310 also provides a quantitative measure of model performance that can be tracked over time to assess training progress, compare different model versions, or establish acceptance criteria for model deployment. A decreasing trend in the loss value typically indicates improving model quality, though validation on held-out data remains essential to confirm generalization capability.

The trained model 3312 represents the final output of the training system 3300-a machine-learning model whose parameters have been optimized through the training process and which is ready for deployment in operational RF sensing systems for container monitoring. This component encapsulates the architecture, weights, and computational graph of the machine-learning model 3304 after training completion. The trained model 3312 may be serialized in a format suitable for persistent storage and efficient loading into inference systems, such as TensorFlow SavedModel, ONNX, or platform-specific binary formats. Depending on the implementation, it may also include metadata about the training process, performance metrics, expected input formats, and output interpretations to facilitate integration with broader monitoring infrastructure. In RF-based container monitoring applications, the trained model 3312 embodies the learned relationships between RF signal characteristics and internal container parameters. Once deployed, it enables real-time or periodic inference of liquid levels, alcohol content, or other parameters of interest without requiring direct access to container contents, thereby preserving the integrity of aging processes or other sensitive operations.

The effectiveness of trained model 3312 depends on various factors, including the representativeness of training data, the appropriateness of the model architecture for the specific RF sensing task, and the thoroughness of the training process. Before operational deployment, the trained model may undergo additional validation on test datasets or controlled field trials to ensure it meets accuracy, robustness, and computational performance requirements. In some implementations, trained model 3312 may be designed for deployment on resource-constrained edge devices mounted directly on or near containers, requiring optimization techniques such as quantization, pruning, or knowledge distillation to reduce model size and computational demands while maintaining prediction quality. Alternatively, for centralized monitoring of multiple containers, the trained model may be deployed on server infrastructure that processes RF measurements collected from distributed sensing nodes. The trained model 3312 may be periodically retrained or fine-tuned as new data becomes available, environmental conditions change, or operational requirements evolve. This continuous improvement process helps maintain prediction accuracy and extends the model's utility across changing container characteristics or aging conditions.

RF signal features 3314 serve as primary inputs to the machine-learning model 3304. These features are derived from measurements collected by RF-responsive elements attached externally on containers, and they capture how radio frequency signals interact with the container and its contents. As depicted in FIG. 33, the RF signal features 3314 include multiple categories of measurements that provide complementary information about container internals. RSSI measurements quantify the power level of signals received after interaction with the container environment, with variations in signal strength potentially indicating changes in the dielectric properties associated with different fill levels or content compositions. Phase measurements capture the timing relationships between transmitted and received signals, which can be affected by propagation paths and material boundaries within the container. CSI (Channel State Information) metrics provide detailed frequency-domain profiles of the RF channel, including amplitude and phase information across multiple subcarriers, potentially revealing spectral signatures associated with specific internal conditions. Frequency domain characteristics describe how certain frequency components of RF signals are affected differently by container contents, potentially revealing resonance patterns or absorption characteristics that correlate with parameters of interest. These RF signal features may be collected through various sensing modalities, including passive backscatter from RFID tags, active measurements from wireless transceivers, or specialized RF sensing structures designed specifically for container monitoring applications. The features may exhibit different sensitivities to various aspects of container contents; for example, RSSI measurements might be particularly responsive to liquid level transitions, while frequency-domain characteristics might better capture compositional variations such as alcohol content. The data source(s) 3306 may provide these features in raw form or preprocessed through operations such as normalization, filtering, or feature extraction to enhance their utility for model training. During model development, the relative importance of different feature types may be evaluated through techniques such as feature importance analysis or ablation studies, potentially informing future sensor designs or measurement protocols to emphasize the most informative signal characteristics. The bottom portion of the RF signal features 3314 section in FIG. 33 may indicate the target parameters that the system aims to predict, including liquid level and % ABV (alcohol by volume). These parameters may represent the ground truth labels paired with RF features in the training dataset, enabling supervised learning of the relationships between external RF measurements and internal container conditions.

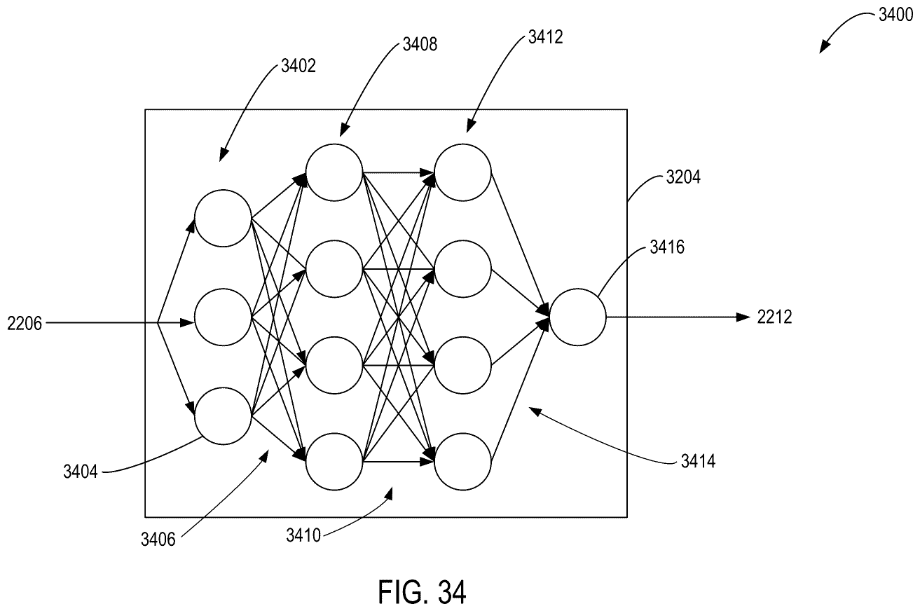

Referring to FIG. 34, an artificial neural network 3400 is illustrated for processing radio-frequency (RF)-derived input data and generating predictions relating to characteristics of contents within a sealed container in accordance with aspects of the present disclosure. The neural network 3400 may be implemented as part of a machine-learning system for analyzing radio frequency signal characteristics associated with a container such as a barrel, cask, tank, or other vessel. The artificial neural network 3400 represents a computational architecture configured to transform input features derived from RF signal interactions with a container and its contents into predicted values corresponding to internal characteristics of the container. In the illustrated aspect, the artificial neural network 3400 comprises multiple interconnected layers including an input layer 3402, multiple hidden layers 3406, 3408, 3412, and an output layer 3416. These layers are connected via weighted edges 3404, 3410, and 3414, which enable signal propagation throughout the network. The artificial neural network 3400 may be structured as a feedforward multilayer perceptron (MLP), although other architectural configurations may be implemented depending on the specific RF sensing application requirements.

The input layer 3402 is configured to receive data values derived from RF signal measurements collected by one or more RF-responsive elements positioned externally on a container. The input layer 3402 comprises multiple input neurons, each corresponding to a specific feature or parameter extracted from the RF signal data. These features may include, but are not limited to, RSSI values, phase measurements, CSI metrics, or frequency-domain characteristics such as resonant frequency shifts or amplitude variations. The input layer 3402 acts as the entry point for data into the neural network, where each input neuron may be assigned a weight representing the relative importance of its corresponding feature. The input neurons may apply initial transformations to normalize or scale the incoming data values to facilitate subsequent processing within the network.

In some aspects, the input layer 3402 may receive multiple features extracted from a single RF signal measurement, while in other aspects, the input layer 3402 may receive distinct features from multiple RF measurements taken at different positions on the container or at different time points. The dimensionality of the input layer 3402 may be configured based on the richness and complexity of the RF signal features being utilized for the specific container monitoring application. For instance, when analyzing RSSI patterns from multiple RF-responsive elements positioned at different heights on a container, the input layer 3402 may include neurons corresponding to each element's signal strength value.

The input layer 3402 connects to a first hidden layer 3406 via a first set of edges 3404. These edges 3404 represent weighted connections between the nodes of the input layer 3402 and the nodes of the first hidden layer 3406. Each edge carries a weight value that modulates the strength of the signal transmitted along that connection. During forward propagation, the input values received at layer 3402 are multiplied by the corresponding edge weights of edges 3404 before being passed to the neurons in the first hidden layer 3406. These weights effectively determine how strongly each input feature influences the activations in the subsequent layer. The edges 3404 impact the learning capability of the neural network, as their weight values are adjusted during training to optimize the network's predictive performance. The collection of weights associated with edges 3404 forms a weight matrix that encodes the learned relationships between input features and higher-level abstractions. In some implementations, the edges 3404 may also incorporate additional parameters such as bias terms, which allow the network to learn additive offsets that can improve modeling flexibility.

The first hidden layer 3406 comprises multiple neurons that receive weighted inputs from the input layer 3402 via edges 3404. In some aspects, each neuron in the first hidden layer 3406 may compute a weighted sum of its inputs, apply a non-linear activation function (such as ReLU, sigmoid, or tanh), and produce an output value that represents a transformed feature or abstraction derived from the original input data. The neurons in the first hidden layer 3406 enable the network to learn and represent complex, non-linear relationships between RF signal characteristics and container properties.

The first hidden layer 3406 begins the process of feature extraction and transformation, identifying patterns or relationships in the RF data that may be indicative of container conditions such as liquid level, content composition, or internal characteristics. The number of neurons in the first hidden layer 3406 may be configured based on the complexity of the modeling task, with more neurons potentially enabling the capture of more intricate patterns at the cost of increased computational requirements and potential overfitting risk. The first hidden layer 3406 connects to a second hidden layer 3408 via edges 3410. Similar to edges 3404, the edges 3410 represent weighted connections that determine how strongly each neuron in the first hidden layer 3406 influences the neurons in the second hidden layer 3408. The second hidden layer 3408 continues the hierarchical feature extraction process, potentially identifying more abstract or higher-level patterns in the data based on the transformed features from the first hidden layer 3406.

The second hidden layer 3408 may comprise multiple neurons that further refine the representations learned in the first hidden layer 3406. Such neurons may apply additional non-linear transformations, enabling the network to model increasingly complex relationships between RF signal characteristics and container properties. The second hidden layer 3408 may extract features that correspond to specific aspects of the container's internal state, such as liquid-air interface positions, content density variations, or other physically meaningful characteristics.

Continuing through the network, the second hidden layer 3408 connects to a third hidden layer 3412, which may perform additional feature refinement and abstraction. The connections 3410 between the second hidden layer 3408 and the third hidden layer 3412 function similarly to the previously described edges, carrying weighted signals that encode learned relationships. The third hidden layer 3412 may separate or isolate features related to different physical phenomena within the container, potentially preparing the network to make specific predictions about distinct container characteristics. The third hidden layer 3412 connects to the output layer 3416 via edges 3414. These final weighted connections determine how the high-level features extracted by the hidden layers are combined to generate predictions about the container's internal state. The edges 3414 effectively encode the learned relationships between abstract feature representations and specific output parameters of interest, such as liquid level, alcohol content, or other container characteristics.

The output layer 3416 comprises one or more neurons that generate the final predictions or classifications produced by the neural network. Each neuron in the output layer 3416 may correspond to a specific parameter or characteristic being predicted, such as liquid level height, alcohol concentration percentage, presence of seepage, or classification of container conditions. The output neurons may apply different activation functions depending on the prediction task-linear activations for regression tasks (e.g., predicting continuous values like liquid level) or softmax activation for classification tasks (e.g., categorizing container conditions). The output layer 3416 translates the abstract internal representations learned by the network into quantitative predictions or qualitative assessments that can be used for monitoring, control, or decision-making purposes related to the container and its contents. The predictions generated by the output layer 3416 may be used to track aging processes, detect anomalies, estimate evaporation rates, or provide other insights into container conditions without requiring direct access to the container contents.

Input data 2206 represents the features or parameters extracted from RF signal measurements that are provided to the artificial neural network 3400. These inputs may be derived from various RF sensing modalities, including passive backscatter from RFID tags, active measurement from wireless transceivers, or other RF sensing techniques appropriate for non-invasive container monitoring. The input data 2206 may undergo preprocessing to enhance signal quality, extract relevant features, or normalize values before being fed into the neural network.

Output data 2212 represents the predictions or classifications generated by the artificial neural network 3400 based on the input RF signal features. These outputs may include quantitative measurements such as liquid level height, alcohol concentration, evaporation rate, or qualitative assessments such as leak detection, char level classification, or quality categorization. The output data 2212 provides insights into the container's internal conditions without requiring direct physical access to the container contents, enabling non-invasive monitoring for applications such as spirit aging, quality control, or regulatory compliance.

In various implementations, the artificial neural network 3400 may be trained using supervised learning techniques, where labeled datasets pair known container states with corresponding RF signal patterns. The training process adjusts the weights associated with the network's edges to minimize prediction errors, resulting in a model capable of accurately inferring container conditions from external RF measurements. The trained network may be deployed as part of a monitoring system that continuously or periodically assesses container conditions, potentially triggering alerts or control actions based on the predicted container states.

The architecture illustrated in FIG. 34 provides a framework for RF-based container monitoring that can be adapted to various container types, content compositions, and monitoring objectives. The neural network approach enables the system to learn complex relationships between RF signal characteristics and container properties, potentially extracting insights that would be difficult to model using traditional analytical methods. The flexibility of the neural network architecture allows for customization based on specific application requirements, potentially incorporating additional features or specialized network structures to enhance performance for particular container monitoring scenarios.

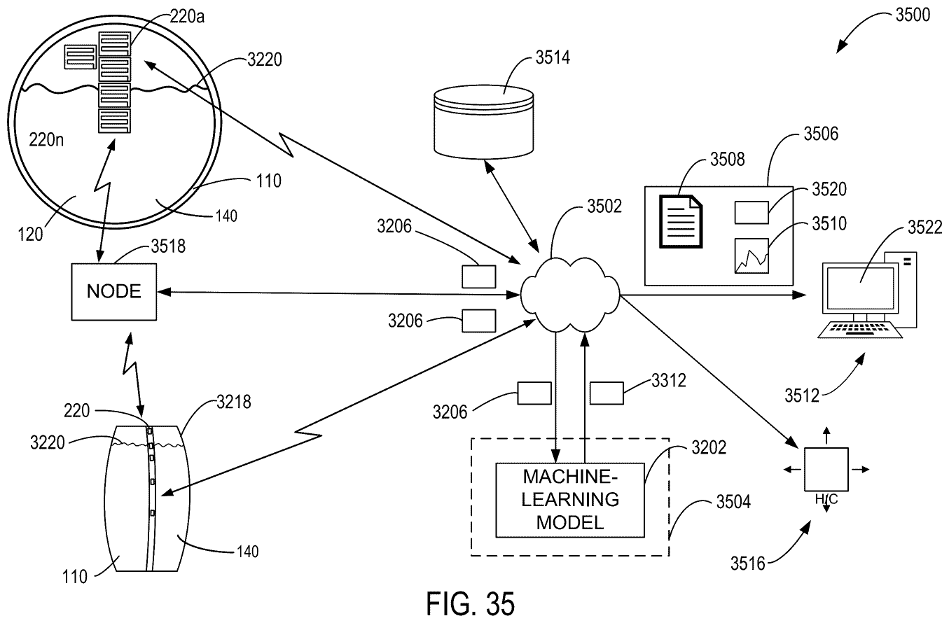

Referring now to FIG. 35, a distributed monitoring and analysis system 3500 is illustrated for determining one or more characteristics of a liquid 120 contained within a container 110 in accordance with at least one aspect of the present disclosure. The container 110 may be implemented as a wooden barrel, cask, or other vessel used in distillation, aging, storage, or similar environments. The distributed monitoring and analysis system 3500 integrates external RF sensor data, networked processing capabilities, machine learning analysis, data storage, and user interface functionality to provide monitoring of container contents without requiring direct access to the interior of container 110.

The container 110 includes an exterior surface 140 to which one or more RF-responsive elements 220u-220n are affixed or positioned in proximity. These RF-responsive elements 220u-220n may be arranged in an array that spans a vertical axis of the container 110, enabling spatial resolution of signal interactions corresponding to changes in the liquid level, composition, or other characteristics of the liquid 120 or surrounding environment within the container 110. The array configuration facilitates detection of the liquid-air interface and monitoring of changes at an interface over time, which may result from processes such as evaporation, absorption, or consumption.

In various implementations, the RF-responsive elements 220u-220n may comprise passive RFID tags, chipless resonators, metasurfaces, energy-harvesting reflective antennas, or other RF-interactive elements capable of reflecting or backscattering RF signals in a manner influenced by the adjacent environment. The RF-responsive elements 220u-220n may be configured to respond to specific RF frequencies or frequency ranges, potentially with distinctive resonance characteristics, phase responses, or backscatter modulation patterns. The RF-responsive elements 220u-220n may be affixed to the container surface 140 using adhesives, brackets, straps, or other attachment mechanisms that ensure stable positioning without penetrating the container wall.

A node 3518 may include one or more antenna arrays or RF interrogators positioned near the container 110. The node 3518 may be implemented as an RF reader, transceiver, or low-power IoT hub that is configured to emit RF signals toward the RF-responsive elements 220u-220n and collect reflected or backscattered responses. The node 3518 may be located proximal to the container 110, such as mounted on a nearby warehouse wall, rack, or other structure, and may include RF front-end hardware, signal processing logic, and wireless communication circuitry. In some implementations, a single node 3518 may monitor multiple containers, potentially using beamforming, scheduled polling, or other techniques to distinguish signals from different containers. In some aspects, the node 3518 may correspond to network equipment configured within a communication system, such as a 3G, 4G, or 5G communication network.

The node 3518 obtains, makes, and/or captures various RF signal measurements from the interactions between transmitted signals and the RF-responsive elements 220u-220n. These measurements may include RSSI, signal phase, CSI, resonant frequency shifts, or other electromagnetic parameters that vary based on the presence, level, or properties of the liquid 120 within the container 110. These signal characteristics may be influenced by the dielectric properties of the liquid, which in turn affect the electromagnetic coupling, impedance, or resonance of the RF-responsive elements 220u-220n.

The node 3518 may be connected to a network 3502, which may include a local area network, wireless mesh network, cellular network, or cloud-based infrastructure such as the Internet. Through the network 3502, signal measurements 3206 from one or more nodes 3518 are transmitted to various system components, enabling distributed processing and analysis. The network 3502 facilitates data transfer, command and control functions, and system integration across potentially disparate physical locations, allowing for centralized monitoring of distributed container environments.

In some aspects, the distributed monitoring and analysis system 3500 may include a machine-learning system 3504 comprising one or more models 3202. These one or more models 3202 may be implemented as neural networks, decision trees, random forests, support vector machines, or other machine learning architectures suitable for processing RF signal data to infer container conditions. The machine-learning system 3504 receives signal data 3206 from one or more sensor nodes via the network 3502 and processes this data using trained models 3202. These models analyze patterns, correlations, or other relationships within the signal data to derive insights about the internal state of the container 110.

The machine-learning models 3202 may be configured to infer one or more characteristics of the liquid contents, including but not limited to liquid fill level, alcohol concentration (expressed as percentage ABV), evaporation or seepage loss rates, char level of the barrel interior, or changes in dielectric properties associated with aging or contamination. The models may be trained using supervised learning approaches, where historical signal data is paired with known ground truth measurements of these characteristics, enabling the model to learn the relationships between RF signal patterns and physical container properties. The machine-learning system 3504 generates inference results 3212, which may be transmitted via the network 3502 to a data visualization module or other system components. These inference results 3212 represent the predictions or determinations by the distributed monitoring and analysis system 3500 regarding the monitored container characteristics, potentially including confidence levels, uncertainty estimates, or other statistical metadata to contextualize the predictions.

The distributed monitoring and analysis system 3500 may include a data storage repository 3514 for persisting raw and processed signal data. This repository may be implemented as a database, data lake, or other storage architecture suitable for handling time-series data, metadata, and analysis results. The data storage repository 3514 enables historical analysis, audit trails, and retention of measurement data for compliance, quality control, or process improvement purposes. The repository may store multiple data types, including raw signal measurements, derived metrics, container metadata (such as fill date, batch number, or contents), and system operational data. One or more remote computing devices 3512 may be connected to the network 3502, providing user access to the distributed monitoring and analysis system 3500. These devices may include desktop computers, laptops, tablets, smartphones, or specialized industrial control terminals. The remote computing devices 3512 may incorporate a graphical user interface 3522 for operator interaction, displaying metrics 3508, logs 3510, and/or other content 3520 related to container monitoring.

The user interface 3522, displayed on the remote computing devices 3512, provides visualization of monitoring data, potentially including real-time measurements, historical trend graphs, alerts, and actionable recommendations based on machine-learning output. The interface 3522 may be implemented as a web dashboard, mobile application, or desktop control panel, offering various visualization modes such as time-series plots, heatmaps, 3D representations, or tabular data. The interface may support customization, filtering, or drill-down capabilities to focus on specific containers, metrics, or time periods of interest. The metrics 3508 displayed on the user interface 3522 may include derived values such as liquid level, alcohol content, evaporation rate, temperature, or other parameters relevant to container monitoring. These metrics may be presented as current values, trend lines, or comparative analyses against reference points, target ranges, or historical norms. The metrics presentation may incorporate visual cues such as color coding, threshold indicators, or status symbols to facilitate rapid assessment of container conditions.

The logs 3510 presented on the user interface 3522 may include chronological records of system events, measurements, alerts, or user interactions. These logs may provide details on system operation, data collection timing, anomaly detection, or actions taken in response to specific conditions. The logs may support filtering, searching, or export capabilities to facilitate troubleshooting, compliance documentation, or operational review. In some implementations, the distributed monitoring and analysis system 3500 may further include a control subsystem 3516 configured to receive inference outputs and generate control signals. This subsystem establishes a feedback loop, where container monitoring data drives automated actions to maintain desired conditions or respond to detected anomalies. The control subsystem 3516 may interface with environmental management systems, production scheduling tools, or other operational technologies to implement its control functions.

Based on detected changes in temperature, alcohol content, evaporation rate, or other monitored parameters, the control subsystem 3516 may adjust heating/cooling (H/C) parameters within the distillation or aging environment. These adjustments might include activating fans, modifying warehouse dampers, opening or closing temperature valves, or triggering other environmental control mechanisms to maintain optimal conditions for product development. The heating/cooling functions may operate on individual containers or affect the ambient environment for multiple containers, potentially with zone-based control for larger storage facilities.

The distributed monitoring and analysis system 3500 architecture allows for scalable deployment across multiple containers, batch monitoring within aging warehouses, and dynamic retraining of the machine-learning model 3202 using incoming measurements and stored data. Multiple nodes 3518 may synchronize via edge computing gateways or feed into a centralized cloud analytics platform. Data may be periodically offloaded to the repository 3514 for audit, compliance, or long-term trend analysis. The distributed monitoring and analysis system 3500 provides non-invasive monitoring of container contents, preserving the integrity of aging processes while delivering valuable insights into product development, inventory management, and quality control. Through integration of RF sensing, networked communication, machine learning, and control systems, the approach enables comprehensive monitoring without requiring direct access to container contents or disruption of traditional production processes.

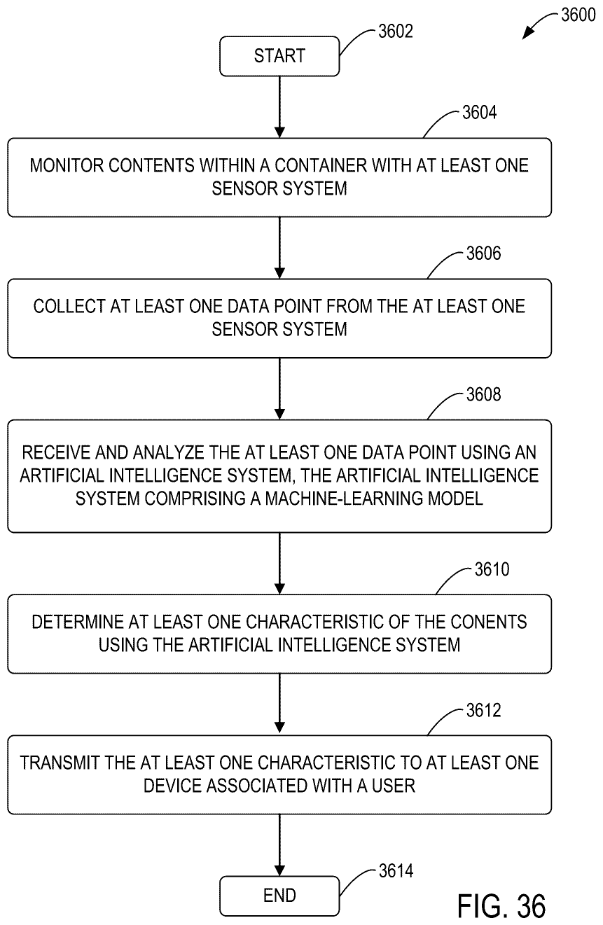

Referring now to FIG. 36, a process 3600 for determining one or more characteristics of a liquid or other contents stored within a container is illustrated in accordance with aspects of the present disclosure. Process 3600 represents a sequence of operations that may be performed to non-invasively monitor liquid characteristics such as level, composition, alcohol content, or other parameters using externally-mounted sensors that do not require direct contact with the monitored liquid. In some aspects, process 3600 may be executed by an integrated monitoring system similar to those described in connection with previous figures, such as the systems illustrated in FIG. 22, 32, or 35.

Process 3600 begins at block 3602, which represents an initialization or starting point for the monitoring sequence. The start block 3602 may correspond to activation of the monitoring system, initialization of sensing hardware, or the beginning of a scheduled monitoring cycle. In some implementations, the process 3600 may initiate based on a timer, an external trigger, or a command received from a control system. The start block 3602 may also encompass preparatory operations such as self-diagnostics, calibration verification, or parameter initialization to ensure proper system operation prior to data collection.

Following initialization, the process 3600 advances to block 3604, which represents monitoring contents within a container with at least one sensor system. Block 3604 encompasses the active sensing operations wherein the monitoring system collects data regarding the container contents using one or more sensing modalities. The sensor system employed at block 3604 may include radio-frequency (RF) based sensors, such as passive or semi-passive RF-responsive elements positioned on the exterior surface of the container. These RF-responsive elements may include RFID tags, resonators, or other RF-interactive devices configured to interact with interrogating radio signals in a manner that is influenced by the adjacent internal environment. The monitoring operation at block 3604 may involve transmitting radio-frequency signals toward the container and RF-responsive elements, then capturing and analyzing the resulting reflected or backscattered signals.

In some aspects, the monitoring at block 3604 may be performed continuously, providing real-time data about container contents. In other aspects, the monitoring may occur periodically according to a predefined schedule, such as hourly, daily, or weekly measurements, to conserve power or computational resources while maintaining sufficient temporal resolution for the application requirements. The monitoring operation may involve multiple sensor types or multiple measurement modalities to capture complementary data, potentially including RSSI measurements, phase information, CSI, resonant frequency shifts, or combinations of these characteristics. The selection of specific sensing parameters and frequencies may be tailored to the particular container material, expected content characteristics, and monitoring objectives.

At block 3606, the process collects at least one data point from the at least one sensor system. This block represents the acquisition and initial processing of measurement data obtained during the monitoring operation. The collected data points may include raw signal values such as reflection amplitudes, phase shifts, or frequency responses measured at multiple locations on the container. These measurements may be taken from a plurality of RF-responsive elements arranged in a spatial pattern, such as a vertical array, to capture information about the internal container state with appropriate spatial resolution. The data collection may also include contextual information such as timestamps, environmental readings (temperature, humidity), or system configuration parameters that may affect signal interpretation.

In some implementations, data collection at block 3606 may involve filtering, normalization, or other signal conditioning operations to prepare the raw sensor readings for subsequent analysis. These operations may include noise reduction, baseline correction, or feature extraction techniques appropriate for the specific sensing modality. The collected data points may be temporarily stored in local memory, buffered for batch processing, or immediately forwarded to the analysis system depending on the implementation architecture.

The process then advances to block 3608, which represents receiving and analyzing the at least one data point using an artificial intelligence system, the artificial intelligence system comprising a machine-learning model. At this block, the collected measurement data is processed through computational algorithms designed to extract meaningful information about the container contents. The artificial intelligence system referenced in block 3608 may be implemented using various machine learning architectures, including but not limited to neural networks, support vector machines, random forests, or ensemble methods that combine multiple modeling approaches. The machine-learning model may have been previously trained using labeled datasets that pair known container states (e.g., verified liquid levels or measured alcohol contents) with corresponding RF signal patterns. This supervised learning approach enables the model to recognize patterns in the signal data that correlate with specific internal conditions, even when these patterns are complex or non-linear. The analysis performed at block 3608 may involve feature extraction from the raw signal data, transformation of these features through one or more computational layers, and ultimately the generation of predictions or classifications regarding the container contents.

In some aspects, the analysis may occur on-device, with the machine-learning model executing locally on processors integrated with or near the sensing hardware. In other aspects, the analysis may be performed remotely, with collected data transmitted to cloud servers or centralized computing infrastructure for processing. The analysis may incorporate temporal information, comparing current readings against historical data to identify trends, anomalies, or changes in the container contents over time. Additionally, the analysis may consider multiple sensor inputs simultaneously, fusing data from different sensing modalities or spatial locations to improve prediction accuracy and consistency.

Following analysis, the process 3600 proceeds to block 3610, where at least one characteristic of the contents is determined using the artificial intelligence system. This block represents the output stage of the analytical process, where specific parameters or properties of the container contents are quantified or classified based on the processed sensor data. The determined characteristics may include, but are not limited to, liquid level, alcohol by volume (ABV) concentration, temperature, color characteristics, char depth, or the presence of contaminants or anomalous conditions. The determination of these characteristics may include not only central value estimates but also confidence intervals, uncertainty metrics, or probability distributions that reflect the reliability of the inferences. For continuous parameters such as liquid level or alcohol content, the system may produce numerical values with appropriate units and precision. For categorical characteristics such as quality classifications or anomaly detection, the system may generate discrete outputs or probability scores associated with each potential classification.

In some implementations, the determination at block 3610 may involve comparing derived measurements against reference values, thresholds, or expected ranges to evaluate whether the container contents are developing according to expectations. The system may also calculate derivative metrics, such as evaporation rates, based on changes in primary characteristics over time. The determination process may incorporate business logic, regulatory requirements, or quality control parameters specific to the application context, such as distillery operations, chemical storage, or food processing.

At block 3612, the process 3600 transmits the at least one determined characteristic to at least one device associated with a user. This block encompasses the communication of results from the monitoring system to individuals or systems responsible for container management, quality control, or operational decision-making. The transmission may occur through various communication channels, including wired networks, wireless protocols (Wi-Fi, Bluetooth, cellular), or specialized industrial communication standards appropriate for the operational environment. The device receiving the transmitted characteristics may include desktop computers, mobile devices, specialized industrial terminals, or automated control systems. The transmitted information may be presented through graphical user interfaces, textual reports, data visualizations, or machine-readable formats suitable for integration with other software systems. In some implementations, the transmission may trigger notifications or alerts when monitored characteristics exceed thresholds or exhibit anomalous patterns that require attention.

The format and content of the transmitted information may be tailored to the specific user role and application context. For example, production managers might receive comprehensive dashboard views with historical trends and forecasts, while maintenance personnel might receive focused alerts about specific containers requiring attention. The transmission may include not only the determined characteristics but also supporting metadata, confidence metrics, or recommended actions based on the monitoring results.

The process 3600 concludes at block 3614, which represents the completion of a single monitoring cycle. After reaching this end block, the system may return to the start block to begin another monitoring iteration, potentially with updated parameters or adjusted scheduling based on the results of the completed cycle. In continuous monitoring implementations, the end block might represent a brief reset or reconfiguration before immediately restarting the process, while in periodic monitoring scenarios, the system might enter a low-power state until the next scheduled measurement interval.

The process 3600 illustrated in FIG. 36 provides a structured approach to non-invasive monitoring of container contents using RF sensing technology and machine learning analysis. This approach enables ongoing assessment of liquid characteristics without disrupting aging processes, compromising container integrity, or requiring manual sampling, thereby supporting quality control, inventory management, and process optimization in various industrial applications.

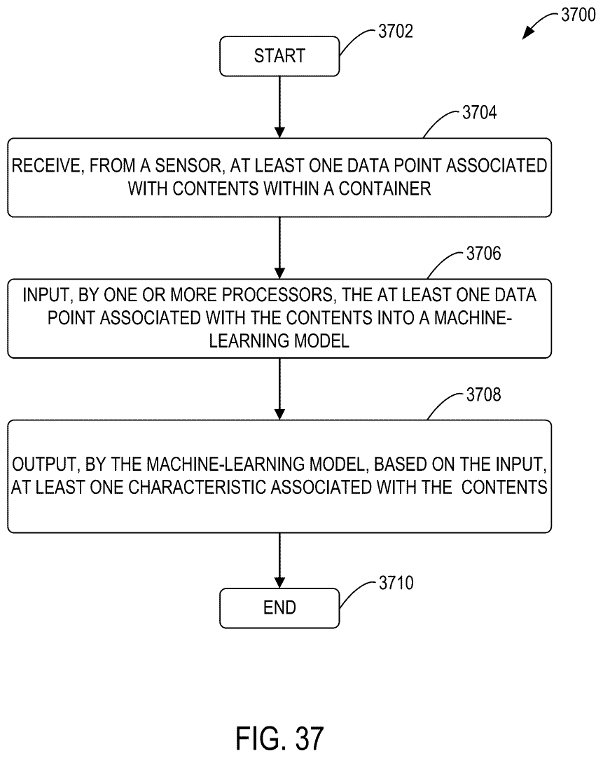

Referring now to FIG. 37, an exemplary process 3700 for determining at least one characteristic of contents within a container using a machine-learning model is illustrated in accordance with aspects of the present disclosure. Process 3700 represents a flowchart depicting a sequence of operations that may be performed to analyze contents of a container such as, but not limited to, a barrel, cask, tank, or other vessel suitable for containing liquid, mash, solid, or other materials. The process 3700 may be implemented in conjunction with the systems described in previous figures, including but not limited to FIGS. 22, 32, and 35.

As shown in FIG. 37, the process 3700 begins at block 3702, which represents an initialization point for the monitoring sequence. Block 3702 may correspond to the activation of a monitoring system, the initialization of sensing hardware, or the beginning of a scheduled monitoring cycle. In some implementations, the process 3700 may initiate based on a timer event, an external trigger signal, or a command received from a control system. The initialization at block 3702 may encompass preparatory operations such as self-diagnostics, calibration verification, or parameter loading to ensure proper system functionality prior to data collection. Following initialization, the process 3700 advances to block 3704, wherein a sensor system acquires at least one data point associated with the contents of a container. The sensor system may comprise one or more sensing modalities capable of non-invasively detecting properties of the container contents. In some embodiments, the sensor system may include radio-frequency (RF) sensing mechanisms, wherein RF signals are transmitted toward and reflected or backscattered by passive or semi-passive RF-responsive elements affixed externally to the container. The RF-responsive elements may include, but are not limited to, RFID tags, chipless resonators, metasurfaces, or other RF-interactive structures configured to produce measurable signal variations when their electromagnetic environment changes due to variations in adjacent materials.

The data point acquired at block 3704 may comprise various signal features extracted from the RF interactions, such as RSSI values, which measure the power level of signals received after interaction with the container environment; phase measurements, which capture timing relationships between transmitted and received signals; CSI, which provides detailed frequency-domain profiles of the RF channel including amplitude and phase information across multiple subcarriers; or resonant frequency shifts, which may correlate with specific properties of the container contents. In alternative aspects, data points may be obtained from complementary sensing modalities such as optical, acoustic, thermal, or other non-invasive technologies that can detect properties of the container contents without requiring direct contact.

The acquisition of data points at block 3704 may involve multiple measurements taken from different spatial positions on the container, potentially using an array of RF-responsive elements arranged to provide information about internal content distribution. The data acquisition may also include preprocessing operations such as signal filtering, normalization, or feature extraction to enhance the signal quality and extract relevant information from the raw measurements.

At block 3706, the process 3700 continues with inputting the acquired data point into a machine-learning model by one or more processors. The machine-learning model represents a computational structure that has been trained to recognize patterns and relationships between RF signal characteristics and internal container conditions. The model may be implemented using various architectures such as neural networks, decision trees, random forests, support vector machines, or other suitable machine learning approaches. The machine-learning model may have been previously trained on historical datasets labeled with ground truth values for liquid characteristics such as fill level, alcohol concentration, evaporation loss, or compositional changes. This supervised learning approach enables the model to generalize from known examples to new, unseen data, effectively mapping signal features to internal container properties. The training process may have involved exposing the model to diverse conditions, container types, and content variations to ensure robust performance across different operational scenarios. In some implementations, the machine-learning model is executed on a local computing device, such as a microcontroller or edge processing node located near the container. This approach may reduce latency and minimize network bandwidth requirements. In other implementations, the acquired data may be transmitted to a cloud-based inference platform, which may provide greater computational resources for complex model execution or enable centralized monitoring of multiple containers. The selection between local and remote processing may depend on various factors including power availability, connectivity options, computational requirements, and monitoring frequency.

The input provided to the machine-learning model at block 3706 may include not only the current measurement data but also contextual information such as container metadata, environmental conditions, or historical measurements that provide additional context for accurate inference. The input format may be tailored to the specific model architecture, potentially requiring normalization, reshaping, or other transformations to match the expected input structure of the trained model.

At block 3708, the machine-learning model outputs, based on the input data, at least one characteristic associated with the contents of the container. This output represents the inference result, translating the measured signal features into meaningful properties of the container contents. The output may take various forms depending on the specific monitoring objectives and model design. For regression tasks, the output may include numerical predictions such as a percentage alcohol by volume (ABV), a precise liquid level measurement in centimeters or inches, a volume estimate in liters or gallons, or an evaporation rate over time. These continuous variables provide quantitative assessments of specific content properties that may be tracked over time to monitor aging processes, detect anomalies, or manage inventory. For classification tasks, the output may comprise categorical determinations such as assigning the container to predefined classes like “full,” “half,” or “low” for fill level assessment, or “normal” versus “abnormal” for anomaly detection. These discrete categorizations may be suitable for triggering specific operational responses based on container status. In probabilistic implementations, the output may include a probability distribution over possible states or parameter values, providing not only a central estimate but also a measure of prediction uncertainty. This approach can enable risk-aware decision-making by communicating the confidence level associated with the inference results. The output produced at block 3708 may include not only the primary characteristic of interest but also related parameters, confidence metrics, or supporting information that contextualizes the prediction. For example, along with a predicted alcohol content, the model might output a confidence interval, an estimated time to target maturation, or a comparison against historical trends.

In certain aspects, the output from block 3708 is used to trigger downstream actions within an integrated monitoring and control system. These actions may include activating environmental controls to maintain optimal aging conditions, notifying operators of containers requiring attention or rotation, scheduling maintenance activities, or logging measurements for compliance reporting, quality control, or regulatory purposes. The specific actions triggered may depend on the application context, such as distillery operations, food processing, chemical storage, or other industrial settings where non-invasive content monitoring provides operational value.

At block 3710, the process 3700 concludes. This terminal block represents the completion of a single monitoring and inference cycle. After reaching this endpoint, the system may return to block 3702 to begin another iteration, potentially with updated parameters or adjusted scheduling based on the results of the completed cycle. In continuous monitoring implementations, the process may restart immediately or after a short delay, while in periodic monitoring scenarios, the system might enter a low-power state until the next scheduled measurement interval. The process 3700 illustrated in FIG. 37 provides a streamlined approach to non-invasive monitoring of container contents using RF sensing technology coupled with machine learning inference. This approach enables ongoing assessment of content characteristics without disrupting aging processes, compromising container integrity, or requiring manual sampling, thereby supporting quality control, inventory management, and process optimization across various industrial applications.

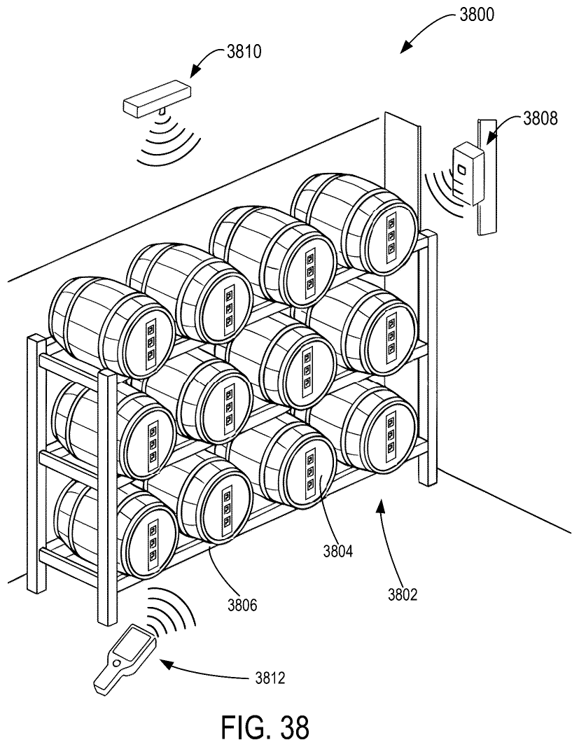

Referring now to FIG. 38, illustrated is an exemplary aspect of a barrel monitoring system 3800 deployed in a warehouse environment 3802, which may represent a rickhouse, aging room, or other storage facility where multiple barrels 3804 are stored. The barrels 3804 are arranged on a racking structure 3806, which provides organized storage and access to the barrels 3804 during aging, fermentation, or other processes. Each barrel 3804 may be the same as or similar to container 110 described in reference to FIGS. 1-2, and may contain liquid content such as distilled spirits, wine, beer, or other fermentable liquids.

The barrel monitoring system 3800 comprises a distributed network of RF reader devices positioned throughout the monitoring environment 3802 to communicate with RF-responsive elements affixed to the barrels 3804. In the illustrated embodiment, these reader devices include a wall-mounted reader 3808 and an overhead reader 3810, both configured to emit RF interrogation signals toward the barrels 3804 and receive reflected or backscattered signals from the RF-responsive elements attached to the barrels. The wall-mounted reader 3808 represents a fixed-position RF interrogation device that may be the same as or similar to the transceiver unit 3230 described in reference to FIG. 32A, and/or the RF reader 2412 described in FIG. 24. The wall-mounted reader 3808 may include an antenna array configured to direct RF signals toward one or more rows or sections of the racking structure 3806, enabling systematic monitoring of multiple barrels simultaneously. The wall-mounted reader 3808 may implement various RF sensing technologies, including but not limited to UHF RFID, FMCW radar, or millimeter-wave sensing modalities as described in connection with FIG. 26.

The wall-mounted reader 3808 may operate continuously, providing ongoing monitoring of barrel conditions, or may activate periodically according to a predetermined schedule similar to the burst transmission patterns described in reference to FIG. 11A. The reader 3808 may include internal processing capabilities similar to processing system 210 described in FIGS. 3-4, or may function primarily as a data acquisition device that forwards collected signals to a central processing system for more comprehensive analysis. The overhead reader 3810 represents another fixed-position RF interrogation device, which may incorporate functionality similar to the node 3518 described in reference to FIG. 35. The overhead reader 3810 may provide complementary coverage to the wall-mounted reader 3808, enabling different signal angles and propagation paths that may enhance detection accuracy or provide redundancy in measurements. The overhead reader 3810 may emit downward-directed RF beams that interrogate multiple barrels across different rows or levels of the racking structure 3806, potentially with wider coverage area than the wall-mounted reader 3808.

In some implementations, the overhead reader 3810 may utilize specialized antenna configurations optimized for top-down interrogation, and may incorporate circular polarization, sectored arrays, or beam-steering capabilities similar to those described in connection with the transmit module 3232 of FIG. 32A. The overhead reader 3810 may operate in coordination with the wall-mounted reader 3808, using time-division multiplexing, frequency diversity, or other coordination mechanisms to avoid interference while maximizing coverage of the barrel monitoring environment. In some aspects, the system 3800 may further include a handheld device 3812, which may function as a portable RF reader or scanner that can be operated by personnel within the warehouse environment. The handheld device 3812 may be the same as or similar to one of the external devices 483, 485, 487, 492, 494, or 495 described in reference to FIG. 4, and/or may incorporate functionality of the remote computing devices 3512 described in reference to FIG. 35. The handheld device 3812 provides flexibility for spot-checking individual barrels, troubleshooting sensing issues, or collecting targeted measurements from specific barrels of interest.

The handheld device 3812 may include a user interface similar to the graphical user interface 3522 described in FIG. 35, displaying measurement results, barrel information, or historical data directly to the operator. The handheld device 3812 may be configured to communicate wirelessly with a central database or processing system, enabling real-time synchronization of collected data with the broader monitoring infrastructure similar to the communication functionality described for communication module 358 in FIG. 3. Each barrel 3804 in the system 3800 may include an RF-responsive element arranged in a pattern on the exterior surface of the barrel. As depicted in FIG. 38, these RF-responsive elements may be arranged in a vertical strip or array on the visible face of each barrel 3804, corresponding to elements 220u-220n as described previously in connection with FIGS. 2, 7A, 7B, and 24. These RF-responsive elements may include passive RFID tags, chipless resonators, metasurfaces, or other RF-interactive structures as described in relation to RF-responsive element 2402 of FIG. 24 and the architecture 2502 of FIG. 25.

The RF-responsive elements attached to each barrel 3804 interact with the RF signals emitted by readers 3808, 3810, and 3812, producing reflected or backscattered signals 3223 as described in reference to FIG. 32A. These RF interactions may be influenced by various factors including the liquid level within the barrel, the dielectric properties of the liquid (which may correlate with alcohol content as described in FIGS. 20A-20D and/or FIG. 29), the presence of vapor in the headspace 3222 described in FIG. 32A, and physical characteristics such as barrel wall moisture or char layer properties as depicted in FIG. 31. When the readers 3808, 3810, or 3812 receive these reflected signals, they may extract various signal features or characteristics for analysis. These features may include RSSI values, phase measurements, CSI, or resonant frequency shifts as described in reference to FIG. 26 and signal characteristics 3314 of FIG. 33.

The racking structure 3806 provides physical support and organization for the barrels 3804 within the warehouse environment 3802, and may be similar to the rack structures described in reference to FIG. 1. The operational workflow of the barrel monitoring system 3800 may involve scheduled or continuous interrogation of the RF-responsive elements by the readers 3808, 3810, with supplementary spot-checks performed using the handheld device 3812. The signal data collected from these interrogations may be transmitted to a processing system which may correspond to the machine-learning system 3504 described in reference to FIG. 35. This processing system may incorporate functionality similar to the AI-driven RF sensing system 3200 described in connection with FIG. 32A, potentially including machine learning models 3202 trained to recognize patterns in RF signal data that correlate with specific barrel conditions. The system may generate outputs including liquid level measurements, alcohol content estimates similar to those described in connection with FIG. 17, evaporation rate calculations, or alerts for anomalous conditions such as leaks or temperature excursions as discussed in relation to FIGS. 24-37. In certain implementations, the barrel monitoring system 3800 may incorporate coordination mechanisms between the various readers to prevent interference during simultaneous operation, similar to the processes described in reference to FIGS. 10-12. The system 3800 enables non-invasive monitoring of barrel contents without requiring direct access to the interior of the barrels, thereby preserving the integrity of aging processes while providing the advantages described throughout the specification.

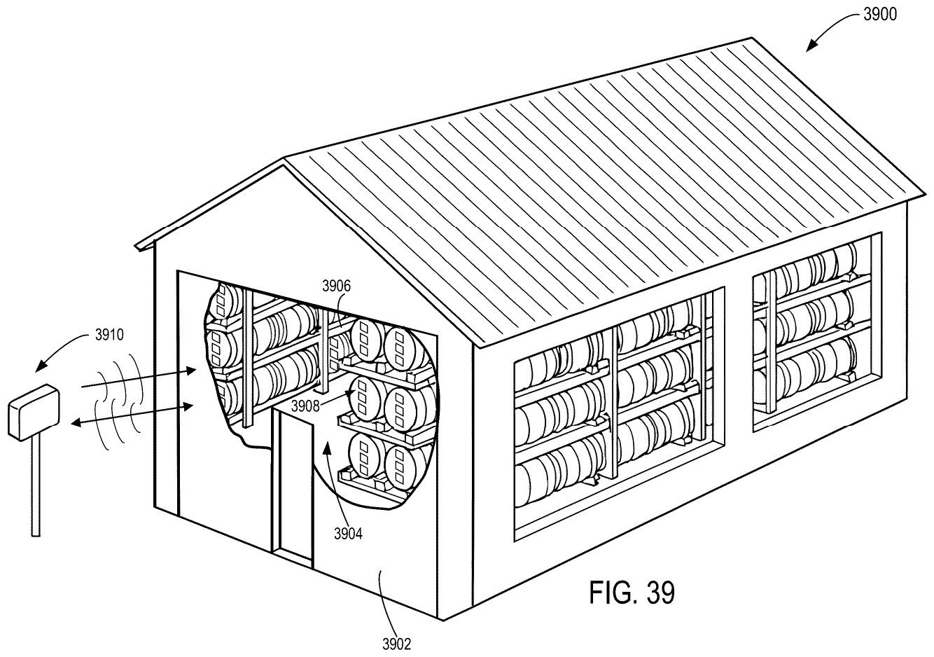

Referring now to FIG. 39, an exemplary rickhouse or barrel-aging facility 3900 is illustrated, configured for non-invasive wireless monitoring of a plurality of containers 3904 in accordance with aspects of the present disclosure. The illustration depicts the rickhouse 3900 with a partially cutaway wall to reveal the internal arrangement of barrels 3904 stored within the facility structure 3902. The facility structure 3902 may comprise various construction materials, which may include wood, metal, concrete, composite materials, or combinations thereof, selected to maintain appropriate environmental conditions for aging processes. The facility structure 3902 may be similar to conventional configurations for storing barrels as discussed with reference to FIG. 1. In this respect, the structure 3902 serves purposes comparable to the storage environments depicted in FIGS. 1 and 9, while accommodating the external monitoring approach described in connection with FIGS. 24-38. The containers 3904, which may be implemented as barrels, casks, or other vessels suitable for storing and aging liquids, correspond functionally to containers 110 described with reference to FIGS. 1-2, 7A-7B, and 24, 32A, 32B, and 35. These containers 3904 are arranged on a multilevel racking system 3906 within the facility structure 3902. The racking system 3906 may be similar to the rack storage configurations depicted in FIGS. 1, 9, and 38, providing support for organizing multiple containers in horizontal or vertical orientations.

Each container 3904 may include one or more externally mounted RF-responsive elements 3908 attached to their exterior surfaces. These elements correspond to the RF-responsive elements 220u-220n described in FIG. 24, the RF-responsive element 2402 of FIG. 24, or may implement the architecture 2502 detailed in FIG. 25. As previously described, these elements may comprise passive RFID tags, chipless resonators, reconfigurable intelligent surfaces, or other RF-interactive structures configured to reflect, backscatter, or otherwise modulate incident RF signals in a manner influenced by the container contents.

Positioned outside the facility structure 3902 is at least one RF interrogation unit 3910. The RF interrogation unit 3910 may be the same as or similar to the RF reader 2412 described in FIG. 24, the transceiver unit 3230 described in FIG. 32A, or the node 3518 described in FIG. 35. Furthermore, the RF interrogation unit 3910 may be implemented as a gNodeB or eNodeB of a 5G or 4G wireless cellular network, leveraging existing telecommunications infrastructure for container monitoring applications as discussed in connection with the communication aspects described with respect to FIG. 24. When implemented as a gNodeB, the RF interrogation unit 3910 may utilize beamforming capabilities native to 5G infrastructure to direct focused energy toward specific regions within the facility, potentially enhancing signal penetration and measurement precision.

The signals emitted by the RF interrogation unit 3910, represented by directional arrows in FIG. 39, correspond to the RF signals 3221 described in FIG. 32A, the transmitted signal 3240 in FIG. 32B, or the interrogation signals described in connection with FIG. 24. These signals pass through structure 3902 and interact with the RF-responsive elements on containers 3904. The resulting backscattered or modulated signals, shown as arrows returning to unit 3910, correspond to the reflected signals 3223 in FIG. 32A, the reflected signal 3244 in FIG. 32B, or the reflected signal 2410 in FIG. 24. These returned signals may contain features or characteristics influenced by the internal conditions of the containers 3904, including RSSI values, phase shifts, resonant frequency variations, or CSI that correlates with specific container parameters as detailed in the signal response profiles of FIG. 26. The RF interrogation unit 3910 may operate in various configurations, including fixed installation as depicted in FIG. 39, or as a mobile unit similar to the handheld device 3812 described in FIG. 38. Data collected by RF interrogation unit 3910 may be processed by systems corresponding to the machine-learning model 3202 described in FIG. 32A, the AI-driven RF sensing system 3200 in FIG. 32A, or the machine-learning system 3504 detailed in FIG. 35. These processing systems implement algorithms similar to those described in process 3600 of FIG. 36 and process 3700 of FIG. 37 to translate RF signal characteristics into meaningful measurements of container contents.

The system depicted in FIG. 39 represents an integrated, facility-scale implementation of the monitoring approaches developed throughout the disclosure, demonstrating how the container-level technologies described in earlier figures may be deployed in a comprehensive monitoring solution for commercial aging facilities. By combining RF sensing elements, cellular or dedicated RF interrogation infrastructure, and advanced signal processing techniques, the system enables non-invasive monitoring across multiple containers without disrupting traditional aging processes or requiring physical access to individual containers.

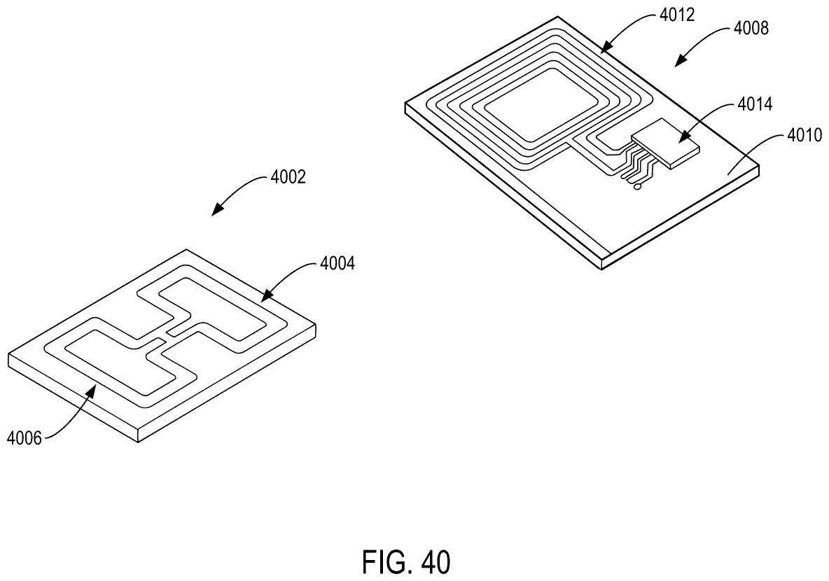

Referring now to FIG. 40, illustrated are two examples of RF-responsive elements that may be affixed to the exterior surface of a container for non-invasively monitoring internal contents. Specifically, FIG. 40 depicts a chipless reflective tag 4002 and a semi-passive RFID tag 4008, both of which may be employed in monitoring systems described in previous figures, such as those illustrated in FIGS. 24, 25, 32, and 35. The chipless reflective tag 4002 represents a passive RF-responsive element that operates without integrated circuits or semiconductors. The tag 4002 comprises a substrate 4004 and a patterned resonator structure 4006 formed thereon. The substrate 4004 may be implemented using various materials including, but not limited to, polyethylene terephthalate (PET), polyimide, FR-4, paper, or other flexible or rigid dielectric materials suitable for supporting RF structures. The substrate 4004 provides mechanical support for the resonator structure while maintaining appropriate dielectric properties that influence the electromagnetic characteristics of the tag. In some implementations, the substrate 4004 may incorporate adhesive backing for attachment to container surfaces, or may feature mounting holes or other attachment mechanisms for securing the tag to curved or irregular container geometries.

The resonator structure 4006 represents a conductive pattern designed to interact with incident RF signals in a frequency-dependent manner. The resonator structure 4006 may be formed of conductive material such as aluminum, copper, silver, gold, or conductive inks and may be fabricated using various manufacturing techniques, including etching, printing, deposition, or laser ablation. The specific geometry of resonator structure 4006 defines its electromagnetic response characteristics, potentially incorporating designs such as split-ring resonators, spiral traces, meandered lines, patch antennas, frequency-selective surfaces, or fractal patterns. These geometries create resonant structures that preferentially reflect, absorb, or scatter RF energy at specific frequencies, with response characteristics that vary based on the dielectric environment adjacent to the tag. When attached to the exterior surface of a container, the resonator structure 4006 interacts with the container wall and internal contents, particularly at the boundary between different materials such as the liquid-air interface in a barrel. This interaction causes measurable shifts in the tag's resonant frequency, amplitude response, phase characteristics, or spectral signature. These shifts may be detected by RF reader devices similar to RF reader 2412 in FIG. 24 or transceiver unit 3230 in FIG. 32A, enabling non-invasive inference of internal container conditions.

The chipless reflective tag 4002 provides several advantages for container monitoring applications. Because it may contain minimal (e.g., or no) semiconductor components, integrated circuits, or power sources, the tag 4002 may be manufactured at a low cost when produced at scale using printing techniques for example. This cost-effectiveness enables high-density deployment across multiple containers or positions on a single container. Additionally, the absence of semiconductors or battery components makes tag 4002 highly resistant to environmental factors such as temperature extremes, humidity fluctuations, or mechanical stress, which may be present in aging warehouses or manufacturing facilities. The tag 4002 may operate effectively across wide temperature ranges from −40° C. to +85° C. or beyond, withstand repeated thermal cycling, and resist degradation from exposure to humidity or corrosive environments. The passive nature of tag 4002 also provides theoretically unlimited operational lifetime, with performance determined primarily by the physical integrity of the substrate and resonator materials rather than battery limitations. In some implementations, the resonator structure 4006 may be designed with multiple resonant elements that respond to different frequency bands, enabling multiplexed sensing of different container parameters or providing redundancy for enhanced reliability.

The semi-passive RFID tag 4008 represents a more sophisticated RF-responsive element that combines passive backscatter communication with limited active functionality. The tag 4008 includes a substrate 4010, an RF antenna structure 4012, and an embedded microchip 4014 mounted or integrated within the tag architecture. The substrate 4010 may be implemented using materials similar to those described for substrate 4004, providing mechanical support and appropriate dielectric properties for the RF components. In some implementations, the substrate 4010 may incorporate multiple layers or regions with different material properties, optimizing performance for both the antenna structure and integrated circuitry. The antenna structure 4012 serves dual purposes in the semi-passive tag architecture. First, it captures RF energy from interrogation signals transmitted by reader devices, potentially harvesting this energy to power the tag's internal circuitry. Second, it enables communication by reflecting or backscattering a portion of the incident RF signal with modulation that encodes the tag's response data. The antenna structure 4012 may be implemented in various geometries including dipole configurations, patch designs, loop structures, or specialized shapes optimized for specific operating frequencies or container geometries. The antenna design may be tuned to maximize performance at particular frequency bands such as 860-960 MHz (UHF RFID), 2.4 GHZ (ISM band), or other frequencies appropriate for container monitoring applications.

The embedded microchip 4014 in tag 4008 provides computational and sensing capabilities beyond those available in the chipless tag 4002. This microchip may include several functional components: a power management circuit that rectifies and regulates harvested RF energy; a digital controller that manages operations and data processing; memory for storing configuration parameters, sensor readings, or unique identification codes; modulation circuitry for controlling backscatter communication; and potentially sensor interfaces for temperature measurement, moisture detection, or other environmental parameters. In some implementations, the microchip may incorporate an analog-to-digital converter (ADC) that measures the strength of received RF signals, enabling the tag to report this value as part of its response. The semi-passive tag 4008 may operate in various modes depending on the application requirements and available energy. In fully passive mode, it relies entirely on harvested RF energy from reader interrogations, operating only when sufficient power is available from the incident signal. In semi-passive mode, it may incorporate an energy storage element such as a capacitor or thin-film battery that accumulates harvested energy over time, enabling periodic sensor measurements or data logging even when reader signals are intermittent. The energy storage element may provide sufficient power for the tag to measure and record parameters such as temperature profiles or signal strength variations over time, creating a history of container conditions that can be retrieved during subsequent reader interrogations.

Both tag architectures 4002 and 4008 may be incorporated into the barrel monitoring systems described in connection with FIGS. 24, 32A, 38, and 39, potentially affixed to barrels 3804 and interrogated by readers 3808, 3810, or handheld device 3812. They may also be employed in the rickhouse monitoring configuration illustrated in FIG. 39, attached to containers 3904 and interrogated by RF unit 3910. When deployed across multiple positions on a container or across multiple containers in a facility, these tags enable comprehensive, non-invasive monitoring of aging or storage processes without requiring direct access to container contents. The selection between chipless tags 4002 and semi-passive tags 4008 for a particular application may depend on various factors including cost constraints, required measurement precision, environmental conditions, and monitoring frequency. In some implementations, both tag types may be deployed in complementary configurations, with chipless tags providing high-density, low-cost coverage and semi-passive tags offering enhanced functionality at specific points of interest. The data collected from these tag deployments may be processed using the machine-learning approaches described in connection with FIGS. 32-37, translating RF signal characteristics into actionable insights about container contents or conditions.

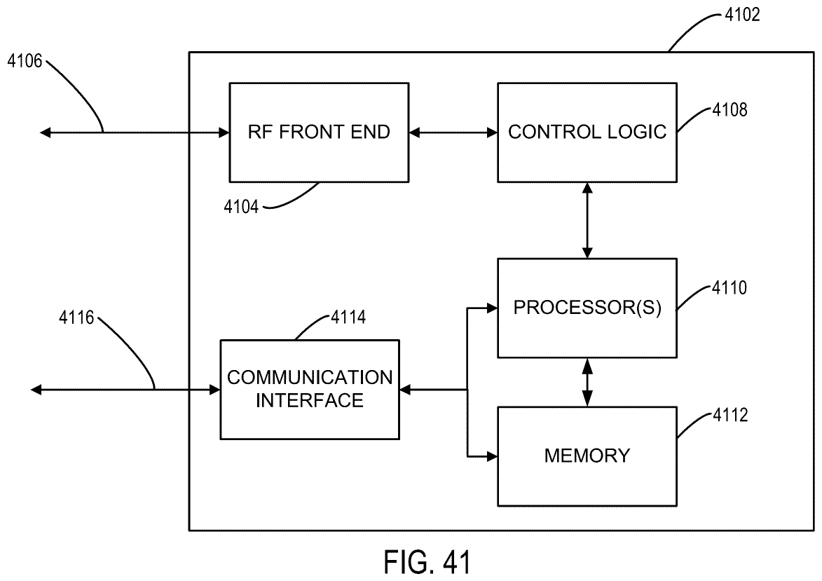

Referring now to FIG. 41, an example of an RF interrogator 4102 is illustrated in accordance with aspects of the present disclosure. The RF interrogator 4102 may be configured to transmit interrogation signals and receive backscattered, reflected, and/or modulated responses from RF-responsive elements positioned externally on containers to monitor internal contents. The RF interrogator 4102 may be implemented as part of a standalone reader device, a gNodeB, a Wi-Fi/IoT access point, or another suitable RF sensing system. In some aspects, the RF interrogator 4102 may correspond to or implement functionality similar to the RF reader 2412, as described with reference to FIG. 24, the transceiver unit 3230 described with reference to FIG. 32A, or the node 3518 described with reference to FIG. 35.

The RF interrogator 4102 includes an RF front-end 4104 operatively connected to an antenna path 4106. The RF front-end 4104 comprises hardware components that manage the transmission and reception of radio frequency signals. In some implementations, the RF front-end 4104 may include multiple transmit and receive chains configured to operate across various frequency bands suitable for penetrating container materials and detecting reflections from internal content boundaries. The RF front-end 4104 may incorporate power amplifiers for boosting outgoing signals to appropriate transmission power levels, low-noise amplifiers (LNAs) for enhancing weak received signals while minimizing added noise, mixers for frequency conversion between baseband and RF domains, filters for rejecting out-of-band interference, switches or duplexers for managing transmit/receive paths, and impedance matching circuitry for optimizing power transfer between components. The RF front-end 4104 may be configured to emit various RF signal types, including continuous wave carriers, frequency-modulated continuous wave (FMCW) signals, pulsed transmissions, or modulated waveforms matching specific sensing protocols. In some aspects, the RF front-end 4104 may support frequency sweeping capabilities, wherein the transmission frequency is varied across a predefined range to obtain spectral response characteristics similar to those described with reference to FIGS. 20A-20D, 29, and 31. Additionally, the RF front-end 4104 may implement beamforming techniques using phased antenna arrays to direct energy toward specific container regions or to simultaneously monitor multiple containers arranged in configurations similar to those illustrated in FIGS. 38 and 39. The RF front-end 4104 may further incorporate phase modulation or manipulation capabilities to enhance sensing accuracy or to support advanced modulation schemes for tag communication.

The antenna path 4106 represents the signal path connecting the RF front-end 4104 to one or more external antennas. This path may comprise transmission lines, connectors, impedance matching networks, or other components that facilitate efficient signal transfer between the RF front-end 4104 and the antenna system. In some implementations, the antenna path 4106 may support multiple antenna connections to enable spatial diversity, MIMO (Multiple-Input Multiple-Output) operations, or beamforming capabilities. The antenna path 4106 may be configured with appropriate isolation and filtering to minimize interference between transmit and receive functions, particularly in continuous-wave backscatter sensing applications.

The RF interrogator 4102 further includes control logic 4108 operatively coupled to the RF front-end 4104. The control logic 4108 may be implemented using digital logic circuits, microcontrollers, field-programmable gate arrays (FPGAs), or application-specific integrated circuits (ASICs) configured to manage the operational parameters of the RF front-end 4104. The control logic 4108 generates timing signals, configures frequency settings, establishes power levels, and coordinates transmit/receive switching to implement specific interrogation protocols. In some aspects, the control logic 4108 may manage duty cycling for power conservation, particularly in battery-powered implementations similar to the power management techniques described with reference to FIGS. 11A-11C. The control logic 4108 may direct the RF front-end 4104 to issue specific frequency bands, modulation patterns, or beam steering configurations optimized for different container materials, content types, or deployment scenarios. For example, when monitoring wooden barrels, the control logic 4108 may select frequencies and power levels appropriate for penetrating oak staves while maximizing reflection from liquid interfaces, as described in connection with the systems of FIGS. 24 and 32.

A set of processor(s) 4110 may be communicatively coupled to the control logic 4108 and configured to perform signal processing, feature extraction, and inference operations. The processor(s) 4110 may be implemented using general-purpose microprocessors, digital signal processors (DSPs), or specialized machine learning processors tailored for RF signal analysis. In some implementations, the processor(s) 4110 may incorporate multiple processing cores or heterogeneous computing architectures to balance performance requirements with power constraints. The processor(s) 4110 may execute algorithms that extract various signal parameters from the received backscattered or reflected signals. These parameters may include RSSI, which measures the power level of received signals as described in connection with FIG. 26; phase angles or phase shifts between transmitted and received signals that may indicate propagation changes due to dielectric boundaries; CSI, which provides detailed frequency-domain profiles of the RF channel including amplitude and phase information across multiple subcarriers; or resonant frequency shifts that correlate with specific properties of container contents as illustrated in FIGS. 29 and 31. Using these extracted signal features, the processor(s) 4110 may implement inference algorithms to estimate physical characteristics within monitored containers. For example, the processor(s) 4110 may determine liquid level based on the transition patterns in RSSI or phase responses across vertically arranged RF-responsive elements, as described with reference to FIGS. 27 and 28. The processor(s) 4110 may estimate alcohol content percentages based on frequency response patterns similar to those illustrated in FIG. 29, or detect anomalous conditions such as leakage using signal signatures similar to those depicted in FIG. 30.

In some implementations, the processor(s) 4110 may execute machine learning models similar to those described with reference to FIGS. 33 and 34, transforming raw signal measurements into actionable insights about container contents. These models may implement neural networks, decision trees, support vector machines, or other machine learning architectures trained to recognize patterns corresponding to specific container states or content properties.

The RF interrogator 4102 further includes memory 4112 coupled to the processor(s) 4110. The memory 4112 may comprise various storage technologies including volatile memory (such as SRAM or DRAM), non-volatile memory (such as Flash, EEPROM, or MRAM), or combinations thereof arranged to provide appropriate capacity and performance characteristics for the application. The memory 4112 may store firmware that defines the operational behavior of the RF interrogator 4102, signal-processing algorithms that implement feature extraction or filtering operations, threshold parameters that define decision boundaries for classification or alerting, or trained machine-learning models that map signal features to container properties. Additionally, the memory 4112 may store reference data that aids in signal interpretation or system calibration. This data may include tag lookup tables that associate tag identifiers with spatial positions or expected responses, historical response curves that establish baselines for comparison, or environmental calibration data that compensates for temperature, humidity, or other ambient factors affecting RF propagation. In some implementations, the memory 4112 may maintain logs of measurement history, enabling trend analysis or anomaly detection based on deviations from established patterns as described with reference to FIGS. 27 and 28.

A communication interface 4114 is provided to enable the RF interrogator 4102 to exchange data with external systems via a communication path 4116. The communication interface 4114 serves as a bridge between the RF sensing domain and broader monitoring or control infrastructure, translating internal data representations into standardized communication protocols. The communication interface 4114 may implement wired connectivity options such as Ethernet, USB, RS-485, or CAN bus; wireless protocols such as Wi-Fi, Bluetooth, ZigBee, or LoRa; or cellular standards such as 5G NR, NB-IoT, or LTE-M, selected based on deployment requirements for range, bandwidth, and power consumption. Through the communication interface 4114, the RF interrogator 4102 may transmit various data types to external systems. This data may include raw or processed signal features extracted from RF interactions, inference results derived from local processing (such as estimated liquid levels or alcohol content), tag identifiers for inventory tracking, or environmental metadata providing context for measurements. The communication interface 4114 may support bidirectional communication, allowing the RF interrogator 4102 to receive configuration updates, adjust operational parameters, or download updated machine learning models from remote management systems.

The communication path 4116 represents the physical or wireless connection between the communication interface 4114 and external systems. This path may be implemented using various technologies depending on the deployment scenario, from direct cable connections in fixed installations to wireless links in mobile or distributed sensing applications. The communication path 4116 may connect the RF interrogator 4102 to monitoring dashboards, databases, control systems, or distributed processing infrastructure as depicted in the system architecture of FIG. 35.

The architecture of RF interrogator 4102 supports both active and passive tag interrogation methods. For passive tag interrogation, the RF front-end 4104 may emit continuous carrier waves and detect modulated backscatter from tags that reflect the incident energy with characteristic variations based on their electromagnetic environment. For active or semi-passive tag communication, the RF interrogator 4102 may implement more complex bidirectional protocols, potentially with dedicated time slots for tag responses or specific modulation schemes optimized for reliable data exchange. The RF interrogator 4102 may be configured to scan arrays of RF-responsive elements similar to those described with reference to FIGS. 2, 7A, 7B, and 24. When these elements are placed on container surfaces such as barrel staves, variations in their response characteristics may indicate dielectric transitions between air and liquid portions within the container. By analyzing these variations across multiple elements or frequencies, the RF interrogator 4102 can detect liquid levels, monitor evaporation rates, or assess compositional changes without requiring direct access to container contents.

The modular design of RF interrogator 4102 enables flexible deployment across various monitoring scenarios. The system may be implemented in warehouse-scale deployments monitoring multiple containers simultaneously, as illustrated in FIGS. 38 and 39; in edge installations providing focused coverage for specific container arrangements; or in handheld devices similar to device 3812 in FIG. 38 for mobile inspection or troubleshooting operations. The RF interrogator 4102 may also be integrated with broader monitoring and control infrastructure, feeding data into distributed analysis systems like those depicted in FIG. 35 to support comprehensive container management across large facilities.

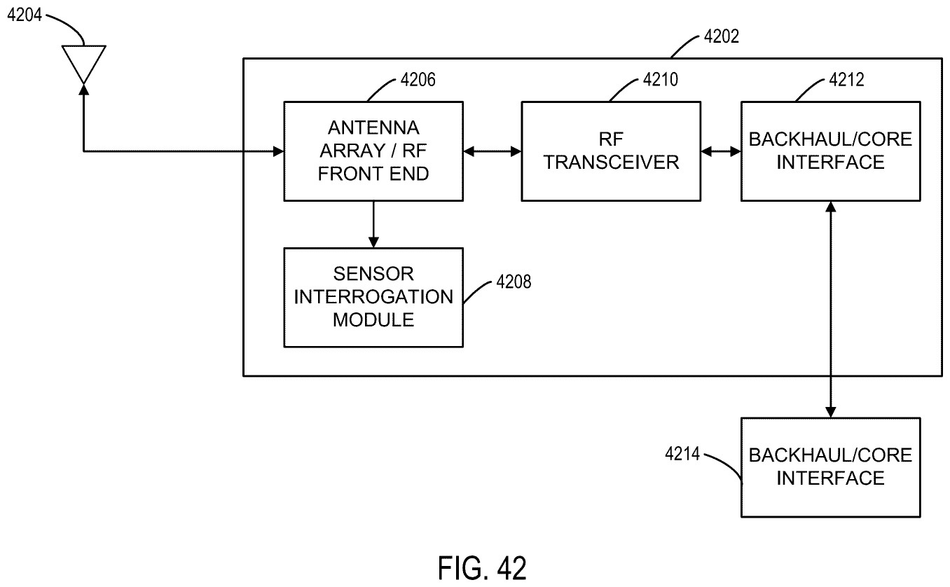

FIG. 42 illustrates an example of a wireless communication node 4202 configured to implement RF-based environmental sensing capabilities within a cellular infrastructure. The wireless communication node 4202 may represent a 5G base station (gNodeB) or similar wireless access point that combines conventional wireless communication functionality with specialized RF sensing capabilities directed toward non-invasive monitoring of container contents as described in previous embodiments. The wireless communication node 4202 may include at least one antenna 4204 configured to transmit and receive radio frequency signals. The antenna 4204 may comprise a single radiating element or, more commonly in cellular implementations, may represent a portion of a larger antenna array capable of directional transmission and reception. The antenna 4204 enables bidirectional wireless communication between the node 4202 and various wireless devices, while also facilitating specialized sensing operations when directed toward RF-responsive elements positioned on containers similar to those described in connection with FIGS. 24-41.

The wireless communication node 4202 may further include an antenna array and RF front end 4206 coupled to the antenna 4204. The antenna array and RF front end 4206 may correspond functionally to the transceiver unit 3230 described with reference to FIG. 32A and/or the RF reader 2412 described with reference to FIG. 24. The antenna array portion of RF front end 4206 may comprise multiple radiating elements arranged in a predefined spatial configuration to support MIMO operations and beamforming capabilities. These capabilities enable the wireless communication node 4202 to direct focused RF energy toward specific spatial regions, which may contain RF-responsive elements affixed to containers as described in previous embodiments. The beamforming functionality allows for enhanced signal penetration through challenging materials like wooden barrel staves or metal-reinforced containers, potentially improving sensing accuracy compared to omnidirectional transmission approaches.

The RF front end portion 4206 incorporates various signal conditioning and routing components that manage the conversion between digital baseband signals and analog RF waveforms suitable for wireless transmission. These components may include power amplifiers that boost outgoing signals to appropriate transmission levels; low-noise amplifiers (LNAs) that enhance the reception of weak reflected signals while minimizing noise contribution; mixers that perform frequency conversion between baseband and RF domains; filters that selectively pass desired frequency components while suppressing unwanted signals; duplexers or switches that enable time-division or frequency-division sharing of antenna resources between transmit and receive functions; and impedance matching networks that optimize power transfer between system components. The RF front end 4206 may be configured to operate across multiple frequency bands, potentially including sub-6 GHz bands commonly used in cellular deployments as well as specialized bands optimized for container sensing applications.

Connected to the antenna array and RF front end 4206 is a sensor interrogation module 4208, which represents a functional block dedicated to RF-based environmental sensing operations. The sensor interrogation module 4208 may correspond functionally to the machine-learning system 3504 described with reference to FIG. 35 and/or the AI-driven RF sensing system 3200 described with reference to FIG. 32A. The sensor interrogation module 4208 is configured to generate interrogation signals suitable for probing RF-responsive elements positioned on containers, direct these signals through the antenna array and RF front end 4206, receive reflected or backscattered responses, and extract meaningful information from these responses.

The sensor interrogation module 4208 may implement various sensing modalities as described in previous embodiments, including passive backscatter detection from RFID tags or chipless resonators, active interrogation of semi-passive sensing elements, or direct radar-based monitoring of liquid-air interfaces within containers. The sensor interrogation module 4208 may generate waveforms optimized for sensing applications, such as frequency-modulated continuous wave (FMCW) signals, ultra-wideband pulses, or frequency-swept interrogation signals that provide enhanced spectral information about the monitored environment. The sensor interrogation module 4208 may analyze various signal features extracted from the reflected or backscattered responses, including RSSI values as described in connection with FIG. 27, phase information as detailed in FIG. 28, CSI metrics, or resonant frequency characteristics similar to those illustrated in FIGS. 29-31. Based on these signal features, the sensor interrogation module 4208 may perform direct inference of container properties, such as determining liquid level, estimating alcohol content, detecting seepage events, or monitoring evaporation rates as described throughout previous embodiments. Alternatively, the module may extract and format signal features for transmission to external processing systems via the backhaul/core interface 4212, potentially leveraging one or more other machine learning models or centralized analytics platforms for enhanced inference accuracy. The sensor interrogation module 4208 may operate independently from the wireless communication functions of the node 4202 or may coordinate with these functions to share resources or minimize potential interference.

The wireless communication node 4202 further includes an RF transceiver 4210 connected to the antenna array and RF front end 4206. The RF transceiver 4210 performs the conventional wireless communication functions of the node, managing the modulation and demodulation of signals according to applicable wireless standards such as 5G NR, LTE, or Wi-Fi. The RF transceiver 4210 may implement various modulation schemes, channel coding techniques, and multiple access methodologies appropriate for the wireless communication standard being supported. The transceiver may further manage frequency hopping, power control, timing synchronization, and other protocol-specific requirements for robust wireless communication.

While the RF transceiver 4210 and sensor interrogation module 4208 are depicted as separate functional blocks, they may share certain hardware resources through the antenna array and RF front end 4206. This resource sharing may be implemented through time-division multiplexing, frequency-division multiplexing, or other coordination mechanisms that enable both communication and sensing functions to operate effectively without mutual interference. The transceiver 4210 may be designed to support multiple simultaneous communication sessions with various user equipment devices while the sensor interrogation module 4208 periodically performs sensing operations, potentially leveraging idle resources during low traffic periods.

A backhaul/core interface 4212 is provided within the wireless communication node 4202, facilitating connectivity between the node and broader network infrastructure. The backhaul/core interface 4212 manages the transmission and reception of data between the wireless communication node 4202 and other network elements, such as a cellular core network, cloud computing resources, or centralized management systems. The interface supports both control plane signaling for node management and user plane data for content delivery, enabling integrated operation of the communication and sensing functions.

The backhaul/core interface 4212 may implement various physical connectivity options, such as fiber optic links, microwave backhaul, or wired Ethernet connections, based on deployment requirements and available infrastructure. The interface may support standardized protocols such as GTP-U, SCTP, or IP-based communication, enabling seamless integration with existing network elements. Through this interface, the wireless communication node 4202 can transmit sensing data, inference results, or extracted signal features to external processing systems, management platforms, or application servers that leverage this information for inventory tracking, quality control, regulatory compliance, or other operational purposes related to container monitoring.

In some deployment scenarios, the wireless communication node 4202 may connect to an external backhaul/core interface 4214, which provides additional connectivity options or interface capabilities beyond those integrated directly within the node. The external backhaul/core interface 4214 may represent a separate physical device or network element that extends the connectivity options available to the wireless communication node 4202, potentially providing additional protocol support, routing capabilities, or security features. The external interface 4214 may be deployed in network architectures that implement functional splits between radio and baseband processing, such as Centralized RAN (C-RAN) configurations where baseband processing occurs at centralized locations separate from radio transmission sites.

The configuration illustrated in FIG. 42 enables dual-use operation of cellular infrastructure, leveraging existing or planned wireless communication deployments for enhanced environmental sensing capabilities. By integrating the sensor interrogation module 4208 within the wireless communication node 4202, the system can provide both conventional wireless connectivity services and specialized container monitoring functions without requiring separate dedicated sensing infrastructure. This integration may be particularly valuable in environments such as distillery warehouses, chemical storage facilities, or production environments where both wireless connectivity and non-invasive container monitoring are desired.

The wireless communication node 4202 may utilize standardized wireless spectrum for both communication and sensing operations, potentially reducing deployment costs compared to dedicated sensing systems operating in specialized frequency bands. In some implementations, the node 4202 may dedicate specific time or frequency resources to sensing operations, ensuring that these functions do not interfere with critical communication services while maintaining adequate monitoring coverage and update frequency. The beamforming capabilities of the antenna array enable directed interrogation of specific container regions, potentially enhancing sensing precision or enabling selective monitoring of containers arranged in dense configurations similar to those illustrated in FIGS. 38 and 39.

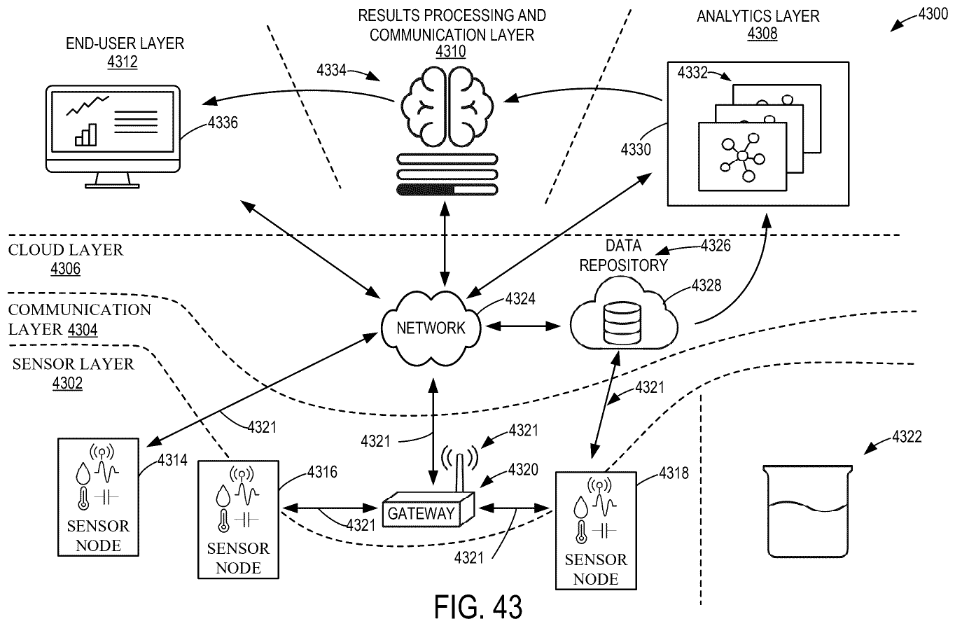

FIG. 43 illustrates an example implementation of an integrity monitoring platform 4300 and other substrates contained within a sealed container, such as a vessel, in accordance with aspects of the present disclosure. In some aspects, the integrity monitoring platform 4300 provides a system architecture that enables real-time monitoring and analysis of fluid properties across diverse industrial applications utilizing multiple functional layers. That is, the integrity monitoring platform 4300 may include a sensor layer 4302, a communication layer 4304, a cloud layer 4306, an analytics layer 4308, a results processing and communication layer 4310, and an end-user layer 110.

The sensor layer 4302 may include one or more sensor nodes, illustrated as sensor nodes 4314, 4316, and 4318. Each sensor node may be positioned to monitor containers or vessels containing target substances. Each sensor node within the sensor layer 4302 may incorporate non-invasive measurement capabilities to measure electromagnetic properties through container walls. The sensor nodes 4314, 4316, and 4318 may be field-deployable units that can be externally mounted to various container types without requiring penetration or modification of the containment vessel. The sensor nodes may incorporate multiple sensing modalities beyond electromagnetic property measurements, including but not limited to temperature sensors, humidity sensors, pressure transducers, and volatile organic compound detectors. The modular design of the sensor nodes enables adaptation to various container geometries and materials while maintaining consistent measurement capabilities. Each sensor node may include local processing capabilities to perform initial signal conditioning and data preprocessing before transmission, reducing bandwidth requirements and enabling edge computing functionality when network connectivity is limited. The sensor nodes may be powered through various means, including, but not limited to, an energy storage device (e.g., a battery, capacitor), energy harvesting from environmental sources, and/or wired power connections, depending on the deployment requirements.

As depicted in FIG. 43, a sensor node, such as sensor node 4318, may be associated with a container 4322. The container 4322 may represent a vessel or containment system that holds liquids or other substances subject to integrity monitoring. The container 4322 may comprise various forms including but not limited to wooden barrels (e.g., for aging spirits and other liquids), steel and/or plastic pipelines (e.g., for transporting liquids), plastic containers (e.g., for liquid storage), and/or concrete tanks (e.g., for use at water treatment facilities). The external mounting of one or more sensor nodes (e.g., 4314, 4316, and 4318) to containers (e.g., container 4322) enables continuous monitoring without compromising container integrity or introducing contamination risks.

The communication layer 4304 provides a transmission infrastructure connecting the sensor layer 4302 to higher-level processing systems. In some aspects, the communication layer 4304 manages multiple communication pathways and protocols to provide reliable data transfer from distributed sensor deployments (e.g., one or more of sensor nodes 4314, 4316, and/or 4318). In some aspects, a gateway 4320 may act as a primary aggregation point within the communication layer 4304, collecting data from one or more sensor nodes through wireless communication links 4321. The wireless communication links 4321 may employ one or more protocols for different deployment scenarios, including but not limited to LoRaWAN for long-range, low-power applications; NB-IoT for cellular network integration; Zigbee or Bluetooth for short-range, high-density deployments; and/or Wi-Fi for high-bandwidth applications. In some aspects, the gateway 4320 performs protocol translation, data buffering, and preliminary data validation before forwarding information to a cloud layer 4306. In certain implementations, the gateway 4320 may include edge computing capabilities to execute lightweight analytics models and enable rapid local responses to detected anomalies while maintaining cloud connectivity for a more comprehensive analysis. The communication layer 4304 may implement security measures, including encryption, device authentication, and/or secure key management to protect data integrity during transmission.

In some aspects, network 4324 connects the gateway 4320 and other communication layer components to cloud-based resources in the cloud layer 4306. In some aspects, a network 4324 represents a broader telecommunications infrastructure, including one or more internet connectivity, private networks, or hybrid cloud connections that enable data flow between field deployments and centralized processing systems. The network architecture supports redundant pathways and failover mechanisms to ensure continuous operation even during connectivity disruptions.

The cloud layer 4306 provides scalable storage and computational infrastructure for the integrity monitoring platform 4300. A data repository 4326 within the cloud layer 4306 stores both raw sensor data and processed information, maintaining historical records for trend analysis, compliance documentation, and forensic investigation. The data repository 4326 implements data lifecycle management policies, which may include retention periods, archival strategies, and data purging protocols to meet industry-specific regulatory requirements. The cloud infrastructure 4328 encompasses the complete cloud computing environment, including virtual machines, container orchestration systems, load balancers, and auto-scaling groups that provide elastic capacity to handle varying computational demands. In some aspects, the cloud layer 4306 may be deployed across multiple geographic regions to comply with data security requirements and to reduce latency for global operations.

The analytics layer 4308 may perform data processing and machine learning operations based on the collected sensor data. In some aspects, an analytics engine 4330 may orchestrate the execution of various analytics models and algorithms 4332. The analytics engine 4330 may implement a model registry system that manages multiple industry-specific machine-learning models, where each model is trained for and/or optimized for one or more use cases. For example, for spirits monitoring, a machine-learning model may be trained to detect adulterants based on deviations in dielectric signatures from aged whiskey baselines. For aviation fuel applications, machine-learning models may identify water contamination or fuel grade mismatches through pattern recognition of dielectric spectra. In some aspects, pharmaceutical implementations utilize machine-learning models that detect counterfeiting or tampering by identifying subtle variations in liquid composition signatures. The analytics layer 4308 may support both batch processing for comprehensive analysis and stream processing for real-time anomaly detection. The analytics models and algorithms 4332 may undergo continuous improvement through federated learning approaches that incorporate field data while maintaining data privacy and security. Transfer learning techniques can be used to facilitate the rapid deployment of new models by leveraging base dielectric models that are adapted to specific industry requirements.

In some aspects, the results processing and communication layer 4310 transforms raw analytics outputs into actionable intelligence for end users. A results processing module 4334 may apply business logic, formatting rules, and/or industry-specific compliance requirements to generate user-ready outputs. The results processing module 4334 may perform alert prioritization, severity classification, and/or notification routing based on configured thresholds and escalation policies. In some aspects, the results processing module 4334 may generate various output formats, including real-time alerts for critical anomalies, periodic compliance reports for regulatory submissions, trend analysis summaries for operational planning, and forensic investigation reports for security incidents. In some aspects, the results processing and communication layer 4310 may implement application programming interfaces (APIs) that enable integration with various enterprise systems, including, but not limited to, manufacturing execution systems, laboratory information management systems, enterprise resource planning platforms, and supply chain management solutions.

The end-user layer 4312 provides a human-machine interface for system interaction and result consumption. A dashboard and visualization interface 4336 presents processed information through intuitive graphical displays, including real-time monitoring dashboards with customizable widgets, geographic information system maps showing distributed sensor deployments, time-series charts displaying historical trends and anomalies, compliance scorecards with regulatory metric tracking, and alert management consoles for incident response. The graphical interface 4336 may support role-based access control policies to provide users with information based on their responsibilities and authorization levels. Mobile applications and responsive web interfaces enable access from various devices, supporting both operational monitoring and executive oversight requirements.

In some aspects, the sensing hardware may be separate from the analytics models, allowing the same physical sensor nodes (e.g., 4314, 4316, and 4318) to serve diverse applications through software configuration changes. Industry-specific customization can occur in the analytics models and results processing logic while the core infrastructure remains consistent across deployments.

In some aspects, data may flow through the integrity monitoring platform 4300 using established pathways while maintaining flexibility for various operational modes. Sensor nodes 4314, 4316, and/or 4318 may continuously or periodically collect dielectric measurements and environmental data from their associated containers. This data may be transmitted via wireless communication links 4321 to the gateway 4320, which aggregates and forwards information through the network 4324 to the cloud infrastructure 4328. The data repository 4326 may store incoming data while simultaneously feeding the analytics engine 4330. Processed results from the analytics layer 4308 flow to the results processing module 4334, which may format and distribute information through the dashboard interface 4336 and other output channels.

In some aspects, the integrity monitoring platform 4300 supports multiple deployment topologies to accommodate diverse operational requirements. In centralized deployments, sensor data flows to a single cloud instance for processing. Distributed deployments may utilize regional cloud instances to reduce latency and meet data residency requirements. Hybrid deployments may combine edge processing at gateway nodes with cloud-based analytics for comprehensive analysis. Offline-capable deployments store data locally during connectivity outages and synchronize it when connections are restored.

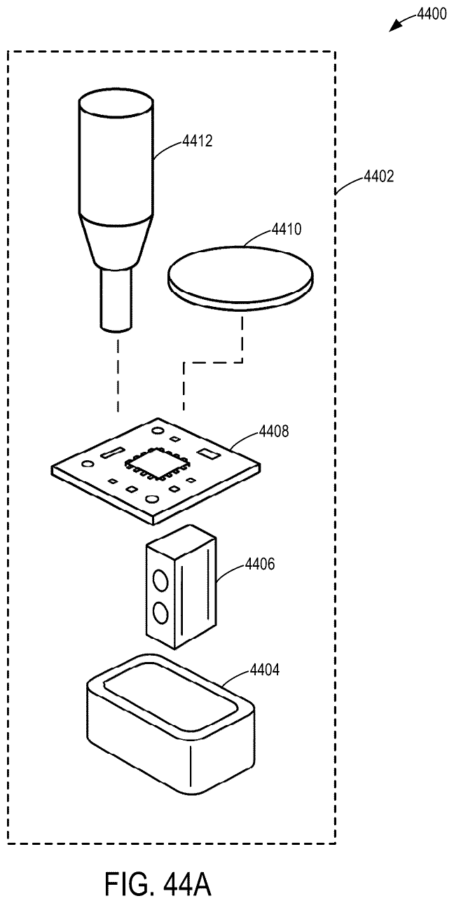

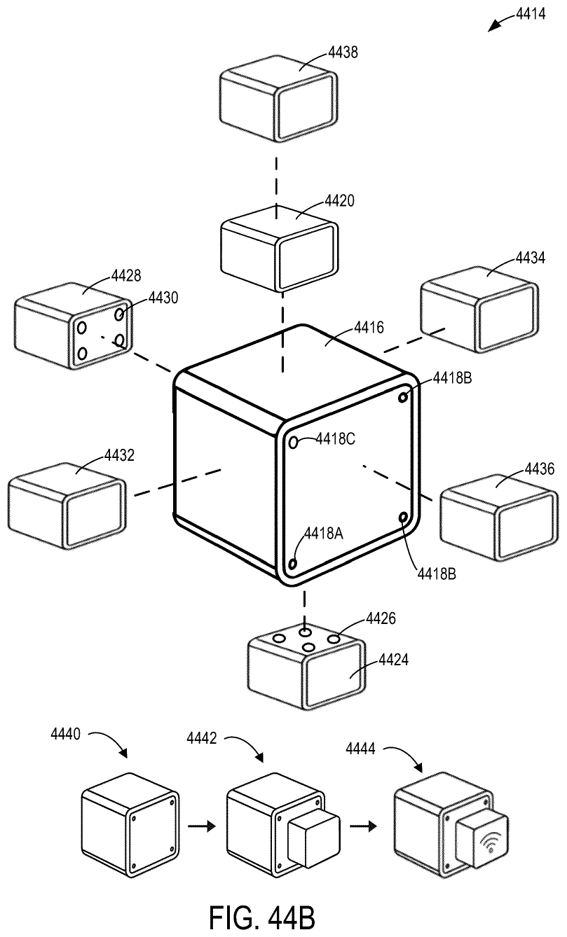

FIG. 44A illustrates an exploded view of a universal sensor node assembly 4400 that forms part of the integrity monitoring platform 4300, in accordance with aspects of the present disclosure. The universal sensor node assembly 4400 may be the same as or similar to the sensor node 4314, 4316, and/or 4318 of FIG. 43. The sensor node assembly 4400 is depicted as a vertically exploded arrangement, showing the spatial relationships and assembly sequence of the various functional components 4402 that collectively enable non-invasive monitoring of liquid integrity within a container, such as a sealed container.

At the uppermost position of the exploded view depicted in FIG. 44A is a dielectric sensor 4412 and a radar antenna 4410. In some aspects, the sensor node assembly 4400 may include a dielectric sensor 4412 and/or the radar antenna 4410. The dielectric sensor 4412 serves as one of the primary sensing elements within the platform architecture, providing a non-invasive measurement of the dielectric properties of liquids contained within sealed vessels. In operation, the dielectric sensor 4412 measures the relative permittivity (Er) or dielectric constant of substances using capacitive or electromagnetic field-penetrating techniques. The dielectric sensor 4412 may generate an electric field that penetrates through a container wall and interacts with the contained liquid, where changes in the dielectric properties of the liquid alter the field characteristics in measurable ways. These alterations correlate with specific liquid compositions, contamination levels, or adulterants, and may enable the system to detect anomalies such as water contamination in aviation fuel, dilution in alcoholic spirits, or tampering in pharmaceutical solutions.

In some aspects, the radar antenna 4410 provides complementary sensing capabilities to the dielectric sensor 4412, enabling non-contact level sensing and material boundary detection through electromagnetic wave transmission and reception. The antenna 4410 may emit controlled electromagnetic pulses or continuous waves that propagate through the container wall and reflect from liquid surfaces or material interfaces within the container. By analyzing the time-of-flight, phase shift, and amplitude characteristics of the reflected signals, one or more components of the integrity monitoring platform 4300 can determine liquid levels, detect stratification layers, identify settled solids, or recognize the presence of multiple phases within the container. In some aspects, the circular configuration of the antenna 4410 provides omnidirectional or focused beam patterns depending on the specific implementation requirements. In some aspects, the radar antenna 4410 further provides dielectric information through analysis of the returned electromagnetic signals. When electromagnetic waves propagate through a liquid medium, the propagation velocity changes according to the relationship v=c/√εᵣ, where c represents the speed of light in vacuum and εᵣ represents the relative permittivity of the medium. This velocity modification affects time-of-flight measurements, enabling the system to infer dielectric properties from timing variations in the returned signals. At interfaces between materials exhibiting different dielectric constants, such as air-liquid boundaries or stratified liquid layers, the reflection coefficient may depend on the dielectric contrast between the materials. The amplitude and phase characteristics of these reflections carry information about the dielectric properties of materials on either side of each interface.

Additionally, radar signals may experience attenuation patterns that vary based on the dielectric loss tangent (tan δ) of the medium. Analysis of signal strength decay through the medium enables the characterization of both real and imaginary components of the complex permittivity. When employing wideband or swept-frequency radar techniques, the frequency-dependent behavior of returned signals provides a dielectric spectrum signature unique to different liquid compositions.

In some aspects, the sensor node assembly 4400 may include a microcontroller and printed circuit board (PCB) assembly 4408, positioned centrally within the sensor node stack. The microcontroller and PCB assembly 4408 provides the computational and control hub functions of the sensor node, orchestrating sensing, processing, and communication operations. The microcontroller and PCB assembly 4408 may interface with both the dielectric sensor 4412 and radar antenna 4410 through suitable analog-to-digital conversion circuitry, collecting raw sensor data at configurable sampling rates. Beyond simple data collection, the microcontroller and PCB assembly 4408 may perform preprocessing functions, which may include but are not limited to, signal conditioning, digital filtering, feature extraction, and data compression. The microcontroller and PCB assembly 4408 may implement one or more algorithms for extracting dielectric information from both sensor inputs, including fast Fourier transform (FFT) processing for frequency-domain analysis of radar returns, phase detection circuits for measuring propagation delays, and correlation algorithms for identifying material boundaries from reflection patterns. In implementations supporting edge deployments, the microcontroller and PCB assembly 4408 may execute lightweight machine learning models (TinyML) directly on the sensor node, enabling local anomaly detection and reducing the volume of data requiring transmission to cloud infrastructure. The microcontroller and PCB assembly 4408 may incorporate circuitry such as voltage regulation, sensor interface electronics, memory for data buffering, and communication protocol stacks.

The RF front-end module 4406 may provide an interface between the digital domain of the microcontroller 4408 and the analog RF domain utilized for wireless communication. The RF front-end 4406 may incorporate multiple functional blocks, including power amplifiers for signal transmission, low-noise amplifiers for reception, frequency synthesizers for channel selection, and impedance-matching networks for optimal power transfer. The RF front-end 4406 may support multiple wireless communication protocols relevant to industrial IoT deployments, which may include but are not limited to, LoRaWAN for long-range low-power communication, Wi-Fi for high-bandwidth local connectivity, and Bluetooth Low Energy (BLE) for short-range device configuration and diagnostics. Additionally, the RF front-end 4406 may integrate with the radar antenna 4410 to provide signal generation and processing capabilities for radar-based measurements, utilizing techniques such as frequency-modulated continuous wave (FMCW) or pulse compression to achieve the required range resolution and accuracy. The RF front-end 4406 may include quadrature demodulation circuitry for extracting in-phase and quadrature components from radar returns, enabling the determination of phase shifts attributable to the dielectric properties of the monitored liquid.

At the base of the sensor node assembly 4400 is the battery and energy harvester module 4404. The battery and energy harvester module 4404 may provide a power management subsystem that enables the autonomous operation of the sensor node for extended periods. The module 4404 may incorporate various power source technologies, depending on deployment requirements, such as lithium batteries, rechargeable batteries, or energy harvesting mechanisms, including photovoltaic cells, thermoelectric generators, and vibration harvesters. Power management circuitry within the battery and energy harvester module 4404 may implement energy optimization strategies, including dynamic voltage scaling, selective component shutdown, and adaptive duty cycling based on operational requirements. For applications requiring extremely long operational life, the battery and energy harvester module 4404 may employ ultra-low-power sleep modes between measurement cycles, with precise wake-up timing controlled by low-power real-time clock circuits.

The complete sensor node assembly 4400 forms a self-contained monitoring unit that can be readily deployed across diverse industrial applications. The modular architecture of the sensor node assembly 4400 provides configuration flexibility, where specific sensor combinations can be selected based on application requirements while maintaining a standard platform design. In some aspects, the sensor node assembly 4400 may implement one or more components as described with respect to FIGS. 1-42.

FIG. 44B illustrates a modular sensor node assembly 4414 for the integrity monitoring platform 4300 of FIG. 43, depicting the plug-and-play architecture that enables field-configurable deployment across diverse liquid monitoring applications in accordance with aspects of the present disclosure. This modular configuration extends the integrated design shown in FIG. 44A by providing interchangeable sensor modules that connect to a central hub architecture. The assembly 4414 comprises a central processing unit 4416 that serves as the primary computational and control hub for the sensor node, similar in function to the microcontroller and PCB assembly 4408 described in FIG. 44A. The central processing unit 4416 may include one or more microprocessors, microcontrollers, or system-on-chip (SoC) devices capable of executing firmware instructions, processing sensor data, and managing communication protocols. The central processing unit 4416 implements hardware abstraction layer (HAL) functionality that enables integration with various sensor modules regardless of their specific implementation details. This abstraction layer provides standardized interfaces for sensor discovery, data acquisition, calibration management, and diagnostic functions.

Multiple sensor connection interfaces, designated as 4418A, 4418B, 4418C, and 4418D, are included at the attachment points of the central processing unit, 4416, providing standardized mechanical and electrical connections for attaching various sensor modules. These interfaces may implement a universal connector system that ensures proper alignment, environmental sealing, and reliable electrical contact while preventing incorrect module installation through mechanical keying. In some aspects, the interfaces support hot-swapping capability, allowing sensor modules to be connected or disconnected during system operation without disrupting ongoing monitoring activities. In some aspects, the central processing unit 4416 may include one or more sensing and/or communication capabilities. In some aspects, the central processing unit 4416 may include one or more sensing and/or communication capabilities that are augmented by one or more sensor and/or communication modules. In some aspects, the central processing unit 4416 may be coupled to one or more sensing and/or communication capabilities to provide these capabilities.

A base dielectric sensor module 4424 connects to the central processing unit 4416 using the sensor connection interfaces 4426, representing a fundamental sensing element that may be common across platform deployments. The dielectric sensor module 4424 may include electromagnetic sensing elements designed to measure the permittivity of liquids through non-invasive interrogation, providing similar functionality to the dielectric sensor 4412 shown in FIG. 44A but in a modular, interchangeable format. The module 4424 may incorporate parallel plate capacitor structures, coaxial probe geometries, or resonant cavity configurations optimized for different frequency ranges and sensitivity requirements. In some aspects, the dielectric sensor module 4424 operates across frequencies from 100 Hz to 40 GHZ, enabling the comprehensive characterization of liquid dielectric properties and their frequency-dependent behavior. However, the dielectric sensor module 4424 may operate at frequencies above and/or below those previously mentioned. The dielectric sensor module 4424 may include integrated temperature compensation circuitry, electromagnetic shielding to minimize external interference, and calibration data stored in non-volatile memory that automatically loads upon connection to the central processing unit 4416.

A radar antenna module 4420 may be separate from the dielectric sensor module 4424 and may attach at a different location, such as the top interface of the central processing unit 4416. The radar antenna module 4420 may provide volumetric and/or dielectric liquid assessment capabilities through electromagnetic wave propagation analysis. The radar antenna module 4420 may provide similar radar-based sensing capabilities as the radar antenna 4410 described in FIG. 44A, but in a modular configuration that enables field replacement or upgrade. The radar antenna module 4420 may implement various antenna configurations, including horn antennas for focused beam patterns, patch arrays for electronic beam steering, or dielectric rod antennas for compact installations. The radar antenna module 4420 may operate in conjunction with radar transceiver circuitry that may be integrated within the module itself or interface with RF front-end capabilities within the central processing unit 4416, similar to the RF front-end module 4406 shown in FIG. 44A. In certain configurations, the radar antenna module 4420 supports frequency-modulated continuous wave (FMCW) operation for high-resolution distance measurements, pulse compression techniques for improved signal-to-noise ratio, and ultra-wideband (UWB) transmission for enhanced material discrimination capabilities.

In some aspects, an auxiliary sensor module 4428 is connected to the central processing unit 4416 via one or more interfaces 4430. The auxiliary sensor module 4428 may provide extensibility for specialized monitoring requirements beyond the core dielectric and radar sensing capabilities shown in the integrated design of FIG. 44A. The auxiliary sensor module 4428 may comprise various sensor types, depending on the specific application, including temperature and humidity sensors for environmental monitoring, pressure transducers for verifying sealed container integrity, acoustic sensors for detecting gas evolution or turbulence, optical sensors for turbidity or color measurements, or gas-phase sensors for detecting volatile organic compounds. Each auxiliary sensor module 4428 may include self-description capabilities, transmitting its sensor type, measurement range, calibration parameters, and operational requirements to the central processing unit 4416 upon connection.

Additional sensor modules 4432, 4434, 4436, and 4438 may interface with one or more modules, providing additional capacity for multi-modal sensing configurations that extend beyond the dielectric sensor 4412 and radar antenna 4410 arrangement shown in FIG. 44A. These modules may include specialized variants optimized for particular deployment scenarios. For instance, module 4432 might comprise a high-temperature dielectric sensor for industrial process monitoring, while module 4434 could contain intrinsically safe circuitry for deployment in explosive atmospheres. Module 4436 may incorporate sanitary-rated construction for pharmaceutical applications, and module 4438 might include extended-range radar capabilities for monitoring large storage tanks.

In some aspects, a power module may attach to the central processing unit 4416, providing power for the sensor node assembly 4400. In some aspects, such a module may provide power management functionality of the battery and energy harvester module 4404, as shown in FIG. 44A. The power module may implement multiple power input options, including 12-24 VDC industrial power, Power over Ethernet (POE) for single-cable installation, battery operation with intelligent power management similar to module 4404, and energy harvesting from environmental sources. In some aspects, a communication module may attach to the central processing unit 4416, providing communication capability for the sensor node assembly 4400. Such a module may include enhanced communication capabilities that may extend beyond the RF front-end module 4406 of FIG. 44A. The communication capabilities of such modules may encompass various wireless and wired protocols such as IEEE 802.11 Wi-Fi for high-bandwidth data transmission, LoRaWAN for long-range, low-power deployments, cellular connectivity (4G/5G) for remote installations, and industrial protocols, including Modbus, HART, or PROFIBUS for integration with existing plant infrastructure. In some aspects, the modular sensor node assembly 4414 may implement one or more components as described with respect to FIGS. 1-42.

The lower portion of FIG. 44B demonstrates how sensor modules combine to create application-specific configurations. For example, in a first state 4440, an individual sensor module is depicted in isolation, highlighting a self-contained design with integrated electronics, environmental protection, and a standardized interface connector. In a second state, two modules may be joined to obtain additional sensing and/or communication capabilities. After a discovery and calibration process, a third state, 4444, may be achieved, where a fully assembled and working configuration is ready to be presented.

The modular architecture is illustrated in FIG. 44B enables rapid reconfiguration for different monitoring applications without hardware redesign. For military fuel monitoring applications, the assembly may include a dielectric sensor module for contamination detection, a radar antenna module at 4420 for level measurement and/or contamination detection, and a temperature sensor module 4428 for thermal compensation. The same hardware platform, when deployed for pharmaceutical quality assurance, might utilize high-precision dielectric sensors in modules 4428 and 4432 for concentration verification, along with conductivity sensors in module 4434 for ionic purity assessment. For example, when a sensor module connects to the central processing unit 4416, it broadcasts a capability descriptor containing one or more of a sensor type identifier (dielectric sensor, radar antenna, temperature sensor, etc.); measurement range and resolution specifications; calibration coefficients and temperature compensation parameters; power consumption profiles for different operational modes; and/or firmware version and compatibility requirements. In some aspects, the central processing unit 4416 may automatically recognizes the connected sensor configuration and load appropriate drivers from its firmware library, enabling plug-and-play operation without manual configuration.

In some aspects, a unified data interface may provide a standardized data protocol for communication between a central processing unit 4416 and one or more external modules, such as the 4420, 4426, and/or the 4432. An example of a standardized data protocol may include:

SensorData {
timestamp: uint64_t; //Microseconds since epoch
sensor_id: uint32_t; //Unique sensor identifier
measurement_type: enum; //DIELECTRIC, TEMPERATURE, etc.
value: float; //Primary measurement value
uncertainty: float; //Measurement uncertainty
metadata: {//Additional context
temperature_comp: float;
quality_score: uint8_t;
diagnostic_flags: uint32_t;
}
   }

In some aspects, a firmware architecture may support the sensor node assembly using a layered design that separates hardware-specific drivers from application logic. The base firmware layer handles low-level hardware interfaces, boot loading, and security functions. A middleware layer implements the hardware abstraction layer, communication protocols, and data management services. The application layer may include industry-specific measurement algorithms, logic for detecting contamination, and reporting functions. This layered approach enables firmware updates to be applied to specific layers without affecting others.

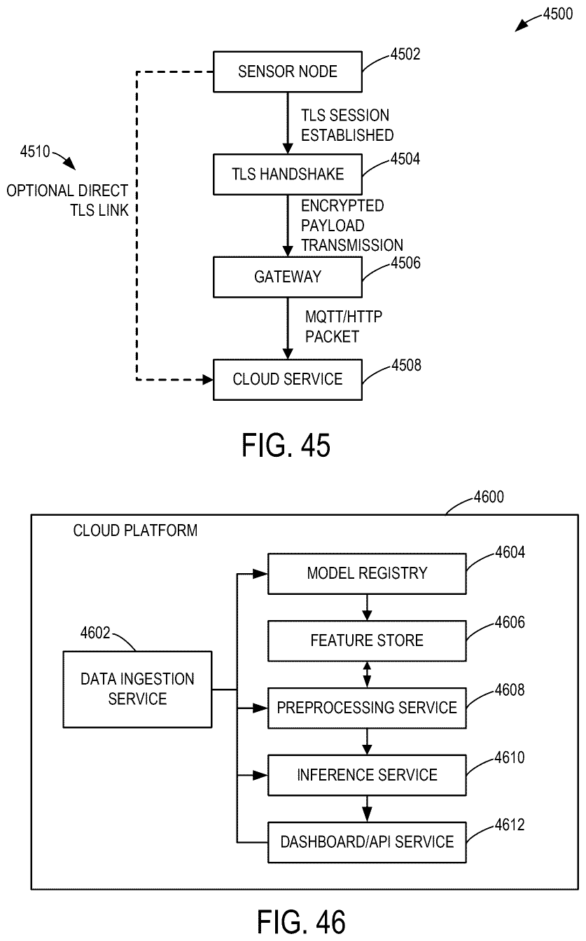

FIG. 45 illustrates a communication architecture 4500 for secure data transmission utilized by the integrity monitoring platform 4300 of FIG. 43 in accordance with at least one aspect of the present disclosure. The sensor node 4502 represents the primary data acquisition device within the system architecture and may be the same as or similar to the sensor node 4314, 4316, and/or 4318 of FIG. 43. Within the context of the integrity monitoring platform 4300 of FIG. 43, the sensor node 4502 functions as the origination for sensor-derived data, including dielectric measurements, environmental parameters, and derived integrity metrics. The sensor node 4502 may be configured with various communication capabilities to support different deployment environments. In implementations where direct internet connectivity is available, such as deployments utilizing cellular modems (e.g., NB-IoT, LTE-M) or Wi-Fi infrastructure, the sensor node 4502 may establish direct communication paths to cloud services. In other implementations, such as those in bandwidth-constrained or security-restricted environments, the sensor node 4502 may utilize local area protocols such as LoRaWAN, Zigbee, or Bluetooth Low Energy to communicate with intermediate infrastructure. The sensor node 4502 may incorporate security features at the hardware and firmware levels, including secure key storage, cryptographic co-processors, and tamper-detection mechanisms to ensure the integrity of transmitted data from the point of origin.

The gateway 4506 serves as an intermediate communication and aggregation point within the architecture 4500. In some aspects, the gateway 4506 may be the same as or similar to the gateway 4320 of FIG. 43. In many deployment scenarios, the gateway 4506 provides functionality for managing multiple sensor nodes 4502 within a localized area. The gateway 4506 may aggregate data from numerous sensor nodes 4502, providing local buffering and store-and-forward capabilities to accommodate intermittent network connectivity. In industrial deployments, such as Forward Arming and Refueling Points (FARPs) or municipal water distribution systems, a single gateway, 4506, may service dozens or hundreds of individual sensor nodes, 4502, distributed across the facility. The gateway 4506 may perform protocol translation between local area wireless protocols used by sensor nodes 4502 and wide area network protocols suitable for cloud communication.

Additionally, the gateway 4506 may implement edge computing capabilities, performing initial data validation, compression, or preliminary anomaly detection before forwarding data to cloud services. The gateway 4506 may maintain its security context, managing device authentication for connected sensor nodes 4502 while establishing secure uplink connections to cloud infrastructure. In certain implementations, the gateway 4506 may also serve as a local command and control point, enabling facility operators to access real-time data without dependency on cloud connectivity.

The cloud service 4508 represents the centralized data processing and analytics infrastructure of the platform. The cloud service 4508 may be the same as or similar to the cloud infrastructure 4328 in FIG. 43. The cloud service 4508 may comprise multiple functional components, including data ingestion services, storage systems, analytics engines, and API endpoints for external system integration. Upon receiving data from the gateway 4506 and/or directly from sensor nodes 4502, the cloud service 4508 performs data validation, normalization, and storage operations. The cloud service 4508 may host a machine-learning model specific to each industry application, executing inference operations on incoming sensor data to generate integrity assessments, contamination alerts, and predictive maintenance recommendations. The scalability of the cloud service 4508 enables the platform to accommodate growing numbers of sensor deployments without compromising performance or responsiveness.

The TLS session establishment 4504 represents the security handshake process that occurs when initiating communication between system components. To ensure communications are encrypted and authenticated, the establishment of a TLS session 4504 may involve the exchange of certificates, negotiation of cipher suites, and generation of session keys. The platform may utilize TLS 1.3 or subsequent versions to benefit from handshake performance and enhanced security features. The TLS session establishment 4504 process may include mutual authentication, where both the sensor node 4502 (or gateway 4506) and the cloud service 4508 verify each other's identity through certificate validation. This mutual authentication prevents man-in-the-middle attacks and ensures that sensor data is transmitted only to authorized cloud endpoints.

The optional direct TLS link 4510, shown as a dashed connection in FIG. 45, represents an alternative communication path that bypasses the gateway 4506. This direct link 4510 enables sensor nodes 4502 equipped with appropriate communication capabilities to establish direct secure connections to the cloud service 4508. The availability of this optional path 4510 provides deployment flexibility and redundancy. For example, in scenarios where a sensor node 4502 has access to cellular or Wi-Fi connectivity, utilizing the direct path 4510 may reduce latency and eliminate potential single points of failure associated with gateway-dependent architectures. The decision to use the direct path 4510 versus the gateway path may be made dynamically based on factors such as network availability, bandwidth costs, power constraints, or security policies.

The TLS handshake process, indicated in the diagram, encompasses the cryptographic negotiations and key exchanges necessary to establish secure communication channels. During the handshake, the communicating parties agree upon encryption algorithms, exchange certificates for identity verification, and generate session-specific encryption keys.

The encrypted payload transmission represents the secure transfer of sensor data and control messages between system components. Following the successful establishment of the TLS session at 4504, all data transmissions are encrypted using the negotiated cipher suite. The encrypted payloads may contain various types of information, including raw sensor measurements, preprocessed feature vectors, alert notifications, configuration updates, or firmware patches. The encryption ensures that even if network traffic is intercepted, the confidentiality and integrity of the sensor data remain protected.

The MQTT/HTTP packet notation indicates example application-layer protocols utilized for data transmission within the secure TLS tunnel. MQTT (Message Queuing Telemetry Transport) provides a lightweight publish-subscribe messaging protocol particularly suited for IoT deployments with constrained bandwidth or intermittent connectivity. In some aspects, HTTP (Hypertext Transfer Protocol) or its secure variant HTTPS may be utilized for RESTful API communications, particularly for configuration management, bulk data transfers, or integration with enterprise systems. The integrity monitoring platform 4300 of FIG. 43 may select between MQTT and HTTP based on the specific requirements of each data transmission.

The architecture 4500 is illustrated in FIG. 45 demonstrates the platform's ability to adapt to diverse deployment environments while maintaining consistent security standards. Whether deployed in military fuel monitoring systems that require air-gapped networks with gateway-based communication or in commercial applications that leverage direct cloud connectivity, the architecture 4500 ensures that data integrity and confidentiality are preserved throughout the transmission path. The integrity monitoring platform 4300 may include blockchain technology as an optional feature to boost its data integrity and audit trail abilities. This is particularly useful for regulated industries that need reliable record-keeping and clear supply chain transparency. Blockchain integration operates as a complementary layer to the existing secure communication architecture, creating an immutable ledger of critical events, measurements, and system actions for scrutiny in legal proceedings, regulatory audits, and forensic investigations.

FIG. 46 illustrates an exemplary embodiment of a cloud platform 4600 configured to process sensor data and generate integrity metrics for monitored liquids in accordance with at least some aspects of the present disclosure. In some aspects, the cloud platform 4600 comprises a plurality of interconnected microservices arranged to facilitate data flow from initial ingestion through analytical processing to final output generation.

In some aspects, a data ingestion service 4602 represents the entry point for sensor data received from gateway devices in communication with distributed sensor nodes. The data ingestion service 4602 may be configured to receive streaming sensor data packets that may include dielectric measurements, temperature readings, environmental parameters, and metadata such as sensor identifiers, timestamps, and location information. In some implementations, the data ingestion service 4602 supports multiple ingestion protocols to accommodate various communication standards, including, but not limited to, MQTT, HTTP/HTTPS, and WebSocket, among others. The data ingestion service 4602 may perform initial data validation, including verification of data packet integrity, authentication of sensor sources, and filtering of duplicate or malformed data. The data ingestion service 4602 uses buffering to address fluctuating data speeds and brief network issues. This helps it reliably capture data, even when the network isn't at its best. The system also includes tools that help manage the speed and volume of data from various sensor networks simultaneously. The data ingestion service 4602 may utilize compression and decompression techniques to optimize bandwidth utilization.

In some implementations, a preprocessing service 4608 receives raw sensor data from the data ingestion service 4602 and performs various transformations to prepare the data for analytical processing. The preprocessing service 4608 may execute operations including but not limited to data normalization, outlier detection, missing value imputation, and temporal alignment of multi-sensor data streams. In one example, the preprocessing service 4608 applies industry-specific preprocessing routines based on metadata indicating the source application domain. For instance, aviation fuel monitoring data may undergo temperature compensation algorithms specific to the characteristics of JP-8 fuel, while pharmaceutical liquid monitoring data may be processed using algorithms that account for the properties of the container material and storage conditions. The preprocessing service 4608 may implement signal processing techniques such as filtering, smoothing, and noise reduction to enhance data quality. Feature engineering operations performed by the preprocessing service 4608 may include the calculation of statistical properties, frequency domain transformations, and extraction of time-series characteristics relevant to anomaly detection. The preprocessing service 4608 may output processed data to both a feature store 4606 and an inference service 4610, enabling parallel paths for data persistence and real-time analysis.

In some aspects, a feature store 4606 provides persistent storage and management of processed feature data generated by the preprocessing service 4608. The feature store 4606 can be configured as a repository that enables quick access to feature vectors during model inference. Depending on the specific implementation, this feature store may use different storage approaches, such as columnar formats, time-series databases, or a combination of both. Thus, the feature store 4606 may optimize both the speed of data retrieval and the efficiency of storage space utilization. In some aspects, the feature store 4606 maintains feature versioning capabilities, enabling tracking of feature evolution and supporting model reproducibility requirements. Feature data stored within the model registry 4604 may include derived and/or raw dielectric parameters, statistical aggregations, temporal patterns, and domain-specific indicators calculated from raw sensor measurements. The feature store 4606 may implement indexing strategies optimized for common query patterns, such as retrieval of historical features for specific sensor nodes, time-range queries for trend analysis, and similarity searches for anomaly detection. Access control mechanisms within the feature store 4606 ensure data segregation between different industrial applications while enabling authorized cross-domain analytics when beneficial.

In some aspects, a model registry 4604 maintains a repository of machine learning models trained for various liquid monitoring applications. The model registry 4604 stores model artifacts, including trained weights, model architectures, preprocessing specifications, and metadata describing model performance characteristics, training datasets, and deployment configurations. In one embodiment, the model registry 4604 implements version control for models, enabling tracking of model lineage and facilitating rollback capabilities. The model registry 4604 may store multiple model variants for each application domain, such as separate models optimized for different liquid types, container materials, or environmental conditions. For example, the registry 4604 may contain distinct models for monitoring aviation fuel in underground storage tanks versus above-ground pipeline segments, each trained on domain-specific dielectric response patterns. The model registry 4604 provides model-serving capabilities to the inference service 4610 through standardized interfaces, enabling dynamic model loading and switching based on the characteristics of incoming data. The bidirectional connection between the model registry 4604 and the inference service 4610 facilitates model performance monitoring and automated retraining workflows.

In some aspects, an inference service 4610 executes one or more machine-learning models to generate integrity metrics from processed sensor data. The inference service 4610 receives inputs from multiple sources, including the preprocessing service 4608, the feature store 4606, and the model registry 4604, orchestrating these inputs to perform model inference operations. In various aspects, the inference service 4610 implements ensemble methods that combine predictions from multiple models to enhance detection accuracy and reduce false favorable rates. The inference service 4610 may execute different model types, including but not limited to convolutional neural networks for spectral analysis, recurrent neural networks for temporal pattern recognition, and gradient-boosting models for anomaly scoring. In certain aspects, the inference service 4610 implements inference optimization techniques, including model quantization, batch processing, and caching of intermediate results, to achieve target latency requirements for real-time monitoring applications. The inference service 4610 may generate various output types, including contamination probability scores, substance identification results, deviation metrics from baseline conditions, and confidence intervals for predictions. In some implementations, the inference service 4610 implements explainability features that provide insights into model decisions, particularly valuable for regulatory compliance and forensic analysis applications.

The cloud platform 4600 may include a dashboard/API service 4612 representing the output interface of the cloud platform 4600 and providing both human-readable visualizations and programmatic access to analytical results. The dashboard/API service 4612 may generate interactive user interfaces displaying real-time monitoring status, historical trends, alert notifications, and detailed analytical reports. Visualization capabilities may include heat maps showing sensor network status, time-series plots of integrity metrics, statistical distributions of contamination events, and geospatial representations of distributed monitoring networks. The API service component may implement RESTful and GraphQL interfaces, enabling integration with external systems such as enterprise resource planning systems, regulatory reporting platforms, and mobile applications. In some aspects, the dashboard/API service 4612 implements webhook mechanisms for push-based alert delivery, enabling notification of critical events such as contamination detection or system anomalies. The output formatting capabilities of the dashboard/API service 4612 may accommodate various compliance requirements, generating reports in formats specified by regulatory bodies, such as the FDA for pharmaceutical applications or the EPA for water quality monitoring. The dashboard/API service 4612 may implement role-based access control, ensuring appropriate data visibility based on user authorization levels and regulatory requirements.

The interconnections between services within the cloud platform 4600 implement various communication patterns optimized for different operational requirements. Synchronous communication paths, such as between the preprocessing service 4608 and the inference service 4610, enable low-latency processing for real-time monitoring applications. Asynchronous communication patterns, such as between the data ingestion service 4602 and the preprocessing service 4608, provide buffering capabilities to handle variable processing loads. Message queuing mechanisms may be implemented between services to ensure reliable data delivery and enable horizontal scaling of individual service components.

In alternative implementations, the cloud platform 4600 may include additional services not depicted in FIG. 46. An alert management service can transform inference results into actionable alerts based on predefined thresholds and customized business rules. Meanwhile, a data archival service plays a role in long-term storage, ensuring compliance with regulations and facilitating historical analysis. A configuration management service may maintain sensor network topologies, calibration parameters, and operational settings across distributed deployments. The platform architecture enables deployment across various cloud infrastructure providers and supports hybrid cloud configurations where sensitive data processing occurs on-premises while leveraging cloud resources for scalable analytics.

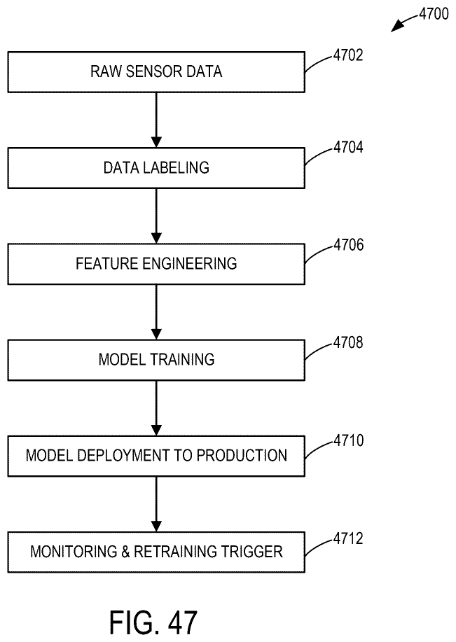

FIG. 47 illustrates a model-training pipeline 4700 for developing and maintaining machine learning models utilized by the integrity monitoring platform 4300 of FIG. 43. The model-training pipeline 4700 represents a systematic workflow for transforming raw sensor measurements into deployable analytical models capable of detecting anomalies, verifying composition, and confirming product identity across various regulated liquid systems. The model-training pipeline 4700 begins with raw sensor data 4702, which comprises dielectric measurements, environmental readings, and associated metadata collected from sensor nodes deployed across various liquid containment systems. The raw sensor data 4702 may include time-series measurements and/or derivations of dielectric values, temperature readings, humidity levels, and other relevant parameters captured during monitoring operations. In some aspects, this data encompasses measurements from diverse deployment scenarios. For example, data may include measurements from horizontally stored whiskey barrels, vertically oriented fuel tanks, pharmaceutical storage vessels, or municipal water distribution systems. The raw sensor data 4702 may consist of both historical measurements accumulated over extended periods and real-time data streams from active deployments. The data format may vary based on the specific sensor configuration and communication protocol employed, with typical representations including structured time-series arrays containing timestamp information, sensor identification codes, dielectric constant measurements across multiple frequencies, temperature compensation values, and quality indicators for each measurement point.

Following data acquisition, the model-training pipeline 4700 proceeds to a data labeling process 4704. The data labeling process 4704 involves associating ground truth information with corresponding sensor measurements to enable supervised learning approaches. This labeling may be performed through various methodologies, including manual annotation by domain experts who identify specific conditions such as regular operation, contamination events, dilution occurrences, or equipment malfunctions. Semi-automated labeling techniques may also be employed, where algorithmic preprocessing identifies potential anomalies or patterns for subsequent human verification. The labeling process may incorporate reference measurements from laboratory analysis, historical incident reports, or controlled experimental data where known adulterants or contaminants are introduced under monitored conditions. Labels applied may include categorical classifications (e.g., “pure,” “contaminated,” “diluted”), continuous values representing concentration levels, temporal markers indicating the onset and duration of anomalous conditions, and confidence scores reflecting the certainty of the assigned labels.

The labeled data then undergoes feature engineering 4706, wherein relevant characteristics are extracted from the raw sensor measurements to facilitate practical model training. Feature engineering 4706 transforms time-domain sensor readings into representations that capture meaningful patterns and relationships within the data. This transformation process may include frequency-domain analysis to identify spectral signatures associated with different liquid states, statistical feature extraction calculating measures such as mean, variance, skewness, and kurtosis over defined time windows, derivative features capturing rates of change in dielectric properties, and cross-correlation features examining relationships between multiple sensor channels or environmental parameters. Feature engineering 4706 may also incorporate domain-specific transformations, such as temperature-normalized dielectric constants, volumetric loss calculations based on integrated sensor readings, or composite indices combining multiple measurement modalities. Feature selection techniques may be applied to identify the most informative features while reducing dimensionality and computational requirements.

The engineered features serve as input to the model training stage 4708, where machine-learning algorithms develop predictive models tailored to specific monitoring applications. The model training stage 4708 may employ various algorithmic approaches depending on the target application and available training data. Supervised learning techniques, including support vector machines, random forests, gradient boosting machines, and deep neural networks, may be utilized for classification tasks such as contamination detection or product authentication. Regression models may be developed for quantitative predictions of concentration levels or degradation rates. Anomaly detection algorithms, such as one-class support vector machines, isolation forests, or autoencoder neural networks, can be trained to identify deviations from normal operating conditions without requiring extensive labeled examples of all possible fault conditions. The model training stage 4708 may incorporate cross-validation techniques to assess model generalization, hyperparameter optimization to tune algorithm settings and ensemble methods that combine multiple models to improve robustness and accuracy.

Upon successful training and validation, models proceed to the deployment stage 4710, where the model may be integrated into the production monitoring platform. The deployment process 4710 may involve packaging trained models into deployable formats compatible with the platform's inference infrastructure, configuring model-specific parameters such as decision thresholds and confidence intervals, establishing version control mechanisms to track model iterations and enable rollback capabilities, and implementing performance monitoring to track inference latency and resource utilization. The deployment stage 4710 may support multiple deployment targets, including cloud-based inference services for centralized processing, edge computing implementations where models execute directly on gateway devices, and hybrid architectures combining local preprocessing with cloud-based analysis. Model deployment 4710 may also include the generation of model documentation, performance benchmarks, and operational guidelines for platform operators.

The model-training pipeline 4700 may conclude with a monitoring and retraining trigger stage 4712 that evaluates deployed model performance and initiates retraining when suitable. The monitoring component may track various performance metrics, including prediction accuracy on newly collected data, false positive and false negative rates for anomaly detection, drift indicators comparing current data distributions to training distributions, and computational efficiency metrics such as inference time and memory usage. Retraining triggers may be activated based on predetermined criteria, such as performance degradation below established thresholds, accumulation of sufficient new labeled data to improve model accuracy, detection of systematic prediction errors indicating concept drift, or scheduled periodic updates to incorporate recent operational data. The retraining process may involve incremental learning approaches that update existing models with new data, complete retraining from expanded datasets or transfer learning techniques that adapt models trained on one liquid type or deployment scenario to new applications.

The model-training pipeline 4700 may incorporate feedback loops wherein operational insights inform subsequent iterations of the training process. Performance data from deployed models may guide adjustments to feature engineering strategies, labeling protocols, or algorithm selection. The pipeline 4700 may also support parallel training workflows for various industry applications, enabling the simultaneous development of models for fuel quality monitoring, spirits authentication, pharmaceutical integrity verification, and water safety assessment while leveraging shared infrastructure and methodologies.

In some aspects, the predictive analytic capabilities of the integrity monitoring platform 4300 extend beyond real-time anomaly detection to include forecasting liquid degradation trajectories, maintenance requirements, and operational risks. These predictive functions may leverage the continuous stream of fluid characteristic measurements, environmental data, and historical patterns to generate actionable intelligence about future system states. Such actional intelligence may enable proactive maintenance strategies that may be used to prevent costly failures and ensure operational continuity.

The predictive maintenance algorithms employed by the integrity monitoring platform 4300 may utilize multiple approaches specific to temporal characteristics and degradation patterns of different liquids. For gradual degradation processes, such as fuel oxidation or spirits evaporation, the integrity monitoring platform 4300 may implement an autoregressive integrated moving average (ARIMA) model that captures at least one of the trend or seasonal components related to one or more liquid characteristics. For example, in aviation fuel monitoring applications, the ARIMA model may track an increase in a dielectric constant associated with the formation of oxidation products. The model may update its parameters based on recent measurements, thereby adapting to changes in storage conditions or variations in fuel batches. When the projected dielectric trajectory intersects one or more specification limits, the integrity monitoring platform 4300 may generate predictive alerts such as “Based on current oxidation rate of 0.3 Er units per month, fuel in Tank #5 will exceed water contamination limits in 47 days. Recommend fuel polishing by Day 35 to maintain operational specifications.” Of course, other models and model types may be used as a predictive maintenance algorithm. For example, in pharmaceutical liquid monitoring, a long short-term memory (LSTM) network may predict stability degradation by learning complex interactions between temperature excursions, dielectric changes, and time-dependent chemical reactions. The model might generate insights such as “Detected accelerated degradation pattern in Batch #A2847. Current dielectric trend indicates 15% potency loss within 60 days if storage conditions remain unchanged. Recommend immediate transfer to climate-controlled storage to extend shelf life by 180 days.” In some aspects, one or more ensemble forecasting methods that combine predictions from multiple models may be used to enhance accuracy and provide uncertainty quantification. For example, a weighted ensemble approach may integrate ARIMA projections, LSTM forecasts, and physics-based degradation models, with weights dynamically adjusted based on recent prediction performance. The ensemble model may generate not only point predictions but also confidence intervals, enabling risk-aware decision-making. For instance, a fuel quality forecast might indicate: “90% confidence that water content will remain below 150 ppm for the next 30 days, 75% confidence for 45 days, 50% confidence for 60 days.”

In some aspects, a trend analysis capability may employ change point detection algorithms to identify shifts in degradation patterns that may indicate process changes or emerging issues. When changepoints are detected with high probability, the system triggers a re-evaluation of predictive models and may generate an alert about altered degradation dynamics. For example, one or more actionable predictions can be generated; such as “Based on similar historical cases, there is an 85% probability that Pipeline Segment #7 will require cleaning intervention within the next 500 operating hours to prevent paraffin-induced flow restrictions.” Thus, a predictive analytics engine can integrate with maintenance scheduling systems through standardized interfaces, including CMMS (Computerized Maintenance Management System) protocols, REST APIs for work order generation, and calendar synchronization for optimized maintenance windows. When predictive models forecast maintenance requirements, the system may automatically generate draft work orders with specific maintenance recommendations, calculate optimal intervention timing balancing risk and operational constraints, estimate required resources and spare parts based on predicted failure modes, coordinate maintenance schedules across multiple assets to minimize disruptions, and provide cost-benefit analysis comparing preventive intervention versus run-to-failure scenarios.

As an example, for military fuel operations at Forward Arming and Refueling Points (FARPs), the integration might generate: “Predictive analysis indicates Fuel Bladder #3 at FARP-Alpha requires preventive water removal in 72-96 hours. Optimal maintenance window identified during scheduled ground operations on Day 3, 0600-0800 hours. Estimated prevention cost: $2,400. Potential mission impact if ignored: Critical fuel contamination risk with $150,000+equipment damage potential.”

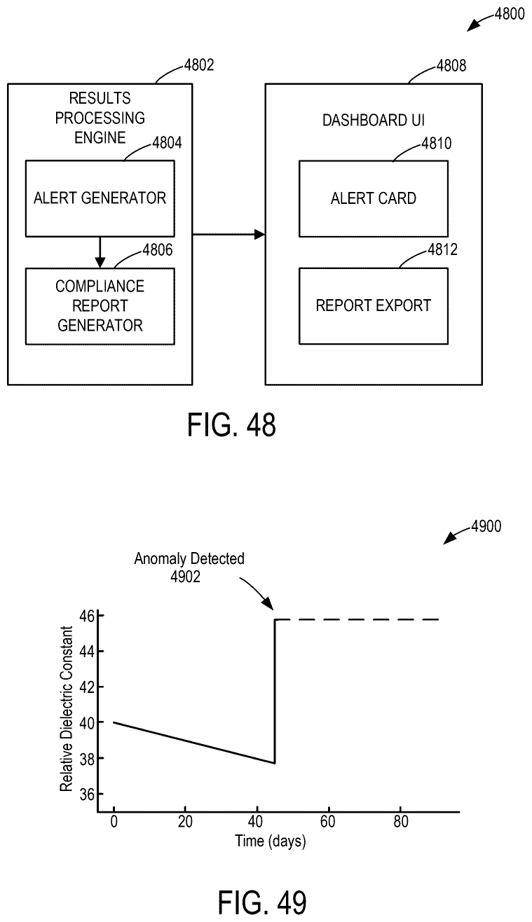

FIG. 48 illustrates a results processing and user interface architecture for transforming analytical outputs into actionable insights within an integrity monitoring platform 4300 of FIG. 43, in accordance with at least one aspect of the present disclosure. In some aspects, the results processing and visualization subsystem 4800 may receive processed integrity metrics from one or more upstream analytics components and transform them into user-accessible formats suitable for operational decision-making. In some aspects, the results processing engine 4802 interprets machine learning outputs and generates actionable intelligence. The results processing engine 4802 may perform multiple functions, including threshold evaluation, temporal correlation of events, and classification of detected anomalies according to severity and type. The results processing engine 4802 may operate on integrity metrics received from the analytics layer 4308 of FIG. 43, which may include dielectric constant deviations, pattern anomalies, or composition changes detected through the sensor network. The results processing engine 4802 may apply one or more configurable rule sets that can be adapted to specific industry requirements without modification to the underlying processing architecture. For instance, when deployed in aviation fuel monitoring applications, the results processing engine 4802 may evaluate water contamination thresholds specific to jet fuel specifications. In contrast, in pharmaceutical applications, the same results processing engine 4802 may evaluate deviations indicative of product tampering or degradation.

Within the results processing engine 4802, the alert generator 4804 provides real-time notification capabilities for detected anomalies. The alert generator 4804 may implement a multi-tiered alerting framework that categorizes detected events according to severity, urgency, and operational impact. The alert generator 4804 may process incoming integrity metrics against defined threshold values and generate corresponding alert objects when deviations exceed acceptable ranges. The alert generator 4804 may incorporate temporal filtering to minimize false positives, requiring persistent anomalies across multiple measurement cycles before triggering notifications. The alert generator 4804 may support numerous alert types, including immediate notifications for critical contamination events, warning alerts for trending deviations, and informational alerts for minor variations within acceptable tolerances. Each generated alert may include contextual metadata that includes, but is not limited to, timestamp, sensor identification, deviation magnitude, and suggested remediation actions. The alert generator 4804 may interface with external communication systems through standardized protocols, enabling integration with existing enterprise notification infrastructure such as SCADA systems, mobile push notifications, or email alerts.

In some aspects, a compliance report generator 4806 may be directed to regulatory documentation and audit trail generation. The compliance report generator 4806 may aggregate integrity measurements over defined reporting periods and format such aggregations according to industry-specific compliance requirements. The compliance report generator 4806 may maintain a persistent record of monitored parameters, detected anomalies, and system responses, creating an immutable audit trail suitable for regulatory review. The report generation may follow configurable templates that align with various regulatory frameworks, such as FDA requirements for pharmaceutical manufacturing, EPA standards for water quality monitoring, or military specifications for fuel integrity. The compliance report generator 4806 may implement automated scheduling capabilities, generating periodic reports at intervals defined by regulatory requirements or operational policies. Generated reports may include statistical summaries of monitored parameters, graphical representations of trends over time, and detailed logs of any detected anomalies with corresponding system responses.

The dashboard user interface 4808 provides the primary visualization and interaction layer for system operators and stakeholders. The dashboard user interface 4808 may implement a responsive design architecture that adapts to various display devices and user contexts, from control room displays to mobile devices used by field personnel. Within the dashboard interface 4808, one or more alert cards 4810 serve as the primary mechanism for displaying active system notifications. Each alert card 4810 may present a concise summary of a detected anomaly. The report export functionality 4812 provides one or more mechanisms for extracting compliance reports and analytical data from the system in various formats suitable for external consumption. This component supports multiple export formats, including PDF documents for regulatory submission, CSV files for data analysis, and structured XML or JSON formats for integration with enterprise systems.

FIG. 49 illustrates an exemplary time-series anomaly detection output generated by the integrity monitoring platform 4300 of FIG. 43, in accordance with at least one aspect of the present disclosure. The graph 4900 depicts the platform's capability to monitor the dielectric properties of liquids and other substrates contained within sealed containers and identify deviations from the expected baseline fingerprints through the analytics layer 4308.

The graph 4900 comprises a horizontal axis representing time, measured in days, extending from zero to approximately ninety days, and a vertical axis representing relative dielectric constant (Er) values, ranging from approximately thirty to forty-eight. As previously described, the relative dielectric constant represents a dielectric fingerprint parameter that acts as an indicator of the substrate's molecular composition, purity, and integrity. As established in the integrity monitoring platform 4300 of FIG. 43, different substances exhibit characteristic dielectric properties that create unique signatures, enabling the detection of contamination, tampering, degradation, dilution, or substitution events.

The primary trace depicted within graph 4900 shows the temporal evolution of the dielectric constant over a monitoring period. During the initial period, which extends from day zero to approximately day forty-five, the trace shows a gradual downward trend, declining from an initial value of roughly thirty-eight to a value near thirty-six. This gradual decline represents expected changes in the substrate's dielectric properties within normal operational parameters. The analytics engine 4330, utilizing an industry-specific machine learning model from the model registry 4604, recognizes such patterns as baseline behavior. For spirits monitoring applications, this decline may correspond to natural aging processes and ethanol-water dynamics. For oil and gas applications at FARPs, similar trends may indicate expected oxidation or component evolution processes.

At approximately day forty-eight, the trace exhibits an abrupt deviation from the established baseline fingerprint, characterized by a sharp increase in the relative dielectric constant to a value exceeding 46. The anomaly 4902 represents a sudden change in the substrate's dielectric fingerprint that exceeds threshold parameters established by the machine learning models 4332 of FIG. 43 for the particular liquid type and container configuration. That is, the analytics layer 4308 may process incoming sensor data from the sensor layer 4302, communication layer 4304, and/or cloud layer 4306 to maintain one or more baseline models of expected dielectric behavior. When incoming measurements deviate from the predicted baseline fingerprint by a threshold, the integrity monitoring platform 4300 of FIG. 43 may trigger an anomaly detection protocol through the results processing and communication layer 4310 of FIG. 43.

In some aspects, the nature and magnitude of the anomaly 4902 may provide diagnostic intelligence regarding potential integrity compromises. For example, a sharp increase in a dielectric constant may indicate different events depending on the deployment context. For military fuel monitoring at FARPs, an increase of this magnitude strongly suggests water contamination (or other adulteration), as water typically exhibits a dielectric constant of approximately 80 compared to typical fuel values. For whiskey barrel monitoring, similar patterns indicate potential dilution or tampering. In pharmaceutical applications, such changes signal potential contamination or degradation of the active ingredient.

Accordingly, based on the temporal signature of a detected anomaly, such as anomaly 4902, the integrity monitoring platform 4300 of FIG. 43 may differentiate between contamination, tampering, leakage, degradation, dilution, substitution, and hazard events through pattern analysis. The sharp transition signature may correspond to discrete events such as malicious tampering, sudden seal failures, or unauthorized substance introduction.

Following the detection of anomaly 4902, the sustained elevation of the dielectric constant throughout the remainder of the monitoring period may indicate a persistent compromise rather than transient conditions. The integrity monitoring platform's 520 (e.g., FIG. 43) predictive analytics capabilities, as implemented through the machine learning models, may utilize this persistence data to classify anomaly severity and predict future impacts. In some aspects, a proactive response, such as “Based on current contamination signature, recommend immediate intervention to prevent specification breach within 72 hours,” may also be generated.

In some aspects, graph 4900 may incorporate multi-modal data streams. For example, radar-based dielectric measurements at multiple frequencies may reveal frequency-dependent signatures, providing additional information traces from auxiliary sensor modules 4428 that may be used to distinguish environmental effects from actual liquid integrity changes. As another example, machine-learning confidence intervals from the inference service 4610 may be used to quantify detection certainty.

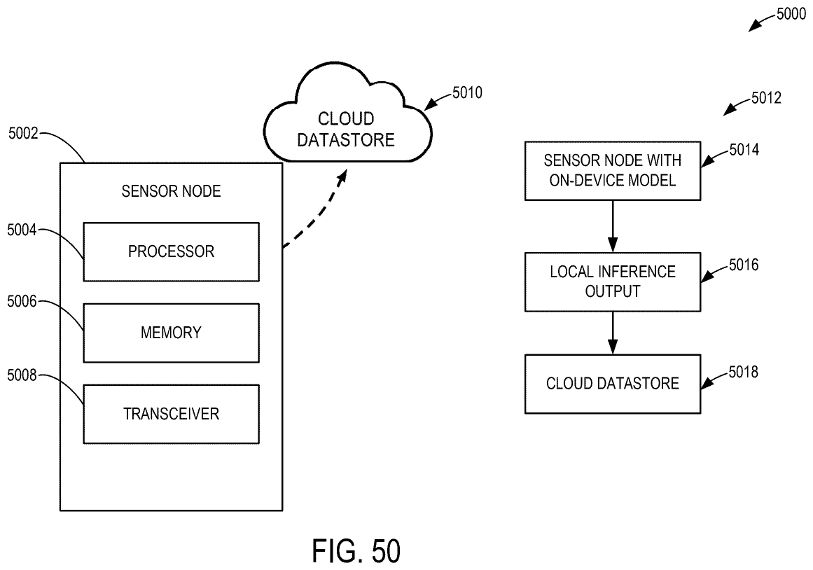

FIG. 50 illustrates an edge-only inference implementation of the integrity monitoring platform 4300 of FIG. 43, wherein analytical processing occurs locally within the sensor node rather than relying on the cloud layer 4306, in accordance with at least one aspect of the present disclosure. In some aspects, this configuration represents an alternative deployment architecture that enables autonomous operation in bandwidth-constrained, security-sensitive, and/or intermittent connectivity environments while maintaining the integrity monitoring platform's 520 capability to detect contamination, tampering, degradation, and other integrity compromises.

In some aspects, the sensor node 5002 may include a processor 5004, memory 5006, and/or a transceiver 5008, and may generally correspond to one or more sensor nodes 4314, 4316, and 4318 depicted in FIG. 43, but with enhanced local processing capabilities. The processor 5004 may execute both operational control functions and machine learning inference operations typically performed by the analytics engine 4330 in cloud-based deployments. The processor 5004 may comprise architectures optimized for embedded TinyML applications, including ARM Cortex-M series microcontrollers with neural network accelerators, edge AI processors, or specialized inference chips. In military deployments at FARPs or other mission-critical applications, the processor 5004 may implement secure boot mechanisms and hardware-based encryption to ensure tamper-resistant operation. The processor 5004 interfaces with dielectric sensors and radar antennas through a standardized protocol described in the modular sensor architecture 4414.

In some aspects, the memory component 5006 provides hierarchical storage for operational firmware, dielectric fingerprint baselines, and compressed machine-learning models derived from the model registry 4604. Non-volatile memory may retain industry-specific machine learning (ML) models trained to recognize dielectric signatures of various substrates, such as whiskey in barrels, aviation fuel in bladders, or pharmaceutical liquids in storage vessels. Such models represent versions of the full-scale models maintained in the cloud platform 4600, compressed through quantization and/or pruning techniques to fit embedded constraints (e.g., processing and/or storage) while aiming to preserve detection accuracy, though it is to be understood that such a model may provide a detection accuracy that is different from a detection accuracy for a model run and/or executed in cloud layer 4306 and/or analytics layer 4308. The memory 5006 may store rolling buffers of recent dielectric measurements, enabling temporal pattern analysis for distinguishing between gradual degradation and acute tampering events. Volatile memory provides a workspace for real-time feature extraction and inference operations on incoming sensor data streams.

The transceiver component 5008 implements the communication layer 4304 functionality within the edge device, supporting the same or similar multi-protocol capabilities as the gateway 4320 but optimized for sporadic rather than continuous transmission. For some military deployments, the transceiver 5008 may implement advanced encryption and frequency-hopping spread spectrum techniques to ensure secure, jam-resistant communications. In commercial implementations across the oil and gas, water utilities, or food and beverage industries, the transceiver 5008 may adapt its protocol selection based on available infrastructure (for example, LoRaWAN for remote pipeline monitoring, NB-IoT for urban water distribution systems, or Wi-Fi for facility-based pharmaceutical storage). The transceiver may operate in adaptive duty-cycling modes, remaining dormant during routine monitoring but activating upon anomaly detection.

The cloud datastore 5010 represents a modified role for the data repository 4326 in edge-computing scenarios. Rather than receiving continuous streams of raw dielectric measurements, the cloud datastore 5010 may aggregate processed integrity assessments, anomaly reports, and predictive maintenance recommendations generated locally by distributed edge nodes. The intermittent connection, indicated by the dashed line, reflects the integrity monitoring platform's 520 capability to operate autonomously while maintaining consistency with centralized systems. When connectivity permits, the edge node may synchronize detected anomalies, updated baseline fingerprints, and operational metrics, enabling fleet-wide visibility without compromising local responsiveness.

As indicated by process 5012, a sensor node with an on-device machine-learning model 5014 may perform anomaly detection algorithms as previously described, where fluids may have unique dielectric fingerprints, and deviations may signal integrity compromises. Accordingly, the edge-optimized model 5014 may represent a distilled version of the comprehensive models maintained in the analytics layer 4308, retaining substrate-specific pattern recognition capabilities while operating within embedded constraints. The model 5014 may process incoming dielectric constant measurements and compare them against learned baseline fingerprints to detect contamination signatures (water in fuel showing εᵣ jumps toward 80), dilution patterns (unexpected shifts in whiskey proof correlating with dielectric changes), or tampering indicators (sudden step changes inconsistent with natural evolution). Lightweight temporal models may be deployed and optimized for streaming data, such as gated recurrent units (GRUs) or dilated causal convolutions.

The local inference output 5016 generates similar actionable intelligence as the results processing module 4334 but may have the characteristic of sub-second latency. These outputs may include contamination probability scores with substance identification (“95% confidence: water detected in aviation fuel”), trend-based predictions (“current oxidation rate indicates specification breach in 72 hours”), and tamper alerts with forensic timestamps (“unauthorized access detected at 14:32:17 UTC”). The edge inference may maintain the integrity monitoring platform's 520 multi-tiered alert framework, distinguishing between informational notifications, warnings requiring attention, and alerts demanding prioritized intervention. Output formats may remain compatible with the dashboard/API service 4612 specifications, ensuring integration capabilities whether inference occurs at the edge or in the cloud.

In some aspects, the reference character 5018 represents a bidirectional synchronization pathway that supports the integrity monitoring platform's 520 improvement cycle. During synchronization windows, edge nodes may upload aggregated anomaly patterns and edge-case detections that enhance the training datasets for next-generation models. Conversely, nodes download updated model parameters reflecting fleet-wide learning, industry-specific threshold adjustments, and newly identified threat signatures. This learning approach may maintain model operationality while respecting data locality requirements. For regulated industries, synchronization may include cryptographically signed audit trails that provide compliance documentation and ensure integrity.

FIG. 9 illustrates a functional block diagram of an exemplary workflow for detecting adulteration and analyzing compositional anomalies in monitored substrates based on deviations in dielectric fingerprints in accordance with at least one aspect of the present disclosure. The workflow may be performed at the analytics layer 4308 within the integrity monitoring platform 4300. In certain aspects, the workflow demonstrates how the system converts raw dielectric measurements into actionable insights regarding contamination, tampering, degradation, dilution, substitution, or hazardous events.

The workflow may begin with a measured dielectric value 5102, representing real-time sensor data captured by the dielectric sensor 4412 or radar antenna 4410 of a sensor node deployed on a sealed container. The measured dielectric value 5102 comprises the actual relative permittivity (Er) value obtained through non-invasive electromagnetic interrogation at a specific timestamp, where permittivity (Er) may represent the ratio of a material's permittivity to the permittivity of vacuum (co). This measured dielectric value 5102 reflects the current dielectric fingerprint of the monitored substrate, which serves as a unique identifier based on molecular composition and state. Whether monitoring whiskey in barrels, aviation fuel at FARPs, pharmaceutical liquids, or water in treatment facilities, the measured dielectric value 5102 captures the substrate's instantaneous dielectric signature. The sensor nodes 4314, 4316, or 4318 may acquire these measurements, transmitting them through the secure communication layer 4304 to the analytics engine 4330 for processing.

Concurrent with obtaining the measured dielectric value 5102, the system accesses an expected dielectric value 5104 from the feature store 4606 or model registry 4604. The expected dielectric value 5104 represents the baseline dielectric fingerprint for the specific substrate under current environmental conditions, where a material has a unique, predictable dielectric signature. This baseline incorporates the substrate-specific models trained through the model training pipeline 4700, accounting for known variables such as temperature effects captured by auxiliary sensor module 4428, natural aging curves, and acceptable compositional variations. For spirits monitoring, the expected value may reflect ethanol-water equilibrium dynamics; for fuel applications, it may represent specification-compliant dielectric ranges; for pharmaceutical implementations, it may embody validated product fingerprints.

The system performs a comparison operation 5106 between the measured dielectric value 5102 and the expected dielectric constant 5104 to compute a dielectric differential value (Acr). This differential calculation 5106 quantifies the deviation from the substrate's baseline fingerprint. The differential may be used as an anomaly signal, with its magnitude and temporal characteristics revealing the nature of the integrity event. A gradual drift might indicate natural degradation, while a step change may suggest contamination or tampering. The comparison operation 5106 implements the feature engineering 4706 techniques developed during model training and may normalize the differential based on substrate type and operational context.

The computed dielectric differential is provided to a machine learning model 5108 retrieved from the model registry 4604. The machine learning model 5108 represents an industry-specific algorithm trained to distinguish between benign variations and genuine integrity compromises. For military fuel monitoring, the machine learning model 5108 may identify patterns indicating water contamination, microbial growth, or the presence of malicious additives. For spirits applications, the machine learning model 5108 may identify dilution signatures or unauthorized barrel access. The machine learning model 5108 processes the differential alongside temporal patterns and multi-frequency signatures when available, implementing analytical processing and providing a continuous audit layer that traditional manual testing does not contemplate.

The machine learning model 5108 may generate two outputs aligned with the platform's integrity monitoring objectives. First, an anomaly determination 5112 flags whether the observed deviation represents a significant departure from the substrate's expected fingerprint. This determination implements a multi-tiered classification framework, categorizing events as normal variations, warnings that require attention, or critical alerts that may necessitate immediate intervention. The anomaly detection leverages the temporal analysis capabilities described with respect to FIG. 49, distinguishing between different degradation patterns and tampering signatures based on the rate and magnitude of dielectric change.

When anomalous conditions are detected, the machine learning model 5108 generates an inferred adulterant identification 5110, leveraging the platform's capability not only to detect “tampered” but potentially to classify the tampering agent. This inference draws upon the known dielectric constants of common adulterants: water (εᵣ≈80), ethanol (εᵣ≈24.3), methanol (εᵣ≈33), glycerol (εᵣ≈42), and various sugars (εᵣ≈60-70+). The ML-based substance classification may back-calculate likely foreign substances based on the observed dielectric shift. For example, in aviation fuel with a nominal εᵣ of approximately 2, a change toward εᵣ of approximately 10 strongly indicates water contamination. In contrast, in whiskey with an expected εᵣ of roughly 35, a jump to εᵣ of approximately 50 suggests dilution with water or sugar solutions.

Both outputs may feed into a logging module 5114 that creates an immutable audit trail. The logging function 5114 may implement the compliance report generator 4806 functionality, capturing forensic data, including timestamps, sensor identifications, dielectric measurements, ML predictions with confidence scores, and/or environmental context. For regulated industries, whether FDA-compliant pharmaceuticals, EPA-monitored water systems, or ITAR-restricted military fuels, these logs provide tamper-evident documentation for audits and investigations. The platform's optional blockchain integration can securely anchor these logs, ensuring data integrity for legal proceedings or supply chain verification.

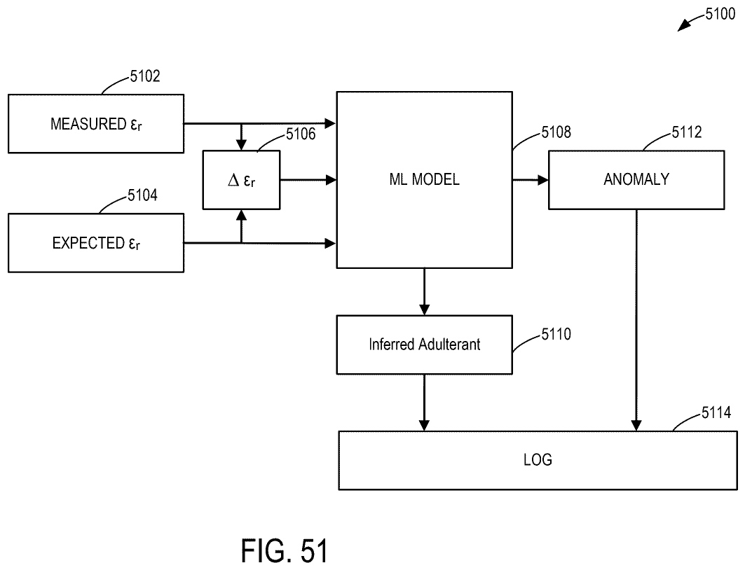

In some aspects, and cloud-based deployments, the workflow depicted in FIG. 51 runs within the inference service 4610, processing streams of sensor data with sub-second response times. The modular design enables the same workflow to adapt across industries simply by loading the appropriate models from the registry 4604.

Alternative implementations support the platform's flexible deployment architecture in edge computing scenarios described in FIG. 50, this workflow executes directly on the sensor node processor 5004, with the on-device model performing local inference. Multi-modal implementations incorporating VOC sensors or additional auxiliary modules 4430 may generate richer feature sets for enhanced feature discrimination. The workflow may also integrate with the predictive analytics capabilities, forecasting future dielectric evolution based on detected trends: “Based on current oxidation rate, fuel will exceed contamination limits in 47 days.”

The outputs of the workflow depicted in FIG. 51 may integrate with the results processing and communication layer 4310, triggering appropriate responses through the dashboard interface 4336. For military FARP applications, detection of fuel tampering can automatically close valves and generate security alerts. In pharmaceutical manufacturing, the identification of unexpected dielectric signatures can halt production lines pending investigation. Water utilities receive immediate notifications of contamination events, enabling rapid isolation of affected distribution segments.

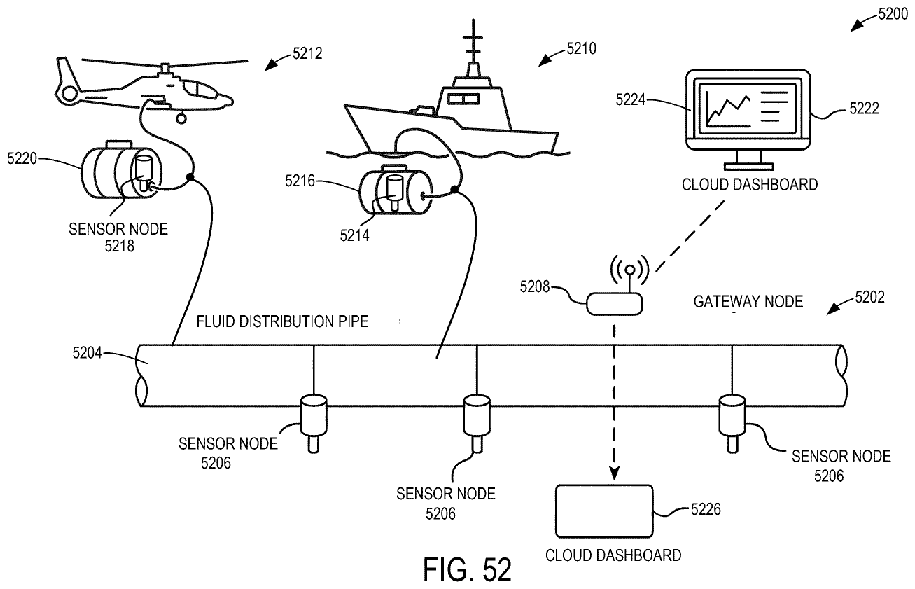

FIG. 52 depicts an exemplary deployment schematic for the integrity monitoring platform 4300 configured for real-time substrate integrity assessment across the distributed infrastructure in accordance with at least one aspect of the present disclosure. FIG. 10 demonstrates the scalability of the integrity monitoring platform, showing that standard hardware, when combined with industry-specific software, can be effectively deployed across a range of industrial applications, from fuel monitoring at Forward Arming and Refueling Points (FARPs) to water safety systems.

As an example, a fluid distribution pipe 5204 extends horizontally across a deployment area 5202, representing a sealed container system for transporting liquids. The fluid distribution pipe 5204 is an example of the various container 4322 types compatible with the integrity monitoring platform 4300, including steel pipelines for oil and gas transport, pharmaceutical distribution lines, municipal water mains, or military fuel systems. Thus, the fluid distribution pipe 5204 demonstrates the integrity monitoring platform's 520 capability to monitor substrates in the wild rather than through traditional laboratory sampling, providing a continuous audit layer that distinguishes the integrity monitoring platform 4300 from conventional methods.

A plurality of sensor nodes 5206 may be positioned at strategic intervals along the fluid distribution pipe 5204, each implementing the universal sensor node assembly 4400 or modular configuration 4414 and may be the same as or similar to one or more sensor nodes 4314, 4316, and/or 4318. The sensor nodes incorporate the dielectric sensors 4412 and/or radar antennas 4410 that measure the substrate's dielectric fingerprint through, for example, non-invasive electromagnetic interrogation. Each sensor node 5206 may operate autonomously, capturing the unique dielectric constant (Er) signatures that enable the detection of contamination, tampering, leakage, degradation, dilution, substitution, or hazardous events. The sensor nodes 5206 may implement the same core sensing technology, whether monitoring JP-8 fuel at military installations, detecting water ingress in crude oil pipelines, or identifying pharmaceutical tampering, where the analytics models 4332 and alert thresholds may differ between applications.

A gateway node 5208 may function as a regional implementation of gateway 4320 within the communication layer 4304, aggregating data from the distributed sensor array. The gateway 5208 may establish secure wireless communication links 4321 (e.g., FIG. 43) with each sensor node using protocols for sensitive deployments or commercial IoT standards for civilian applications. Beyond data aggregation, the gateway 5208 can execute edge analytics when configured for bandwidth-constrained environments, performing initial anomaly detection using lightweight models before forwarding significant events to the cloud layer 4306. This hierarchical architecture enables deployment flexibility ranging from single-barrel whiskey monitoring to transcontinental pipeline networks.

The cloud dashboard infrastructure 5226 represents the integrated cloud platform 4600 hosting the data repository 4326, analytics engine 4330, and dashboard/API service 4612. Within this infrastructure, the preprocessing service 4608 normalizes incoming dielectric measurements, while the inference service 4610 may execute industry-specific models from the model registry 4604. For fuel oil applications shown here, these models may be trained to recognize dielectric signatures of water contamination (Er shifts toward 80), microbial growth, oxidation products, or malicious additives. The same or similar cloud infrastructure, when receiving data from water system deployments, would execute models trained on pathogen indicators, chemical contaminants, or markers of treatment efficacy.

Monitoring scenarios 5210 and 5212 demonstrate the platform's versatility across a range of operational contexts, consistently delivering reliable detection capabilities. In scenario 5210, the vessel deployment depicts naval applications where the integrity monitoring platform 4300 plays a pivotal role in safeguarding mission-critical equipment. By facilitating early detection of contamination within fuel systems, this platform helps prevent damage that could lead to operational failures or compromised missions. This functionality is particularly relevant for military applications, where the integrity of fuel sources directly affects mission success and safety. For instance, a sensor node 5214 can be strategically associated with vessel 5216 to monitor fuel quality and detect any potential contaminants in real time. This proactive monitoring allows naval forces to take preventative measures, ensuring that only clean, uncontaminated fuel is used, thereby extending the life of critical systems and maintaining operational readiness. Furthermore, the information gathered from the sensor node 5214 can be analyzed for trends over time, helping to inform maintenance schedules and fuel management strategies, ultimately leading to more efficient operations and reduced costs.

The helicopter deployment monitoring scenario 5212 plays an important role in ensuring the integrity of aviation fuel at Forward Arming and Refueling Points (FARPs). In military operations, FARPs are pivotal for maintaining operational readiness, as they enable the swift refueling of helicopters close to the front lines. Ensuring the quality of fuel at these locations is important; contamination or degradation can lead to operational failures, which in turn could have catastrophic consequences in mission-critical situations. By implementing real-time quality assurance measures, the integrity monitoring platform 4300 may reduce the likelihood of failures that could arise from using compromised fuel. Traditional methods, which often rely on periodic manual sampling, can leave significant gaps in oversight, potentially allowing dangerous conditions to go undetected for extended periods.

In contrast, continuous monitoring provides a proactive approach, enabling immediate detection of any anomalies in fuel quality or integrity. This rapid response capability can help maintain the readiness and safety of aviation operations. Moreover, the implications of this monitoring extend beyond operational efficiency; they also touch upon national security. A reliable and secure aviation supply chain has a direct impact on the success of military missions. The ability to continuously oversee fuel integrity contributes to strategic military advantages, ensuring that forces remain agile and capable in the face of threats. As illustrated in FIG. 52, the deployment of sensor nodes, such as 5218, that are linked to fuel storage vessels, like vessel 5220, highlights aspects of the technology. These sensor nodes, such as 5218, may be configured to monitor various parameters, including temperature, pressure, and the presence of contaminants. By transmitting real-time data to command centers, they enable responsive adjustments and interventions, further enhancing the reliability of fuel supplies during operations. This level of monitoring and data collection represents an advancement over traditional monitoring methods, illustrating the ongoing evolution of military logistics in support of national defense objectives.

The visualization display 5222 implements the dashboard user interface 4808, transforming analytical outputs into actionable intelligence. Through alert cards 4810, operators receive immediate notifications 5224 of integrity events: “Water contamination detected in Pipeline Segment #7-95% confidence based on Er shift from 2.1 to 8.3.” The display 5222 may integrate predictive analytics, presenting forecasts such as “Current paraffin buildup rate indicates flow restriction within 500 operating hours.” This real-time visibility enables the proactive maintenance strategies that distinguish the platform from reactive, sample-based approaches.

The wireless communication paths utilize a secure architecture 4500 with TLS encryption 4504, protecting data integrity throughout transmission. Whether using direct sensor-to-cloud links 4510 or gateway-mediated paths, the integrity monitoring platform 4300 provides security for sensitive deployments while maintaining flexibility for commercial applications. The wireless infrastructure reduces cabling requirements, enabling rapid deployment and reconfiguration as operational needs evolve.

For water infrastructure monitoring, a similar deployment architecture addresses municipal safety through software adaptation alone. The sensor nodes 5206 may retain their dielectric and radar sensing capabilities, but load models trained on water quality signatures from the model training pipeline 4700. The substrate's dielectric fingerprint reveals contamination events, as heavy metals alter conductivity patterns, biological agents modify dielectric dispersion, and treatment chemicals create characteristic signatures. The gateway 5208 and cloud dashboard 5222 operate similarly, merely processing different ML models optimized for water safety rather than fuel integrity.

This deployment exemplifies the universal hardware platform paired with industry-specific analytics. The transition from fuel monitoring to water safety requires updating the machine learning models-no hardware modifications, no sensor replacements, no infrastructure changes. Whether protecting military assets from contaminated fuel, ensuring the authenticity of pharmaceutical products, detecting whiskey tampering, or safeguarding public water supplies, the technology of the integrity monitoring platform 4300 remains constant, while software intelligence adapts to each industry's unique requirements.

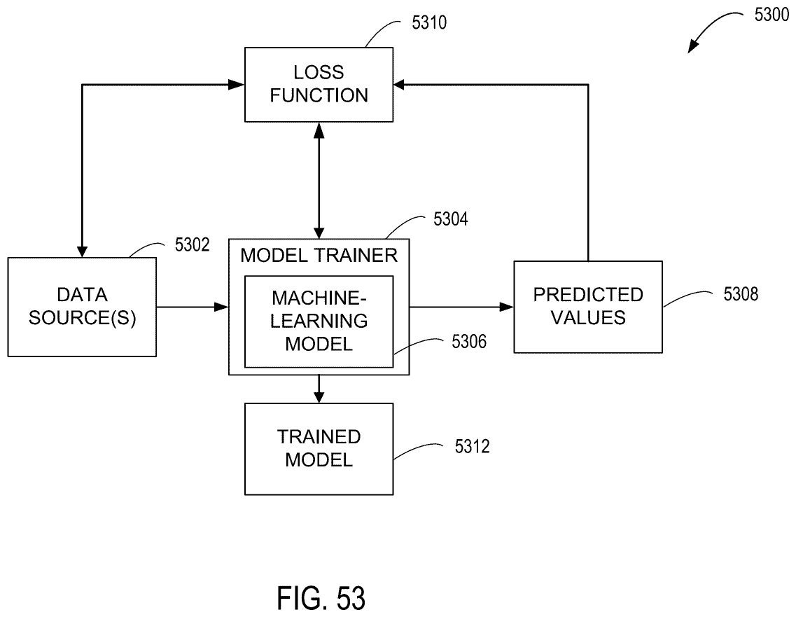

FIG. 53 illustrates an exemplary machine learning model training pipeline 5300 that enables the integrity monitoring platform 4300 to develop industry-specific analytical models within the analytics layer 4308 in accordance with at least some aspects of the present disclosure. The training pipeline 5300 provides a systematic approach, as described in the model training pipeline 4700, creating specialized models that transform dielectric fingerprints into actionable intelligence about contamination, tampering, degradation, dilution, substitution, or hazard events across diverse industrial applications.

Data source(s) 5302 represent comprehensive repositories of dielectric fingerprint data. These sources aggregate the raw sensor data 4702 collected from sensor nodes 4314, 4316, and 4318, which are deployed across various liquid containment systems. For the platform's multi-industry applications, data source(s) 5302 may encompass dielectric constant (Er) measurements that capture each substrate's unique electromagnetic signature. For example, in whiskey barrel monitoring, these sources may contain data describing the evolution of dielectric spectra as spirits age, including standard maturation patterns and documented instances of tampering. As another example, for military fuel applications at FARPs, the data may include dielectric signatures of JP-8 with varying contamination levels (e.g., water content showing characteristic shifts toward εᵣ≈80, microbial growth patterns, and malicious adulterants). In some aspects, the data source(s) 5302 may collect both baseline signatures of pure substrates and deviation patterns indicating integrity compromise. The data source(s) 5302 may include controlled laboratory data where known adulterants are introduced in measured quantities, establishing ground truth for the substance classification capabilities that enable the platform to identify specific contaminants based on dielectric shifts.

In some aspects, the model trainer 5304 represents a computational engine that transforms collected dielectric fingerprints into predictive models capable of real-time integrity assessment. Operating within the cloud platform 4600, the model trainer 5304 implements the training stage 4708 of the model development pipeline. This component processes the labeled data that associates dielectric measurements with verified liquid states, enabling supervised learning of contamination signatures. The model trainer 5304 may employ various algorithmic approaches optimized for providing continuous audit capabilities that manual sampling cannot achieve. For each industry vertical, the model trainer 5304 may extract relevant features through the feature engineering 4706 process; for example, spectral peaks indicating water contamination in fuel, phase shifts revealing pharmaceutical degradation, or amplitude variations signaling whiskey dilution. In some aspects, the model trainer 5304 may implement transfer learning techniques, allowing base models trained on one substrate to be adapted for related applications.

Machine-learning model 5306 represents the algorithmic architecture being trained to analyze the substrate's dielectric fingerprint and detect deviations indicating integrity events. Aligned with the models maintained in the model registry 4604, these architectures are selected based on specific monitoring requirements. For continuous contamination detection in pipeline applications, model 5306 may implement temporal convolutional networks that identify evolving patterns in streaming dielectric data. For batch-based quality verification in pharmaceutical manufacturing, the model 5306 may employ deep neural networks that map complex spectral signatures to purity metrics. The model 5306 structure reflects the platform's multi-modal sensing capabilities, incorporating inputs from dielectric sensors 4412, radar antennas 4410, and/or auxiliary sensor module 4428 when deployed in the modular configuration 4414. In some aspects, a multi-channel architecture enables comprehensive integrity assessment; for example, dielectric measurements provide compositional analysis, radar returns indicate volumetric changes, and environmental sensors supply temperature compensation data.

Predicted values 5308 represent the model's interpretation of dielectric fingerprints into specific integrity metrics aligned with industry requirements. For example, for oil and gas applications, predicted values 5308 may include water cut percentages, paraffin buildup indicators, or identification of specific adulterants that threaten FARP operations. In pharmaceutical monitoring, predictions may consist of active ingredient concentrations, detection of counterfeit formulations, or forecasts of stability degradation. The predicted values 5308 implement the platform's anomaly determination 5112 and adulterant identification 5110 capabilities, providing not just binary contamination flags but detailed characterization of detected integrity events. These outputs may include confidence scores reflecting the ML model's certainty, enabling risk-aware operational decisions.

The loss function 5310 may quantify the model's 5306 prediction accuracy, implementing weighted objectives that reflect real-world operational priorities. For example, for mission-critical military fuel monitoring, the loss function 5310 may heavily penalize false negatives, where undetected contamination could damage aircraft engines while accepting higher false favorable rates that trigger precautionary testing. The loss function 5310 may incorporate industry-specific constraints: pharmaceutical applications emphasize precision in concentration measurements, while water safety deployments prioritize rapid detection of any contamination regardless of specific identification. The feedback mechanism from loss function 5310 to model trainer 5304 drives iterative optimization, progressively improving the model's ability to distinguish between benign variations and genuine integrity threats.

In some aspects, the trained model 5312 represents the deployment-ready output that is added to the model registry 4604 for operational use. This finalized model encapsulates the learned patterns that link dielectric fingerprints to integrity states. The trained model 5312 may include different versions based on various deployment scenarios. For example, a full-precision version may be used for cloud-based inference with maximum accuracy, while quantized variants may be used for edge deployment on sensor node processors 5004. In some aspects, an ensemble configuration may combine multiple models for critical applications. Each trained model 5312 may include metadata describing its target industry, substrate types, detection capabilities, and performance benchmarks.

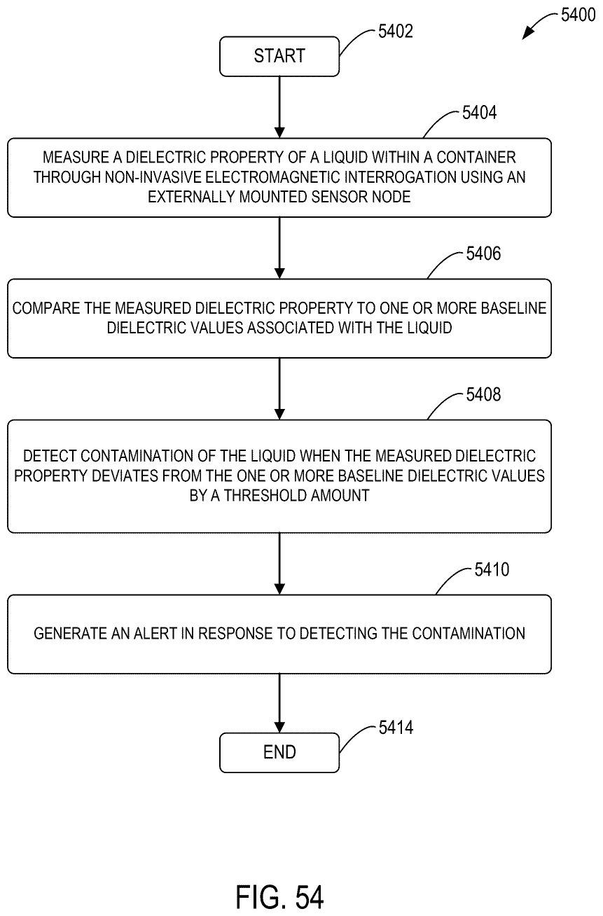

FIG. 54 depicts an example method 5400 for monitoring liquid integrity through non-invasive dielectric property measurement. In one aspect, method 5400 can be implemented by the integrity monitoring platform 4300 of FIG. 43 and/or the universal sensor node assembly 4400/4414 of FIGS. 44A/44B.

Method 5400 starts at block 5402 with measuring a dielectric property of a liquid within a container through non-invasive electromagnetic interrogation using an externally mounted sensor node. This measurement operation corresponds to the functionality of the dielectric sensor 4412 and/or radar antenna 4410 described in FIG. 44A, which may generate an electromagnetic field that penetrates through the container wall to interact with the contained liquid. The sensor nodes 4314, 4316, and/or 4318 of FIG. 43 may capture the relative permittivity (Er) or dielectric constant of the liquid, creating a dielectric fingerprint that serves as an indicator of the liquid's molecular composition, purity, and integrity. As described in connection with FIG. 51, this measured dielectric value 5102 reflects the current dielectric signature of the monitored substrate.

Method 5400 continues to block 5404 with comparing the measured dielectric property to one or more baseline dielectric values associated with the liquid. This comparison operation may be performed by the analytics engine 4330 within the analytics layer 4308 of FIG. 43, which accesses expected dielectric values 5104 from the feature store 4606 or model registry 4604 as illustrated in FIG. 46. The baseline values represent the expected dielectric fingerprint for the specific liquid under current environmental conditions, incorporating substrate-specific models trained through the model training pipeline 4700 of FIG. 47. As shown in the comparison operation 5106 of FIG. 51, this step may quantify the deviation (Δεᵣ) from the liquid's baseline fingerprint.

Method 5400 proceeds to block 5408 with detecting contamination of the liquid when the measured dielectric property deviates from the one or more baseline dielectric values by a threshold amount. This detection corresponds to the anomaly determination 5112 generated by the machine learning model 5108 of FIG. 51, which processes the dielectric differential alongside temporal patterns. As illustrated in FIG. 49, anomalies such as 4902 represent sudden changes in the substrate's dielectric fingerprint that exceed threshold parameters established by the machine learning models 4332. The detection leverages the integrity monitoring platform's 4300 of FIG. 43 capability to distinguish between different types of integrity compromises based on the magnitude and temporal characteristics of the dielectric deviation, as water contamination typically causes shifts toward εᵣ≈80, while other adulterants produce distinct dielectric signatures.

Method 5400 concludes at block 5410 with generating an alert in response to detecting the contamination. This alert generation is may be performed by the alert generator 4804 within the results processing engine 4802 of FIG. 48 and/or the results processing and communication layer 4310 of FIG. 43, which implements a multi-tiered alerting framework. The generated alerts may be displayed through alert cards 4810 on the dashboard user interface 4808 and/or at the end-user layer 4312 of FIG. 43, providing operators with immediate notifications such as “Water contamination detected in Pipeline Segment #7—95% confidence based on εᵣ shift from 2.1 to 8.3.” As shown in the deployment schematic of FIG. 52, these alerts 5224 enable real-time operational responses across distributed infrastructure.

Method 5400 provides beneficial technical effects by enabling continuous, non-invasive monitoring of liquid integrity without requiring container penetration or manual sampling. This technical solution addresses the fundamental problem of detecting contamination, tampering, or degradation in sealed containers while preserving product integrity. By leveraging the unique dielectric fingerprints of liquids and machine learning-based pattern recognition, method 5400 transforms electromagnetic measurements into actionable intelligence about contamination events. The method's integration with the modular sensor architecture of FIGS. 44A-44B enables deployment across diverse industries, from military fuel monitoring at FARPs to pharmaceutical quality assurance, using the same core technology with industry-specific analytical models. Combined with real-time detection capabilities, a proactive monitoring solution is provided that prevents costly failures, ensures regulatory compliance, and maintains product quality across critical liquid storage and distribution systems.

While examples of the operation of the invention have been presented, it would be understood that the examples using specific values are solely made for the purpose of providing information to those skilled in the art regarding the operation of the invention claimed and do not represent limiting values as to the scope of the subject matter of the invention as is recited in the claims.

The device disclosed, while discussed with regard to a barrel or container associated with alcohol production, it would be recognized that the system and processing disclosed, herein, is applicable to other configurations, such as septic tank, waste management systems, water towers, or other similar type containers that are used to retain one or more content that may be a liquid, a mash and/or a solid (and combination thereof) to determine a level of the contained content, whether liquid, mash or solid within the configuration.

For example, the invention disclosed may determine a level of a solid content within a septic tank, wherein the system comprises at least one transmitting antenna positioned on a face (or the lid) of the septic tank, the transmitting antenna configured to transmit into the tank a frequency modulated signal, wherein a starting frequency and a modulation of the signal is selected based on the material within the tank; and at least one receiving antenna configured to receive return signals corresponding to the transmitted frequency modulated signal, wherein the return signals are received within a time window associated with a corresponding transmitted signal, determining from a time of the received return signal with respect to the corresponding transmitted signal, a distance from the at least one transmitting antenna to the fill level of the solid content; and provide an indication of the fill level to at least one monitoring system, the monitoring system being one of: local to the system and remote from the system.

In still another example, the invention disclosed may monitor content within a container by transmitting signal downwardly into a container from a source positioned external to the container, at least one signal into the container, wherein the frequency of the at least one signal varies over time, determining a time of reception of a response with respect to the transmission, wherein the response is received within an expected time window after a time of transmission of a corresponding transmitted signal, determining a distance to at least one content within the container generating the based on the time of the return; and provide an indication of a level of fill of at least one of the at least one content within the container.

In one aspect of the invention, the frequency of transmission of each of the at least one signal is based, in part, of the content within the container, wherein a starting frequency of each of the at least one transmitted signal is based, in part, on the expected content with the container.

In one aspect of the invention, the variation of the frequency of the at least one transmitted signal is based, in part, on the expected content of the container, wherein the variation of frequency transmission is one of: continuous and patterned.

In one aspect of the invention, the transmission of the signal into the container may be arranged substantially perpendicular to a face of the container or transmitted at an angle to the expected content. In one aspect of the invention, an alarm may be triggered when a steepness of the angle of transmission falls below a known threshold.

In one aspect of the invention, providing an indication of a level of fill comprises transmitting the fill level to one of: a locally monitored memory storage device and to a remote location, wherein the transmission is through one of: a wired connection and a wireless connection.

In still another example, the invention disclosed may determine a level of at least one content within a container, wherein the system comprises at least one transmitting antenna and at least one receiving antenna positioned both of which may be positioned jointly or separately on a face of the barrel, wherein each of the at least one transmitting antenna is configured to transmit a signal in at least one frequency range with at least frequency variation during the transmission, each of the at least one receiving antenna configured to receive a corresponding response to the transmitted signal and a processing system comprising a processor and a memory containing therein processor readable instruction, which when accessed by the processor cause the processor to instruct each of the transmitting antenna to transmit the at least one signal, receive from a selected one of the plurality of receiving antenna a response from the transmitted at least one signal, wherein the response is received within an expected time window; determine a level of at least one content with the container; and provide an indication of the determined level for each of the at least one content within the container.

In accordance with at least one aspect of the present disclosure, a method for measuring changes in a fluid stored within a container is described. In some aspects, the method may include: generating, by a radio-frequency sensing element, a signal indicative of at least one dielectric property associated with a fluid stored within a container, the fluid being subject to at least one of a composition-based or an environment-driven change resulting from interactions with the container; and generating an output representative of at least one time-varying fluid characteristic of the fluid.

In some aspects, the method comprises: receiving the signal from the radio-frequency sensing element at a plurality of measurement intervals; and analyzing changes in the signal across the plurality of measurement intervals to detect at least one fermentation parameter of the fluid that changes over time.

In some aspects, the method comprises: storing, in a memory device, signals received across the plurality of measurement intervals; detecting a threshold change in the at least one fermentation parameter; and providing a notification when the threshold change is reached.

In some aspects, the method comprises: measuring, via an environmental sensor proximate to the container, at least one of temperature or humidity; and adjusting the at least one time-varying fluid characteristic based on the at least one of temperature or humidity.

In some aspects, the method comprises: inputting, to a machine-learning model, the signal indicative of the at least one dielectric property; and receiving, from the machine-learning model, data representing the at least one time-varying fluid characteristic of the fluid.

In some aspects of the method, the at least one time-varying fluid characteristic comprises at least one of a fluid level or a fluid volume.

In some aspects, a system for measuring changes of a fluid stored within a container is described. In some aspects, the system may include: a radio-frequency sensing element disposed externally on a container, the radio-frequency sensing element configured to generate a signal indicative of at least one dielectric property associated with a fluid stored within the container, the fluid being subject to at least one of a composition-based or an environment-driven change resulting from interactions with the container; and a processing component to generate an output representative of at least one time-varying fluid characteristic of the fluid based on the signal from the radio-frequency sensing element.

In some aspects, the container comprises a wooden barrel having an interior surface configured to impart at least one of a flavor or a chemical change to the fluid.

In some aspects, the fluid is a beverage comprising at least one of a wine, beer, or distilled spirits, and the at least one time-varying fluid characteristic of the fluid includes at least one fermentation parameter that changes over time.

In some aspects, the processing component is further to receive the output from the radio-frequency sensing element at a plurality of measurement intervals.

In some aspects, the processing component is further to: store each output received across multiple measurement intervals in a memory device, analyze each output to detect a threshold change in the at least one fermentation parameter, and provide a notification when the threshold change is reached.

In some aspects, the radio-frequency sensing element includes an antenna array adhered to an external surface of the container.

In some aspects, the processing component is further to: obtain at least two output signals per measurement interval, each output signal of the at least two output signals corresponding to a different sub-band within a frequency range; and obtain the at least one time-varying fluid characteristic based on a combination of at least two output signals.

In some aspects, to generate the signal indicative of the at least one dielectric property associated with the fluid stored within the container comprises to receive a radio-frequency interrogation signal and generate the signal based on the radio-frequency interrogation signal.

In some aspects of the system, the system further comprises an environmental sensor configured to measure at least one of temperature or humidity proximate to the container, wherein the processing component is configured to adjust the at least one time-varying fluid characteristic for ambient conditions based on data from the environmental sensor.

In some aspects, the processing component is configured to vary a measurement interval based on a rate of change in the at least one dielectric property.

In some aspects, to generate the output representative of the at least one time-varying fluid characteristic of the fluid comprises to: input the signal indicative of the at least one dielectric property of the fluid stored within the container into a machine-learning model; and output, by the machine-learning model, based on the input, data representative of the at least one time-varying fluid characteristic of the fluid.

In some aspects, the machine-learning model is trained on training data comprising a plurality of data points associated with one or more fluids, and wherein the machine-learning model is trained to predict, based on the training data, one or more time-varying fluid characteristics associated with one or more fluids.

In some aspects, the at least one time-varying fluid characteristic of the fluid includes at least one of fluid level or fluid volume.

In some aspects, a method for monitoring liquid integrity is described. The method may include measuring a dielectric property of a liquid within a container through non-invasive electromagnetic interrogation using an externally mounted sensor node; comparing the measured dielectric property to one or more baseline dielectric values associated with the liquid; detecting contamination of the liquid when the measured dielectric property deviates from the one or more baseline dielectric values by a threshold amount; and generating an alert in response to detecting the contamination.

In some aspects, the method may include inputting the measured dielectric property to a machine learning model trained to identify contaminant types; and determining a specific contaminant present in the liquid based on dielectric property deviation patterns.

In some aspects, the method may include calculating a confidence score for the determined specific contaminant; comparing a dielectric property deviation pattern to a library of known contaminant dielectric property deviation patterns; identifying potential secondary contaminants when the confidence score is below a first threshold; and generating a contamination profile listing identified contaminants with respective contamination probabilities.

In some aspects, the method may include measuring the dielectric property at multiple time intervals to create a time series; applying a change point detection algorithm to identify an initial point in time associated with the contamination; calculating a contamination rate based on a temporal evolution of deviations of the dielectric property; predicting a point in time when the contamination will exceed a threshold value; and scheduling preventive maintenance based on the point in time.

In some aspects, measuring the dielectric property of the liquid within the container comprises: transmitting electromagnetic waves at multiple frequencies through a wall of the container; measuring amplitude and phase changes at each frequency; constructing a dielectric spectrum based on the measured amplitude and phase changes at each frequency; extracting frequency-dependent features from the dielectric spectrum; and using the features to distinguish between different contamination types.

In some aspects, the method may include establishing secure communication between the sensor node and a cloud platform; encrypting the measured dielectric property; transmitting the encrypted measured dielectric property to a data repository; and creating an immutable audit trail of all measurements and alerts.

In some aspects, the method may include receiving an indication that an additional sensing capability is enabled for the sensor node; detecting connection of an auxiliary sensor module to the sensor node; discovering sensor type and measurement parameters of the auxiliary sensor module; receiving supplemental measurements from the auxiliary sensor module; and correlating the supplemental measurements with the measured dielectric property to enhance contamination detection accuracy.

In some aspects, a system is disclosed. The system may include: a sensor node configured for external mounting to a container, the sensor node configured to measure a dielectric property of liquid within the container through non-invasive electromagnetic interrogation; one or more processors configured to: receive a dielectric measurement from the sensor node; compare the dielectric measurement to a baseline dielectric value for the liquid; detect contamination of the liquid when the dielectric measurement deviates from the baseline dielectric value by a threshold amount; and generate an alert in response to a detection of contamination of the liquid.

In some aspects, to detect contamination, the one or more processors are configured to: provide the dielectric measurement to a machine learning model trained on patterns of contaminated and uncontaminated liquid samples; receive an anomaly score from the machine learning model indicating likelihood of contamination; compare the anomaly score to a contamination threshold; and determine contamination is present when the anomaly score exceeds the contamination threshold; wherein the machine learning model is configured to compare the dielectric measurement to the baseline dielectric value for the liquid learned during training.

In some aspects, the system may further comprise a machine learning model executable by the one or more processors, wherein the one or more processors are configured to: input the dielectric measurement to the machine learning model; and identify a type of contaminant based on deviation patterns analyzed by the machine learning model.

In some aspects, the one or more processors are further configured to: calculate a confidence score for the identified type of contaminant; compare the deviation patterns to a library of known contaminant signatures stored in memory; and flag potential secondary contaminants when the confidence score falls below a first threshold.

In some aspects, the sensor node comprises a radar antenna configured to transmit electromagnetic pulses through a wall of the container.

In some aspects, the sensor node comprises modular sensor interfaces, and wherein the one or more processors are configured to: detect connection of an auxiliary sensor module; identify a type of auxiliary sensor module and a sensor capability; receive supplemental measurements from the auxiliary sensor module; adjust the baseline dielectric value based on the supplemental measurements; and modify the threshold amount according to environmental conditions indicated by the auxiliary sensor module.

In some aspects, the liquid is fuel oil.

In some aspects, the system further comprises: a wireless transceiver configured to establish secure communication between the sensor node and a cloud platform; wherein the one or more processors are configured to: encrypt the measured dielectric property; transmit the encrypted measured dielectric property to a data repository; and create an immutable audit trail of all measurements and alerts.

In some aspects, the one or more processors are configured to: track temporal patterns of dielectric measurements over multiple days; apply predictive analytics algorithms to the temporal patterns to forecast liquid degradation trajectories and future liquid contamination levels; calculate a maintenance window based on the forecasted liquid degradation trajectories and future liquid contamination levels.

In some aspects, a sensor node for liquid integrity monitoring is described. The sensor node may comprise: a sensor configured to measure a dielectric property of liquid within a container through non-invasive electromagnetic interrogation when externally mounted to the container; one or more processors configured to execute a machine learning model to detect liquid contamination based on the measured dielectric property; and a transceiver configured to transmit a contamination alert based on the detected liquid contamination.

In some aspects, the sensor node further comprises: a modular connection interface for attaching an auxiliary sensor module; wherein the one or more processors are configured to: detect connection of an auxiliary temperature sensor module; and receive a temperature measurement from the auxiliary sensor module.

In some aspects, the one or more processors are configured to: operate in a low-power sleep mode between measurements; wake at predetermined intervals to measure the dielectric property; activate the transceiver when at least one of contamination is detected or during scheduled synchronization windows; and download a portion of an updated machine learning model during the synchronization windows.

In some aspects, liquid is fuel oil.

In some aspects, a method for non-invasive liquid integrity monitoring using dielectric fingerprinting is described. The method may comprise: establishing a baseline dielectric fingerprint for a liquid within a sealed container, wherein the baseline dielectric fingerprint comprises a unique electromagnetic signature based on molecular composition of the liquid; continuously measuring dielectric properties of the liquid through non-invasive electromagnetic interrogation to generate real-time dielectric fingerprints; comparing the real-time dielectric fingerprints to the baseline dielectric fingerprint to identify deviations; determining a specific contaminant identity based on a magnitude and pattern of the deviations, wherein different contaminants produce distinct deviation signatures in the dielectric fingerprint; and generating a contamination profile including the specific contaminant identity and a confidence score.

In some aspects, a method for identifying liquid contamination through multi-frequency dielectric analysis is described. The method may comprise: transmitting electromagnetic signals at a plurality of frequencies through a container wall into a liquid; measuring dielectric constant values at each frequency to create a dielectric spectrum; detecting frequency-dependent deviations from an expected dielectric spectrum for the liquid; applying a trained machine learning model to the frequency-dependent deviations to classify contamination type, wherein the model is trained on known dielectric spectra of contaminants including water (εᵣ≈80), methanol (εᵣ≈33), and glycerol (εᵣ≈42); and outputting both a contamination determination and a specific contaminant identification with associated probability.

In some aspects, a software-defined liquid monitoring system is described. The system may include: a standardized sensor hardware platform configured to measure dielectric properties of liquids in sealed containers; a model registry storing a plurality of industry-specific machine learning models, each model trained to recognize dielectric fingerprint patterns specific to a different industry application; a model selection module that automatically loads an appropriate industry-specific model based on deployment configuration; and an inference engine that applies the selected model to measured dielectric properties to detect contamination patterns specific to the industry application, wherein the same sensor hardware platform monitors different liquid types by loading different software models without hardware modification.

In some aspects, a modular sensor system for container monitoring is described. The system may comprise: a central processing unit having a plurality of standardized sensor connection interfaces; a base dielectric sensor module removably connectable to a first interface, the base dielectric sensor module configured to measure dielectric properties through electromagnetic interrogation; at least one auxiliary sensor module removably connectable to a second interface; wherein each sensor module includes: a self-description capability that broadcasts sensor type, measurement range, and calibration parameters upon connection; a standardized electrical and mechanical connector ensuring proper alignment and environmental sealing; and a hardware abstraction layer in the central processing unit that automatically recognizes connected sensor modules and loads appropriate drivers without manual configuration, wherein the system adapts to different monitoring applications by connecting different combinations of sensor modules to the same central processing unit.

In some aspects, a field-configurable liquid monitoring system is described. The system may comprise: a central hub having multiple sensor interfaces supporting hot-swapping capability; a plurality of interchangeable sensor modules, each module comprising: a sensor element; non-volatile memory storing calibration data and sensor capabilities; a plug-and-play connector with mechanical keying to prevent incorrect installation; a module discovery protocol that: detects connection of a sensor module during system operation; retrieves sensor capabilities and calibration data from the connected module; dynamically reconfigures measurement algorithms based on the connected sensor configuration; and wherein sensor modules can be added, removed, or replaced during operation without system disruption.

In some aspects, a distributed container monitoring system is described. The system may comprise: a plurality of modular sensor nodes, each comprising: a central processing unit with modular sensor interfaces; at least one dielectric sensor module measuring liquid dielectric properties; an edge computing module executing compressed machine learning models locally; a communication module supporting multiple protocols; wherein each sensor node operates in one of: an autonomous mode executing local inference when network connectivity is unavailable; a connected mode transmitting data to cloud infrastructure when connectivity exists; a hybrid mode performing edge preprocessing before selective cloud transmission; a synchronization mechanism that: uploads detected anomalies and refined baselines when connectivity is restored; downloads updated machine learning models adapted to specific sensor configurations; and wherein the modular architecture enables field adaptation to different container types and liquid monitoring requirements through sensor module selection.

In some aspects, a universal liquid integrity monitoring platform is described. The system may include a modular sensor assembly having: a base platform with standardized mechanical mounting and electrical interfaces; a core dielectric sensing module providing fundamental liquid measurement; industry specific auxiliary modules selected from: a high-temperature module for industrial process monitoring; an intrinsically safe module for explosive atmospheres; a sanitary-rated module for pharmaceutical applications; an extended-range radar module for large tank monitoring; a unified data interface providing consistent data formatting regardless of connected modules; a multi-layer firmware architecture comprising: a base layer handling hardware interfaces common to all configurations; a middleware layer implementing module-specific drivers; an application layer executing industry-specific algorithms; wherein transitioning between industry applications requires only connecting appropriate modules and loading corresponding firmware, without modifying the base platform.

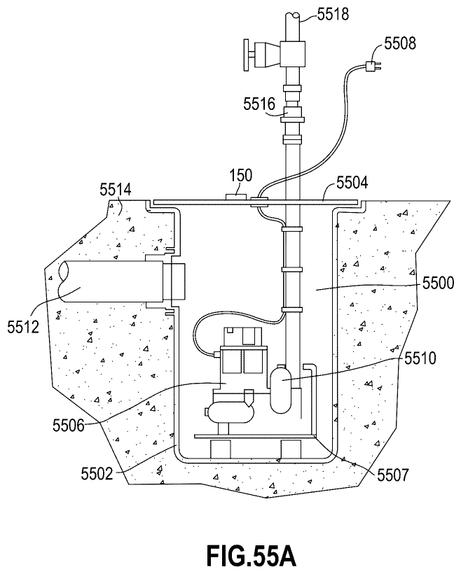

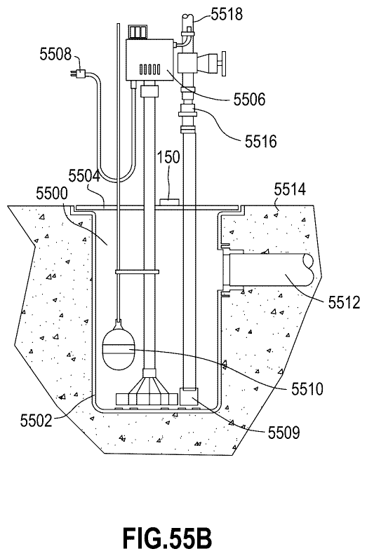

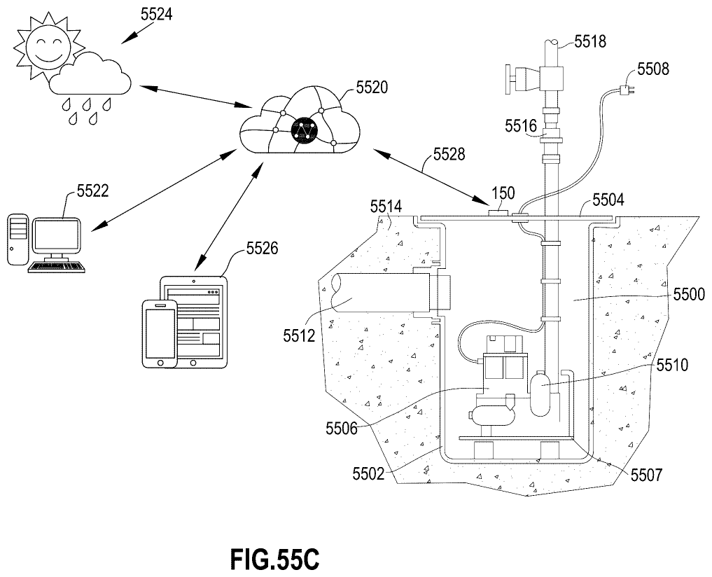

FIG. 55A illustrates an exemplary aspect of a sump monitoring system equipped with a submersible pump in accordance with at least one aspect of the present disclosure. Container 5500 of the sump system is configured to fill with fluid introduced to the container 5500 via inlet 5512. Once sufficient fluid has been introduced to the container 5500, float 5510 may trigger, causing pump 5506 (optionally positioned on pump stand 5507) to activate. Pump 5506 may pump the fluid out of the container 5500 through outlet 5518, which may include one or more valves 5516. The container 5500 may include one or more container walls 5502 and one or more lids 5504. In certain embodiments, the container may be positioned such that some or all of the container is positioned level with or below the floor 5514 of a structure or site. Pump 5506 is powered by a power source 5508.

At a high level, a sump system is generally designed to manage and remove unwanted liquid (often groundwater or wastewater) from a low point in a structure or site to prevent flooding, water damage, or operational disruption. Fluid collects in a designated low area (often a basin or pit, e.g., container 5500) and, when it reaches a certain condition such as a threshold level, is automatically detected and expelled by a pump (e.g., pump 5506) to a discharge location away from the protected area (e.g., via outlet 5518). Sump systems are widely used in buildings, infrastructure, and industrial settings to control water intrusion, manage seepage, and maintain dry, safe, and functional environments where gravity drainage alone is insufficient.

In most cases, pumps of sump systems are controlled via a float (e.g., float 5510). When the fluid reaches a first level, the float rises due to buoyancy, and the geometry of the rising float mechanically causes the pump to engage. When the fluid reaches a second level (lower than the first level), the geometry of the falling float mechanically causes the pump to disengage. The first level is set at a higher fluid level than the second level (hysteresis) to keep the pump from short cycling.

Floats are used for most sump level sensing due to the simplicity of the system and the low likelihood of degradation in most contexts. If the sensor that activates a pump of a sump system fails, the structure or site will eventually flood, potentially causing significant damage to the structure or site. If an electrical solution like an interior RF level sensor is used to control the pump, several failure cases have the potential to occur. For example, the interior RF level sensor may be corroded or otherwise damaged by the unclean fluids in the container 5500. Alternatively, the interior RF level sensor may lose connection to its power source, or its battery may run out of charge. In other cases, the interior RF level sensor may lose its connection (either wired or wireless) to the pump. In still other cases, the interior RF level sensor may incorrectly sense the fluid level due to the geometric complexity and relatively narrow shape of most sump containers (e.g., sump basins). In short, factors like the corrosive environment, narrow shape, geometric complexity, and need for sensory certainty associated with most sump systems are ill-suited to most interior RF level sensors. Interior RF level sensors are used by way of example; other types of electronic sensors, such as conductivity sensors, capacitive sensors, electrochemical sensors, optical sensors, ultrasonic sensors, pressure sensors, magnetic sensors, or other electronic sensors could theoretically be used, but would suffer from one or more of the shortcomings discussed above with respect to interior RF level sensors.

However, despite being the best available previously known sensing modality in most contexts, floats have several shortcomings of their own. First, most sump systems are inherently dirty, corrosive, and otherwise geometrically and chemically unpredictable. Due to the introduction of excess gunk or foreign objects via the sump container inlet (e.g., inlet 5512), float sensors may become jammed or stuck. Furthermore, because float sensors are generally configured to mechanically actuate the pump, most float sensors are ill-suited to data collection and electronic diagnostics. Moreover, for the same reason, it is impossible to modify the hysteresis of most float systems without physically modifying the geometry of the float sensor or the pump. Lastly, some contexts (like certain acidic chemical process sumps, caustic or alkaline sumps, solvent or hydrocarbon sumps, high-temperature chemically active sumps, electroplating or surface plating sumps, and wastewater sumps with aggressive chemical byproducts) have sufficient chemical activity and/or extreme temperature to degrade a float sensor or otherwise disable the float sensor.

Therefore, there exists a need for an electronic level sensor able to overcome the shortcomings of existing electronic sensors such that it can be effectively used either in addition to a float sensor or in the place of a float sensor.

Turning back to FIG. 55A, FIG. 55A depicts a monitoring system 150 (discussed at length above, e.g., with respect to FIGS. 1, 2, 7A, and 9, in addition to description through the present specification) positioned on the lid 5504 of the container 5500. Monitoring system 150 may communicate via a wired or wireless connection with pump 5506. Moreover, monitoring system 150 may be powered via any appropriate power source, such as a wired source, battery power, or any combination thereof. In certain embodiments, pump 5506 and monitoring system 150 may share a power source (e.g., a wired power source such as the power source 5508 depicted in FIG. 55A). Monitoring system 150 may be configured to detect a level of fluid within container 5500 by transmitting a signal through a surface of the container 5500 (e.g., lid 5504), receiving a response associated with the transmitted signal, detecting a change of a characteristic between the transmitted signal and the response, and determining a fluid level factor (e.g., one or more of fluid level, the rate of change of fluid level, or both) based on the change of the characteristic. Monitoring system 150 may also cause a pump (e.g., pump 5506) of the sump system to activate or deactivate based on the fluid level factor. In certain embodiments, monitoring system 150 may be used in addition to float 5510 (and/or any other additional sensors) to provide sensory redundancy. In certain other embodiments, monitoring system 150 may be used without float 5510. Any number of monitoring systems 150 may be used without departing from the scope of the present disclosure. Though FIG. 55A depicts a single monitoring system on the lid 5504 of container 5500, one or more monitoring systems may be positioned at any suitable one or more locations relative to the container 5500, such as on one or more container walls 5502. Monitoring system 150 may utilize any suitable electronic sensor technology disclosed herein.

As used herein with respect to the sump system embodiments, monitoring system 150 may comprise one or more sensor nodes positioned external to the container 5500. The term “sensor node” as used herein may refer to any sensing element, transceiver, antenna, RF-responsive element, or combination thereof configured to transmit signals into or through a surface of a container and receive a response associated with the transmitted signals. The one or more sensor nodes of monitoring system 150 may include, without limitation: processing system 210 and one or more of antennas 220a-n arranged relative to center point 260 as described with respect to FIG. 2; RF-responsive elements 220u-220n and/or RF-responsive element 2402 configured to interact with environment 2404 as described with respect to FIG. 24; sensor architecture 2502 including antenna 2504, MCU 2510, demodulator 2508, modulator 2512, energy harvester 2516, and sensors 2524 as described with respect to FIG. 25; and/or sensor nodes 4314, 4316, and 4318 of sensor layer 4302 as described with respect to FIG. 43. Signal characteristics detected by the one or more sensor nodes may include one or more of RSSI, phase, CSI, resonant frequency, or any combination thereof, as described with respect to FIGS. 26-28 and signal characteristics 3314 of FIG. 33. The one or more sensor nodes may be positioned at any suitable location external to the container 5500, including on lid 5504, on one or more container walls 5502, proximate to but spaced apart from the container 5500 at a standoff distance, or any combination of such positions. In embodiments where the one or more sensor nodes are spaced apart from the container 5500, the sensor nodes may be mounted on a nearby structure such as a wall, rack, ceiling, or floor, and may correspond to node 3518 as described with respect to FIG. 35 or readers 3808, 3810, or handheld device 3812 as described with respect to FIG. 38.

The one or more sensor nodes of monitoring system 150 may operate using any suitable sensing modality disclosed herein, including but not limited to: radar-based sensing wherein the sensor node transmits RF signals directly toward the container and analyzes reflections; backscatter-based sensing wherein the sensor node interrogates passive or semi-passive RF-responsive elements affixed to the container surface; active transponder-based sensing wherein RF-responsive elements with integrated processing capabilities (such as RF-responsive element 2414 with MCU 2416 as described with respect to FIG. 24) measure and report signal characteristics; or hybrid approaches combining multiple sensing modalities. The sensor nodes may transmit signals through any surface of the container 5500, including through lid 5504, through one or more container walls 5502, or through a bottom surface of the container 5500. The response associated with the transmitted signals may include reflected signals, backscattered signals, retransmitted signals, or any combination thereof, and the one or more processors may detect changes in characteristics including signal strength, frequency, phase, or other electromagnetic parameters as disclosed herein.

The above system embodies several advantages. By being positioned outside the container (e.g., container 5500), monitoring system 150 avoids direct exposure to the potentially bio-active, chemically active, dirty, high-or-low-temperature, or otherwise problematic fluid environment of the container. Moreover, in embodiments having both a monitoring system 150 and a float 5510, the multi-level sensing provides redundancy to decrease the risk of total sensory failure and subsequent flooding. As an additional advantage, the data-related capabilities of an electronic sensory system enable pumping based on a rate of change of fluid level rather than solely based on the current fluid level; that is, if a system detected that a rate of change of the fluid level in the pump system's container exceeds a given threshold (e.g., in a flash flood), the system could begin pumping the fluid out preemptively, before the fluid level reaches what would otherwise be the activation level for the pump. Furthermore, monitoring system 150 is capable of multi-level sensing; that is, the monitoring system 150 may be configured to adjust the levels at which the pump 5506 activates or deactivates, or the system may be configured to detect more than two levels. For example, in certain embodiments, the monitoring system 150 may be configured to adjust the power of the pump 5506 (e.g., via a variable frequency drive (VFD)) based at least in part on one or more of the fluid level, the rate of change of the fluid level, or both; if the level is especially high, rising quickly, or any combination thereof, the pump 5506 may be activated at a high power level to remove fluid quickly. Conversely, if the level is not especially high and is not rising quickly but is nevertheless higher than the preferred level, the monitoring system 150 may activate the pump 5506 at a low power level to remove fluid more slowly. As a simpler application of this multi-level advantage, a user or engineer of the system may modify the hysteresis of the system. Furthermore, the data-related advantages of electronic sensors allow for improved data collection and diagnostics. As a final example, in contexts in which it is optimal to have no float sensor inside the container (e.g., especially harsh chemical environments), the present system provides the ability to detect fluid level from outside the container.

The present application recites only a few of the many advantages of the disclosed electronic sensing technology. The advantage associated with redundancy may be of particular value to insurance companies. Insurance companies may offer discounts or provide monitoring systems 150 to insured customers in order to significantly reduce the risk of flooding at a low cost.

As mentioned above, existing electronic sensors generally fail to operate effectively in the field of sump fluid level technology due to the combination of harsh environmental conditions (e.g., chemicals or grime) and due to the moderately complex, narrow geometry of the container, which can distort a signal before it returns to the sensor. Monitoring system 150 overcomes these limitations through the systems and methods disclosed herein. For example, and without limitation, by correlating signal signatures with reference data sets and/or machine-learning models, monitoring system 150 may accurately determine fluid level despite sending the initial signal through a surface of the container 5500, and despite the container's geometry.

FIG. 55A is given by way of example rather than by way of limitation. Elements may be added, removed, altered, and/or rearranged without departing from the scope of the present disclosure.

In certain embodiments, monitoring system 150 may be configured to communicate sump data to one or more computers located on-site or located remotely. In certain embodiments, one or more alerts may be generated based at least in part on fluid level and/or a rate of change of fluid level (e.g., if fluid level and/or a rate of change exceed a corresponding threshold). Alerts may be communicated to one or more devices to inform individuals of a risk of flooding. In certain embodiments, monitoring system 150 may receive sump-relevant data from one or more computers located on-site or located remotely. Sump-relevant data (in addition to fluid level factors) may be used to inform pump activation levels, pump deactivation levels, and/or alert levels. For example, one or more computers (which may optionally comprise one or more servers) may communicate weather data to monitoring system 150, which may use the weather data (either in a hard-coded manner, via a machine learning model, or any combination thereof) to inform pump activation, pump deactivation, and/or alert decisions. In one illustrative example, if monitoring system 150 receives data informing it of a coming flash flood or storm, monitoring system 150 may engage the pump at a lower fluid level than it would have without the weather data. Monitoring system 150 may make processing decisions regarding pump activation or deactivation on-site or via one or more remote processors. In certain embodiments, data from monitoring system 150 may be displayed on a screen to inform one or more human operators for the purpose of modifying hysteresis values and/or for performing diagnostics. Hysteresis values may be modified manually, automatically, or any combination thereof.

Certain aspects of the present disclosure may include a hysteresis machine learning model. The hysteresis machine learning model may be trained on training data comprising a plurality of data points associated with one or more of weather data, fluid level data, or both. The hysteresis machine learning model may be configured to automatically adjust one or more hysteresis values; that is, the machine learning model may control the level at which the pump 5506 activates and/or the level at which the pump 5506 deactivates. The hysteresis machine learning model may run on computer equipment located on-site or remote from the sump system.

For example, certain aspects of the present disclosure may use one or more of the hysteresis machine learning model or hard-coded instructions to execute predictive hysteresis; that is, software instructions may cause a processor to modify the hysteresis of the pumping system in anticipation of future projected changes. In one embodiment, weather data projecting a dry period may be used to increase one or both of the lower and upper hysteresis thresholds (that is, allowing the amount of fluid to reach a higher total level before pumping). In another embodiment, weather data projecting an increase of precipitation may decrease one or both of the lower and upper hysteresis thresholds (that is, limiting the amount of fluid in the container to a lower total level). In yet another embodiment, a rate of change of the fluid level can be used for predictive hysteresis; for example, if the rate of change of the fluid level is increasing rapidly, the lower hysteresis threshold may be reduced to allow the pump to begin pumping earlier than it would have without predictive hysteresis. These examples are given by way of illustration rather than limitation, and other data may be used to enable predictive hysteresis without departing from the scope of the present disclosure.

Furthermore, certain aspects of the present disclosure may include a fluid level machine learning model. The fluid level machine learning model may be trained on training data comprising a plurality of data points associated with one or more of sensor signal data and fluid level data. The fluid level machine learning model may be configured to determine the level of the fluid in the container based on one or more of the signal transmitted by monitoring system 150 and the signal received by monitoring system 150. The fluid level machine learning model may run on computer equipment located on-site or remote from the sump system.

Container 5500 may comprise any structure, cavity, enclosure, reservoir, basin, pit, chamber, vault, trench, tank, housing, or collection volume configured to receive, retain, channel, or temporarily store fluid. The container 5500 may be formed as a discrete manufactured component, a prefabricated assembly, a cast-in-place structure, a lined excavation, or an integrated portion of a building, infrastructure, vehicle, vessel, or industrial installation. Container 5500 may be rigid, semi-rigid, or flexible, may be sealed or unsealed, and may be configured for below-grade, at-grade, or above-grade installation. In some embodiments, container 5500 may be open to the surrounding environment, while in other embodiments it may be substantially enclosed, vented, or pressure-rated. The container 5500 may define a single continuous volume or multiple interconnected volumes and may be sized, shaped, or oriented based on fluid capacity, footprint constraints, structural loading, or environmental conditions.

Container walls 5502 may comprise any surfaces, boundaries, liners, shells, partitions, or structural elements that define at least a portion of container 5500. Container walls 5502 may be formed from plastic, polymer, composite, metal, ceramic, concrete, masonry, coated substrates, multilayer laminates, or combinations thereof. The walls may be rigid, corrugated, ribbed, reinforced, insulated, chemically resistant, abrasion-resistant, or flexible. In some embodiments, container walls 5502 may be integral with a surrounding structure, such as a foundation wall, slab, or industrial vessel, while in other embodiments they may be separate liners or inserts. In certain embodiments, container walls 5502 may include apertures, ports, channels, ribs, mounting features, sensor interfaces, or fluid passages and may be configured to resist hydrostatic pressure, soil loading, vibration, thermal cycling, or chemical exposure. Container walls 5502 may include any surface of the container, including the top or bottom. Thus, certain embodiments may include a lid 5504 that forms a portion of the container walls 5502.

Lid 5504 may comprise any cover, cap, hatch, plate, closure, or barrier configured to partially or fully close container 5500. Lid 5504 may be removable, hinged, latched, sealed, bolted, threaded, or permanently affixed. In some embodiments, lid 5504 may provide structural support, load-bearing capability, environmental sealing, vapor containment, or safety isolation. Lid 5504 may include one or more access openings, ports, vents, grommets, cable pass-throughs, sensor mounts, inspection windows, or service interfaces. The lid 5504 may be formed from materials similar to or different from container walls 5502 and may incorporate gaskets, seals, filters, or membranes. In some embodiments, lid 5504 may be omitted entirely or replaced by a grate, mesh, or partial cover.

Pump 5506 may comprise any device configured to impart energy to a fluid to move, evacuate, circulate, or discharge the fluid from container 5500. Pump 5506 may be submersible, non-submersible, partially submerged, inline, or remotely located relative to the container. The pump may operate using centrifugal, positive displacement, diaphragm, peristaltic, screw, vortex, jet, air-lift, or other pumping mechanisms. Pump 5506 may be electrically driven, pneumatically driven, hydraulically driven, mechanically driven, or driven by stored energy. In some embodiments, pump 5506 may be a single pump, while in other embodiments multiple pumps may be provided for redundancy, staging, load sharing, or fault tolerance. Pump 5506 may be configured for intermittent or continuous operation and may include integrated or external controls, sensors, or protective features.

Pump stand 5507 may comprise any base, support, platform, pedestal, bracket, frame, suspension, spacer, or mounting structure configured to position, elevate, stabilize, isolate, or orient pump 5506 relative to container 5500 or floor 5514. Pump stand 5507 may be integral with the pump, integral with the container, or a separate component. The stand may be fixed, adjustable, removable, collapsible, or modular and may be configured to prevent sediment ingestion, reduce vibration, accommodate fluid flow, or align the pump with an outlet. In some embodiments, pump stand 5507 may be omitted entirely, and pump 5506 may rest directly on a container wall 5502, floor 5514, or be suspended from above.

Power source 5508 may comprise any source of energy capable of operating pump 5506 or associated system components. Power source 5508 may include grid-supplied electrical power, battery power, stored electrical energy, mechanical energy, pneumatic energy, hydraulic energy, fuel-powered generators, renewable energy sources, or combinations thereof. In some embodiments, power source 5508 may include backup, redundant, or auxiliary power sources configured to operate during outages, faults, or emergency conditions. Power source 5508 may be located within container 5500, external to the container, or remotely located and may include power conditioning, conversion, storage, or distribution components. In certain embodiments, a first one or more power sources may power pump 5506, and a second one or more power sources may power monitoring system 150. In certain other embodiments, monitoring system 150 and pump 5506 may share one or more power sources 5508.

Pump 5506 may be configured to activate or deactivate based on float 5510. In certain embodiments, float 5510 may be the primary sensory factor that controls pump 5506, and monitoring system 150 may act as a backup in the event that the system detects that the float 5510 is malfunctioning (e.g., jammed) and/or in the event the fluid in the container 5500 reaches a level above the float's activation level without the pump 5506 activating. In certain other embodiments, monitoring system 150 may be the primary sensory factor that controls pump 5506, and float 5510 may act as a backup in the event that the system detects that monitoring system 150 is malfunctioning and/or in the event the fluid in the container 5500 reaches a level above the monitoring system's activation level without the pump 5506 activating. Float 5510 may comprise any buoyant, semi-buoyant, or fluid-responsive element configured to change position, orientation, state, or output in response to fluid presence, fluid level, or fluid motion. Float 5510 may be mechanical, electromechanical, or hybrid and may actuate a switch, sensor, magnet, or control signal. The float may be spherical, cylindrical, elongated, hinged, tethered, guided, or free-floating and may be configured for single-level or multi-level actuation. In some embodiments, float 5510 may be replaced or supplemented by non-buoyant sensing elements that emulate float behavior via electronic, magnetic, optical, or other means. Certain embodiments may lack a float 5510 altogether.

In certain embodiments, additional sensors may be used either in place of one or more depicted sensors or in addition to one or more depicted sensors (e.g., monitoring system 150 or float 5510). For example, a fluid level sensor may be attached to a top surface (e.g., lid 5504) of container 5500 to detect an overflow of container 5500. In certain other embodiments, one or more fluid level sensors may be attached at other locations, such as to one or more container walls 5502, to communicate that the fluid has reached given levels or to assist in predicting a potential future overflow. Additionally or alternatively, one or more flow sensors may be used to detect flow rate. Flow rate may be used to assist in predicting future fluid level states (e.g., for predictive hysteresis) or to measure the efficiency of pump 5506. Additionally or alternatively, monitoring system 150 may integrate one or more dielectric sensors according to the systems and methods disclosed herein to determine if the contents or status of fluid within container 5500. Other sensors may be used in addition to or in the place of sensors disclosed herein.

Inlet 5512 may comprise any opening, conduit, channel, passage, port, pipe, or interface through which fluid enters container 5500. Inlet 5512 may be configured to receive fluid via gravity, pressure, capillary action, or forced flow and may comprise any combination of one or more drains, pipes, trenches, equipment, surfaces, or environmental sources. The inlet 5512 may include filters, screens, grates, valves, traps, or flow-conditioning features and may be oriented horizontally, vertically, or at an angle. In some embodiments, multiple inlets 5512 may be provided. In certain embodiments, inlet 5512 may be integrated into container walls 5502.

Floor 5514 may comprise any slab, deck, base surface, foundation, structural floor, ground surface, platform, or supporting substrate of a structure, site, or installation in which container 5500 is positioned or supported. Floor 5514 may be formed from concrete, masonry, metal, composite, wood, compacted soil, or other structural materials and may be above grade, at grade, or below grade. In some embodiments, container 5500 may be embedded within, mounted on, recessed into, suspended above, or otherwise coupled to floor 5514. Floor 5514 may include openings, recesses, sumps, channels, drains, cutouts, or penetrations configured to receive or interface with container 5500, inlet 5512, outlet 5518, pump 5506, or associated components. Floor 5514 may be planar, sloped, stepped, or contoured to direct fluid toward container 5500 or inlet 5512 and may be configured to bear structural loads, resist moisture intrusion, tolerate chemical exposure, or accommodate vibration and thermal expansion.

Valve 5516 may comprise any flow-control device configured to permit, restrict, regulate, or prevent fluid flow within the sump system. Valve 5516 may include check valves, gate valves, ball valves, flap valves, diaphragm valves, solenoid valves, pressure-relief valves, or other valve types. Valve 5516 may be passive or actively controlled and may respond to pressure, flow direction, fluid level, electrical signals, or control logic. The valve 5516 may be located at the inlet, outlet, pump discharge, and/or any intermediate location and may be integrated with piping or provided as a discrete component. Any one or more valves 5516 may be used without departing from the scope of the present disclosure, and certain embodiments may have no valves 5516.

Outlet 5518 may comprise any discharge opening, conduit, pipe, hose, channel, or interface through which fluid exits container 5500. Outlet 5518 may direct fluid to a drain, sewer, treatment system, storage vessel, environmental discharge point, or secondary system. The outlet may be rigid or flexible, fixed or movable, and may include fittings, couplings, valves, air gaps, or backflow-prevention features. Outlet 5518 may be configured for pressurized discharge, gravity discharge, or intermittent flow and may be oriented vertically, horizontally, or at an angle relative to container 5500.

The present disclosure is not limited to submersible pumps as illustrated in FIG. 55A; rather, any suitable pump may be used without departing from the scope of the present disclosure. For example, and without limitation, FIG. 55B illustrates an exemplary aspect of a sump monitoring system equipped with a pedestal pump in accordance with at least one aspect of the present disclosure. Here, pump 5506 is supported by pump pedestal 5509 rather than pump stand 5507 (though, to be more accurate, pump pedestal 5509 is a specific embodiment of pump stand 5507). In certain embodiments like that of FIG. 55B, pump 5506 may be positioned outside container 5500.

Pump pedestal 5509 may comprise any elongated support, column, shaft, riser, housing, or structural member configured to position at least a portion of pump 5506 outside container 5500 while mechanically, fluidly, or operatively coupling the pump to fluid within the container. In pedestal pump embodiments, pump pedestal 5509 may support a motor or drive assembly at a location outside container 5500 while a pumping element, intake, or drive linkage extends into the container. Pump pedestal 5509 may be formed as a single integral structure or as multiple coupled components and may be rigid or adjustable in height, orientation, or length. The pedestal may house or route a drive shaft, coupling, wiring, control lines, or fluid passages and may provide structural support, alignment, environmental separation, or vibration isolation. In some embodiments, pump pedestal 5509 may be mounted to floor 5514, lid 5504, container walls 5502, or another structural element, and in other embodiments the pedestal may be omitted or replaced by alternative support arrangements.

FIG. 55B is given by way of example rather than by way of limitation. Elements may be added, removed, altered, and/or rearranged without departing from the scope of the present disclosure.

FIG. 55C may further depict a network 5520 in communication with monitoring system 150. Network 5520 may include any suitable communication infrastructure, including but not limited to the Internet, a local area network (LAN), a wide area network (WAN), a wireless network (e.g., Wi-Fi, cellular, LoRa, Zigbee), a mesh network, or any combination thereof. Monitoring system 150 may communicate with network 5520 via communication link 5528, which may be a wired connection (e.g., Ethernet, powerline communication) or a wireless connection (e.g., Wi-Fi, Bluetooth, cellular, or other RF communication protocols). Communication link 5528 may correspond to network connection 480 and/or communication module 358 as described with respect to FIGS. 3 and 4.

A remote computer 5522 may be in communication with monitoring system 150 via network 5520. Remote computer 5522 may comprise one or more servers, cloud computing resources, edge computing devices, or any combination thereof. Remote computer 5522 may host one or more of the fluid level machine learning model and the hysteresis machine learning model described herein. In certain embodiments, monitoring system 150 may transmit fluid level data, rate of change data, sensor data, or other sump-related data to remote computer 5522 for processing, storage, analysis, or display. In certain other embodiments, remote computer 5522 may transmit control signals, configuration parameters, updated machine learning models, or other data to monitoring system 150. Remote computer 5522 may correspond to one or more of external devices 483, 485, 487, server 495, machine-learning system 3504, cloud layer 4306, analytics layer 4308, or any combination thereof, as described elsewhere herein.

A weather data source 5524 may be in communication with monitoring system 150 and/or remote computer 5522 via network 5520. Weather data source 5524 may comprise a weather service API, a government weather data feed (e.g., National Weather Service, NOAA), a commercial weather data provider, a local weather station, one or more environmental sensors, or any combination thereof. Weather data from weather data source 5524 may include current weather conditions, weather forecasts, precipitation data, flood warnings, storm alerts, barometric pressure, humidity, temperature, or any other meteorological information relevant to sump system operation. Monitoring system 150 and/or remote computer 5522 may use weather data to adjust pump activation levels, pump deactivation levels, hysteresis values, alert thresholds, or other operational parameters as described herein.

One or more user devices 5526 may be in communication with monitoring system 150 and/or remote computer 5522 via network 5520. User device 5526 may comprise a smartphone, tablet, laptop computer, desktop computer, dedicated display, control panel, or any other device capable of displaying information and/or receiving user input. User device 5526 may display fluid level data, alerts, diagnostics, historical data, or other information related to the sump system. User device 5526 may also allow a user to modify hysteresis values, activation thresholds, alert settings, or other operational parameters of monitoring system 150. Alerts generated by monitoring system 150 and/or remote computer 5522 may be communicated to user device 5526 via push notification, SMS, email, audible alarm, visual indicator, or any other suitable alert mechanism. Though FIG. 55C depicts a single user device 5526 and a single remote computer 5522, any number of user devices 5526 and/or remote computers 5522 may be connected via the network 5520 without departing from the scope of the present disclosure.

In certain embodiments, at least one user device 5526 may be a device owned by an insurance organization. As mentioned above, insurance organizations may offer discounts to customers who use the disclosed monitoring system 150. A user may transmit evidence (e.g., photo evidence) to an insurance organization to show that monitoring system 150 is installed. Additionally or alternatively, to confirm continued use and function of disclosed monitoring system 150, in certain embodiments, disclosed monitoring system 150 may communicate to at least one user device 5526 a “heartbeat signal” over regular increments of time via network 5520; for example, disclosed monitoring system 150 may communicate to the insurance organization that the monitoring system 150 is currently operating on a daily basis, weekly basis, monthly basis, or at any other suitable increment of time. In certain embodiments, the signal used to communicate with the at least one user device 5526 of an insurance organization may be transmitted by a remote computer 5522 rather than being transmitted by monitoring system 150.

Furthermore, in certain embodiments, one or more of the monitoring system 150 and the remote computer 5522 may communicate to a user device 5526 owned by an insurance organization if an overflow of the container is detected or projected. A flood may be detected or projected based on one or more of fluid level data, fluid level rate of change data, weather data, data from a fluid sensor attached to a top surface of the container, or any other suitable data. The system may send the overflow signal immediately upon detection or after a delay. Optionally, the insurance organization may cancel coverage (e.g., via a smart contract, traditional contract, or any other mechanism) if an overflow is detected and not addressed or if monitoring system 150 becomes disabled and is not addressed.

In certain embodiments, an insurance organization may wish to receive information related to monitoring system 150 without receiving all of the data from monitoring system 150 (e.g., to avoid incurring a responsibility to monitor the system while maintaining assurance that the system is operational). Thus, in certain embodiments, one or more of monitoring system 150 and remote computer 5522 may communicate only a portion of the data related to monitoring system 150 to a user device 5526 owned by an insurance organization via network 5520. In certain other embodiments, a third-party organization such as an insurance organization may monitor the monitoring system 150 and may accordingly receive the data necessary to do so.

Monitoring system 150 may be activated or triggered to transmit signals by various mechanisms. In certain embodiments, monitoring system 150 may operate in a continuous monitoring mode, periodically transmitting signals and analyzing responses at regular intervals (e.g., every second, every minute, every hour). In certain other embodiments, monitoring system 150 may operate in an event-triggered mode, transmitting signals in response to a trigger event such as a signal from float 5510, a command from remote computer 5522, a command from user device 5526, detection of fluid at inlet 5512, a change in environmental conditions, or expiration of a timer. In still other embodiments, monitoring system 150 may operate in a hybrid mode combining continuous and event-triggered operation. The monitoring interval or trigger conditions may be adjusted manually by a user, automatically by remote computer 5522, or automatically by monitoring system 150 based on factors such as current fluid level, rate of change of fluid level, weather data, time of day, historical patterns, or power availability. In certain embodiments, monitoring system 150 may increase its monitoring frequency in response to rising fluid levels, incoming weather alerts, or other conditions indicating elevated flood risk.

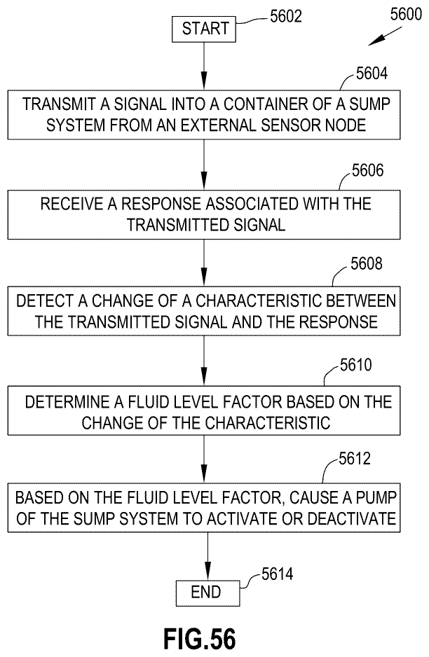

FIG. 56 depicts an example method for controlling a pump of a sump system in accordance with at least one aspect of the present disclosure. In one aspect, method 5600 can be implemented by the monitoring system 150 of FIG. 55A and/or FIG. 55B.

Method 5600 starts at block 5602 which represents an initialization point for the monitoring sequence 5600.

Method 5600 continues to block 5604 with transmitting a signal into a container of a sump system from an external sensor node. The signal may be transmitted by at least one external sensor node of monitoring system 150 of FIG. 55A and/or 55B. Block 5604 encompasses the active sensing operations wherein the monitoring system collects data regarding the container contents using one or more sensing modalities. The monitoring system employed at block 5604 may include radio-frequency (RF) based sensors, such as passive or semi-passive RF-responsive elements positioned on the exterior surface of the container. These RF-responsive elements may include RFID tags, resonators, or other RF-interactive devices configured to interact with interrogating radio signals in a manner that is influenced by the adjacent internal environment. The monitoring operation at block 5604 may involve transmitting radio-frequency signals toward the container and RF-responsive elements, and subsequent method steps may involve capturing and analyzing the resulting reflected or backscattered signals.

At block 5606, the process receives a response associated with the transmitted signal, and at block 5608, the process detects a change of a characteristic between the transmitted signal and the response. These blocks represent the acquisition and initial processing of measurement data obtained during the monitoring operation. The collected data points may include raw signal values such as reflection amplitudes, phase shifts, or frequency responses measured at multiple locations on the container. These measurements may be taken from a plurality of RF-responsive elements arranged in a spatial pattern, such as a vertical array, to capture information about the internal container state with appropriate spatial resolution. The data collection may also include contextual information such as timestamps, environmental readings (temperature, humidity), or system configuration parameters that may affect signal interpretation.

In some implementations, data collection at blocks 5606-5608 may involve filtering, normalization, or other signal-conditioning operations to prepare the raw sensor readings for subsequent analysis. These operations may include noise reduction, baseline correction, or feature extraction techniques appropriate for the specific sensing modality. The collected data points may be temporarily stored in local memory, buffered for batch processing, or immediately forwarded to the analysis system depending on the implementation architecture.

At block 5610, the process determines a fluid level factor based on the change of the characteristic. The fluid level factor may include any one or more of fluid level and a rate of change of the fluid level. In certain embodiments, analysis may be performed using one or more of the machine learning methods and/or hard-coded methods disclosed herein. In some aspects, the analysis may occur on-device, with program instructions executing locally on processors integrated with or near the sensing hardware. In other aspects, the analysis may be performed remotely, with collected data transmitted to cloud servers or centralized computing infrastructure for processing. The analysis may incorporate temporal information, comparing current readings against historical data to identify trends, anomalies, or changes in the container contents over time. Additionally, the analysis may consider multiple sensor inputs simultaneously, fusing data from different sensing modalities or spatial locations to improve prediction accuracy and consistency.

In some implementations, the determination at block 5610 may involve comparing derived measurements against reference values, thresholds, or expected ranges to evaluate whether the container contents are developing according to expectations. The system may also calculate derivative metrics, such as evaporation rates, based on changes in primary characteristics over time. In certain embodiments, the system may incorporate weather data into its evaluations.

In some aspects, the monitoring at blocks 5604-5610 may be performed continuously, providing real-time data about container contents. In other aspects, the monitoring may occur periodically according to a predefined schedule, such as hourly, daily, or weekly measurements, to conserve power or computational resources while maintaining sufficient temporal resolution for the application requirements. The monitoring operation may involve multiple sensor types or multiple measurement modalities to capture complementary data, potentially including RSSI measurements, phase information, CSI, resonant frequency shifts, or combinations of these characteristics. The selection of specific sensing parameters and frequencies may be tailored to the particular container material, expected content characteristics, and monitoring objectives.

At block 5612, the process causes a pump of the sump system to activate or deactivate based on the fluid level factor. The transmission used to activate or deactivate the pump may occur through various communication channels, including wired connections, wireless protocols (Wi-Fi, Bluetooth, cellular), or specialized industrial communication standards appropriate for the operational environment.

The process 5600 concludes at block 5614, which represents the completion of a single monitoring cycle. After reaching this end block, the system may return to the start block to begin another monitoring iteration, potentially with updated parameters or adjusted scheduling based on the results of the completed cycle. In continuous monitoring implementations, the end block might represent a brief reset or reconfiguration before immediately restarting the process, while in periodic monitoring scenarios, the system might enter a low-power state until the next scheduled measurement interval.

FIG. 56 is given by way of example rather than by way of limitation. Elements may be added, removed, altered, and/or rearranged without departing from the scope of the present disclosure.

In accordance with at least one aspect of the present disclosure, a method for controlling a pump of a sump system is described. In some aspects, the method may include transmitting, into a container of the sump system, at least one signal from one or more sensor nodes positioned external to the container. In some aspects, the method may further include receiving a response associated with the transmitted at least one signal. In some aspects, the method may further include detecting, via one or more processors, a change of at least one characteristic between the transmitted at least one signal and a corresponding received response. In some aspects, the method may further include, based on the change of the at least one characteristic, determining, via the one or more processors, a fluid level factor. In some aspects, the fluid level factor may include one or more of a fluid level of the container and a rate of change of the fluid level of the container. In some aspects, the method may further include, based on the fluid level factor, causing, via the one or more processors, the pump of the sump system to activate or deactivate.

In some aspects, the method may further include causing the pump of the sump system to activate or deactivate based at least in part on a float sensor.

In some aspects, the method may further include determining that the fluid level of the container is increasing at a rate of change exceeding a threshold value. In some aspects, the method may further include, based on determining that the fluid level of the container is increasing at the rate of change exceeding the threshold value, activating the pump of the sump system at a lower fluid level than would otherwise be sufficient to cause activation of the pump.

In some aspects, the method may further include communicating fluid level data to a computer located remote from the sump system.

In some aspects, the method may further include automatically adjusting one or more hysteresis values based on one or more of the fluid level factor and weather data. In some aspects, the one or more hysteresis values may automatically be adjusted based at least in part on a hysteresis machine learning model.

In some aspects, determining the fluid level factor based on the change of the at least one characteristic may be performed using a fluid level machine learning model.

In accordance with at least one aspect of the present disclosure, a system for controlling a pump of a sump system is described. In some aspects, the system may include one or more sensor nodes positioned external to a container of the sump system and one or more processors. In some aspects, the one or more sensor nodes may be configured to transmit at least one signal through a surface of the container and to receive a response associated with the transmitted at least one signal. In some aspects, the one or more processors may be configured to detect a change of at least one characteristic between the at least one transmitted signal and a corresponding received response. In some aspects, the one or more processors may further be configured to, based on the change of the at least one characteristic, determine a fluid level factor. In some aspects, the fluid level factor may include one or more of a fluid level of the container and a rate of change of the fluid level of the container. In some aspects, the one or more processors may further be configured to, based on the fluid level factor, cause the pump of the sump system to activate or deactivate.

In some aspects, the system may further include a float sensor, and the pump of the sump system may be configured to activate or deactivate based at least in part on the float sensor.

In some aspects, the one or more processors may be further configured to determine that the fluid level of the container is increasing at a rate of change exceeding a threshold value. In some aspects, the one or more processors may be further configured to, based on determining that the fluid level of the container is increasing at the rate of change exceeding the threshold value, activate the pump of the sump system at a lower fluid level than would otherwise be sufficient to cause activation of the pump.

In some aspects, the one or more processors may be further configured to communicate fluid level data to a computer located remote from the sump system.

In some aspects, the one or more processors may be further configured to automatically adjust one or more hysteresis values based on one or more of the fluid level factor and weather data. In some aspects, the one or more hysteresis values may be automatically adjusted based at least in part on a hysteresis machine learning model.

In some aspects, the one or more processors may be further configured to determine the fluid level factor based on the change of the at least one characteristic using a fluid level machine learning model.

In accordance with at least one aspect of the present disclosure, a non-transitory computer-readable medium is described. In some aspects, the non-transitory computer-readable medium may be provisioned with program instructions that, when executed by one or more processors, cause the one or more processors to receive sensor data from one or more sensor nodes positioned external to a container of a sump system. In some aspects, the sensor data may correspond to at least one signal transmitted from the one or more sensor nodes through a surface of the container and a subsequent response associated with the transmitted at least one signal. In some aspects, the program instructions may be executable by the one or more processors to detect a change of at least one characteristic based on the sensor data. In some aspects, the program instructions may be executable by the one or more processors to, based on the change of the at least one characteristic, determine a fluid level factor. The fluid level factor may include one or more of a fluid level of the container and a rate of change of the fluid level of the container. In some aspects, the program instructions may be executable by the one or more processors to, based on the fluid level factor, cause a pump of the sump system to activate or deactivate.

In some aspects, the program instructions may be executable by the one or more processors to determine that the fluid level of the container is increasing at a rate of change exceeding a threshold value. In some aspects, the program instructions may be executable by the one or more processors to, based on determining that the fluid level of the container is increasing at the rate of change exceeding the threshold value, activate the pump of the sump system at a lower fluid level than would otherwise be sufficient to cause activation of the pump.

In some aspects, the program instructions may be executable by the one or more processors to communicate fluid level data to a computer located remote from the sump system.

In some aspects, the program instructions may be executable by the one or more processors to automatically adjust one or more hysteresis values based on one or more of the fluid level factor and weather data.

In some aspects, the one or more hysteresis values may be automatically adjusted based at least in part on a hysteresis machine learning model.

In some aspects, determining the fluid level factor based on the change of the at least one characteristic may be performed using a fluid level machine learning model.

As used throughout this disclosure, the phrase “one or more of” followed by a list of items separated by “or” (e.g., “one or more of A, B, or C”) is intended to mean one or more items selected from the list, including any single item alone, any combination of items, or all items together, as well as any combination with multiples of the same element. For example, “one or more of A, B, or C” includes: A alone; B alone; C alone; A and B together; A and C together; B and C together; or A, B, and C together. This interpretation applies equally to the phrase “at least one of” followed by a list of items. As an example, “at least one of: a, b, or c” is intended to cover a, b, c, a-b, a-c, b-c, and a-b-c, as well as any combination with multiples of the same element (e.g., a-a, a-a-a, a-a-b, a-a-c, a-b-b, a-c-c, b-b, b-b-b, b-b-c, c-c, and c-c-c or any other ordering of a, b, and c). This clarification is provided to ensure consistent interpretation of claim scope and is not intended to limit the disclosure in any way.

The invention has been described with reference to specific embodiments. One of ordinary skill in the art, however, appreciates that various modifications and changes can be made without departing from the scope of the invention as set forth in the claims. Accordingly, the specification is to be regarded in an illustrative manner, rather than with a restrictive view, and all such modifications are intended to be included within the scope of the invention.

Benefits, other advantages, and solutions to problems have been described above regarding specific embodiments. The benefits, advantages, and solutions to problems, and any element(s) that may cause any benefits, advantages, or solutions to occur or become more pronounced, are not to be construed as a critical, required, or an essential feature or element of any or all of the claims.

The preceding description is provided to enable any person skilled in the art to practice the various embodiments described herein. The examples discussed herein are not limiting of the scope, applicability, or embodiments set forth in the claims. Various modifications to these embodiments will be readily apparent to those skilled in the art, and the generic principles defined herein may be applied to other embodiments. For example, changes may be made in the function and arrangement of elements discussed without departing from the scope of the disclosure. Various examples may omit, substitute, or add various procedures or components as appropriate. For instance, the methods described may be performed in an order different from that described, and various steps may be added, omitted, or combined. Also, features described with respect to some examples may be combined in some other examples. For example, an apparatus may be implemented or a method may be practiced using any number of the aspects set forth herein. In addition, the scope of the disclosure is intended to cover such an apparatus or method that is practiced using other structure, functionality, or structure and functionality in addition to, or other than, the various aspects of the disclosure set forth herein. It should be understood that any aspect of the disclosure disclosed herein may be embodied by one or more elements of a claim.

As used herein, the term “determining” encompasses a wide variety of actions. For example, “determining” may include calculating, computing, processing, deriving, investigating, looking up (e.g., looking up in a table, a database or another data structure), ascertaining and the like. Also, “determining” may include receiving (e.g., receiving information), accessing (e.g., accessing data in a memory) and the like. Also, “determining” may include resolving, selecting, choosing, establishing and the like.

The methods disclosed herein comprise one or more steps or actions for achieving the methods. The method steps and/or actions may be interchanged with one another without departing from the scope of the claims. In other words, unless a specific order of steps or actions is specified, the order and/or use of specific steps and/or actions may be modified without departing from the scope of the claims. Further, the various operations of methods described above may be performed by any suitable means capable of performing the corresponding functions. The means may include various hardware and/or software component(s) and/or module(s), including, but not limited to a circuit, an application specific integrated circuit (ASIC), or processor. Generally, where there are operations illustrated in figures, those operations may have corresponding counterpart means-plus-function components with similar numbering.

The following claims are not intended to be limited to the embodiments shown herein, but are to be accorded the full scope consistent with the language of the claims. Within a claim, reference to an element in the singular is not intended to mean “one and only one” unless specifically so stated, but rather “one or more.” Unless specifically stated otherwise, the term “some” refers to one or more. No claim element is to be construed under the provisions of 35 U.S.C. § 112(f) unless the element is expressly recited using the phrase “means for” or, in the case of a method claim, the element is recited using the phrase “step for.” All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims.

What is claimed is:

1. A method for controlling a pump of a sump system, the method comprising:
transmitting, through a surface of a container of the sump system, at least one signal from one or more sensor nodes positioned external to the container;
receiving a response associated with the transmitted at least one signal;
detecting, via one or more processors, a change of at least one characteristic between the transmitted at least one signal and a corresponding received response;
based on the change of the at least one characteristic, determining, via the one or more processors, a fluid level factor, wherein the fluid level factor comprises at least one of:
a fluid level of the container; or
a rate of change of the fluid level of the container; and
based on the fluid level factor, causing, via the one or more processors, the pump of the sump system to activate or deactivate.

2. The method of claim 1, further comprising causing the pump of the sump system to activate or deactivate based at least in part on a float sensor.

3. The method of claim 1, further comprising:
determining that the fluid level of the container is increasing at a rate of change exceeding a threshold value; and
based on determining that the fluid level of the container is increasing at the rate of change exceeding the threshold value, activating the pump of the sump system at a lower fluid level than would otherwise be sufficient to cause activation of the pump.

4. The method of claim 1, further comprising communicating fluid level data to a computer located remote from the sump system.

5. The method of claim 1, further comprising automatically adjusting one or more hysteresis values based on at least one of the fluid level factor or weather data.

6. The method of claim 5, wherein the one or more hysteresis values are automatically adjusted based at least in part on a hysteresis machine learning model.

7. The method of claim 1, wherein determining the fluid level factor based on the change of the at least one characteristic is performed using a fluid level machine learning model.

8. A system for controlling a pump of a sump system, the system comprising:
one or more sensor nodes positioned external to a container of the sump system, the one or more sensor nodes configured to:
transmit at least one signal through a surface of the container; and
receive a response associated with the transmitted at least one signal; and
one or more processors configured to:
detect a change of at least one characteristic between the at least one transmitted signal and a corresponding received response;
based on the change of the at least one characteristic, determine a fluid level factor, wherein the fluid level factor comprises at least one of:
a fluid level of the container; or
a rate of change of the fluid level of the container; and
based on the fluid level factor, cause the pump of the sump system to activate or deactivate.

9. The system of claim 8, further comprising a float sensor, wherein the pump of the sump system is configured to activate or deactivate based at least in part on the float sensor.

10. The system of claim 8, wherein the one or more processors are further configured to:
determine that the fluid level of the container is increasing at a rate of change exceeding a threshold value; and
based on determining that the fluid level of the container is increasing at the rate of change exceeding the threshold value, activate the pump of the sump system at a lower fluid level than would otherwise be sufficient to cause activation of the pump.

11. The system of claim 8, wherein the one or more processors are further configured to communicate fluid level data to a computer located remote from the sump system.

12. The system of claim 8, wherein the one or more processors are further configured to automatically adjust one or more hysteresis values based on at least one of the fluid level factor or weather data.

13. The system of claim 12, wherein the one or more hysteresis values are automatically adjusted based at least in part on a hysteresis machine learning model.

14. The system of claim 8, wherein determining the fluid level factor based on the change of the at least one characteristic is performed using a fluid level machine learning model.

15. A non-transitory computer-readable medium, wherein the non-transitory computer-readable medium is provisioned with program instructions that, when executed by one or more processors, cause the one or more processors to:
receive sensor data from one or more sensor nodes positioned external to a container of a sump system, wherein the sensor data corresponds to at least one signal transmitted from the one or more sensor nodes through a surface of the container and a subsequent response associated with the transmitted at least one signal;
detect a change of at least one characteristic based on the sensor data;
based on the change of the at least one characteristic, determine a fluid level factor, wherein the fluid level factor comprises at least one of:
a fluid level of the container; or
a rate of change of the fluid level of the container; and
based on the fluid level factor, cause a pump of the sump system to activate or deactivate.

16. The non-transitory computer-readable medium of claim 15, wherein the program instructions are executable by the one or more processors to further cause the one or more processors to:
determine that the fluid level of the container is increasing at a rate of change exceeding a threshold value; and
based on determining that the fluid level of the container is increasing at the rate of change exceeding the threshold value, activate the pump of the sump system at a lower fluid level than would otherwise be sufficient to cause activation of the pump.

17. The non-transitory computer-readable medium of claim 15, wherein the program instructions are executable by the one or more processors to further cause the one or more processors to communicate fluid level data to a computer located remote from the sump system.

18. The non-transitory computer-readable medium of claim 15, wherein the program instructions are executable by the one or more processors to further cause the one or more processors to automatically adjust one or more hysteresis values based on at least one of the fluid level factor or weather data.

19. The non-transitory computer-readable medium of claim 18, wherein the one or more hysteresis values are automatically adjusted based at least in part on a hysteresis machine learning model.

20. The non-transitory computer-readable medium of claim 15, wherein determining the fluid level factor based on the change of the at least one characteristic is performed using a fluid level machine learning model.
